(19) United States
(12) Patent Application Publication
(10) Pub. No.: US 2024/0270821 A1
(43) Pub. Date: Aug. 15, 2024

(54) GIP/GLP1/GCG TRI-RECEPTOR AGONISTS AND USES THEREOF

(71) Applicant: Eli Lilly and Company, Indianapolis, IN (US)

(72) Inventors: Milata Mary Abraham, Indianapolis, IN (US); Robert Andrew BROWN, Carmel, IN (US); Tamer COSKUN, Carmel, IN (US); David Benjamin FLORA, Greenfield, IN (US); Paul Joseph KLEINDL, Lebanon, IN (US); Fucheng QU, Carmel, IN (US); Hongchang QU, Carmel, IN (US); James Lincoln WALLIS, Indianapolis, IN (US)

(21) Appl. No.: 18/428,102

(22) Filed: Jan. 31, 2024

Related U.S. Application Data
(60) Provisional application No. 63/482,404, filed on Jan. 31, 2023.

(51) Int. Cl. C07K 14/72; A61P 3/04; A61K 38/00

(52) U.S. Cl. CPC C07K 14/72; A61P 3/04; A61K 38/00

(57) ABSTRACT

Polypeptides are provided that have activity at each of the GIP, GLP-1 and glucagon receptors. The polypeptides have structural features resulting in activity and extended duration of action at each of these receptors. Methods also are provided for treating diseases and/or conditions such as obesity, chronic weight management, type 2 diabetes mellitus, non-alcoholic fatty liver disease (NAFLD), non-alcoholic steatohepatitis (NASH), dyslipidemia, metabolic syndrome, Chronic Kidney Disease (CKD), osteoarthritis (OA), obesity-related sleep apnea (OSA) and polycystic ovary syndrome (PCOS).

### SEQUENCE LISTING

The present application is being filed along with a Sequence Listing in ST.26 XML format. The Sequence Listing is provided as a file titled “30475_US_SL” created 24 Jan. 2024 and is 4.84 megabytes in size. The Sequence Listing information in the ST.26 XML format is incorporated herein by reference in its entirety.

### FIELD OF INVENTION

This disclosure relates to polypeptides having activity at each of a glucose-dependent insulinotropic polypeptide (GIP), glucagon-like peptide-1 (GLP-1) and glucagon (GCG) receptors. The polypeptides described herein have structural features that provide appropriate activity levels and extended duration of action at each of these receptors. Furthermore, the present invention relates to compounds that may be administered orally or subcutaneously. Such polypeptides may be useful for treating disorders or conditions such as obesity, chronic weight management, type 2 diabetes mellitus (T2DM), dyslipidemia, metabolic syndrome, non-alcoholic fatty liver disease (NAFLD), non-alcoholic steatohepatitis (NASH), chronic kidney disease (CKD), osteoarthritis (OA), obesity-related sleep apnea (OSA) and/or polycystic ovary syndrome (PCOS).

### BACKGROUND

Over the past several decades, the prevalence of diabetes has continued to rise. T2DM is the most common form of diabetes accounting for about 90% of all diabetes. T2DM is characterized by high blood glucose levels caused by insulin resistance. The current standard of care for T2DM includes diet and exercise, as well as treatment with oral medications and injectable glucose-lowering drugs including incretin-based therapies, such as GLP-1 receptor agonists. A variety of GLP-1 analogs are currently available for treating T2DM, including dulaglutide, exenatide and liraglutide. Many currently marketed GLP-1 receptor agonists, however, are dose-limited by gastrointestinal side effects, such as nausea and vomiting. Subcutaneous injection is the typical route of administration for the available GLP-1 receptor agonists. When treatment with available oral medications and incretin-based therapies is insufficient, insulin is considered. Despite the treatment options available, significant numbers of individuals receiving approved therapies are not reaching glycemic control goals (see. e.g., Casagrande et al. (2013) Diabetes Care 36:2271-2279). Uncontrolled diabetes can lead to one or more conditions that impact morbidity and mortality of such individuals.

One of the main risk factors for T2DM is obesity, and a majority of individuals with T2DM (˜90%) are overweight or obese. Obesity is a complex medical disorder resulting in excessive accumulation of adipose tissue mass. Today obesity is a global public health concern that is associated with undesired health outcomes and morbidities. Desired treatments for patients with obesity strive to reduce excess body weight, improve obesity-related co-morbidities, and maintain long-term weight reduction. Available treatments for obesity are particularly unsatisfactory for patients with severe obesity. There is a need for alternative treatment options to induce therapeutic weight loss in patients in need of such treatment.

In view thereof, new therapies being studied include compounds having not only activity at a GLP-1 receptor but also activity at one or more other receptors, such as the GIP and/or glucagon receptors.

For example, Int'l Patent Application Publication No. WO2013/164483 and WO2016/111971 describe polypeptides stated to have GLP-1 and GIP receptor activity. WO2011/075393, WO2012/177444, and WO2016/209707 describe polypeptides stated to have GCG and GIP receptor activity.

Furthermore, certain compounds have been described as having triple agonist activity (i.e., activity at each of the GIP, GLP-1 and glucagon receptors). For example, WO2015/067716 describes glucagon analogs having triple agonist activity. Similarly, WO2016/198624 describes analogs of exendin-4, itself a GLP-1 analog, having triple agonist activity. Likewise, WO2014/049610 and WO2017/116204 each describe a variety of analogs having triple agonist activity. Moreover, Int'l Patent Application No. WO2017/153375 describes glucagon and GLP-1 co-agonists that also are stated to have GIP activity. Furthermore, WO2019/125938, WO2019/125929 and WO2021/126695 each describe a variety of polypeptides having triple agonist activity.

Nevertheless, a need remains for compounds that are capable of providing effective glucose control with weight loss benefits and a favorable side effect profile. There is also a need for alternate treatment options to provide therapeutic weight loss or chronic weight management in a patient in need of such treatment. There also is a need for therapeutic agents available for use with sufficiently extended duration of action to allow for dosing as infrequently as once a day, thrice-weekly, twice-weekly, or once a week. Furthermore, there is a desire and need for compounds that are amenable to convenient modes of administration, such as subcutaneous or oral route. In particular, there is a desire for compounds that exhibit sufficient efficacy with a favorable side effect profile, and/or stability and bioavailability so that they can be administered orally.

### SUMMARY

The polypeptides described herein seek to meet one or more of the needs above. Accordingly, this disclosure describes polypeptides with activity at each of the GIP, GLP-1 and glucagon receptors. The polypeptides described herein allow for administration of doses that provide sufficient activity at each receptor to provide the benefits of agonism of that receptor while avoiding unwanted side effects associated with too much activity. Moreover, the polypeptides described herein have extended duration of action at the GIP, GLP-1 and glucagon receptors allowing for dosing as infrequently as once-a-day, thrice-weekly, twice-weekly, or once-a-week. In this manner, the polypeptides result in enhanced glucose control, metabolic benefits such as body weight lowering and/or improved body composition, lipid benefits, and/or other benefits such as an increase in bone mass or bone formation or a decrease in bone resorption. Furthermore, the polypeptides described herein are suitable for subcutaneous or oral administration. This disclosure also describes effective treatments for disorders or conditions, including obesity, chronic weight management, type 2 diabetes mellitus, NAFLD, NASH, dyslipidemia, metabolic disorder, CKD, OA, OSA and PCOS.

In one embodiment, a polypeptide is provided that includes the formula I (SEQ ID NO:4):

X₁X₂QGTX₆TSDX₁₀X₁₁X₁₂X₁₃LDX₁₆X₁₇AX₁₉X₂₀X₂₁
FX₂₃X₂₄X₂₅LX₂₇X₂₈X₂₉X₃₀X₃₁SSX₃₄X₃₅X₃₆X₃₇
X₃₈X₃₉X₄₀X₄₁X₄₂,

wherein:
   X₁ is Y, NMeY or H,
   X₂ is Aib,
   X₆ is F, αMeF or αMeF(2F),
   X₁₀ is F, 4-Pal, F(4CN), 3-Pal, F(4NO₂) or Y,
   X₁₁ is S or αMeS,
   X₁₂ is Orn, K, R, Q, Dap, Dab, S, E or I,
   X₁₃ is αMeL, I or L,
   X₁₆ is K, Orn, A or E,
   X₁₇ is any amino acid with a functional group available for conjugation to a fatty acid, A, I or Q,
   X₁₉ is Q or A,
   X₂₀ is any amino acid with a functional group available for conjugation to a fatty acid, Aib, αMe-4-Pal, Q or R,
   X₂₁ is A, Q, Orn, Aad, Aib, S, N, E or T,
   X₂₃ is I or V,
   X₂₄ is any amino acid with a functional group available for conjugation to a fatty acid, E, Q, D-Glu or N,
   X₂₅ is W, Y, F, 4-Pal, αMeY or αMe-4-Pal,
   X₂₇ is L, I, E, V, A, Q or S,
   X₂₈ is any amino acid with a functional group available for conjugation to a fatty acid, E or A,
   X₂₉ is G, D-Ala, Aib, T or A,
   X₃₀ is A or G,
   X₃₁ is P, H, S, 4-Pal, E, T, K(Ac) or Hyp (hydroxyproline),
   X₃₄ is G or Aib,
   X₃₅ is A, Aib, E, H or 4-Pal,
   X₃₆ is P or Hyp,
   X₃₇ is P, Hyp or E,
   X₃₈ is P or Hyp,
   X₃₉ is E, S, G, T, H, 4-Pal, γE or A,
   X₄₀ is absent or G, E, S, A or T,
   wherein if X₄₀ is G, E, S, A or T, then X₄₁ is absent or E, S, T, 4-Pal, D, G, Q or H,
   wherein if X₄₁ is E, S, T, 4-Pal, D, G, Q or H, then X₄₂ is absent or G, E or γE,
   wherein if X₄₀ is absent, then X₄₁ and X₄₂ are also absent,
   wherein if X₄₁ is absent, then X₄₂ is also absent,
   wherein at least one of X₁₇, X₂₀, X₂₄ and X₂₈ is an amino acid with a functional group available for conjugation to a fatty acid,
   wherein the C-terminal amino acid is optionally amidated;
   or a pharmaceutically acceptable salt thereof.

In some embodiments, the amino acid at position X₁₇, X₂₀, X₂₄ or X₂₈ with a functional group available for conjugation to a fatty acid is K, C, E or D. In some embodiments, the amino acid at position X₁₇, X₂₀, X₂₄ or X₂₈ with a functional group available for conjugation to a fatty acid is K. In some embodiments, X₁₀ is F or 4-Pal. In some embodiments, X₁₂ is Om, K, R or Q.

In some embodiments, only one of X₁₇, X₂₀, X₂₄ and X₂₈ is an amino acid with a functional group available for conjugation to a fatty acid.

In some embodiments, only one of X₁₇, X₂₀, X₂₄, or X₂₈ is conjugated to a fatty acid, via a direct bond or via a linker between the amino acid and the fatty acid. In some embodiments, only one of X₁₇, X₂₀, X₂₄, or X₂₈ is conjugated to a fatty acid via a linker between the amino acid and the fatty acid. In some embodiments, the fatty acid is a C₁₆-C₂₂ fatty acid.

In one embodiment, a polypeptide is provided that includes the formula l′ (SEQ ID NO:1243):

X₁X₂QX₄TX₆TSDX₁₀X₁₁X₁₂X₁₃LX₁₅X₁₆X₁₇AX₁₉X₂₀
X₂₁FX₂₃X₂₄X₂₅LX₂₇X₂₈X₂₉X₃₀X₃₁X₃₂SX₃₄X₃₅X₃₆
X₃₇X₃₈X₃₉X₄₀X₄₁X₄₂X₄₃X₄₄X₄₅X₄₆,

wherein:
X₁ is Y, NMeY or H,
X₂ is Aib,
X₄ is G or D-Ala,
X₆ is F, αMeF or αMeF(2F),
X₁₀ is F, 4-Pal, F(4CN), 3-Pal, F(4NO₂) or Y,
X₁₁ is S or αMeS,
X₁₂ is Orn, K, R, Q, Dap, Dab, S, E or I,
X₁₃ is αMeL, I or L,
X₁₅ is D or E,
X₁₆ is K, Orn, A or E,
X₁₇ is any amino acid with a functional group available for conjugation to a fatty acid, A, I, Q or Orn,
X₁₉ is Q or A,
X₂₀ is any amino acid with a functional group available for conjugation to a fatty acid, Aib, αMe-4-Pal, Q, R or L-Iva,
X₂₁ is A, Q, Om, Aad, Aib, S, N, E or T,
X₂₃ is I or V,
X₂₄ is any amino acid with a functional group available for conjugation to a fatty acid, E, Q, D-Glu, N or D-Gln,
X₂₅ is W, Y, F, 4-Pal, αMeY or αMe-4-Pal,
X₂₇ is L, I, E, V, A, Q, S, T or Aad,
X₂₈ is any amino acid with a functional group available for conjugation to a fatty acid, E, Q or A,
X₂₉ is G, D-Ala, Aib, T or A,
X₃₀ is A, S or G,
X₃₁ is P, H, S, 4-Pal, E, T, K(Ac) or Hyp (hydroxyproline),
X₃₂ is S or P
X₃₄ is G or Aib,
X₃₅ is A, Aib, E, D, H or 4-Pal or Orn,
X₃₆ is P or Hyp,
X₃₇ is P, Hyp or E,
X₃₈ is P or Hyp,
X₃₉ is E, S, G, T, H, 4-Pal, γE or A or Orn,
X₄₀ is absent or G, E, S, A, T, or D-Glu,
wherein if X₄₀ is G, E, S, A, T or D-Glu, then X₄₁ is absent or A, E, S, T, 4-Pal, D, G, γE, D-Glu(e), Q or H,
wherein if X₄₁ is A, E, S, T, 4-Pal, D, G, γE, D-Glu, Q or H, then X₄₂ is absent or G, E, γE, D-Glu, or AEEA,
wherein if X₄₂ is G, E, γE, D-Glu(e), or AEEA, then X₄₃ is absent or E, γE, or D-Glu,
wherein if X₄₃ is E, γE, or D-Glu, then X₄₄ is absent or E, wherein if X₄₄ is E then X₄₅ is absent or E, wherein if X₄₅ is E then X₄₆ is absent or E,
wherein if X₄₀ is absent, then X₄₁ through X₄₆ are also absent,
wherein if X₄₁ is absent, then X₄₂, through X₄₆ are also absent,
wherein if X₄₂ is absent, then X₄₃ through X₄₆ are also absent,
wherein if X₄₃ is absent, then X₄₄ through X₄₆ are also absent, wherein if X₄₄ is absent, then X₄₅ and X₄₆ are also absent; wherein if X₄₅ is absent then X₄₆ is also absent,
wherein at least one of X₁₇, X₂₀, X₂₄ and X₂₈ is an amino acid with a functional group available for conjugation to a fatty acid,
   wherein the C-terminal amino acid is optionally amidated; or a pharmaceutically acceptable salt thereof.

In some embodiments, the amino acid at position X₁₇, X₂₀, X₂₄ or X₂₈ with a functional group available for conjugation to a fatty acid is K, C, E, D or Orn. In some embodiments, the amino acid at position X₁₇, X₂₀, X₂₄ or X₂₈ with a functional group available for conjugation to a fatty acid is K. In some embodiments, the amino acid at position X₁₇, X₂₀, X₂₄ or X₂₈ with a functional group available for conjugation to a fatty acid is Orn. In some embodiments, X₁₀ is F or 4-Pal. In some embodiments, X₁₂ is Orn, K, R or Q.

In some embodiments, only one of X₁₇, X₂₀, X₂₄ and X₂₈ is an amino acid with a functional group available for conjugation to a fatty acid.

In some embodiments, one of X₁₇, X₂₀, X₂₄, or X₂₈ is conjugated to a fatty acid, via a direct bond or via a linker between the amino acid and the fatty acid. In some embodiments, one of X₁₇, X₂₀, X₂₄, or X₂₈ is conjugated to a fatty acid via a linker between the amino acid and the fatty acid. In some embodiments, the fatty acid is a C₁₆-C₂₂ fatty acid.

In some embodiments, none of X₁₇, X₂₀, X₂₄, or X₂₈ is conjugated to a fatty acid.

In one embodiment, a polypeptide is provided that includes the formula II (SEQ ID NO:5):

YX₂QGTFTSDX₁₀SX₁₂X₁₃LDX₁₆X₁₇AQX₂₀X₂₁FIX₂₄X₂₅LX₂₇
X₂₈X₂₉X₃₀X₃₁SSX₃₄X₃₅X₃₆X₃₇X₃₈X₃₉X₄₀X₄₁X₄₂,

wherein
   X₂ is Aib,
   X₁₀ is F, 4-Pal or F(4CN),
   X₁₂ is Orn, K, R, Q, Dap or Dab,
   X₁₃ is αMeL,
   X₁₆ is K or Orn,
   X₁₇ is any amino acid with a functional group available for conjugation to a fatty acid,
   X₂₀ is Aib, αMe-4-Pal or Q,
   X₂₁ is A, Q or Orn,
   X₂₄ is E or Q,
   X₂₅ is W, Y, 4-Pal, αMeY or αMe-4-Pal,
   X₂₇ is L or I,
   X₂₈ is E or A,
   X₂₉ is G, D-Ala or Aib,
   X₃₀ is A or G,
   X₃₁ is P, H, S, 4-Pal, E, T, K(Ac) or Hyp,
   X₃₄ is G or Aib,
   X₃₅ is A, Aib, E, H or 4-Pal,
   X₃₆ is P or Hyp,
   X₃₇ is P or Hyp,
   X₃₈ is P or Hyp,
   X₃₉ is E, S, G, T, H, 4-Pal or γE,
   X₄₀ is absent or G, E or S,
   wherein if X₄₀ is G, E or S, then X₄₁ is absent or E, S, T, 4-Pal, D, G, Q or H,
   wherein if X₄₁ is E, S, T, 4-Pal, D, G, Q or H, then X₄₂ is absent or G, E or γE,
   wherein if X₄₀ is absent, then X₄₁ and X₄₂ are also absent,
   wherein if X₄₁ is absent, then X₄₂ is also absent,
   wherein the C-terminal amino acid is optionally amidated; or a pharmaceutically acceptable salt thereof.

In some embodiments of polypeptides of formula II, X₁₇ is K, C, E or D. In some embodiments, X₁₇ is K. In some embodiments, X₁₀ is F or 4-Pal. In some embodiments, X₁₂ is Orn, K, R or Q.

In some embodiments, X₁₇ is K and is conjugated to a fatty acid via a direct bond or via a linker between the amino acid and the fatty acid. In some embodiments, X₁₇ is K and is conjugated to a fatty acid via a linker between the amino acid and the fatty acid. In some embodiments, the fatty acid is a C₁₆-C₂₂ fatty acid.

In some embodiments, X₁₇ is K and is conjugated to a C₁₆-C₂₂ fatty acid via a linker between the X₁₇ amino acid and the C₁₆-C₂₂ fatty acid.

In one embodiment, a polypeptide is provided that includes the formula II′ (SEQ ID NO:1244):

X₁X₂QGTFTSDX₁₀SX₁₂X₁₃LDX₁₆X₁₇AQX₂₀X₂₁FIX₂₄X₂₅LX₂₇
X₂₈X₂₉X₃₀X₃₁SSX₃₄X₃₅X₃₆X₃₇X₃₈X₃₉X₄₀X₄₁X₄₂X₄₃X₄₄,

wherein
X₁ is Y or NMeY
X₂ is Aib,
X₁₀ is F, 4-Pal or F(4CN),
X₁₂ is Orn, K, R, I, Q, Dap or Dab,
X₁₃ is αMeL,
X₁₆ is K or Orn,
X₁₇ is any amino acid with a functional group available for conjugation to a fatty acid,
X₂₀ is Aib, αMe-4-Pal, Q or L-Iva,
X₂₁ is A, Q or Orn,
X₂₄ is E or Q,
X₂₅ is W, Y, 4-Pal, αMeY or αMe-4-Pal,
X₂₇ is L or I,
X₂₈ is E, Q or A,
X₂₉ is G, D-Ala or Aib,
X₃₀ is A, S or G,
X₃₁ is P, H, S, 4-Pal, E, T, K(Ac) or Hyp,
X₃₄ is G or Aib,
X₃₅ is A, Aib, E, H or 4-Pal,
X₃₆ is P or Hyp,
X₃₇ is P or Hyp,
X₃₈ is P or Hyp,
X₃₉ is E, S, G, T, H, 4-Pal or γE,
X₄₀ is absent or G, E, S or D-Glu,
wherein if X₄₀ is G, E, S or D-Glu, then X₄₁ is absent or E, S, T, 4-Pal, D, G, Q or H,
wherein if X₄₁ is E, S, T, 4-Pal, D, G, Q or H, then X₄₂ is absent or G, E or γE,
wherein if X₄₂ is G, E, or γE, then X₄₃ is absent or E,
wherein if X₄₃ is E, then X₄₄ is absent or E,
wherein if X₄₀ is absent, then X₄₁, X₄₂, X₄₃, and X₄₄ are also absent,
wherein if X₄₁ is absent, then X₄₂, X₄₃ and X₄₄ are is also absent,
wherein if X₄₃ is absent then X₄₄ is also absent,
   wherein the C-terminal amino acid is optionally amidated; or a pharmaceutically acceptable salt thereof.

In some embodiments of polypeptides of formula II′, X₁₇ is K, C, E or D. In some embodiments, X₁₇ is K. In some embodiments, X₁₀ is F or 4-Pal. In some embodiments, X₁₂ is Orn, K, R or Q.

In some embodiments, X₁₇ is K and is conjugated to a fatty acid via a direct bond or via a linker between the amino acid and the fatty acid. In some embodiments, X₁₇ is K and is conjugated to a fatty acid via a linker between the amino acid and the fatty acid. In some embodiments, the fatty acid is a C₁₆-C₂₂ fatty acid.

In some embodiments, X₁₇ is K and is conjugated to a C₁₆-C₂₂ fatty acid via a linker between the X₁₇ amino acid and the C₁₆-C₂₂ fatty acid.

In some embodiments, none of X₁₇, X₂₀, X₂₄, or X₂₈ is conjugated to a fatty acid.

In one embodiment, a polypeptide is provided that includes the formula III (SEQ ID NO:6):

X₁X₂QGTX₆TSDX₁₀X₁₁X₁₂X₁₃LDX₁₆X₁₇AX₁₉X₂₀X₂₁FX₂₃X₂₄
X₂₅LX₂₇X₂₈X₂₉GX₃₁SSX₃₄X₃₅X₃₆X₃₇X₃₈X₃₉X₄₀X₄₁,

wherein
X₁ is Y, NMeY or H,
X₂ is Aib,
X₆ is F, αMeF or αMeF(2F),
X₁₀ is F, 3-Pal, 4-Pal, F(4CN), F(4NO₂) or Y,
X₁₁ is S or αMeS,
X₁₂ is Orn, K, R, Q, Dap, S, E or I,
X₁₃ is αMeL, I or L,
X₁₆ is K, Orn, A or E,
X₁₇ is any amino acid with a functional group available for conjugation to a fatty acid, A, I or Q,
X₁₉ is A or Q,
X₂₀ is any amino acid with a functional group available for conjugation to a fatty acid Aib, Q, R or αMe-4-Pal,
X₂₁ is A, Aad, Aib, S, N, Q, E, T or Orn,
X₂₃ is I or V,
X₂₄ is any amino acid with a functional group available for conjugation to a fatty acid E, D-Glu, Q or N,
X₂₅ is W, Y, F, 4-Pal, αMeY or αMe-4-Pal,
X₂₇ is L, I, E, V, A, Q or S,
X₂₈ is any amino acid with a functional group available for conjugation to a fatty acid, E or A,
X₂₉ is G, Aib, T, D-Ala or A,
X₃₁ is P or E,
X₃₄ is G or Aib,
X₃₅ is A or E,
X₃₆ is P,
X₃₇ is P or E,
X₃₈ is P,
X₃₉ is E, S, G or A,
X₄₀ is absent or G, E, S, A or T,
wherein if X₄₀ is G, E, S, A or T, then X₄₁ is absent or is E, S, D or G,
wherein if X₄₁ is E, S, D or G, then X₄₂ is absent or G, E or γE,
wherein if X₄₀ is absent, then X₄₁ and X₄₂ are also absent,
wherein if X₄₁ is absent, then X₄₂ is also absent,
wherein if X₁₀ is F, 3-Pal, 4-Pal, F(4CN), F(4NO₂), then X₁₂ is I,
wherein at least one of X₁₇, X₂₀, X₂₄ or X₂₈ is an amino acid with a functional group available for conjugation to a fatty acid,
wherein the C-terminal amino acid is optionally amidated;
or a pharmaceutically acceptable salt thereof.

In some embodiments of Formula III, the amino acid at position X₁₇, X₂₀, X₂₄ Or X₂₈ with a functional group available for conjugation to a fatty acid is K, C, E or D. In some embodiments, the amino acid at position X₁₇, X₂₀, X₂₄ or X₂₈ with a functional group available for conjugation to a fatty acid is K. In some embodiments, X₁₀ is F, 3-Pal, 4-Pal, F(4CN) or F(4NO₂) and X₁₂ is I. In some embodiments, X₁₀ is Y and X₁₂ is Om, K, R, Q, Dap, S, E or I.

In some embodiments, only one of X₁₇, X₂₀, X₂₄ and X₂₈ is an amino acid with a functional group available for conjugation to a fatty acid.

In some embodiments, only one of X₁₇, X₂₀, X₂₄, or X₂₈ is conjugated to a fatty acid, via a direct bond or via a linker between the amino acid and the fatty acid. In some embodiments, only one of X₁₇, X₂₀, X₂₄, or X₂₈ is conjugated to a fatty acid via a linker between the amino acid and the fatty acid. In some embodiments, the fatty acid is a C₁₆-C₂₂ fatty acid.

In some embodiments, only one of X₁₇, X₂₀, X₂₄, or X₂₈ is K and is conjugated to a C₁₆-C₂₂ fatty acid via a linker between the amino acid and the C₁₆-C₂₂ fatty acid.

In one embodiment, a polypeptide is provided that includes the formula III′ (SEQ ID NO:1245):

X₁X₂QX₄TX₆TSDX₁₀X₁₁X₁₂X₁₃LX₁₅X₁₆X₁₇AX₁₉X₂₀X₂₁FX₂₃
X₂₄X₂₅LX₂₇X₂₈X₂₉GX₃₁X₃₂SX₃₄X₃₅PX₃₇PX₃₉X₄₀X₄₁X₄₂
X₄₃X₄₄X₄₅X₄₆,

wherein
X₁ is Y, NMeY or H,
X₂ is Aib,
X₄ is G or D-Ala,
X₆ is F, αMeF or αMeF(2F),
X₁₀ is F, 3-Pal, 4-Pal, F(4CN), F(4NO₂) or Y,
X₁₁ is S or αMeS,
X₁₂ is Or, K, R, Q, Dap, S, E or I,
X₁₃ is αMeL, I or L,
X₁₅ is D or E,
X₁₆ is K, Orn, A or E,
X₁₇ is any amino acid with a functional group available for conjugation to a fatty acid, A, I or Q or Orn,
X₁₉ is A or Q,
X₂₀ is any amino acid with a functional group available for conjugation to a fatty acid, Aib, Q, R or αMe-4-Pal,
X₂₁ is A, Aad, Aib, S, N, Q, E, T or Orn,
X₂₃ is I or V, X₂₄ is any amino acid with a functional group available for conjugation to a fatty acid E, D-Glu, Q, N or D-Gln,
X₂₅ is W, Y, F, 4-Pal, αMeY or αMe-4-Pal,
X₂₇ is L, I, E, V, A, Aad, T, Q or S,
X₂₈ is any amino acid with a functional group available for conjugation to a fatty acid, E or A,
X₂₉, is G, Aib, T, D-Ala or A,
X₃₁ is P or E,
X₃₂ is S or P,
X₃₄ is G or Aib,
X₃₅ is A, D or E,
X₃₇ is P or E,
X₃₉ is E, S, G, A, T or Orn,
X₄₀ is absent or G, E, S, A or T,
wherein if X₄₀ is G, E, S, A, T or D-Glu, then X₄₁ is absent or is E, S, D, G, Q, T, A, γE or D-Glu,
wherein if X₄₁ is E, S, D, G, Q, T, A, γE or D-Glu, then X₄₂ is absent or G, E, D-Glu or γE,
wherein if X₄₂ is G, E, D-Glu or γE, then X₄₃ is absent or E, γE, or D-Glu,
wherein if X₄₃ is E, γE, or D-Glu, then X₄₄ is absent or E,
wherein if X₄₄ is E then X₄₅ is absent or E,
wherein if X₄₅ is E then X₄₆ is absent or E,
wherein if X₄₀ is absent, then X₄₁ through X₄₆ are also absent,
wherein if X₄₁ is absent, then X₄₂ through X₄₆ are also absent,
wherein if X₄₂ is absent, then X₄₃ through X₄₆ are also absent,
wherein if X₄₃ is absent, then X₄₄ through X₄₆ are also absent,
wherein if X₄₄ is absent, then X₄₅ and X₄₆ are also absent,
wherein if X₄₅ is absent then X₄₆ is also absent,
wherein the polypeptide comprises at least one of the following: X₆ is αMeF or αMeF(2F); X₁₀ is F, 3-Pal, 4-Pal, F(4CN) or F(4NO₂); X₁₁ is αMeS; X₁₃ is αMeL; X₂₄ is D-Glu; and/or X₂₅ is αMeY,
wherein if X₁₀ is F, 3-Pal, 4-Pal, F(4CN), or F(4NO₂), then X₁₂ is I,
and wherein at least one of X₁₇, X₂₀, X₂₄ Or X₂₈ is an amino acid with a functional group available for conjugation to a fatty acid,
wherein the C-terminal amino acid is optionally amidated; or a pharmaceutically acceptable salt thereof.

In some embodiments of Formula III′, the amino acid at position X₁₇, X₂₀, X₂₄ or X₂₈ with a functional group available for conjugation to a fatty acid is K, C, E, D or Om. In some embodiments, the amino acid at position X₁₇, X₂₀, X₂₄ or X₂₈ with a functional group available for conjugation to a fatty acid is K. In some embodiments, the amino acid at position X₁₇, X₂₀, X₂₄ or X₂₈ with a functional group available for conjugation to a fatty acid is Orn. In some embodiments, X₁₀ is F, 3-Pal, 4-Pal, F(4CN) or F(4NO₂) and X₁₂ is I. In some embodiments, X₁₀ is Y and X₁₂ is Orn, K, R, Q, Dap, S, E or I.

In some embodiments, only one of X₁₇, X₂₀, X₂₄ and X₂₈ is an amino acid with a functional group available for conjugation to a fatty acid.

In some embodiments, one of X₁₇, X₂₀, X₂₄, or X₂₈ is conjugated to a fatty acid, via a direct bond or via a linker between the amino acid and the fatty acid. In some embodiments, one of X₁₇, X₂₀, X₂₄, or X₂₈ is conjugated to a fatty acid via a linker between the amino acid and the fatty acid. In some embodiments, the fatty acid is a C₁₆-C₂₂ fatty acid.

In some embodiments, only one of X₁₇, X₂₀, X₂₄, or X₂₈ is K and is conjugated to a C₁₆-C₂₂ fatty acid via a linker between the amino acid and the C₁₆-C₂₂ fatty acid. In some embodiments, none of X₁₇, X₂₀, X₂₄, or X₂₈ is conjugated to a fatty acid.

In another embodiment, provided herein is a pharmaceutical composition that comprises a polypeptide described herein or a pharmaceutically acceptable salt thereof and at least one pharmaceutically acceptable carrier, diluent, or excipient. In some embodiments, the pharmaceutical composition is formulated for subcutaneous administration. In some embodiments, the pharmaceutical composition is formulated for oral administration.

In another embodiment, a method is provided for treating a disease or disorder including obesity, chronic weight management, type 2 diabetes mellitus, NAFLD, NASH, dyslipidemia, metabolic disorder, CKD, OA, OSA and PCOS. Another embodiment provides a method for providing non-therapeutic weight loss comprising administering to a subject in need thereof, an effective amount of a polypeptide described herein or a pharmaceutically acceptable salt thereof. Such methods can include at least a step of administering to an individual in need thereof an effective amount of a polypeptide a pharmaceutically acceptable salt thereof described herein.

In another embodiment, a polypeptide as described herein is provided for use in therapy. For example, a polypeptide as described herein is provided for use in treating a disease or disorder including obesity, chronic weight management, type 2 diabetes mellitus, NAFLD, NASH, dyslipidemia, metabolic disorder, CKD, OA, OSA and/or PCOS.

In another embodiment, there is provided the use of a polypeptide as described herein in the manufacture of a medicament for treating a disease or disorder including obesity, chronic weight management, type 2 diabetes mellitus, NAFLD, NASH, dyslipidemia, metabolic disorder, CKD, OA, OSA and/or PCOS.

### DETAILED DESCRIPTION

Unless defined otherwise, all technical and scientific terms used herein have the same meaning as commonly understood by one of skill in the art to which the disclosure pertains. Although any methods and materials similar to or equivalent to those described herein can be used in the practice or testing of the polypeptides, pharmaceutical compositions, and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

GIP is a 42-amino acid peptide (SEQ ID NO:1) and is an incretin, which plays a physiological role in glucose homeostasis by stimulating insulin secretion from pancreatic beta cells in the presence of glucose.

GLP-1 is a 36-amino acid peptide and also is an incretin, which stimulates glucose-dependent insulin secretion and which has been shown to prevent hyperglycemia in diabetics. The major biologically active fragment of GLP-1 is produced as a 30-amino acid, C-terminal amidated peptide (GLP-17-36) (SEQ ID NO:2).

Glucagon is a 29-amino acid peptide (SEQ ID NO:3) that helps maintain blood glucose by binding to and activating glucagon receptors on hepatocytes, causing the liver to release glucose—stored in the form of glycogen—through a process called glycogenolysis.

In addition to T2DM, incretins and analogs thereof having activity at one or more of the GIP, GLP-1 and/or glucagon receptors have been described as having a potential for therapeutic value in a number of other conditions, diseases or disorders, including, for example, obesity, NAFLD and NASH, dyslipidemia, metabolic syndrome, bone-related disorders, and neurodegenerative and/or cognitive disorders such as Alzheimer's disease and Parkinson's disease. See. e.g., Jall et al. (2017) Mol. Metab. 6:440-446; Carbone et al. (2016) J. Gastroenterol. Hepatol. 31:23-31: Finan et al. (2016) Trends Mol. Med. 22:359-376; Choi et al. (2017) Potent body weight loss and efficacy in a NASH animal model by a novel long-acting GLP-1 Glucagon GIP triple-agonist (HM15211), ADA Poster 1139-P; Ding (2008) J. Bone Miner. Res. 23:536-543: Tai et al. (2018) Brain Res. 1678:64-74; Müller et al. (2017) Physiol. Rev. 97:721-766; Finan et al. (2013) Sci. Transl. Med. 5:209; Hölscher (2014) Biochem. Soc. Trans. 42:593-600.

As used herein, “about” means within a statistically meaningful range of a value or values such as, for example, a stated concentration, length, molecular weight, pH, sequence identity, time frame, temperature or volume. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, and in reference to one or more of the GIP, GLP-1 or glucagon receptors, “activity,” “activate,” “activating” and the like means a capacity of a compound, such as the polypeptides described herein, to bind to and induce a response at the receptor(s), as measured using assays known in the art, such as the in vitro assays described below:

As used herein, “amino acid with a functional group available for conjugation” means any natural (coded) or non-natural (non-coded) amino acid with a functional group that may be conjugated to fatty acid directly or by way of, for example, a linker. Examples of such functional groups include, but are not limited to, alkynyl, alkenyl, amino, azido, bromo, carboxyl, chloro, iodo, and thiol groups. Examples of natural amino acids including such functional groups include K (amino), C (thiol), E (carboxyl) and D (carboxyl).

As used herein, “conservative amino acid substitution” means substitution of an amino acid with an amino acid having similar characteristics (e.g., charge, side-chain size, hydrophobicity/hydrophilicity, backbone conformation and rigidity, etc.) and having minimal impact on the biological activity of the resulting substituted peptide or polypeptide. Conservative substitutions of functionally similar amino acids are well known in the art and thus need not be exhaustively described herein.

As used herein, “C₁₆-C₂₂ fatty acid” means a carboxylic acid having between 16 and 22 carbon atoms. The C₁₆-C₂₂ fatty acid suitable for use herein can be a saturated monoacid or a saturated diacid. As used herein, “saturated” means the fatty acid contains no carbon-carbon double or triple bonds.

As used herein, “effective amount” means an amount, concentration or dose of one or more polypeptides described herein, or a pharmaceutically acceptable salt thereof which, upon single or multiple dose administration to an individual in need thereof, provides a desired effect in such an individual under diagnosis or treatment. An effective amount can be readily determined by one of skill in the art through the use of known techniques and by observing results obtained under analogous circumstances. In determining the effective amount for an individual, a number of factors are considered including, but not limited to, the species of mammal: its size, age and general health: the specific disease or disorder involved: the degree of or involvement of or the severity of the disease or disorder: the response of the individual patient: the particular polypeptide administered: the mode of administration; the bioavailability characteristics of the preparation administered: the dose regimen selected: the use of concomitant medication; and other relevant circumstances.

As used herein, “extended duration of action” means that binding affinity and activity for a polypeptide continues for a period of time greater than native human GIP, GLP-1 and glucagon peptides, allowing for dosing at least as infrequently as once daily or even thrice-weekly, twice-weekly or once-weekly. The time action profile of the polypeptide may be measured using known pharmacokinetic test methods such as those utilized in the examples below.

As used herein, “polypeptide” or “peptide” means a polymer of amino acid residues. The term applies to polymers comprising naturally occurring amino acids and polymers comprising one or more non-naturally occurring amino acids.

As used herein, “individual in need thereof” means a mammal, such as a human, with a condition, disease, disorder or symptom requiring treatment or therapy, including for example, those listed herein.

As used herein, “treat,” “treating,” “to treat” and the like mean restraining, slowing, stopping or reversing the progression or severity of an existing condition, disease, disorder or symptom.

As used herein, and with reference to a polypeptide, “triple agonist activity” means a polypeptide with activity at each of the GIP, GLP-1 and glucagon receptors, especially a polypeptide having sufficient activity at each receptor to provide the benefits of agonism of that receptor while avoiding unwanted side effects associated with too much activity.

The polypeptides having triple agonist activity (Also referred herein as “GGG polypeptides”) have extended duration of action at the GIP, GLP-1 and glucagon receptors, which advantageously allows for dosing as infrequently as once-a-day, thrice-weekly, twice-weekly or once-a-week.

As used herein, the term “Sequence identity” refers to the degree of similarity between two sequences. The degree of sequence identity between two polypeptides may be expressed as a percent, calculated as follows:

% Sequence identity=100%*(number of identical amino acids)/(length of the shortest common sequence).

The structural features of the polypeptides described herein result in them having appropriate activity at each of the GIP, GLP-1 and glucagon receptors to obtain the favorable effects of activity at each receptor (i.e., triple agonist activity), but not so much activity at any one receptor to either overwhelm the activity at the other two receptors or result in undesirable side effects when administered at a dose sufficient to result in activity at all three receptors. In some embodiments, the polypeptides described herein are partial agonists at the GLP-1 receptor showing agonism of 80% or less compared to the native GLP-1₇₋₃₆ (SEQ ID NO:2) as demonstrated by the HEK293 cell GLP-1 receptor internalization assay described herein. In other embodiments, the polypeptides described herein are full agonists at the GLP-1 receptor showing agonism of ≥80% compared to the native GLP-1₇₋₃₆ (SEQ ID NO:2) as demonstrated by the HEK293 cell GLP-1 receptor internalization assay described herein. In some embodiments, the polypeptides described herein have greater potency at each of the glucagon, GIP and GLP-1 receptors as compared to native glucagon (SEQ ID NO:3), GIP (SEQ ID NO:1) and GLP-1₇₋₃₆ (SEQ ID NO:2).

The structural features of the polypeptides described herein also result in polypeptides having many other beneficial attributes relevant to their developability as therapeutic treatments, including for improving solubility of the analogs in near neutral pH aqueous solutions, improving chemical and physical formulation stability, improving peptide membrane permeability in the presence of a permeation enhancer, extending the pharmacokinetic profile, and minimizing potential for injection site reaction or immunogenicity.

It should be noted that the combination of beneficial characteristics of exemplary analogs described herein is not the result of any single modification in isolation but is instead achieved through the novel combinations of the structural features described herein.

### Polypeptide Sequences

In one embodiment, provided herein is a polypeptide that comprises formula I (SEQ ID NO:4):

X₁X₂QGTX₆TSDX₁₀X₁₁X₁₂X₁₃LDX₁₆X₁₇AX₁₉X₂₀X₂₁FX₂₃X₂₄
X₂₅LX₂₇X₂₈X₂₉X₃₀X₃₁SSX₃₄X₃₅X₃₆X₃₇X₃₈X₃₉X₄₀X₄₁X₄₂,

wherein
X₁ is Y, NMeY or H,
X₂ is Aib,
X₆ is F, αMeF or αMeF(2F),
X₁₀ is F, 4-Pal, F(4CN), 3-Pal, F(4NO₂) or Y,
X₁₁ is S or αMeS,
X₁₂ is Orn, K, R, Q, Dap, Dab, S, E or I,
X₁₃ is αMeL, I or L,
X₁₆ is K, Orn, A or E,
X₁₇ is any amino acid with a functional group available for conjugation to a fatty acid, A, I or Q,
X₁₉ is Q or A,
X₂₀ is any amino acid with a functional group available for conjugation to a fatty acid, Aib, αMe-4-pal, Q or R,
X₂₁ is A, Q, Orn, Aad, Aib, S, N, E or T,
X₂₃ is I or V,
X₂₄ is any amino acid with a functional group available for conjugation to a fatty acid, E, Q, D-Glu or N,
X₂₅ is W, Y, F, 4-Pal, αMeY or αMe-4-pal,
X₂₇ is L, I, E, V, A, Q or S,
X₂₈ is any amino acid with a functional group available for conjugation to a fatty acid, E or A,
X₂₉ is G, D-Ala, Aib, T or A,
X₃₀ is A or G,
X₃₁ is P, H, S, 4-Pal, E, T, K(Ac) or Hyp,
X₃₄ is G or Aib,
X₃₅ is A, Aib, E, H or 4-Pal,
X₃₆ is P or Hyp,
X₃₇ is P, Hyp or E,
X₃₈ is P or Hyp,
X₃₉ is E, S, G, T, H, 4-Pal, γE or A,
X₄₀ is absent or G, E, S, A or T,
wherein if X₄₀ is G, E, S, A or T, then X₄₁ is absent or E, S, T, 4-Pal, D, G, Q or H,
wherein if X₄₁ is E, S, T, 4-Pal, D, G, Q or H, then X₄₂ is absent or G, E or γE,
wherein if X₄₀ is absent, then X₄₁ and X₄₂ are also absent,
wherein if X₄₁ is absent, then X₄₂ is also absent,
wherein at least one of X₁₇, X₂₀, X₂₄ and X₂₈ is an amino acid with a functional group available for conjugation to a fatty acid,
and wherein the C-terminal amino acid is optionally amidated; or a pharmaceutically acceptable salt thereof.

If X₄₀ absent, then X₄₁ and X₄₂ are also absent and the polypeptide comprises a 39 amino acid sequence. If X₄₁ absent, then X₄₂ is also absent and the polypeptide comprises a 40 amino acid sequence. If X₄₂ is absent, then the polypeptide comprises a 41 amino acid sequence. If none of X₄₀, X₄₁ and X₄₂ are absent (in other words all of X₄₀, X₄₁ and X₄₂ are present), the polypeptide comprises a 42 amino acid sequence.

In one embodiment, X₄₀ is G, E, S, A or T, X₄₁ is absent, and X₄₂ is absent. In such embodiments, the polypeptide comprises a 40 amino acid sequence. In one embodiment, X₄₀ is G, E, S, A or T, X₄₁ is E, S, T, 4-Pal, D, G, Q or H, and X₄₂ is absent. In such embodiments, the polypeptide comprises a 41 amino acid sequence. In one embodiment, X₄₀ is G, E, S, A or T, X₄₁ is E, S, T, 4-Pal, D, G, Q or H, and X₄₂ is G, E or γE. In such embodiments, the polypeptide comprises a 42 amino acid sequence.

In some embodiments, polypeptides of the present invention include at position X₁₇, X₂₀, X₂₄ or X₂₈ any amino acid (natural or non-natural) with a functional group available for conjugation to a fatty acid. In certain embodiments, the amino acid with a functional group available for conjugation to a fatty acid is K, C, E or D. In particularly preferred embodiments the amino acid is K and the conjugation is to the epsilon-amino group of the K side-chain.

Thus, in some embodiments, the amino acid at position X₁₇, X₂₀, X₂₄ or X₂₈ with a functional group available for conjugation to a fatty acid is K, C, E or D. In some embodiments, the amino acid at position X₁₇, X₂₀, X₂₄ or X₂₈ with a functional group available for conjugation to a fatty acid is K.

In some embodiments, X₁₀ is F or 4-Pal. In some embodiments, X₁₀ is F. In some embodiments, X₁₀ is 4-Pal. In some embodiments, X₁₂ is Orn, K, R or Q. In some embodiments, X₁₂ is Orn. In some embodiments, X₁₂ is K. In some embodiments, X₁₂ is R. In some embodiments, X₁₂ is Q.

In one embodiment, only one of X₁₇, X₂₀, X₂₄ and X₂₈ is an amino acid with a functional group available for conjugation to a fatty acid. In one embodiment, the conjugation is an acylation.

In some embodiments, only one of X₁₇, X₂₀, X₂₄ and X₂₈ is conjugated to a fatty acid. In some embodiments, only one of X₁₇, X₂₀, X₂₄ and X₂₈ is conjugated to a fatty acid optionally via a linker between the amino acid and the fatty acid. Thus, in some embodiments, only one of X₁₇, X₂₀, X₂₄ and X₂₈ is conjugated to a fatty acid via a direct bond between the amino acid and the fatty acid or via a linker between the amino acid and the fatty acid. In some embodiments, the fatty acid is a C₁₆-C₂₂ fatty acid.

Thus, in one embodiment, X₁₇ is K and is conjugated to a C₁₆-C₂₂ fatty acid via a direct bond or via a linker between the amino acid and the C₁₆-C₂₂ fatty acid. In one embodiment, X₁₇ is K and is conjugated to a C₁₆-C₂₂ fatty acid via a linker between the amino acid and the C₁₆-C₂₂ fatty acid. In such embodiments, X₂₀ is Aib, αMe-4-pal, Q or R, X₂₄ is E, Q, D-Glu or N, and X₂₈ is E or A. In an embodiment, the conjugation is to the epsilon-amino group of the K side-chain.

In one embodiment, X₂₀ is K and is conjugated to a C₁₆-C₂₂ fatty acid via a direct bond or via a linker between the amino acid and the C₁₆-C₂₂ fatty acid. In one embodiment, X₂₀ is K and is conjugated to a C₁₆-C₂₂ fatty acid via a linker between the amino acid and the C₁₆-C₂₂ fatty acid. In such embodiments, X₁₇ is A, I or Q, X₂₄ is E, Q, D-Glu or N, and X₂₈ is E or A. In an embodiment, the conjugation is to the epsilon-amino group of the K side-chain.

In one embodiment, X₂₄ is K and is conjugated to a C₁₆-C₂₂ fatty acid via a direct bond or via a linker between the amino acid and the C₁₆-C₂₂ fatty acid. In one embodiment, X₂₄ is K and is conjugated to a C₁₆-C₂₂ fatty acid via a linker between the amino acid and the C₁₆-C₂₂ fatty acid. In such embodiments, X₁₇ is A, I or Q, X₂₀ is Aib, αMe-4-pal, Q or R, and X₂₈ is E or A. In an embodiment, the conjugation is to the epsilon-amino group of the K side-chain.

In one embodiment, X₂₈ is K and is conjugated to a C₁₆-C₂₂ fatty acid via a direct bond or via a linker between the amino acid and the C₁₆-C₂₂ fatty acid. In one embodiment, X₂₈ is K and is conjugated to a C₁₆-C₂₂ fatty acid via a linker between the amino acid and the C₁₆-C₂₂ fatty acid. In such embodiments, X₁₇ is A, I or Q, X₂₀ is Aib, αMe-4-pal, Q or R, and X₂₄ is E, Q, D-Glu or N. In an embodiment, the conjugation is to the epsilon-amino group of the K side-chain.

In one embodiment, provided herein is a polypeptide that comprises Formula I′ (SEQ ID NO:1243):

X₁X₂QX₄TX₆TSDX₁₀X₁₁X₁₂X₁₃LX₁₅X₁₆X₁₇AX₁₉X₂₀X₂₁FX₂₃
X₂₄X₂₅LX₂₇X₂₈X₂₉X₃₀X₃₁X₃₂SX₃₄X₃₅X₃₆X₃₇X₃₈X₃₉X₄₀
X₄₁X₄₂X₄₃X₄₄X₄₅X₄₆,

wherein:
X₁ is Y, NMeY or H,
X₂ is Aib,
X₄ is G or D-Ala,
X₆ is F, αMeF or αMeF(2F),
X₁₀ is F, 4-Pal, F(4CN), 3-Pal, F(4NO₂) or Y,
X₁₁ is S or αMeS,
X₁₂ is Orn, K, R, Q, Dap, Dab, S, E or I,
X₁₃ is αMeL, I or L,
X₁₅ is D or E,
X₁₆ is K, Orn, A or E,
X₁₇ is any amino acid with a functional group available for conjugation to a fatty acid, A, I, Q or Orn,
X₁₉ is Q or A,
X₂₀ is any amino acid with a functional group available for conjugation to a fatty acid, Aib, αMe-4-Pal, Q, R or L-Iva,
X₂₁ is A, Q, Orn, Aad, Aib, S, N, E or T,
X₂₃ is I or V,
X₂₄ is any amino acid with a functional group available for conjugation to a fatty acid, E, Q, D-Glu, N or D-Gln,
X₂₅ is W, Y, F, 4-Pal, αMeY or αMe-4-Pal,
X₂₇ is L, I, E, V, A, Q, S, T or Aad,
X₂₈ is any amino acid with a functional group available for conjugation to a fatty acid, E, Q or A,
X₂₉ is G, D-Ala, Aib, T or A,
X₃₀ is A, S or G,
X₃₁ is P, H, S, 4-Pal, E, T, K(Ac) or Hyp (hydroxyproline),
X₃₂ is S or P
X₃₄ is G or Aib,
X₃₅ is A, Aib, E, D, H or 4-Pal or Om,
X₃₆ is P or Hyp,
X₃₇ is P, Hyp or E,
X₃₈ is P or Hyp,
X₃₉ is E, S, G, T, H, 4-Pal, γE or A or Orn,
X₄₀ is absent or G, E, S, A, T, or D-Glu,
wherein if X₄₀ is G, E, S, A or T, then X₄₁ is absent or A, E, S, T, 4-Pal, D, G, γE, D-Glu, Q or H,
wherein if X₄₁ is A, E, S, T, 4-Pal, D, G, γE, D-Glu, Q or H, then X₄₂ is absent or G, E, γE, D-Glu, or AEEA,
wherein if X₄₂ is G, E, γE, D-Glu(e), or AEEA, then X₄₃ is absent or E, γE, or D-Glu,
wherein if X₄₃ is E, γE, or D-Glu, then X₄₄ is absent or E, wherein if X₄₄ is E then X₄₅ is absent or E, wherein if X₄₅ is E then X₄₆ is absent or E,
wherein if X₄₀ is absent, then X₄₁ through X₄₆ are also absent,
wherein if X₄₁ is absent, then X₄₂, through X₄₆ are also absent,
wherein if X₄₂ is absent, then X₄₃ through X₄₆ are also absent,
wherein if X₄₃ is absent, then X₄₄ through X₄₆ are also absent, wherein if X₄₄ is absent, then X₄₅ and X₄₆ are also absent; wherein if X₄₅ is absent then X₄₆ is also absent,
wherein at least one of X₁₇, X₂₀, X₂₄ and X₂₈ is an amino acid with a functional group available for conjugation to a fatty acid,
wherein the C-terminal amino acid is optionally amidated; or a pharmaceutically acceptable salt thereof.

If X₄₀ absent, then X₄₁ through X₄₆ are also absent and the polypeptide comprises a 39 amino acid sequence backbone. If X₄₁ absent, then X₄₂ through X₄₆ are also absent and the polypeptide comprises a 40 amino acid sequence backbone. If X₄₂ is absent, then the polypeptide comprises a 41 amino acid sequence backbone. If X₄₃ is absent, then the polypeptide comprises a 42 amino acid sequence backbone. If X₄₄ is absent, then the polypeptide comprises a 43 amino acid sequence backbone. If X₄₅ is absent, then the polypeptide comprises a 44 amino acid sequence backbone. If X₄₆ is absent, then the polypeptide comprises a 45 amino acid sequence backbone. If none of X₄₀ through X₄₆ are absent (in other words all of X₄₀ through X₄₆ are present), the polypeptide comprises a 46 amino acid sequence backbone.

In one embodiment, X₄₀ is G, E, S, A or T, X₄₁ is absent, and X₄₂ is absent. In such embodiments, the polypeptide comprises a 40 amino acid sequence. In one embodiment, X₄₀ is G, E, S, A or T, X₄₁ is E, S, T, 4-Pal, D, G, Q or H, and X₄₂ is absent. In such embodiments, the polypeptide comprises a 41 amino acid sequence. In one embodiment, X₄₀ is G, E, S, A or T, X₄₁ is E, S, T, 4-Pal, D, G, Q or H, and X₄₂ is G, E or γE. In such embodiments, the polypeptide comprises a 42 amino acid sequence.

In some embodiments, polypeptides of the present invention include at position X₁₇, X₂₀, X₂₄ or X₂₈ any amino acid (natural or non-natural) with a functional group available for conjugation to a fatty acid. In certain embodiments, the amino acid with a functional group available for conjugation to a fatty acid is K, C, E or D. In certain embodiments the amino acid is K, and a fatty acid is conjugated to the epsilon-amino group of the K side-chain.

Thus, in some embodiments, the amino acid at position X₁₇, X₂₀, X₂₄ or X₂₈ with a functional group available for conjugation to a fatty acid is K, C, E or D. In some embodiments, the amino acid at position X₁₇, X₂₀, X₂₄ or X₂₈ with a functional group available for conjugation to a fatty acid is K.

In some embodiments, X₁₀ is F or 4-Pal. In some embodiments, X₁₀ is F. In some embodiments, X₁₀ is 4-Pal. In some embodiments, X₁₂ is Orn, K, R or Q. In some embodiments, X₁₂ is Orn. In some embodiments, X₁₂ is K. In some embodiments, X₁₂ is R. In some embodiments, X₁₂ is Q.

In one embodiment, only one of X₁₇, X₂₀, X₂₄ and X₂₈ is an amino acid with a functional group available for conjugation to a fatty acid. In one embodiment, the conjugation is an acylation.

In some embodiments, only one of X₁₇, X₂₀, X₂₄ and X₂₈ is conjugated to a fatty acid. In some embodiments, only one of X₁₇, X₂₀, X₂₄ and X₂₈ is conjugated to a fatty acid optionally via a linker between the amino acid and the fatty acid. Thus, in some embodiments, only one of X₁₇, X₂₀, X₂₄ and X₂₈ is conjugated to a fatty acid via a direct bond between the amino acid and the fatty acid or via a linker between the amino acid and the fatty acid. In some embodiments, the fatty acid is a C₁₆-C₂₂ fatty acid.

Thus, in one embodiment, X₁₇ is K and is conjugated to a C₁₆-C₂₂ fatty acid via a direct bond or via a linker between the amino acid and the C₁₆-C₂₂ fatty acid. In one embodiment, X₁₇ is K and is conjugated to a C₁₆-C₂₂ fatty acid via a linker between the amino acid and the C₁₆-C₂₂ fatty acid. In such embodiments, X₂₀ is Aib, αMe-4-pal, Q or R, X₂₄ is E, Q, D-Glu or N, and X₂₈ is E or A. In an embodiment, the conjugation is to the epsilon-amino group of the K side-chain.

In one embodiment, X₂₀ is K and is conjugated to a C₁₆-C₂₂ fatty acid via a direct bond or via a linker between the amino acid and the C₁₆-C₂₂ fatty acid. In one embodiment, X₂₀ is K and is conjugated to a C₁₆-C₂₂ fatty acid via a linker between the amino acid and the C₁₆-C₂₂ fatty acid. In such embodiments, X₁₇ is A, I or Q, X₂₄ is E, Q, D-Glu or N, and X₂₈ is E or A. In an embodiment, the conjugation is to the epsilon-amino group of the K side-chain.

In one embodiment, X₂₄ is K and is conjugated to a C₁₆-C₂₂ fatty acid via a direct bond or via a linker between the amino acid and the C₁₆-C₂₂ fatty acid. In one embodiment, X₂₄ is K and is conjugated to a C₁₆-C₂₂ fatty acid via a linker between the amino acid and the C₁₆-C₂₂ fatty acid. In such embodiments, X₁₇ is A, I or Q, X₂₀ is Aib, αMe-4-pal, Q or R, and X₂₈ is E or A. In an embodiment, the conjugation is to the epsilon-amino group of the K side-chain.

In one embodiment, X₂₈ is K and is conjugated to a C₁₆-C₂₂ fatty acid via a direct bond or via a linker between the amino acid and the C₁₆-C₂₂ fatty acid. In one embodiment, X₂₈ is K and is conjugated to a C₁₆-C₂₂ fatty acid via a linker between the amino acid and the C₁₆-C₂₂ fatty acid. In such embodiments, X₁₇ is A, I or Q, X₂₀ is Aib, αMe-4-pal, Q or R, and X₂₄ is E, Q, D-Glu or N. In an embodiment, the conjugation is to the epsilon-amino group of the K side-chain.

In some embodiments, none of X₁₇, X₂₀, X₂₄ and X₂₈ is conjugated to a fatty acid.

In one embodiment, provided herein is a polypeptide that includes the formula II (SEQ ID NO:5):

YX₂QGTFTSDX₁₀SX₁₂X₁₃LDX₁₆X₁₇AQX₂₀X₂₁FIX₂₄X₂₅LX₂₇
X₂₈X₂₉X₃₀X₃₁SSX₃₄X₃₅X₃₆X₃₇X₃₈X₃₉X₄₀X₄₁X₄₂,

wherein:
X₂ is Aib,
X₁₀ is F, 4-Pal, F(4CN),
X₁₂ is Om, K, R, Q, Dap or Dab,
X₁₃ is αMeL,
X₁₆ is K or Orn,
X₁₇ is any amino acid with a functional group available for conjugation to a fatty acid,
X₂₀ is Aib, αMe-4-pal or Q,
X₂₁ is A, Q or Orn,
X₂₄ is E or Q,
X₂₅ is W, Y, 4-Pal, αMeY or αMe-4-Pal,
X₂₇ is L or I,
X₂₈ is E or A,
X₂₉ is G, D-Ala or Aib,
X₃₀ is A or G,
X₃₁ is P, H, S, 4-Pal, E, T, K(Ac) or Hyp,
X₃₄ is G or Aib,
X₃₅ is A, Aib, E, H or 4-Pal,
X₃₆ is P or Hyp,
X₃₇ is P or Hyp,
X₃₈ is P or Hyp,
X₃₉ is E, S, G, T, H, 4-Pal or γE,
X₄₀ is absent or G, E or S,
wherein if X₄₀ is G, E or S, then X₄₁ is absent or E, S, T, 4-Pal, D, G, Q or H,
wherein if X₄₁ is E, S, T, 4-Pal, D, G, Q or H, then X₄₂ is absent or G, E or γE,
wherein if X₄₀ is absent, then X₄₁ and X₄₂ are also absent,
wherein if X₄₁ is absent, then X₄₂ is also absent,
wherein the C-terminal amino acid is optionally amidated;
or a pharmaceutically acceptable salt thereof.

As noted before, if X₄₀ absent, then X₄₁ and X₄₂ are also absent and the polypeptide comprises a 39 amino acid sequence. If X₄₁ absent, then X₄₂ is also absent and the polypeptide comprises a 40 amino acid sequence. If X₄₂ is absent, then the polypeptide comprises a 41 amino acid sequence. If none of X₄₀, X₄₁ and X₄₂ are absent (in other words all of X₄₀, X₄₁ and X₄₂ are present), the polypeptide comprises a 42 amino acid sequence.

In one embodiment, X₄₀ is G, E or S, X₄₁ is absent, and X₄₂ is absent. In such embodiments, the polypeptide comprises a 40 amino acid sequence. In one embodiment, X₄₀ is G, E or S, X₄₁ is E, S, T, 4-Pal, D, G, Q or H, and X₄₂ is absent. In such embodiments, the polypeptide comprises a 41 amino acid sequence. In one embodiment, X₄₀ is G, E or S, X₄₁ is E, S, T, 4-Pal, D, G, Q or H, and X₄₂ is G, E or γE. In such embodiments, the polypeptide comprises a 42 amino acid sequence.

In some embodiments of polypeptides of formula II, the amino acid at position X₁₇ with a functional group available for conjugation to a fatty acid is K, C, E or D. In preferred embodiments, the amino acid at position X₁₇ with a functional group available for conjugation to a fatty acid is K and the conjugation is to the epsilon-amino group of the K side-chain.

In some embodiments of polypeptides of formula II, X₁₀ is F or 4-Pal. In some embodiments, X₁₀ is F. In some embodiments, X₁₀ is 4-Pal. In some embodiments, X₁₂ is Orn, K, R or Q. In some embodiments, X₁₂ is Orn. In some embodiments, X₁₂ is K. In some embodiments, X₁₂ is R. In some embodiments, X₁₂ is Q. In some embodiments, X₁₀ is F or 4-Pal and X₁₂ is Orn, K, R or Q. In some embodiments, X₁₀ is F and X₁₂ is Q. In some embodiments, X₁₀ is F and X₁₂ is Orn. In some embodiments, X₁₀ is F and X₁₂ is K. In some embodiments, X₁₀ is F and X₁₂ is R. In some embodiments, X₁₀ is 4-Pal and X₁₂ is Q. In some embodiments, X₁₀ is 4-Pal and X₁₂ is Orn. In some embodiments, X₁₀ is 4-Pal and X₁₂ is K. In some embodiments, X₁₀ is 4-Pal and X₁₂ is R.

In further embodiments, X₁₀ is selected from F or 4-Pal. In some embodiments, X₁₂ is selected from Orn, K, R or Q. In some embodiments, X₁₆ is K. In some embodiments, X₁₇ is K. In some embodiments, X₂₀ is selected from Aib or αMe-4-pal. In some embodiments, X₂₄ is E. In some embodiments, X₂₈ is selected from E or A. In some embodiments, X₃₁ is selected from P, H, S, 4-Pal, T or E. In some embodiments, X₃₅ is selected from A, Aib or E. In some embodiments, X₃₆ is P. In some embodiments, X₃₇ is P. In some embodiments, X₃₈ is P. In some embodiments, X₃₉ is selected from E, S or G. In some embodiments, X₄₀ is selected from G, E or S. In some embodiments, X₄₁ is selected from E, S, T, 4-Pal or H.

In some embodiments, X₁₀ is selected from the group consisting of F and 4-Pal. In some embodiments, X₁₂ is selected from the group consisting of Orn, K, R and Q. In some embodiments, X₁₆ is K. In some embodiments, X₁₇ is K. In some embodiments, X₂₀ is selected from the group consisting of Aib and αMe-4-pal. In some embodiments, X₂₄ is E. In some embodiments, X₂₈ is selected from the group consisting of E and A. In some embodiments, X₃₁ is selected from the group consisting of P, H, S, 4-Pal, T and E. In some embodiments, X₃₅ is selected from the group consisting of A, Aib and E. In some embodiments, X₃₆ is P. In some embodiments, X₃₇ is P. In some embodiments, X₃₈ is P. In some embodiments, X₃₉ is selected from the group consisting of E, S and G. In some embodiments, X₄₀ is selected from the group consisting of G, E and S. In some embodiments, X₄₁ is selected from the group consisting of E, S, T, 4-Pal and H.

In some embodiments, X₁₀ is selected from F or 4-Pal; X₁₂ is Orn, K, R or Q; X₁₆ is K; X₁₇ is K; X₂₀ is selected from Aib or αMe-4-pal; X₂₄ is E; X₂₈ is selected from E or A; X₃₁ is selected from P, H, S, 4-Pal, T or E; X₃₅ is selected from A, Aib or E; X₃₆ is P; X₃₇ is P; X₃₈ is P; X₃₉ is selected from E, S or G; X₄₀ is selected from G, E or S; and X₄₁ is selected from E, S, T, 4-Pal or H.

In some embodiments, X₁₀ is selected from the group consisting of F and 4-Pal; X₁₂ is selected from the group consisting of Orn, K, R and Q; X₁₆ is K; X₁₇ is K; X₂₀ is selected from the group consisting of Aib and αMe-4-pal; X₂₄ is E; X₂₈ is selected from the group consisting of E and A; X₃₁ is selected from the group consisting of P, H, S, 4-Pal, T and E; X₃₅ is selected from the group consisting of A, Aib and E; X₃₆ is P; X₃₇ is P; X₃₈ is P; X₃₉ is selected from the group consisting of E, S and G; X₄₀ is selected from the group consisting of G, E and S; and X₄₁ is selected from the group consisting of E, S, T, 4-Pal and H.

In some embodiments, amino acid X₁₇ is conjugated to a fatty acid. In some embodiments, amino acid X₁₇ is conjugated to a fatty acid optionally via a linker between the amino acid and the fatty acid. Thus, in some embodiments, amino acid X₁₇ is conjugated to a fatty acid via a direct bond between the amino acid and the fatty acid or via a linker between the amino acid and the fatty acid. In some embodiments, the fatty acid is a C₁₆-C₂₂ fatty acid.

Thus, in some embodiments, X₁₇ is K and is conjugated to a C₁₆-C₂₂ fatty acid via a direct bond between the amino acid and the C₁₆-C₂₂ fatty acid or via a linker between the amino acid and the C₁₆-C₂₂ fatty acid. In one embodiment, X₁₇ K is conjugated to a C₁₆-C₂₂ fatty acid via a linker between the amino acid and the C₁₆-C₂₂ fatty acid. In an embodiment, the conjugation is to the epsilon-amino group of the X₁₇ K side-chain.

In one embodiment, provided herein is a polypeptide that includes the formula II′ (SEQ ID NO:1244):

X₁X₂QGTFTSDX₁₀SX₁₂X₁₃LDX₁₆X₁₇AQX₂₀X₂₁FIX₂₄X₂₅LX₂₇
X₂₈X₂₉X₃₀X₃₁SSX₃₄X₃₅X₃₆X₃₇X₃₈X₃₉X₄₀X₄₁X₄₂X₄₃X₄₄,

wherein
X₁ is Y or NMeY
X₂ is Aib,
X₁₀ is F, 4-Pal or F(4CN),
X₁₂ is Orn, K, R, I, Q, Dap or Dab,
X₁₃ is αMeL,
X₁₆ is K or Orn,
X₁₇ is any amino acid with a functional group available for conjugation to a fatty acid,
X₂₀ is Aib, αMe-4-Pal, Q or L-Iva,
X₂₁ is A, Q or Orn,
X₂₄ is E or Q,
X₂₅ is W, Y, 4-Pal, αMeY or αMe-4-Pal,
X₂₇ is L or I,
X₂₈ is E, Q or A,
X₂ is G, D-Ala or Aib,
X₃₀ is A, S or G,
X₃₁ is P, H, S, 4-Pal, E, T, K(Ac) or Hyp,
X₃₄ is G or Aib,
X₃₅ is A, Aib, E, H or 4-Pal,
X₃₆ is P or Hyp,
X₃₇ is P or Hyp,
X₃₈ is P or Hyp,
X₃₉ is E, S, G, T, H, 4-Pal or γE,
X₄₀ is absent or G, E, S or D-Glu,
wherein if X₄₀ is G, E, S or D-Glu, then X₄₁ is absent or E, S, T, 4-Pal, D, G, Q or H,
wherein if X₄₁ is E, S, T, 4-Pal, D, G, Q or H, then X₄₂ is absent or G, E or γE,
wherein if X₄₂ is G, E, or γE, then X₄₃ is absent or E,
wherein if X₄₃ is E, then X₄₄ is absent or E,
wherein if X₄₀ is absent, then X₄₁, X₄₂, X₄₃, and X₄₄ are also absent,
wherein if X₄₁ is absent, then X₄₂, X₄₃ and X₄₄ are is also absent,
wherein if X₄₃ is absent then X₄₄ is also absent,
wherein the C-terminal amino acid is optionally amidated; or a pharmaceutically acceptable salt thereof.

As noted before, if X₄₀ absent, then X₄₁ through X₄₄ are also absent and the polypeptide comprises a 39 amino acid sequence backbone. If X₄₁ absent, then X₄₂ through X₄₄ are also absent and the polypeptide comprises a 40 amino acid sequence backbone. If X₄₂ is absent, then X₄₃ through X₄₄ are also absent and the polypeptide comprises a 41 amino acid sequence backbone. If X₄₃ is absent, then X₄₄ is also absent and the polypeptide comprises a 42 amino acid sequence backbone. If none of X₄₀ through X₄₄ are absent (in other words all of X₄₀ through X₄₄ are present), the polypeptide comprises a 44 amino acid sequence backbone.

In one embodiment, X₄₀ is G, E or S, X₄₁ is absent, and X₄₂ is absent. In such embodiments, the polypeptide comprises a 40 amino acid sequence. In one embodiment, X₄₀ is G, E or S, X₄₁ is E, S, T, 4-Pal, D, G, Q or H, and X₄₂ is absent. In such embodiments, the polypeptide comprises a 41 amino acid sequence. In one embodiment, X₄₀ is G, E or S, X₄₁ is E, S, T, 4-Pal, D, G, Q or H, and X₄₂ is G, E or γE. In such embodiments, the polypeptide comprises a 42 amino acid sequence backbone.

In some embodiments of polypeptides of formula II′, the amino acid at position X₁₇ with a functional group available for conjugation to a fatty acid is K, C, E or D. In some embodiments, the amino acid at position X₁₇ with a functional group available for conjugation to a fatty acid is K and the conjugation is to the epsilon-amino group of the K side-chain.

In some embodiments of polypeptides of formula II′, X₁₀ is F or 4-Pal. In some embodiments, X₁₀ is F. In some embodiments, X₁₀ is 4-Pal. In some embodiments, X₁₂ is Orn, K, R or Q. In some embodiments, X₁₂ is Orn. In some embodiments, X₁₂ is K. In some embodiments, X₁₂ is R. In some embodiments, X₁₂ is Q. In some embodiments, X₁₀ is F or 4-Pal and X₁₂ is Orn, K, R or Q. In some embodiments, X₁₀ is F and X₁₂ is Q. In some embodiments, X₁₀ is F and X₁₂ is Orn. In some embodiments, X₁₀ is F and X₁₂ is K. In some embodiments, X₁₀ is F and X₁₂ is R. In some embodiments, X₁₀ is 4-Pal and X₁₂ is Q. In some embodiments, X₁₀ is 4-Pal and X₁₂ is Orn. In some embodiments, X₁₀ is 4-Pal and X₁₂ is K. In some embodiments, X₁₀ is 4-Pal and X₁₂ is R.

In further embodiments, X₁₀ is selected from F or 4-Pal. In some embodiments, X₁₂ is selected from Orn, K. R or Q. In some embodiments, X₁₆ is K. In some embodiments, X₁₇ is K. In some embodiments, X₂₀ is selected from Aib or αMe-4-pal. In some embodiments, X₂₄ is E. In some embodiments, X₂₈ is selected from E or A. In some embodiments, X₃₁ is selected from P, H, S, 4-Pal, T or E. In some embodiments, X₃₅ is selected from A, Aib or E. In some embodiments, X₃₆ is P. In some embodiments, X₃₇ is P. In some embodiments, X₃₈ is P. In some embodiments, X₃₉ is selected from E, S or G. In some embodiments, X₄₀ is selected from G, E or S. In some embodiments, X₄₁ is selected from E, S, T, 4-Pal or H.

In some embodiments, X₁₀ is selected from the group consisting of F and 4-Pal. In some embodiments, X₁₂ is selected from the group consisting of Orn, K, R and Q. In some embodiments, X₁₆ is K. In some embodiments, X₁₇ is K. In some embodiments, X₂₀ is selected from the group consisting of Aib and αMe-4-pal. In some embodiments, X₂₄ is E. In some embodiments, X₂₈ is selected from the group consisting of E and A. In some embodiments, X₃₁ is selected from the group consisting of P, H, S, 4-Pal, T and E. In some embodiments, X₃₅ is selected from the group consisting of A, Aib and E. In some embodiments, X₃₆ is P. In some embodiments, X₃₇ is P. In some embodiments, X₃₈ is P. In some embodiments, X₃₉ is selected from the group consisting of E, S and G. In some embodiments, X₄₀ is selected from the group consisting of G, E and S. In some embodiments, X₄₁ is selected from the group consisting of E, S, T, 4-Pal and H.

In some embodiments, X₁₀ is selected from F or 4-Pal; X₁₂ is Orn, K, R or Q; X₁₆ is K; X₁₇ is K; X₂₀ is selected from Aib or αMe-4-pal; X₂₄ is E; X₂₈ is selected from E or A; X₃₁ is selected from P, H, S, 4-Pal, T or E; X₃₅ is selected from A, Aib or E; X₃₆ is P; X₃₇ is P; X₃₈ is P; X₃₉ is selected from E, S or G; X₄₀ is selected from G, E or S; and X₄₁ is selected from E, S, T, 4-Pal or H.

In some embodiments, X₁₀ is selected from the group consisting of F and 4-Pal; X₁₂ is selected from the group consisting of Orn, K, R and Q; X₁₆ is K; X₁₇ is K; X₂₀ is selected from the group consisting of Aib and αMe-4-pal; X₂₄ is E; X₂₈ is selected from the group consisting of E and A; X₃₁ is selected from the group consisting of P, H, S, 4-Pal, T and E; X₃₅ is selected from the group consisting of A, Aib and E; X₃₆ is P; X₃₇ is P; X₃₈ is P; X₃₉ is selected from the group consisting of E, S and G; X₄₀ is selected from the group consisting of G, E and S; and X₄₁ is selected from the group consisting of E, S, T, 4-Pal and H.

In some embodiments, amino acid X₁₇ is conjugated to a fatty acid. In some embodiments, amino acid X₁₇ is conjugated to a fatty acid optionally via a linker between the amino acid and the fatty acid. Thus, in some embodiments, amino acid X₁₇ is conjugated to a fatty acid via a direct bond between the amino acid and the fatty acid or via a linker between the amino acid and the fatty acid. In some embodiments, the fatty acid is a C₁₆-C₂₂ fatty acid.

Thus, in some embodiments, X₁₇ is K and is conjugated to a C₁₆-C₂₂ fatty acid via a direct bond between the amino acid and the C₁₆-C₂₂ fatty acid or via a linker between the amino acid and the C₁₆-C₂₂ fatty acid. In one embodiment, X₁₇ K is conjugated to a C₁₆-C₂₂ fatty acid via a linker between the amino acid and the C₁₆-C₂₂ fatty acid. In an embodiment, the conjugation is to the epsilon-amino group of the X₁₇ K side-chain.

In one embodiment, provided herein is a polypeptide that includes the formula III (SEQ ID NO:6):

YX₂QGTFTSDX₁₀SX₁₂X₁₃LDX₁₆X₁₇AQX₂₀X₂₁FIX₂₄X₂₅LX₂₇
X₂₈X₂₉X₃₀X₃₁SSX₃₄X₃₅X₃₆X₃₇X₃₈X₃₉X₄₀X₄₁,

wherein
X₁ is Y, NMeY or H,
X₂ is Aib,
X₆ is F, αMeF or αMeF(2F),
X₁₀ is F, 3-Pal, 4-Pal, F(4CN), F(4NO₂) or Y,
X₁₁ is S or αMeS,
X₁₂ is Om, K, R, Q, Dap, S, E or I,
X₁₃ is αMeL, I or L,
X₁₆ is K, Orn, A or E,
X₁₇ is any amino acid with a functional group available for conjugation to a fatty acid, A, I or Q,
X₁₉ is A or Q,
X₂₀ is any amino acid with a functional group available for conjugation to a fatty acid Aib, Q, R or αMe-4-pal,
X₂₁ is A, Aad, Aib, S, N, Q, E, T or Om,
X₂₃ is I or V,
X₂₄ is any amino acid with a functional group available for conjugation to a fatty acid E, D-Glu, Q or N,
X₂₅ is W, Y, F, 4-Pal, αMeY or αMe-4-pal,
X₂₇ is L, I, E, V, A, Q or S,
X₂₈ is any amino acid with a functional group available for conjugation to a fatty acid, E or A,
X₂₉ is G, Aib, T, D-Ala or A,
X₃₁ is P or E,
X₃₄ is G or Aib,
X₃₅ is A or E,
X₃₆ is P,
X₃₇ is P or E,
X₃₈ is P,
X₃₉ is E, S, G or A,
X₄₀ is absent or G, E, S, A or T,
wherein if X₄₀ is G, E, S, A or T, then X₄₁ is absent or is E, S, D or G,
wherein if X₄₁ is E, S, D or G, then X₄₂ is absent or G, E or γE,
wherein if X₄₀ is absent, then X₄₁ and X₄₂ are also absent,
wherein if X₄₁ is absent, then X₄₂ is also absent, wherein if X₁₀ is F, 3-Pal, 4-Pal, F(4CN), F(4NO₂), then X₁₂ is I and
wherein at least one of X₁₇, X₂₀, X₂₄ or X₂₈ is any amino acid with a functional group available for conjugation to a C₁₆-C₂₂ fatty acid,
wherein the C-terminal amino acid is optionally amidated;
or a pharmaceutically acceptable salt thereof.

As noted before, if X₄₀ absent, then X₄₁ and X₄₂ are also absent and the polypeptide comprises a 39 amino acid sequence. If X₄₁ absent, then X₄₂ is also absent and the polypeptide comprises a 40 amino acid sequence. If X₄₂ is absent, then the polypeptide comprises a 41 amino acid sequence. If none of X₄₀, X₄₁ and X₄₂ are absent (in other words all of X₄₀, X₄₁ and X₄₂ are present), the polypeptide comprises a 42 amino acid sequence.

In one embodiment, X₄₀ is G, E, S, A, or T, and X₄₁ is absent. In such embodiment, the polypeptide comprises a 40 amino acid sequence. In one embodiment, X₄₀ is G, E, S, A or T, X₄₁ is E, S, D or G. In such embodiment, the polypeptide comprises a 41 amino acid sequence. In one embodiment, X₄₀ is G, E, S, A or T, X₄₁ is E, S, D or G, and X₄₂ is G, E or γE. In such embodiment, the polypeptide comprises a 42 amino acid sequence.

In some embodiments of polypeptides of formula III, the amino acid at position X₁₇, X₂₀, X₂₄ or X₂₈ with a functional group available for conjugation to a fatty acid is K, C, E or D. In some embodiments, the amino acid at position X₁₇, X₂₀, X₂₄ or X₂₈ with a functional group available for conjugation to a fatty acid is K and the conjugation is to the epsilon-amino group of the K side-chain.

In some embodiments, X₁₀ is F, 3-Pal, 4-Pal, F(4CN) or F(4NO₂) and X₁₂ is I. In some embodiments of the polypeptide of formula III, X₁₀ is Y and X₁₂ is Orn, K. R. Q. Dap, S, E or I.

In some embodiments, only one of X₁₇, X₂₀, X₂₄ and X₂₈ is an amino acid with a functional group available for conjugation to a C₁₆-C₂₂ fatty acid.

In some embodiments, only one of X₁₇, X₂₀, X₂₄ and X₂₈ is conjugated to a fatty acid optionally via a linker between the amino acid and the fatty acid. Thus, in some embodiments, only one of X₁₇, X₂₀, X₂₄ and X₂₈ is conjugated to a fatty acid via a direct bond between the amino acid and the fatty acid or via a linker between the amino acid and the fatty acid. In one embodiment, only one of X₁₇, X₂₀, X₂₄ and X₂₈ is conjugated to a fatty acid via a linker between the amino acid and the fatty acid. In some embodiments, the fatty acid is a C₁₆-C₂₂ fatty acid.

Thus, in one embodiment, X₁₇ is K and is conjugated to a C₁₆-C₂₂ fatty acid via a direct bond or via a linker between the amino acid and the C₁₆-C₂₂ fatty acid. In one embodiment, X₁₇ is K and is conjugated to a C₁₆-C₂₂ fatty acid via a linker between the amino acid and the C₁₆-C₂₂ fatty acid. In such embodiments, X₂₀ is Aib, αMe-4-pal, Q or R, X₂₄ is E, Q, D-Glu or N, and X₂₈ is E or A. In an embodiment, the conjugation is to the epsilon-amino group of the K side-chain.

In one embodiment, X₂₀ is K and is conjugated to a C₁₆-C₂₂ fatty acid via a direct bond or via a linker between the amino acid and the C₁₆-C₂₂ fatty acid. In one embodiment, X₂₀ is K and is conjugated to a C₁₆-C₂₂ fatty acid via linker between the amino acid and the C₁₆-C₂₂ fatty acid. In such embodiments, X₁₇ is A, I or Q, X₂₄ is E, Q, D-Glu or N, and X₂₈ is E or A. In an embodiment, the conjugation is to the epsilon-amino group of the K side-chain.

In one embodiment, X₂₄ is K and is conjugated to a C₁₆-C₂₂ fatty acid via a direct bond or via a linker between the amino acid and the C₁₆-C₂₂ fatty acid. In one embodiment, X₂₄ is K and is conjugated to a C₁₆-C₂₂ fatty acid via linker between the amino acid and the C₁₆-C₂₂ fatty acid. In such embodiments, X₁₇ is A, I or Q, X₂₀ is Aib, αMe-4-pal, Q or R, and X₂₈ is E or A. In an embodiment, the conjugation is to the epsilon-amino group of the K side-chain.

In one embodiment, X₂₈ is K and is conjugated to a C₁₆-C₂₂ fatty acid via a direct bond or via a linker between the amino acid and the C₁₆-C₂₂ fatty acid. In one embodiment, X₂₈ is K and is conjugated to a C₁₆-C₂₂ fatty acid via a linker between the amino acid and the C₁₆-C₂₂ fatty acid. In such embodiments, X₁₇ is A, I or Q, X₂₀ is Aib, αMe-4-pal, Q or R, and X₂₄ is E, Q, D-Glu or N. In an embodiment, the conjugation is to the epsilon-amino group of the K side-chain.

In one embodiment, provided herein is a polypeptide that includes the formula III′ (SEQ ID NO:1245):

X₁X₂QX₄TX₆TSDX₁₀X₁₁X₁₂X₁₃LX₁₅X₁₆X₁₇AX₁₉X₂₀X₂₁FX₂₃
X₂₄X₂₅LX₂₇X₂₈X₂₉GX₃₁X₃₂SX₃₄X₃₅PX₃₇PX₃₉X₄₀X₄₁X₄₂
X₄₃X₄₄X₄₅X₄₆;

wherein
X₁ is Y, NMeY or H,
X₂ is Aib,
X₄ is G or D-Ala,
X₆ is F, αMeF or αMeF(2F),
X₁₀ is F, 3-Pal, 4-Pal, F(4CN), F(4NO₂) or Y,
X₁₁ is S or αMeS,
X₁₂ is Om, K, R, Q, Dap, S, E or I,
X₁₃ is αMeL, I or L,
X₁₅ is D or E,
X₁₆ is K, Orn, A or E,
X₁₇ is any amino acid with a functional group available for conjugation to a fatty acid, A, I or Q or Orn,
X₁₉ is A or Q,
X₂₀ is any amino acid with a functional group available for conjugation to a fatty acid, Aib, Q, R or αMe-4-Pal,
X₂₁ is A, Aad, Aib, S, N, Q, E, T or Orn,
X₂₃ is I or V,
X₂₄ is any amino acid with a functional group available for conjugation to a fatty acid E, D-Glu, Q, N or D-Gln,
X₂₅ is W, Y, F, 4-Pal, αMeY or αMe-4-Pal,
X₂₇ is L, I, E, V, A, Aad, T, Q or S,
X₂₈ is any amino acid with a functional group available for conjugation to a fatty acid, E or A,
X₂₉ is G, Aib, T, D-Ala or A,
X₃₁ is P or E,
X₃₂ is S or P,
X₃₄ is G or Aib,
X₃₅ is A, D or E,
X₃₇ is P or E,
X₃₉ is E, S, G, A, T or Orn,
X₄₀ is absent or G, E, S, A or T,
wherein if X₄₀ is G, E, S, A, T or D-Glu, then X₄₁ is absent or is E, S, D, G, Q, T, A, γE or D-Glu,
wherein if X₄₁ is E, S, D, G, Q, T, A, γE or D-Glu, then X₄₂ is absent or G, E, D-Glu, or γE,
wherein if X₄₂ is G, E, D-Glu or γE, then X₄₃ is absent or E, γE, or D-Glu,
wherein if X₄₃ is E, γE, or D-Glu, then X₄ is absent or E,
wherein if X₄₄ is E then X₄₅ is absent or E, wherein if X₄₅ is E then X₄₆ is absent or E,
wherein if X₄₀ is absent, then X₄₁ through X₄₆ are also absent,
wherein if X₄₁ is absent, then X₄₂, through X₄₆ are also absent,
wherein if X₄₂ is absent, then X₄₃ through X₄₆ are also absent,
wherein if X₄₃ is absent, then X₄₄ through X₄₆ are also absent,
wherein if X₄₄ is absent, then X₄₅ and X₄₆ are also absent,
wherein if X₄₅ is absent then X₄₆ is also absent,
wherein the polypeptide comprises at least one of the following: X₆ is αMeF or αMeF(2F); X₁₀ is F, 3-Pal, 4-Pal, F(4CN) or F(4NO₂); X₁₁ is αMeS; X₁₃ is αMeL; X₂₄ is D-Glu; and/or X₂₅ is αMeY,
wherein if X₁₀ is F, 3-Pal, 4-Pal, F(4CN), or F(4NO₂), then X₁₂ is I,
and wherein at least one of X₁₇, X₂₀, X₂₄ or X₂₈ is an amino acid with a functional group available for conjugation to a fatty acid,
wherein the C-terminal amino acid is optionally amidated; or a pharmaceutically acceptable salt thereof.

As noted before, if X₄₀ absent, then X₄₁ through X₄₆ are also absent and the polypeptide comprises a 39 amino acid sequence backbone. If X₄₁ absent, then X₄₂ through X₄₆ are also absent and the polypeptide comprises a 40 amino acid sequence backbone. If X₄₂ is absent, then the polypeptide comprises a 41 amino acid sequence backbone. If X₄₃ is absent, then the polypeptide comprises a 42 amino acid sequence backbone. If X₄₄ is absent, then the polypeptide comprises a 43 amino acid sequence backbone. If X₄₅ is absent, then the polypeptide comprises a 44 amino acid sequence backbone. If X₄₆ is absent, then the polypeptide comprises a 45 amino acid sequence backbone. If none of X₄₀ through X₄₆ are absent (in other words all of X₄₀ through X₄₆ are present), the polypeptide comprises a 46 amino acid sequence backbone.

In one embodiment, X₄₀ is G, E, S, A, or T, and X₄₁ is absent. In such embodiment, the polypeptide comprises a 40 amino acid sequence. In one embodiment, X₄₀ is G, E, S, A or T, X₄₁ is E, S, D or G. In such embodiment, the polypeptide comprises a 41 amino acid sequence. In one embodiment, X₄₀ is G, E, S, A or T, X₄₁ is E, S, D or G, and X₄₂ is G, E or γE. In such embodiment, the polypeptide comprises a 42 amino acid sequence.

In some embodiments of polypeptides of formula III′, the amino acid at position X₁₇, X₂₀, X₂₄ or X₂₈ with a functional group available for conjugation to a fatty acid is K, C, E or D. In some embodiments, the amino acid at position X₁₇, X₂₀, X₂₄ or X₂₈ with a functional group available for conjugation to a fatty acid is K and the conjugation is to the epsilon-amino group of the K side-chain.

In some embodiments, X₁₀ is F, 3-Pal, 4-Pal, F(4CN) or F(4NO₂) and X₁₂ is I. In some embodiments of the polypeptide of formula III, X₁₀ is Y and X₁₂ is Orn, K, R, Q, Dap, S, E or I.

In some embodiments, the polypeptide comprises at least three of the following: X₁₀ is F, 3-Pal, 4-Pal, F(4CN) or F(4NO₂); X₁₁ is αMeS; X₁₃ is αMeL; X₁₆ is Orn; X₂₄ is D-Glu; and/or X₂₅ is αMeY.

In some embodiments, X₁₀ is F, 3-Pal, 4-Pal, F(4CN) or F(4NO₂).

In some embodiments, X₁ is Y, X₂ is Aib, X₄ is G, X₆ is αMeF(2F), X₁₀ is 4-Pal, X₁₂ is I, X₁₃ is αMeL, X₁₅ is D, X₁₆ is Orn, X₁₉ is Q, X₂₀ is αMe-4-Pal, X₂₁ is E or Om, X₂₃ is I, X₂₄ is D-Glu, X₂₅ is αMeY, X₂₇ is I or V, X₂₈ is E, X₂₉ is G, X₃₁ is P, X₃₄ is G, X₃₅ is A or E, X₃₇ is P, X₃₉ is E or S, X₄₀ is G or T, X₄₁ is E, S, or G, X₄₂ is absent, X₄₃ is absent, and X₄₄ is absent.

In some embodiments, X₁₁ is S, X₂₁ is Orn, X₂₇ is I, X₃₅ is E, X₃₉ is E, X₄₀ is T and X₄₁ is E.

In some embodiments, X₁₁ is αMeS, X₂₁ is E, X₂₇ is V, X₃₅ is A, X₃₉ is S, X₄₀ is G and X₄₁ is S.

In some embodiments, only one of X₁₇, X₂₀, X₂₄ and X₂₈ is an amino acid with a functional group available for conjugation to a C₁₆-C₂₂ fatty acid.

In some embodiments, only one of X₁₇, X₂₀, X₂₄ and X₂₈ is conjugated to a fatty acid optionally via a linker between the amino acid and the fatty acid. Thus, in some embodiments, only one of X₁₇, X₂₀, X₂₄ and X₂₈ is conjugated to a fatty acid via a direct bond between the amino acid and the fatty acid or via a linker between the amino acid and the fatty acid. In one embodiment, only one of X₁₇, X₂₀, X₂₄ and X₂₈ is conjugated to a fatty acid via a linker between the amino acid and the fatty acid. In some embodiments, the fatty acid is a C₁₆-C₂₂ fatty acid.

Thus, in one embodiment, X₁₇ is K and is conjugated to a C₁₆-C₂₂ fatty acid via a direct bond or via a linker between the amino acid and the C₁₆-C₂₂ fatty acid. In one embodiment, X₁₇ is K and is conjugated to a C₁₆-C₂₂ fatty acid via a linker between the amino acid and the C₁₆-C₂₂ fatty acid. In such embodiments, X₂₀ is Aib, αMe-4-pal, Q or R, X₂₄ is E, Q, D-Glu or N, and X₂₈ is E or A. In an embodiment, the conjugation is to the epsilon-amino group of the K side-chain.

In one embodiment, X₂₀ is K and is conjugated to a C₁₆-C₂₂ fatty acid via a direct bond or via a linker between the amino acid and the C₁₆-C₂₂ fatty acid. In one embodiment, X₂₀ is K and is conjugated to a C₁₆-C₂₂ fatty acid via linker between the amino acid and the C₁₆-C₂₂ fatty acid. In such embodiments, X₁₇ is A, I or Q, X₂₄ is E, Q, D-Glu or N, and X₂₈ is E or A. In an embodiment, the conjugation is to the epsilon-amino group of the K side-chain.

In one embodiment, X₂₄ is K and is conjugated to a C₁₆-C₂₂ fatty acid via a direct bond or via a linker between the amino acid and the C₁₆-C₂₂ fatty acid. In one embodiment, X₂₄ is K and is conjugated to a C₁₆-C₂₂ fatty acid via linker between the amino acid and the C₁₆-C₂₂ fatty acid. In such embodiments, X₁₇ is A, I or Q, X₂₀ is Aib, αMe-4-pal, Q or R, and X₂₈ is E or A. In an embodiment, the conjugation is to the epsilon-amino group of the K side-chain.

In one embodiment, X₂₈ is K and is conjugated to a C₁₆-C₂₂ fatty acid via a direct bond or via a linker between the amino acid and the C₁₆-C₂₂ fatty acid. In one embodiment, X₂₈ is K and is conjugated to a C₁₆-C₂₂ fatty acid via a linker between the amino acid and the C₁₆-C₂₂ fatty acid. In such embodiments, X₁₇ is A, I or Q, X₂₀ is Aib, αMe-4-pal, Q or R, and X₂₄ is E, Q, D-Glu or N. In an embodiment, the conjugation is to the epsilon-amino group of the K side-chain.

The amino acid sequences of polypeptides described herein incorporate naturally occurring amino acids, typically depicted herein using standard one letter codes (e.g., L=leucine), as well as alpha-methyl substituted residues of natural amino acids (e.g., α-methyl leucine (αMeL), and certain other non-natural amino acids, such as alpha amino isobutyric acid (Aib). The structures of these amino acids are depicted below:

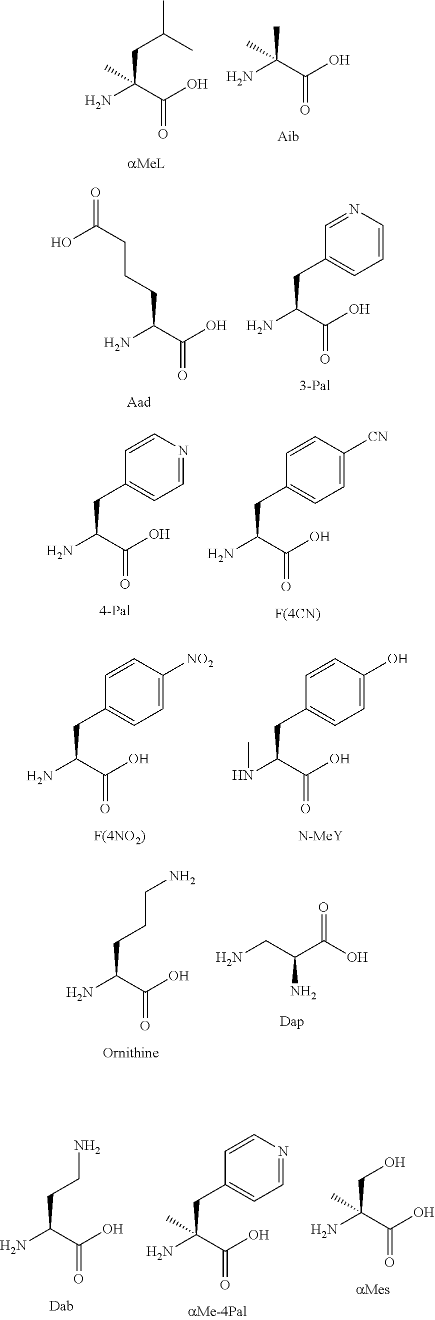

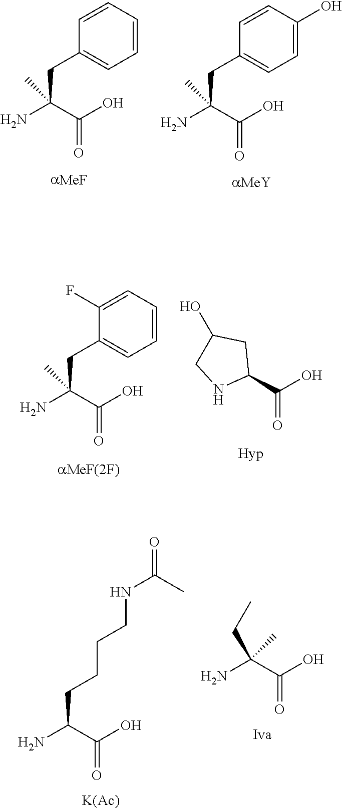

As used herein “Or” means L-ornithine. As used herein “4-Pal” or “4Pal” means 3-(4-Pyridyl)-L-alanine or (S)-2-amino-3-(pyridin-4-yl)propanoic acid. As used herein “3-Pal” or “3Pal” means 3-(3-Pyridyl)-L-alanine or (S)-2-amino-3-(pyridin-3-yl)propanoic acid. As used herein “αMe-4-Pal” or “αMe4Pal” means alpha-methyl-3-(4-Pyridyl)-L-alanine. As used herein “αMeY” means alpha-methyl-L-tyrosine. As used herein “αMeL” means alpha-methyl-leucine. As used herein “D-Ala” and “a” each means D-alanine. As used herein “D-Glu” and “e” each means D-glutamic acid. As used herein “Aib” means 2-Aminoisobutyric Acid. As used herein, “NMeY” means N-methyl-tyrosine. As used herein “Dap” means (S)-2,3-diaminopropanoic acid. As used herein “Dab” means (S)-2,4-diaminobutanoic acid. As used herein “Hyp” means Hydroxy-L-proline. As used herein “K(Ac)” means Ne-acetyl-L-lysine. As used herein “γGlu” means gamma L-glutamic acid. As used herein “Aad” means (S)-2-aminohexanedioic acid. As used herein “F(4CN)” means 4-cyano-L-phenylalanine or (S)-2-amino-3-(4-cyanophenyl)propanoic acid. As used herein “F(4NO₂)” means 4-nitro-L-phenylalanine or (S)-2-amino-3-(4-nitrophenyl)propanoic acid. As used herein “αMeS” means alpha-methyl-L-serine. As used herein “αMeF” means alpha-methyl-L-phenylalanine. As used herein “αMeF(2F)” means alpha-methyl-2-fluoro-L-phenylalanine or (S)-2-amino-3-(2-fluorophenyl)-2-methylpropanoic acid. As used herein “L-Iva” and “Iva” mean L-isovaline. As used herein “D-Gln” and “q” each means D-glutamine.

As noted before, in some embodiments, the polypeptides described herein include a fatty acid moiety conjugated, for example, by way of a direct bond or a linker to a natural or non-natural amino acid with a functional group available for conjugation. Such a conjugation is sometimes referred to as acylation. In certain instances, the amino acid with a functional group available for conjugation can be K, C, E and D. In particular instances, the amino acid with a functional group available for conjugation is K, where the conjugation is to an epsilon-amino group of a K side-chain.

The acylation of the polypeptides described herein is at position X₁₇ or X₂₀ or X₂₄ or X₂₈ in SEQ ID NO:4 or 6, or at position X₁₇ in SEQ ID NO:5. The fatty acid, and in certain embodiments the linker and/or amino acid sequence backbone, may act as albumin binders, and provide a potential to generate long-acting compounds.

In some embodiments, the polypeptides described herein utilize a C₁₆-C₂₂ fatty acid chemically conjugated to the functional group of an amino acid either via a direct bond or via a linker. The length and composition of the fatty acid impacts half-life of the polypeptides, their potency in in vivo animal models, and their solubility and stability. Conjugation to a C₁₆-C₂₂ saturated fatty monoacid or diacid results in polypeptides that exhibit desirable half-life, desirable potency in in vivo animal models, and desirable solubility and stability characteristics.

Examples of saturated C₁₆-C₂₂ fatty acids for use herein include, but are not limited to, palmitic acid (hexadecanoic acid) (C₁₆ monoacid), hexadecanedioic acid (C₁₆ diacid), margaric acid (heptadecanoic acid)(C₁₇ monoacid), heptadecanedioic acid (C₁₇ diacid), stearic acid (C₁₈ monoacid), octadecanedioic acid (C₁₈ diacid), nonadecylic acid (nonadecanoic acid)(C₁₉ monoacid), nonadecanedioic acid (C₁₉ diacid), arachadic acid (eicosanoic acid)(C₂₀ monoacid), eicosanedioic acid (C₂₀ diacid), heneicosylic acid (heneicosanoic acid)(C₂₁ monoacid), heneicosanedioic acid (C₂₁ diacid), behenic acid (docosanoic acid)(C₂₂ monoacid), docosanedioic acid (C₂₂ diacid), including branched and substituted derivatives thereof.

In certain instances, the C₁₆-C₂₂ fatty acid can be a saturated C₁₈ monoacid, a saturated C₁₈ diacid, a saturated C₁₉ monoacid, a saturated C₁₉ diacid, a saturated C₂₀ monoacid, a saturated C₂₀ diacid, and branched and substituted derivatives thereof. In more particular instances, the C₁₆-C₂₂ fatty acid can be octadecanedioic (C₁₈ diacid) or eicosanedioic acid (C₂₀ diacid).

In certain instances, the linker can have one or more (2-[2-(2-amino-ethoxy)-ethoxy]-acetyl) moieties or EK, optionally in combination with one to four amino acids.

In instances in which the linker includes at least one amino acid, the amino acid can be one to five Glu or γGlu amino acid residues. In some instances, the linker can include one or two or three or four or five Glu or γGlu amino acid residues, including the D-forms thereof. For example, the linker can include either one or two or three or four γGlu amino acid residues. Alternatively, the linker can include one to five amino acid residues (such as, for example, Glu or γGlu amino acids) used in combination with one to five (2-[2-(2-amino-ethoxy)-ethoxy]-acetyl) (“AEEA”) or one to five &K moieties. Specifically, the linker can be combinations of one to five Glu or γGlu amino acids and one to five (2-[2-(2-amino-ethoxy)-ethoxy]-acetyl) moieties, or one to five Glu or γGlu amino acids and one to five &K moieties. In some instances, the linker can be combinations of one or two or three γGlu amino acids and one or two (2-[2-(2-amino-ethoxy)-ethoxy]-acetyl) or EK moieties.

For example, in some embodiments the polypeptides described herein have linker and fatty acid components having the structure of the following formula:

(γGlu)ₐ-(2-[2-(2-amino-ethoxy)-ethoxy]-acetyl)_{b}-(γGlu)_{c}-CO—(CH₂)ₚ—CO₂H, where a is 0, 1 or 2; bis 0, 1 or 2; c is 0, 1, 2 or 3; and p is an integer between 14 to 20.

In some preferred embodiments, a is 0 or 1; b is 0, 1 or 2; c is 1, 2 or 3; and p an integer between 14 to 20.

In some embodiments, a is 0, b is 1, c is 1 or 2 and p is 16 or 18.

For example, in some embodiments, a is 0, bis 1, c is 1 and p is 16, the structure of which is depicted below.

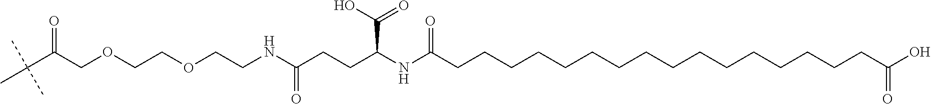

For example, in some embodiments, a is 0, bis 1, c is 1 and p is 18, the structure of which is depicted below.

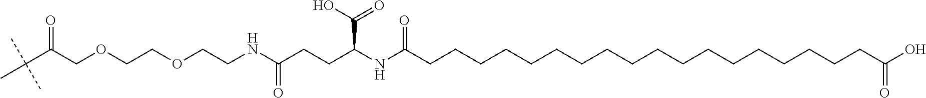

In some embodiments, a is 0, b is 1, c is 2 and p is 16, the structure of which is depicted below.

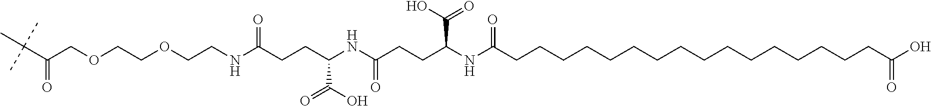

In some embodiments, a is 0, b is 1, c is 2 and p is 18, the structure of which is depicted below.

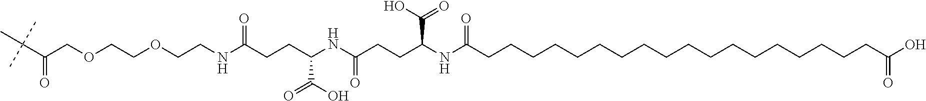

In some embodiments, a is 0, b is 2, c is 1 and p is 16 or 18.

For example, in some embodiments, a is 0, b is 2, c is 1 and p is 16, the structure of which is depicted below.

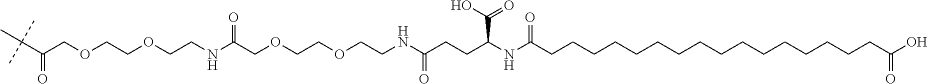

In some embodiments, a is 0, b is 2, c is 1 and p is 18, the structure of which is depicted below.

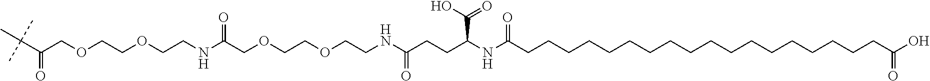

In some embodiments, a is 0, b is 0, c is 2 and p is 16 or 18.

For example, in some embodiments, a is 0, b is 0, c is 2 and p is 16, the structure of which is depicted below.

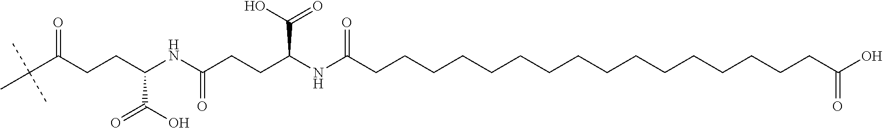

In some embodiments, a is 0, b is 0, c is 2 and p is 18, the structure of which is depicted below.

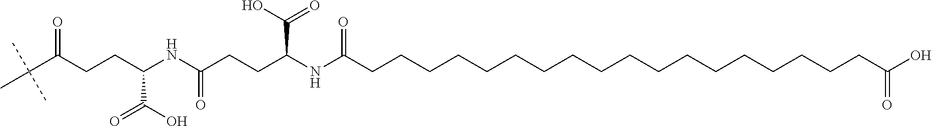

In some embodiments, a is 0, b is 0, c is 3 and p is 16 or 18.

For example, in some embodiments, a is 0, b is 0, c is 3 and p is 16, the structure of which is depicted below.

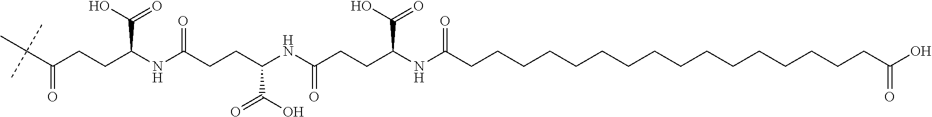

In some embodiments, a is 0, b is 0, c is 3 and p is 18, the structure of which is depicted below.

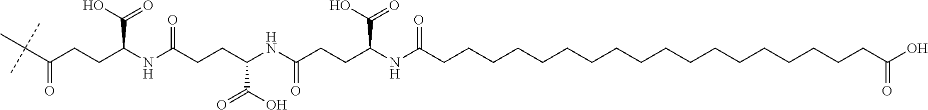

In some embodiments, a is 1, b is 1, c is 1 and p is 16 or 18.

For example, in some embodiments, a is 1, bis 1, c is 1 and p is 16, the structure of which is depicted below.

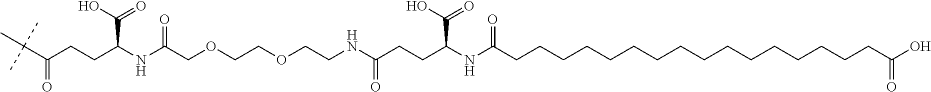

For example, in some embodiments, a is 1, b is 1, c is 1 and p is 18, the structure of which is depicted below.

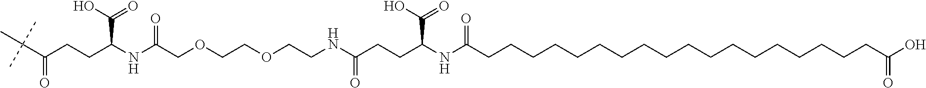

In some embodiments the polypeptides described herein have linker and fatty acid components having the structure of the following formula:

(γGlu)_{d}-(EK)ₑ-(γGlu)_{f}-CO—(CH₂)_{q}—CO₂H, where d is 0, 1 or 2; e is 0, 1 or 2; f is 0, 1, 2 or 3; and q is an integer between 14 to 20.

For example, in one embodiment, d is 0; e is 2; f is 1; and q an integer between 14 to 20. In some embodiments, d is 0; e is 2; f is 1; and q is 16 or 18.

For example, in some embodiments, d is 0; e is 2; f is 1; and q is 16, the structure of which is depicted below.

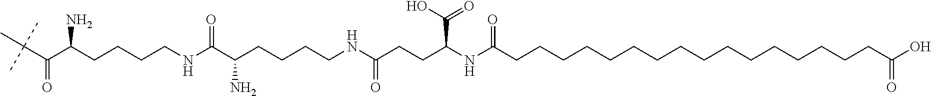

For example, in some embodiments, d is 0; e is 2; f is 1; and q is 18, the structure of which is depicted below.

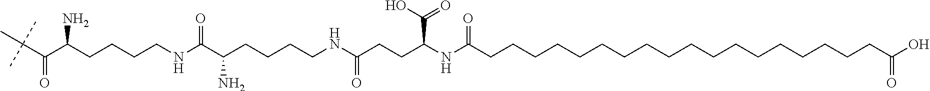

As shown in the chemical structures of Examples 1-1229 below, the linker-fatty acid moieties described above can be linked to amino acid present at positions 17, 20, 24 or 28. In some embodiments, a linker-fatty acid moiety described above is linked or conjugated to amino acid present at position 17, for example to the epsilon (ε)-amino group of the lysine (K) side-chain present at position 17. In some embodiments, a linker-fatty acid moiety described above is linked or conjugated to amino acid present at position 20, for example to the epsilon (ε)-amino group of the lysine (K) side-chain present at position 20. In some embodiments, a linker-fatty acid moiety described above is linked or conjugated to amino acid present at position 24, for example to the epsilon (ε)-amino group of the lysine (K) side-chain present at position 24. In some embodiments, a linker-fatty acid moiety described above is linked or conjugated to amino acid present at position 28, for example to the epsilon (8)-amino group of the lysine (K) side-chain present at position 28.

In some embodiments, the polypeptides described herein comprise a sequence selected from any one of SEQ ID NO'S: 7 to 1242 (described below in examples 1-1236). In some embodiments, the polypeptides described herein consist of a sequence selected from any one of SEQ ID NO'S: 7 to 1242 (described below in examples 1-1236).

In some embodiments, the polypeptides described herein are amidated. In some embodiments, the polypeptides described herein have a modification of the C-terminal group, wherein the modification is NH₂ or absent. In some embodiments, the polypeptides described herein have an OH group at the C-terminal.

In addition to the sequences described herein, the polypeptides described herein may include one or more conservative amino acid substitutions, provided, however, that the polypeptides remain capable of binding to and activating GIP, GLP-1 and Glucagon receptors.

In certain embodiments of polypeptides of any of the formulas described herein, the polypeptide is an isotopic derivative of any one of the polypeptides described herein or a pharmaceutically acceptable salt thereof. It is understood that the isotopic derivative can be prepared using any of a variety of art-recognized techniques. For example, the isotopic derivatives can generally be prepared by carrying out the procedures disclosed in the examples described herein by substituting an isotopically labeled reagent for a non-isotopically labeled reagent. In an embodiment of a polypeptide of any of the formulas described herein, or a pharmaceutically acceptable salt thereof, the polypeptide is a deuterated derivative of any one of the polypeptides described herein.

In the polypeptides of this invention any atom not specifically designated as a particular isotope is meant to represent any stable isotope of that atom. Unless otherwise stated, when an atom is designated specifically as “H” or “hydrogen”, the atom is understood to have hydrogen at its natural abundance isotopic composition. Also, unless otherwise stated, when an atom is designated specifically as “D” or “deuterium”, the atom is understood to have deuterium at an abundance substantially greater than the natural abundance of deuterium, which is 0.015%.

The affinity of the polypeptides described herein for each of the GIP, GLP-1 and glucagon receptors may be measured using techniques known in the art for measuring receptor binding levels and is commonly expressed as an inhibitory constant (Ki) value. The activity of the polypeptides described herein at each of the receptors also may be measured using techniques known in the art, including, for example, the in vitro activity assays described below, and is commonly expressed as an effective concentration 50 (EC 50) value, which is the concentration of compound causing half-maximal simulation in a dose response curve.

The polypeptides described herein may react with any number of inorganic and organic acids/bases to form pharmaceutically acceptable acid/base addition salts. Pharmaceutically acceptable salts and common techniques for preparing them are well known in the art (see. e.g., Stahl et al., Handbook of Pharmaceutical Salts: Properties, Selection and Use, 2^{nd} Revised Edition (Wiley-VCH, 2011)). Pharmaceutically acceptable salts for use herein include sodium, potassium, trifluoroacetate, hydrochloride and/or acetate salts. Thus, in some embodiments, provided herein are pharmaceutically acceptable salt forms of the GGG polypeptides. In some embodiments, the pharmaceutically acceptable forms are selected from sodium or potassium salts. In some embodiments, the pharmaceutically acceptable forms are selected from the group consisting of sodium, potassium salts. In some preferred embodiments, a pharmaceutically acceptable salt is a sodium salt.

The polypeptides described herein are suitable for administration by a parenteral route (e.g., subcutaneous, intravenous, intraperitoneal, intramuscular or transdermal) or oral route (e.g., tablet, capsule). In some preferred embodiments, the polypeptides described herein are suitable for oral administration. The in vitro permeability (Papp) assay and in vivo ileum absorption assay described herein are useful tools for assessing the potential for oral delivery of a polypeptide.

In another embodiment, provided herein is a pharmaceutical composition comprising a polypeptide described herein, or a pharmaceutically acceptable salt thereof, and a pharmaceutically acceptable carrier, diluent, or excipient. Some pharmaceutical compositions and techniques for preparing the same are well known in the art. See, e.g., Remington: The Science and Practice of Pharmacy (Troy, Ed., 21^{st} Edition, Lippincott, Williams & Wilkins, 2006).

In some embodiments, the pharmaceutical composition is suitable for administration by a parenteral route (e.g., subcutaneous, intravenous, intraperitoneal, intramuscular or transdermal). In some embodiments, the pharmaceutical composition is suitable for oral administration (e.g., tablet, capsule). In some embodiments, the pharmaceutical composition is administered parenterally. In some embodiments, the pharmaceutical composition is administered orally.

Physiochemical properties of a polypeptide in addition to anatomical and physiological features of the gastrointestinal tract may pose challenges to efficient oral delivery of a peptide. In an embodiment a pharmaceutical composition for oral administration comprises a polypeptide described herein or a pharmaceutically acceptable salt thereof, and a permeation enhancer. In an embodiment, a pharmaceutical composition for oral administration comprises polypeptide described herein or a pharmaceutically acceptable salt thereof, a permeation enhancer, and a protease inhibitor.

As used herein the term “permeation enhancer” means permeation enhancer that enhances oral absorption of a polypeptide of this invention. As used herein, permeation enhancer means permeation enhancers, such as sodium decanoate (C10), sodium taurodeoxycholate (NaTDC), lauroyl carnitine (LC), dodecyl maltoside, dodecyl phosphatidylcholine, SNAC, a Rhamnolipid, and permeation enhancers reported in the literature, such as for example, Permeant inhibitor of phosphatase, PIP-250 and PIP-640. See. Pharmaceutics. 2019 January: 11(1): 41, (See Biomaterials. 2012; 33; 3464-3474), ZOT (zonula occludens toxin), ΔG (fragment of ZOT) (See Int. J. Pharm. 2009; 365, 121-130). In an embodiment, a permeation enhancer is selected from sodium decanoate, sodium taurodeoxycholate, and lauroyl carnitine. In an embodiment, a permeation enhancer is selected from the group consisting of C10, LC, or NaTDC. In an embodiment, a permeation enhancer is selected from the group consisting of sodium decanoate, sodium taurodeoxycholate, and lauroyl carnitine. In an embodiment, a permeation enhancer is selected from the group consisting of C10, LC, and NaTDC.

As used herein the term “protease inhibitor” means a protease inhibitor that may be selected from the group consisting of protein based, peptide based, and small molecule based. Protease inhibitors are well known and may include, for example, soy bean trypsin inhibitor (“SBTI”), soybean trypsin-chymotrypsin inhibitor (“SBTCI”), ecotin, sunflower trypsin inhibitor (“SFTI”), leupeptin, citric acid, ethylenediaminetetraacetic acid (“EDTA”), sodium glycocholate and 4-(2-aminoethyl) benzenesulfonyl fluoride hydrochloride (“AEBSF”). In an embodiment a protease inhibitor is selected from the group consisting of SBTI, SBTCI and SFTI. In an embodiment, a protease inhibitor is SBTI.

The disclosure also provides and therefore encompasses novel intermediates and methods of synthesizing the polypeptides described herein, or pharmaceutically acceptable salts thereof. The intermediates and polypeptides described herein can be prepared by a variety of techniques known in the art. For example, a method using chemical synthesis is illustrated in the Examples below or using biological expression. The specific synthetic steps for each of the routes described may be combined in different ways to prepare the polypeptides described herein. The reagents and starting materials are readily available to one of skill in the art.

With respect to chemical synthesis, one can use standard manual or automated solid-phase synthesis procedures. For example, automated peptide synthesizers are commercially available from, for example, CEM (Charlotte, North Carolina), CSBio (Menlo Park, California) and Gyros Protein Technologies Inc. (Tucson, AZ). Reagents for solid-phase synthesis are readily available from commercial sources. Solid-phase synthesizers can be used according to the manufacturer's instructions for blocking interfering groups, protecting amino acids during reaction, coupling, deprotecting and capping of unreacted amino acids.

With respect to biological expression, one can use standard recombinant techniques to construct a polynucleotide having a nucleic acid sequence that encodes an amino acid sequence for all or part of a polypeptide, incorporate that polynucleotide into recombinant expression vectors, and introduce the vectors into host cells, such as bacteria, yeast and mammalian cells, to produce the polypeptide. See, e.g., Green & Sambrook, “Molecular Cloning: A Laboratory Manual” (Cold Spring Harbor Laboratory Press, 4th ed. 2012). The polypeptides may readily be produced in mammalian cells such as CHO, NSO, 20 HEK293, BHK, or COS cells: in bacterial cells such as E. coli, Bacillus subtilis, or Pseudomonas fluorescens: in insect cells, or in fungal or yeast cells, which are cultured using techniques known in the art. The vectors containing the polynucleotide sequences of interest can be transferred into the host cell by well-known methods, which vary depending on the type of cellular host. Various methods of protein purification may be employed and such methods are known in the art.

The polypeptides described herein may be used for treating a variety of conditions, disorders, diseases or symptoms. In particular, methods are provided for treating obesity in an individual, where such methods include at least a step of administering to an individual in need of such treatment an effective amount of a polypeptide described herein, or a pharmaceutically acceptable salt thereof.

Additionally, methods are provided for chronic weight management in an individual, where such methods include at least a step of administering to an individual in need of such treatment an effective amount of a polypeptide described herein, or a pharmaceutically acceptable salt thereof.

Additionally, methods are provided for treating type 2 diabetes mellitus (T2DM) in an individual, where such methods include at least a step of administering to an individual in need of such treatment an effective amount of a polypeptide described herein, or a pharmaceutically acceptable salt thereof.

Additionally, methods are provided for treating non-alcoholic fatty liver disease (NAFLD) in an individual, where such methods include at least a step of administering to an individual in need of such treatment an effective amount of a polypeptide described herein, or a pharmaceutically acceptable salt thereof.

Additionally, methods are provided for treating non-alcoholic steatohepatitis (NASH) in an individual, where such methods include at least a step of administering to an individual in need of such treatment an effective amount of a polypeptide described herein, or a pharmaceutically acceptable salt thereof.

Additionally, methods are provided for treating dyslipidemia in an individual, where such methods include at least a step of administering to an individual in need of such treatment an effective amount of a polypeptide described herein, or a pharmaceutically acceptable salt thereof.

Additionally, methods are provided for treating metabolic syndrome in an individual, where such methods include at least a step of administering to an individual in need of such treatment an effective amount of a polypeptide described herein, or a pharmaceutically acceptable salt thereof.

Additionally, methods are provided for treating osteoarthritis (OA) in an individual, where such methods include at least a step of administering to an individual in need of such treatment an effective amount of a polypeptide described herein, or a pharmaceutically acceptable salt thereof.

Additionally, methods are provided for treating obesity-related sleep apnea (OSA) in an individual, where such methods include at least a step of administering to an individual in need of such treatment an effective amount of a polypeptide described herein, or a pharmaceutically acceptable salt thereof.

Additionally, methods are provided for treating polycystic ovary syndrome (PCOS) in an individual, where such methods include at least a step of administering to an individual in need of such treatment an effective amount of a polypeptide described herein, or a pharmaceutically acceptable salt thereof.

Additionally, methods are provided for inducing non-therapeutic weight loss in an individual, where such methods include at least a step of administering to an individual in need of such treatment an effective amount of a polypeptide described herein, or a pharmaceutically acceptable salt thereof.

In these methods, effectiveness of the polypeptides can be assessed by, for example, observing a significant reduction in blood glucose, observing a significant increase in insulin, observing a significant reduction in HbAlc and/or observing a significant reduction in body weight.

Alternatively, the polypeptides described herein or pharmaceutically acceptable salts thereof may be used for improving bone strength in an individual in need thereof. In some instances, the individual in need thereof has hypo-ostosis or hypo-osteoidosis, or is healing from bone fracture, orthotic procedure, prosthetics implant, dental implant, and/or spinal fusion. The polypeptides described herein also may be used for treating other disorders such as Parkinson's disease or Alzheimer's disease.

Additionally, provided is a polypeptide described herein, or a pharmaceutically acceptable salt thereof, for use in therapy. In some embodiments, provided herein is a polypeptide described herein or a pharmaceutically acceptable salt thereof, for use in treating obesity, chronic weight management, type 2 diabetes mellitus, non-alcoholic fatty liver disease (NAFLD), non-alcoholic steatohepatitis (NASH), dyslipidemia, metabolic syndrome, osteoarthritis (OA), obesity-related sleep apnea (OSA) and polycystic ovary syndrome (PCOS). Also provided is a use of a polypeptide described herein, or a pharmaceutically acceptable salt thereof, for inducing non-therapeutic weight loss.

Additionally, provided is a use of a polypeptide described herein, or a pharmaceutically acceptable salt thereof, in the manufacture of a medicament for treating obesity, chronic weight management, type 2 diabetes mellitus, non-alcoholic fatty liver disease (NAFLD), non-alcoholic steatohepatitis (NASH), dyslipidemia, metabolic syndrome, osteoarthritis (OA), obesity-related sleep apnea (OSA) and polycystic ovary syndrome (PCOS). Also provided is a use of a polypeptide described herein, or a pharmaceutically acceptable salt thereof, in the manufacture of a medicament for inducing non-therapeutic weight loss.

The polypeptides or pharmaceutical compositions described herein may be provided as part of a kit. In some instances, the kit includes a device for administering at least one polypeptide (and optionally at least one additional therapeutic agent) to an individual, such as a syringe, automatic injector or pump.

Additional non-limiting embodiments are set forth below:
1. A polypeptide comprising:

X₁X₂QGTX₆TSDX₁₀X₁₁X₁₂X₁₃LDX₁₆X₁₇AX₁₉X₂₀X₂₁FX₂₃X₂₄
X₂₅LX₂₇X₂₈X₂₉X₃₀X₃₁SSX₃₄X₃₅X₃₆X₃₇X₃₈X₃₉X₄₀X₄₁X₄₂,

wherein:
   X₁ is Y, NMeY or H,
   X₂ is Aib,
   X₆ is F, αMeF or αMeF(2F),
   X₁₀ is F, 4-Pal, F(4CN), 3-Pal, F(4NO₂) or Y,
   X₁₁ is S or αMeS,
   X₁₂ is Orn, K, R, Q, Dap, Dab, S, E or I,
   X₁₃ is αMeL, I or L,
   X₁₆ is K, Orn, A or E,
   X₁₇ is any amino acid with a functional group available for conjugation to a fatty acid, A, I or Q,
   X₁₉ is Q or A,
   X₂₀ is any amino acid with a functional group available for conjugation to a fatty acid, Aib, αMe-4-Pal, Q or R,
   X₂₁ is A, Q, Orn, Aad, Aib, S, N, E or T,
   X₂₃ is I or V,
   X₂₄ is any amino acid with a functional group available for conjugation to a fatty acid, E, Q, D-Glu or N,
   X₂₅ is W, Y, F, 4-Pal, αMeY or αMe-4-Pal,
   X₂₇ is L, I, E, V, A, Q or S,
   X₂₈ is any amino acid with a functional group available for conjugation to a fatty acid, E or A,
   X₂₉ is G, D-Ala, Aib, T or A,
   X₃₀ is A or G,
   X₃₁ is P, H, S, 4-Pal, E, T, K(Ac) or Hyp,
   X₃₄ is G or Aib,
   X₃₅ is A, Aib, E, H or 4-Pal,
   X₃₆ is P or Hyp,
   X₃₇ is P, Hyp or E,
   X₃₈ is P or Hyp,
   X₃₉ is E, S, G, T, H, 4-Pal, γE or A,
   X₄₀ is absent or G, E, S, A or T,
   wherein if X₄₀ is G, E, S, A or T, then X₄₁ is absent or E, S, T, 4-Pal, D, G, Q or H,
   wherein if X₄₁ is E, S, T, 4-Pal, D, G, Q or H, then X₄₂ is absent or G, E or γE
   wherein if X₄₀ is absent, then X₄₁ and X₄₂ are also absent,
   wherein if X₄₁ is absent, then X₄₂ is also absent,
   wherein at least one of X₁₇, X₂₀, X₂₄ and X₂₈ is an amino acid with a functional group available for conjugation to a fatty acid,
   wherein the C-terminal amino acid is optionally amidated;
   or a pharmaceutically acceptable salt thereof.
2. The polypeptide of embodiment 1, or a pharmaceutically acceptable salt thereof, wherein
   X₄₀ is G, E, S, A or T,
   X₄₁ is absent, and
   X₄₂ is absent.
3. The polypeptide of embodiment 1, or a pharmaceutically acceptable salt thereof, wherein
   X₄₀ is G, E, S, A or T,
   X₄₁ is E, S, T, 4-Pal, D, G, Q or H, and
   X₄₂ is absent.
4. The polypeptide of embodiment 1, wherein
   X₄₀ is G, E, S, A or T,
   X₄₁ is E, S, T, 4-Pal, D, G, Q or H, and
   X₄₂ is G, E or γE.
5. The polypeptide of any one of embodiments 1 to 4, wherein the amino acid at position X₁₇, X₂₀, X₂₄ or X₂₈ with a functional group available for conjugation to a fatty acid is K, C, E or D.
6. The polypeptide of embodiment 5, wherein the amino acid at position X₁₇, X₂₀, X₂₄ or X₂₈ with a functional group available for conjugation to a fatty acid is K.
7. The polypeptide of any one of embodiments 1 to 6, or a pharmaceutically acceptable salt thereof, wherein X₁₀ is F or 4-Pal.
8. The polypeptide of any one of embodiments 1 to 7, or a pharmaceutically acceptable salt thereof, wherein X₁₂ is Om, K, R or Q.
9. The polypeptide of any one of embodiments 1 to 8, or a pharmaceutically acceptable salt thereof, wherein only one of X₁₇, X₂₀, X₂₄ and X₂₈ is an amino acid with a functional group available for conjugation to a fatty acid.
10. The polypeptide of any one of embodiments 1 to 9, or a pharmaceutically acceptable salt thereof, wherein only one of X₁₇, X₂₀, X₂₄ and X₂₈ is conjugated to a fatty acid via a direct bond or via a linker between the amino acid and the fatty acid.
11. The polypeptide of embodiment 10, or a pharmaceutically acceptable salt thereof, wherein only one of X₁₇, X₂₀, X₂₄ and X₂₈ is conjugated to a fatty acid via a linker between the amino acid and the fatty acid.
12. The polypeptide of any one of embodiments 1 to 11, wherein the fatty acid is a C₁₆-C₂₂ fatty acid.
13. The polypeptide of embodiment 12, or a pharmaceutically acceptable salt thereof, wherein
   X₁₇ is K and is conjugated to a C₁₆-C₂₂ fatty acid via a direct bond or via a linker between the amino acid and the C₁₆-C₂₂ fatty acid,
   X₂₀ is Aib, αMe-4-Pal, Q or R,
   X₂₄ is E, Q, D-Glu or N, and
   X₂₈ is E or A.
14. The polypeptide of embodiment 12, or a pharmaceutically acceptable salt thereof, wherein
   X₁₇ is A, I or Q,
   X₂₀ is K and is conjugated to the C₁₆-C₂₂ fatty acid via a direct bond or via a linker between the amino acid and the C₁₆-C₂₂ fatty acid,
   X₂₄ is E, Q, D-Glu or N, and
   X₂₈ is E or A.
15. The polypeptide of embodiment 12, or a pharmaceutically acceptable salt thereof, wherein
   X₁₇ is A, I or Q,
   X₂₀ is Aib, αMe-4-Pal, Q or R,
   X₂₄ is K and is conjugated to the C₁₆-C₂₂ fatty acid via a direct bond or via a linker between the amino acid and the C₁₆-C₂₂ fatty acid, and
   X₂₈ is E or A.
16. The polypeptide of embodiment 12, or a pharmaceutically acceptable salt thereof, wherein
   X₁₇ is A, I or Q,
   X₂₀ is Aib, αMe-4-Pal, Q or R,
   X₂₄ is E, Q, D-Glu or N, and
   X₂₈ is K and is conjugated to the C₁₆-C₂₂ fatty acid via a direct bond or via a linker between the amino acid and the C₁₆-C₂₂ fatty acid.
17. The polypeptide of embodiment 1 comprising:

YX₂QGTFTSDX₁₀SX₁₂X₁₃LDX₁₆X₁₇AQX₂₀X₂₁FIX₂₄X₂₅LX₂₇
X₂₈X₂₉X₃₀X₃₁SSX₃₄X₃₅X₃₆X₃₇X₃₈X₃₉X₄₀X₄₁X₄₂,

wherein
   X₂ is Aib,
   X₁₀ is F, 4-Pal or F(4CN),
   X₁₂ is Orn, K, R, Q, Dap or Dab,
   X₁₃ is αMeL,
   X₁₆ is K or Orn,
   X₁₇ is any amino acid with a functional group available for conjugation to a fatty acid,
   X₂₀ is Aib, αMe-4-Pal or Q,
   X₂₁ is A, Q or Orn,
   X₂₄ is E or Q,
   X₂₅ is W, Y, 4-Pal, αMeY or αMe-4-Pal,
   X₂₇ is L or I,
   X₂₈ is E or A,
   X₂₉ is G, D-Ala or Aib,
   X₃₀ is A or G,
   X₃₁ is P, H, S, 4-Pal, E, T, K(Ac) or Hyp,
   X₃₄ is G or Aib,
   X₃₅ is A, Aib, E, H or 4-Pal,
   X₃₆ is P or Hyp,
   X₃₇ is P or Hyp,
   X₃₈ is P or Hyp,
   X₃₉ is E, S, G, T, H, 4-Pal or γE,
   X₄₀ is absent or G, E or S,
   wherein if X₄₀ is G, E or S, then X₄₁ is absent or E, S, T, 4-Pal, D, G, Q or H,
   wherein if X₄₁ is E, S, T, 4-Pal, D, G, Q or H, then X₄₂ is absent or G, E or γE,
   wherein the C-terminal amino acid is optionally amidated;
   or a pharmaceutically acceptable salt thereof.
18. The polypeptide of embodiment 17, or a pharmaceutically acceptable salt thereof, wherein
X₄₀ G, E or S,
X₄₁ is absent, and X₄₂ is absent.
19. The polypeptide of embodiment 17, or a pharmaceutically acceptable salt thereof, wherein
   X₄₀ G, E or S,
   X₄₁ is E, S, T, 4-Pal, D, G, Q or H, and
   X₄₂ is absent.
20. The polypeptide of embodiment 17, or a pharmaceutically acceptable salt thereof, wherein
   X₄₀ G, E or S,
   X₄₁ is E, S, T, 4-Pal, D, G, Q or H, and
   X₄₂ is G, E or γE.
21. The polypeptide of any one of embodiments 17 to 20, wherein X₁₇ is K, C, E or D.
22. The polypeptide of embodiment 21, wherein X₁₇ is K.
23. The polypeptide of any one of embodiments 17 to 22, or a pharmaceutically acceptable salt thereof, wherein X₁₀ is F or 4-Pal.
24. The polypeptide of any one of embodiments 17 to 23, or a pharmaceutically acceptable salt thereof, wherein X₁₂ is Om, K, R or Q.
25. The polypeptide of any one of embodiments 17 to 24, or a pharmaceutically acceptable salt thereof, wherein X₁₇ is K and is conjugated to a fatty acid via a direct bond or via a linker between the amino acid and the fatty acid.
26. The polypeptide of embodiment 25, or a pharmaceutically acceptable salt thereof, wherein X₁₇ is K and is conjugated to a fatty acid via a linker between the amino acid and the fatty acid.
27. The polypeptide of any one of embodiments 17 to 26, wherein the fatty acid is a C₁₆-C₂₂ fatty acid.
28. The polypeptide of embodiment 27, or a pharmaceutically acceptable salt thereof, wherein X₁₇ is K and is conjugated to a C₁₆-C₂₂ fatty acid via a direct bond or via a linker between the amino acid and the C₁₆-C₂₂ fatty acid.
29. The polypeptide of embodiment 1 comprising:

X₁X₂QGTX₆TSDX₁₀X₁₁X₁₂X₁₃LDX₁₆X₁₇AX₁₉X₂₀X₂₁FX₂₃X₂₄
X₂₅LX₂₇X₂₈X₂₉GX₃₁SSX₃₄X₃₅X₃₆X₃₇X₃₈X₃₉X₄₀X₄₁,

wherein
   X₁ is Y, NMeY or H,
   X₂ is Aib,
   X₆ is F, αMeF or αMeF(2F),
   X₁₀ is F, 3-Pal, 4-Pal, F(4CN), F(4NO₂) or Y,
   X₁₁ is S or αMeS,
   X₁₂ is Orn, K, R, Q, Dap, S, E or I,
   X₁₃ is αMeL, I or L,
   X₁₆ is K, Om, A or E,
   X₁₇ is any amino acid with a functional group available for conjugation to a fatty acid, A, I or Q,
   X₁₉ is A or Q,
   X₂₀ is any amino acid with a functional group available for conjugation to a fatty acid Aib, Q, R or αMe-4-Pal,
   X₂₁ is A, Aad, Aib, S, N, Q, E, T or Orn,
   X₂₃ is I or V,
   X₂₄ is any amino acid with a functional group available for conjugation to a fatty acid, E, D-Glu, Q or N,
   X₂₅ is W, Y, F, 4-Pal, αMeY or αMe-4-Pal,
   X₂₇ is L, I, E, V, A, Q or S,
   X₂₈ is any amino acid with a functional group available for conjugation to a fatty acid, E or A,
   X₂₉ is G, Aib, T, D-Ala or A,
   X₃₁ is P or E,
   X₃₄ is G or Aib,
   X₃₅ is A or E,
   X₃₆ is P,
   X₃₇ is P or E,
   X₃₈ is P,
   X₃₉ is E, S, G or A,
   X₄₀ is absent or G, E, S, A or T,
   wherein if X₄₀ is G, E, S, A or T, then X₄₁ is absent or is E, S, D or G,
   wherein if X₄₁ is E, S, D or G, then X₄₂ is absent or G, E or γE,
   wherein if X₁₀ is F, 3-Pal, 4-Pal, F(4CN), F(4NO₂), then X₁₂ is I,
   wherein at least one of X₁₇, X₂₀, X₂₄ or X₂₈ is an amino acid with a functional group available for conjugation to a fatty acid,
   wherein the C-terminal amino acid is optionally amidated;
or a pharmaceutically acceptable salt thereof.
30. The polypeptide of embodiment 29, or a pharmaceutically acceptable salt thereof, wherein:
   X₄₀ G, E, S, A or T,
   X₄₁ is absent and
   X₄₂ is absent.
31. The polypeptide of embodiment 29, or a pharmaceutically acceptable salt thereof, wherein:
   X₄₀ G, E, S, A or T,
   X₄₁ is E, S, D or G and
   X₄₂ is absent.
32. The polypeptide of embodiment 29, or a pharmaceutically acceptable salt thereof, wherein:
   X₄₀ G, E, S, A or T,
   X₄₁ is E, S, D or G and
   X₄₂ is G, E or γE.
33. The polypeptide of any one of embodiments 29 to 32, wherein the amino acid at position X₁₇, X₂₀, X₂₄ or X₂₈ with a functional group available for conjugation to a fatty acid is K, C, E or D.
34. The polypeptide of embodiment 5, wherein the amino acid at position X₁₇, X₂₀, X₂₄ or X₂₈ with a functional group available for conjugation to a fatty acid is K.
35. The polypeptide of any one of embodiments 29 to 34, or a pharmaceutically acceptable salt thereof, wherein X₁₀ is F, 3-Pal, 4-Pal, F(4CN) or F(4NO₂) and X₁₂ is I.
36. The polypeptide of any one of embodiments 29 to 34, or a pharmaceutically acceptable salt thereof, wherein X₁₀ is Y and X₁₂ is Om, K, R, Q, Dap, S, E or I.
37. The polypeptide of any one of embodiments 29 to 36, or a pharmaceutically acceptable salt thereof, wherein only one of X₁₇, X₂₀, X₂₄ or X₂₈ is an amino acid with a functional group available for conjugation to a fatty acid.
38. The polypeptide of any one of embodiments 29 to 37, or a pharmaceutically acceptable salt thereof, wherein only one of X₁₇, X₂₀, X₂₄ or X₂₈ is conjugated to a fatty acid via a direct bond or via a linker between the amino acid and the fatty acid.
39. The polypeptide of embodiment 38, or a pharmaceutically acceptable salt thereof, wherein only one of X₁₇, X₂₀, X₂₄ or X₂₈ is conjugated to a fatty acid via a linker between the amino acid and the fatty acid.
40. The polypeptide of any one of embodiments 29 to 39, wherein the fatty acid is a C₁₆-C₂₂ fatty acid.
41. The polypeptide of embodiment 40, or a pharmaceutically acceptable salt thereof, wherein
   X₁₇ is K and is conjugated to a C₁₆-C₂₂ fatty acid via a direct bond or via a linker between the amino acid and the C₁₆-C₂₂ fatty acid,
   X₂₀ is Aib, αMe-4-Pal, Q or R,
   X₂₄ is E, Q, D-Glu or N, and
   X₂₈ is E or A.
42. The polypeptide of embodiment 40, or a pharmaceutically acceptable salt thereof, wherein:
   X₁₇ is A, I or Q,
   X₂₀ is K and is conjugated to a C₁₆-C₂₂ fatty acid via a direct bond or via a linker between the amino acid and the C₁₆-C₂₂ fatty acid,
   X₂₄ is E, Q, D-Glu or N, and
   X₂₈ is E or A.
43. The polypeptide of embodiment 40, or a pharmaceutically acceptable salt thereof, wherein:
   X₁₇ is A, I or Q,
   X₂₀ is Aib, αMe-4-Pal, Q or R,
   X₂₄ is K and is conjugated to a C₁₆-C₂₂ fatty acid via a direct bond or via a linker between the amino acid and the C₁₆-C₂₂ fatty acid, and
   X₂₈ is E or A.
44. The polypeptide of embodiment 40, or a pharmaceutically acceptable salt thereof, wherein:
   X₁₇ is A, I or Q,
   X₂₀ is Aib, αMe-4-Pal, Q or R,
   X₂₄ is E, Q, D-Glu or N, and
   X₂₈ is K and is conjugated to a C₁₆-C₂₂ fatty acid via a direct bond or via a linker between the amino acid and the C₁₆-C₂₂ fatty acid.
45. The polypeptide of any one of embodiments 10, 11, 12, 13, 14, 15, 16, 25, 26, 27, 28, 38, 39, 40, 41, 42, 43 and 44, or a pharmaceutically acceptable salt thereof, wherein the linker comprises one to four amino acids.
46. The polypeptide of embodiment 45, or a pharmaceutically acceptable salt thereof, wherein the amino acids are Glu, γGlu or a combination thereof.
47. The polypeptide of embodiment 45 or 46, or a pharmaceutically acceptable salt thereof, wherein the linker comprises one to four (2-[2-(2-amino-ethoxy)-ethoxy]-acetyl) or EK moieties.
48. The polypeptide of embodiment 47, or a pharmaceutically acceptable salt thereof, wherein the linker comprises one to four (2-[2-(2-amino-ethoxy)-ethoxy]-acetyl) moieties.
49. The polypeptide of embodiment 45, or a pharmaceutically acceptable salt thereof, wherein the linker comprises a structure of (γGlu)ₐ-(2-[2-(2-amino-ethoxy)-ethoxy]-acetyl)_{b}-(γGlu)_{c}-CO—(CH₂)ₚ—CO₂H, wherein a is 0 or 1, b is 0, 1 or 2, c is 1, 2 or 3, and p is an integer between 14 to 20.
50. The polypeptide of embodiment 49, or a pharmaceutically acceptable salt thereof, wherein a is 0.
51. The polypeptide of embodiment 49, or a pharmaceutically acceptable salt thereof, wherein a is 1.
52. The polypeptide of any one of embodiments 49 to 51, or a pharmaceutically acceptable salt thereof, wherein b is 0.
53. The polypeptide of any one of embodiments 49 to 51, or a pharmaceutically acceptable salt thereof, wherein b is 1.
54. The polypeptide of any one of embodiments 49 to 51, or a pharmaceutically acceptable salt thereof, wherein b is 2.
55. The polypeptide of any one of embodiments 49 to 54, or a pharmaceutically acceptable salt thereof, wherein c is 1.
56. The polypeptide of any one of embodiments 49 to 54, or a pharmaceutically acceptable salt thereof, wherein c is 2.
57. The polypeptide of any one of embodiments 49 to 54, or a pharmaceutically acceptable salt thereof, wherein c is 3.
58. The polypeptide of any one of embodiments 49 to 57, or a pharmaceutically acceptable salt thereof, wherein p is 16.
59. The polypeptide of any one of embodiments 49 to 57, or a pharmaceutically acceptable salt thereof, wherein p is 18.
60. A polypeptide selected from the group consisting of SEQ ID NO's: 7 to 504, or a pharmaceutically acceptable salt thereof.
61. The polypeptide of any one of embodiments 1 to 60, or a pharmaceutically acceptable salt thereof, wherein the C-terminal is amidated.
62. The polypeptide of any one of embodiments 1 to 61, or a pharmaceutically acceptable salt thereof, wherein the pharmaceutically acceptable salt is selected from sodium, potassium, trifluoroacetate, hydrochloride or acetate.
63. The polypeptide of embodiment 62, or a pharmaceutically acceptable salt thereof, wherein the pharmaceutically acceptable salt is selected from sodium and potassium.
64. A pharmaceutical composition comprising the polypeptide or a pharmaceutically acceptable salt thereof, of any one of embodiments 1 to 63 and at least one pharmaceutically acceptable carrier, diluent, or excipient.
65. The pharmaceutical composition of embodiment 64, wherein the composition is formulated for oral administration.
66. The pharmaceutical composition of embodiment 64, wherein the composition is formulated for subcutaneous administration.
67. A method of treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA) and polycystic ovary syndrome (PCOS), the method comprising a step of:
administering to an individual in need thereof an effective amount of a polypeptide, or a pharmaceutically acceptable salt thereof, of any one of embodiments 1 to 63.
68. The polypeptide, or a pharmaceutically acceptable salt thereof, as described in by any one of Embodiments 1 to 63 for use in therapy.
69. A polypeptide, or a pharmaceutically acceptable salt thereof, as described in any one of Embodiments 1 to 63 for use in treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA) and polycystic ovary syndrome (PCOS).
70. Use of a polypeptide, or a pharmaceutically acceptable salt thereof, as described in any one of Embodiments 1 to 63 in the manufacture of a medicament for treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA) and polycystic ovary syndrome (PCOS).
71. A polypeptide comprising a sequence having sequence identity of more than 60%, more than 70%, more than 80%, more than 90% or more than 95% to any of SEQ ID NO's. 7-1242.
72. The polypeptide of embodiment 71, comprising a sequence selected from the group consisting of SEQ ID NO's. 7-1242.
73. A polypeptide comprising a sequence having sequence identity of more than 60%, more than 70%, more than 80%, more than 90% or more than 95% to any of SEQ ID NOs. 294 to 775 or 1146 to 1240, or a pharmaceutically acceptable salt thereof.
74. A polypeptide selected from the group consisting of SEQ ID NO's: 294 to 775 or 1146 to 1240, or a pharmaceutically acceptable salt thereof.
75. The polypeptide of embodiment 74 selected from the group consisting of SEQ ID NO's: 692, 700, 702, 705, 706, 716, 718, 743, 747, 749, 767, or a pharmaceutically acceptable salt thereof.
76. The polypeptide of any of the above embodiments, wherein the polypeptide has greater potency at each of the glucagon, GIP and GLP-1 receptors as compared to native glucagon (SEQ ID NO:3), GIP (SEQ ID NO:1) and GLP-17-36 (SEQ ID NO:2).
77. The polypeptide of any of the above embodiments, wherein one or more hydrogen atoms are replaced by deuterium; or a pharmaceutically acceptable salt thereof.

The invention is further illustrated by the following examples, which are not to be construed as limiting.

### Peptide Synthesis

### Example 1

Example 1 is a compound represented by the following description:

(SEQ ID NO: 7)
Y-Aib-QGTFTSDFSK-αMeL-LDKK((2-[2-(2-Amino-ethoxy)-
ethoxy]-acetyl)-(γ-Glu)-CO—(CH₂₎₁₈—CO₂H)AQ-Aib-
AFIEYLLAGGPSSGEPPPSEG-NH₂.

Below is a depiction of the structure of Example 1 using the standard single letter amino acid codes with the exception of residues Aib2, αMeL13 and Aib20, where the structures of these amino acid residues have been expanded:

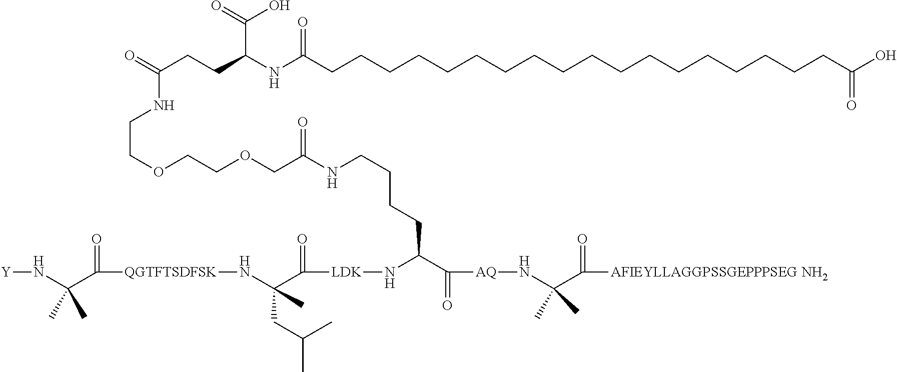

The peptide backbone of Example 1 is synthesized using Fluorenylmethyloxycarbonyl (Fmoc)/tert-Butyl (t-Bu) chemistry on a Symphony 12-Channel Multiplex Peptide Synthesizer (Protein Technologies, Inc. Tucson, AZ).

The resin consists of 1% DVB cross-linked polystyrene (Fmoc-Rink-MBHA Low Loading Resin, 100-200 mesh, EMD Millipore) at a substitution of 0.3-0.4 meq/g. Standard side-chain protecting groups are used. Fmoc-Lys(Mtt)-OH is used for the lysine at position 17, and Boc-Tyr(tBu)-OH is used for the tyrosine at position 1. Fmoc groups are removed prior to each coupling step (2×7 minutes) using 20% piperidine in DMF. All standard amino acid couplings are performed for 1 hour to a primary amine and 3 hour to a secondary amine, using an equal molar ratio of Fmoc amino acid (0.3M), diisopropylcarbodiimide (0.9M) and Oxyma (0.9M), at a 9-fold molar excess over the theoretical peptide loading. Exceptions are couplings to Ca-methylated amino acids, which are coupled for 3 hours. After completion of the synthesis of the peptide backbone, the resin is thoroughly washed with DCM for 6 times to remove residual DMF. The Mtt protecting group on the lysine at position 17 is selectively removed from the peptide resin using two treatments of 30% hexafluoroisopropanol (Oakwood Chemicals) in DCM (2× 40-minute treatment).

Subsequent attachment of the fatty acid-linker moiety is accomplished by coupling of 2-[2-(2-Fmoc-amino-ethoxy)-ethoxy]-acetic acid (Fmoc-AEEA-OH, ChemPep, Inc.), Fmoc-glutamic acid α-t-butyl ester (Fmoc-Glu-OtBu, Ark Pharm, Inc.), mono-OtBu-eicosanedioic acid (WuXi AppTec, Shanghai, China). 3-fold excess of reagents (AA:PyAOP:DIPEA=1:1:1 mol/mol) are used for each coupling that is 1-hour long.

After the synthesis is complete, the peptide resin is washed with DCM, and then thoroughly air-dried. The dry resin is treated with 10 ml of cleavage cocktail (trifluoroacetic acid:water:triisopropylsilane, 95:2.5:2.5 v/v) for 2 hours at room temperature. The resin is filtered off, washed twice each with 2 mL of neat TFA, and the combined filtrates are treated with 5-fold (by volume) cold diethyl ether (−20° C.) to precipitate the crude peptide. The peptide/ether suspension is then centrifuged at 3500 rpm for 2 min to form a solid pellet, the supernatant is decanted, and the solid pellet is triturated with ether two additional times and dried in vacuo. The crude peptide is solubilized in 20% acetonitrile/20% acetic acid/60% water and purified by RP-HPLC on a Luna 5 μm Phenyl-Hexyl Preparative Column (21×250 mm, Phenomenex) with linear gradients of 100% acetonitrile and 0.1% TFA/water buffer system (30-50% acetonitrile in 60 min). The purity of peptide is assessed using analytical RP-HPLC and pooling criteria is >95%. The main pool purity of Example 1 is found to be 98.8%. Subsequent lyophilization of the final main product pool yields the lyophilized peptide TFA salt. The molecular weight is determined by LC-MS (obsd: M+4H⁺/4=1226.8; Calc M+4H⁺/4=1226.9).

### Example 2 Through Example 1236

Polypeptides according to Example 2 (SEQ ID NO:8) through Example 1236 (SEQ ID NO:1242) are prepared substantially as described by the procedures of Example 1. These are listed below in Table 1. Additional depictions of certain examples are provided following Table 1.

TABLE 1

| | | SEQ | |
|---|---|---|---|
| Ex. | | ID | MW |
| No. | Sequence | NO | Calculated |
| 1 | Y-Aib-QGTFTSDFSK-αMeL-LDKK((2-[2-(2-Amino- | 7 | 4916.5 |
| | ethoxy)-ethoxy]-acetyl)-(γ-Glu)-CO—(CH₂)₁₈—CO₂H)AQ- | | |
| | Aib-AFIEYLLAGGPSSGEPPPSEG-NH₂ | | |
| 2 | Y-Aib-QGTFTSDFSK-αMeL-LDKK((2-[2-(2-Amino- | 8 | 4916.5 |
| | ethoxy)-ethoxy]-acetyl)-(γ-Glu)-CO—(CH₂)₁₈—CO₂H)AQ- | | |
| | Aib-AFIEYLLAGGPSSGEPPPSGE-NH₂ | | |
| 3 | Y-Aib-QGTFTSDFSK-αMeL-LDKK((2-[2-(2-Amino- | 9 | 4946.6 |
| | ethoxy)-ethoxy]-acetyl)-(γ-Glu)-CO—(CH₂)₁₈—CO₂H)AQ- | | |
| | Aib-AFIEYLLAGGPSSGEPPPSSE-NH₂ | | |
| 4 | Y-Aib-QGTFTSDFSK-αMeL-LDKK((2-[2-(2-Amino- | 10 | 4988.6 |
| | ethoxy)-ethoxy]-acetyl)-(γ-Glu)-CO—(CH₂)₁₈—CO₂H)AQ- | | |
| | Aib-AFIEYLLAGGPSSGEPPPSEE-NH₂ | | |
| 5 | Y-Aib-QGTFTSDFSK-αMeL-LDKK((2-[2-(2-Amino- | 11 | 5091.7 |
| | ethoxy)-ethoxy]-acetyl)₂-(γ-Glu)-CO—(CH₂)₁₈—CO₂H))AQ- | | |
| | Aib-AFIEYLLAGGPSSGEPPPSSE-NH₂ | | |
| 6 | Y-Aib-QGTFTSDFSK-αMeL-LDKK((2-[2-(2-Amino- | 12 | 4844.5 |
| | ethoxy)-ethoxy]-acetyl)-(γ-Glu)-CO—(CH₂)₁₈—CO₂H)AQ- | | |
| | Aib-AFIEYLLAGGPSSGEPPPSGG-NH₂ | | |
| 7 | Y-Aib-QGTFTSDFSK-αMeL-LDKK((2-[2-(2-Amino- | 13 | 4874.5 |
| | ethoxy)-ethoxy]-acetyl)-(γ-Glu)-CO—(CH₂)₁₈—CO₂H)AQ- | | |
| | Aib-AFIEYLLAGGPSSGEPPPSSG-NH₂ | | |
| 8 | Y-Aib-QGTFTSDFSK-αMeL-LDKK((2-[2-(2-Amino- | 14 | 4874.5 |
| | ethoxy)-ethoxy]-acetyl)-(γ-Glu)-CO—(CH₂)₁₈—CO₂H)AQ- | | |
| | Aib-AFIEYLLAGGPSSGEPPPSGS-NH₂ | | |
| 9 | Y-Aib-QGTFTSDFSK-αMeL-LDKK((2-[2-(2-Amino- | 15 | 4844.5 |
| | ethoxy)-ethoxy]-acetyl)-(γ-Glu)-CO—(CH₂)₁₈—CO₂H)AQ- | | |
| | Aib-AFIEYLLAGGPSSGEPPPGGS-NH₂ | | |
| 10 | Y-Aib-QGTFTSDFSK-αMeL-LDKK((2-[2-(2-Amino- | 16 | 4858.5 |
| | ethoxy)-ethoxy]-acetyl)-(γ-Glu)-CO—(CH₂)₁₈—CO₂H)AQ- | | |
| | Aib-AFIEYLLAGGPSSGAPPPSEG-NH₂ | | |
| 11 | Y-Aib-QGTFTSDFSK-αMeL-LDKK((2-[2-(2-Amino- | 17 | 4858.5 |
| | ethoxy)-ethoxy]-acetyl)-(γ-Glu)-CO—(CH₂)₁₈—CO₂H)AQ- | | |
| | Aib-AFIEYLLAGGPSSGAPPPSGE-NH₂ | | |
| 12 | Y-Aib-QGTFTSDFSK-αMeL-LDKK((2-[2-(2-Amino- | 18 | 4786.4 |
| | ethoxy)-ethoxy]-acetyl)-(γ-Glu)-CO—(CH₂)₁₈—CO₂H)AQ- | | |
| | Aib-AFIEYLLAGGPSSGAPPPSGG-NH₂ | | |
| 13 | Y-Aib-QGTFTSDFSK-αMeL-LDKK((2-[2-(2-Amino- | 19 | 4816.5 |
| | ethoxy)-ethoxy]-acetyl)-(γ-Glu)-CO—(CH₂)₁₈—CO₂H)AQ- | | |
| | Aib-AFIEYLLAGGPSSGAPPPSSG-NH₂ | | |
| 14 | Y-Aib-QGTFTSDFSK-αMeL-LDKK((2-[2-(2-Amino- | 20 | 4816.5 |
| | ethoxy)-ethoxy]-acetyl)-(γ-Glu)-CO—(CH₂)₁₈—CO₂H)AQ- | | |
| | Aib-AFIEYLLAGGPSSGAPPPSGS-NH₂ | | |
| 15 | Y-Aib-QGTFTSDFSK-αMeL-LDKK((2-[2-(2-Amino- | 21 | 4828.5 |
| | ethoxy)-ethoxy]-acetyl)-(γ-Glu)-CO—(CH₂)₁₈—CO₂H)AQ- | | |
| | Aib-AFIEYLLAGGPSSGAPPPGGE-NH₂ | | |
| 16 | Y-Aib-QGTFTSDFSK-αMeL-LDKK((2-[2-(2-Amino- | 22 | 4786.4 |
| | ethoxy)-ethoxy]-acetyl)-(γ-Glu)-CO—(CH₂)₁₈—CO₂H)AQ- | | |
| | Aib-AFIEYLLAGGPSSGAPPPGGS-NH₂ | | |
| 17 | Y-Aib-QGTFTSDFSK-αMeL-LDKK((2-[2-(2-Amino- | 23 | 5031.7 |
| | ethoxy)-ethoxy]-acetyl)₂-(γ-Glu)-CO—(CH₂)₁₈—CO₂H))AQ- | | |
| | Aib-AFIEYLLEGGPSSGAPPPGEG-NH₂ | | |
| 18 | Y-Aib-QGTFTSDFSK-αMeL-LDKK((2-[2-(2-Amino- | 24 | 4844.5 |
| | ethoxy)-ethoxy]-acetyl)-(γ-Glu)-CO—(CH₂)₁₈—CO₂H)AQ- | | |
| | Aib-AFIEYLLEGGPSSGAPPPGGS-NH₂ | | |
| 19 | Y-Aib-QGTFTSDFSK-αMeL-LDKK((2-[2-(2-Amino- | 25 | 4987.6 |
| | ethoxy)-ethoxy]-acetyl)-(γ-Glu)-CO—(CH₂)₁₈— | | |
| | CO₂H)AQQAFIEYLLA-Aib-GPSSGEPPPSEG-NH₂ | | |
| 20 | Y-Aib-QGTFTSDFSK-αMeL-LDKK((2-[2-(2-Amino- | 26 | 4939.6 |
| | ethoxy)-ethoxy]-acetyl)-(γ-Glu)-CO—(CH₂)₁₈—CO₂H)AQ- | | |
| | Aib-AFIEWLLAGGPSSGEPPPSEG-NH₂ | | |
| 21 | Y-Aib-QGTFTSDFSK-αMeL-LDKK((2-[2-(2-Amino- | 27 | 4939.6 |
| | ethoxy)-ethoxy]-acetyl)-(γ-Glu)-CO—(CH₂)₁₈—CO₂H)AQ- | | |
| | Aib-AFIEWLLAGGPSSGEPPPSGE-NH₂ | | |
| 22 | Y -- Aib-QGTFTSDFSK-αMeL-LDKK((2-[2-(2-Amino- | 28 | 4888.5 |
| | ethoxy)-ethoxy]-acetyl)-(γ-Glu)-CO—(CH₂)₁₆—CO₂H)AQ- | | |
| | Aib-AFIEYLLAGGPSSGEPPPSEG-NH₂ | | |
| 23 | Y-Aib-QGTFTSDFSK-αMeL-LDKK((2-[2-(2-Amino- | 29 | 4888.5 |
| | ethoxy)-ethoxy]-acetyl)-(γ-Glu)-CO—(CH₂)₁₆—CO₂H)AQ- | | |
| | Aib-AFIEYLLAGGPSSGEPPPSGE-NH₂ | | |
| 24 | Y-Aib-QGTFTSDFSK-αMeL-LDKK((2-[2-(2-Amino- | 30 | 4930.6 |
| | ethoxy)-ethoxy]-acetyl)-(γ-Glu)-CO—(CH₂)₁₈—CO₂H)AQ- | | |
| | Aib-AFIEYLLAGAPSSGAPPPSEG-NH₂ | | |
| 25 | Y-Aib-QGTFTSDFSK-αMeL-LDKK((2-[2-(2-Amino- | 31 | 5033.6 |
| | ethoxy)-ethoxy]-acetyl)₂-(γ-Glu)-CO—(CH₂)₁₆—CO₂H))AQ- | | |
| | Aib-AFIEYLLAGGPSSGEPPPSEG-NH₂ | | |
| 26 | Y-Aib-QGTFTSDFSK-αMeL-LDKK((2-[2-(2-Amino- | 32 | 4945.5 |
| | ethoxy)-ethoxy]-acetyl)-(γ-Glu)-CO—(CH₂)₁₆—CO₂H)AQ- | | |
| | Aib-QFIEYLLAGGPSSGEPPPSEG-NH₂ | | |
| 27 | Y-Aib-QGTFTSDFSK-αMeL-LDKK((2-[2-(2-Amino- | 33 | 4915.5 |
| | ethoxy)-ethoxy]-acetyl)-(γ-Glu)-CO—(CH₂)₁₈—CO₂H)AQ- | | |
| | Aib-AFIQYLLAGGPSSGEPPPSEG-NH₂ | | |
| 28 | Y-Aib-QGTFTSD-4Pal-SK-αMeL-LDKK((2-[2-(2-Amino- | 34 | 4917.5 |
| | ethoxy)-ethoxy]-acetyl)-(γ-Glu)-CO—(CH₂)₁₈—CO₂H)AQ- | | |
| | Aib-AFIEYLLAGGPSSGEPPPSGE-NH₂ | | |
| 29 | Y-Aib-QGTFTSDFS-Orn-αMeL-LDKK((2-[2-(2-Amino- | 35 | 4902.5 |
| | ethoxy)-ethoxy]-acetyl)-(γ-Glu)-CO—(CH₂)₁₈—CO₂H)AQ- | | |
| | Aib-AFIEYLLAGGPSSGEPPPSEG-NH₂ | | |
| 30 | Y-Aib-QGTFTSDFSK-αMeL-LDKK((2-[2-(2-Amino- | 36 | 4929.6 |
| | ethoxy)-ethoxy]-acetyl)-(γ-Glu)-CO—(CH₂)₁₈— | | |
| | CO₂H)AQQAFIEYLLA-Aib-GPSSGAPPPSEG-NH₂ | | |
| 31 | Y-Aib-QGTFTSDFSK-αMeL-LDKK((2-[2-(2-Amino- | 37 | 4886.5 |
| | ethoxy)-ethoxy]-acetyl)-(γ-Glu)-CO—(CH₂)₁₈—CO₂H)AQ- | | |
| | Aib-AFIEYLLAGGPSSGEPPPGEG-NH₂ | | |
| 32 | Y-Aib-QGTFTSDFSK-αMeL-LDKK((2-[2-(2-Amino- | 38 | 5061.7 |
| | ethoxy)-ethoxy]-acetyl)-(γ-Glu)-CO—(CH₂)₁₈—CO₂H)AQ- | | |
| | Aib-AFIEYLLAGGPSSGEPPPSEG-(2-[2-(2-Amino- | | |
| | ethoxy)-ethoxy]-acetic amide) | | |
| 33 | Y-Aib-QGTFTSDFSK-αMeL-LDKK((2-[2-(2-Amino- | 39 | 5061.7 |
| | ethoxy)-ethoxy]-acetyl)-(γ-Glu)-CO—(CH₂)₁₈—CO₂H)AQ- | | |
| | Aib-AFIEYLLAGGPSSGEPPPSGE-(2-[2-(2-Amino- | | |
| | ethoxy)-ethoxy]-acetic amide) | | |
| 34 | Y-Aib-QGTFTSDFSK-αMeL-LDKK((2-[2-(2-Amino- | 40 | 4900.5 |
| | ethoxy)-ethoxy]-acetyl)-(γ-Glu)-CO—(CH₂)₁₈—CO₂H)AQ- | | |
| | Aib-AFIEYLLEaGPSSGAPPPGEG-NH₂ | | |
| 35 | Y-Aib-QGTFTSDFSK-αMeL-LDKK((2-[2-(2-Amino- | 41 | 5016.6 |
| | ethoxy)-ethoxy]-acetyl)-(γ-Glu)-CO—(CH₂)₁₈—CO₂H)AQ- | | |
| | Aib-AFIEYLLEGGPSSGEPPPEEG-NH₂ | | |
| 36 | Y-Aib-QGTFTSDFSK-αMeL-LDKK((2-[2-(2-Amino- | 42 | 4988.6 |
| | ethoxy)-ethoxy]-acetyl)-(γ-Glu)-CO—(CH₂)₁₈—CO₂H)AQ- | | |
| | Aib-AFIEYLLEGGPSSGEPPPTEG-NH₂ | | |
| 37 | Y-Aib-QGTFTSDFSK-αMeL-LDKK((2-[2-(2-Amino- | 43 | 4939.6 |
| | ethoxy)-ethoxy]-acetyl)-(γ-Glu)-CO—(CH₂)₁₈—CO₂H)AQ- | | |
| | Aib-AFIEWLLEGGPSSGAPPPSEG-NH₂ | | |
| 38 | Y-Aib-QGTFTSDFSK-αMeL-LDKK((2-[2-(2-Amino- | 44 | 4996.6 |
| | ethoxy)-ethoxy]-acetyl)-(γ-Glu)-CO—(CH₂)₁₈—CO₂H)AQ- | | |
| | Aib-QFIEWLLEGGPSSGAPPPSEG-NH₂ | | |
| 39 | Y-Aib-QGTFTSDFSK-αMeL-LDKK((2-[2-(2-Amino- | 45 | 4938.6 |
| | ethoxy)-ethoxy]-acetyl)-(γ-Glu)-CO—(CH₂)₁₈—CO₂H)AQ- | | |
| | Aib-AFIQWLLEGGPSSGAPPPSEG-NH₂ | | |
| 40 | Y-Aib-QGTFTSDFSK-αMeL-LDKK((2-[2-(2-Amino- | 46 | 4939.6 |
| | ethoxy)-ethoxy]-acetyl)-(γ-Glu)-CO—(CH₂)₁₈—CO₂H)AQ- | | |
| | Aib-AFIEWLIEGGPSSGAPPPSEG-NH₂ | | |
| 41 | Y-Aib-QGTFTSDFSR-αMeL-LDKK((2-[2-(2-Amino- | 47 | 4872.5 |
| | ethoxy)-ethoxy]-acetyl)-(γ-Glu)-CO—(CH₂)₁₈—CO₂H)AQ- | | |
| | Aib-AFIEYLLEGGPSSGAPPPGGS-NH₂ | | |
| 42 | Y-Aib-QGTFTSDFSR-αMeL-LDKK((2-[2-(2-Amino- | 48 | 4900.5 |
| | ethoxy)-ethoxy]-acetyl)-(γ-Glu)-CO—(CH₂)₁₈—CO₂H)AQ- | | |
| | Aib-AFIEYLLE-Aib-GPSSGAPPPGGS-NH₂ | | |
| 43 | Y-Aib-QGTFTSDFSK-αMeL-LDKK((2-[2-(2-Amino- | 49 | 4872.5 |
| | ethoxy)-ethoxy]-acetyl)-(γ-Glu)-CO—(CH₂)₁₈—CO₂H)AQ- | | |
| | Aib-AFIEYLLE-Aib-GPSSGAPPPGGS-NH₂ | | |
| 44 | Y-Aib-QGTFTSDFSK-αMeL-LDKK((2-[2-(2-Amino- | 50 | 4902.5 |
| | ethoxy)-ethoxy]-acetyl)-(γ-Glu)-CO—(CH₂)₁₈—CO₂H)AQ- | | |
| | Aib-AFIEYLLE-Aib-GPSSGAPPPSSG-NH₂ | | |
| 45 | Y-Aib-QGTFTSDFSK-αMeL-LDKK((2-[2-(2-Amino- | 51 | 4902.5 |
| | ethoxy)-ethoxy]-acetyl)-(γ-Glu)-CO—(CH₂)₁₈—CO₂H)AQ- | | |
| | Aib-AFIEYLLE-Aib-GPSSGAPPPSGS-NH₂ | | |
| 46 | Y-Aib-QGTFTSDFSK-αMeL-LDKK((2-[2-(2-Amino- | 52 | 4974.6 |
| | ethoxy)-ethoxy]-acetyl)-(γ-Glu)-CO—(CH₂)₁₆—CO₂H)AQ- | | |
| | Aib-AFIEYLLE-Aib-GPSSGEPPPSEG-NH₂ | | |
| 47 | Y-Aib-QGTFTSDFSK-αMeL-LDKK((2-[2-(2-Amino- | 53 | 4930.6 |
| | ethoxy)-ethoxy]-acetyl)-(γ-Glu)-CO—(CH₂)₁₈—CO₂H)AQ- | | |
| | Aib-AFIEYLLEGAPSSGAPPPSEG-NH₂ | | |
| 48 | Y-Aib-QGTFTSDFSK-αMeL-LDKK((2-[2-(2-Amino- | 54 | 4888.5 |
| | ethoxy)-ethoxy]-acetyl)-(γ-Glu)-CO—(CH₂)₁₆—CO₂H)AQ- | | |
| | Aib-AFIEYLLEGGPSSGAPPPSEG-NH₂ | | |
| 49 | Y-Aib-QGTFTSDFSK-αMeL-LDKK((2-[2-(2-Amino- | 55 | 4888.5 |
| | ethoxy)-ethoxy]-acetyl)-(γ-Glu)-COγγ-(CH₂)₁₆—CO₂H)AQ- | | |
| | Aib-AFIEYLIEGGPSSGAPPPSEG-NH₂ | | |
| 50 | Y-Aib-QGTFTSDFS-Orn-αMeL-LDKK((2-[2-(2-Amino- | 56 | 4874.5 |
| | ethoxy)-ethoxy]-acetyl)-(γ-Glu)-CO—(CH₂)₁₆—CO₂H)AQ- | | |
| | Aib-AFIEYLLEGGPSSGAPPPSEG-NH₂ | | |
| 51 | Y-Aib-QGTFTSDFSK-αMeL-LDKK((2-[2-(2-Amino- | 57 | 4945.5 |
| | ethoxy)-ethoxy]-acetyl)-(γ-Glu)-CO—(CH₂)₁₆—CO₂H)AQ- | | |
| | Aib-QFIEYLLEGGPSSGAPPPSEG-NH₂ | | |
| 52 | Y-Aib-QGTFTSDFSK-αMeL-LDKK((2-[2-(2-Amino- | 58 | 4958.6 |
| | ethoxy)-ethoxy]-acetyl)-(γ-Glu)-CO—(CH₂)₁₈—CO₂H)AQ- | | |
| | Aib-AFIEYLLEGGPSSGAPPP-gE-EG-NH₂ | | |
| 53 | Y-Aib-QGTFTSDFSK-αMeL-LDKK((2-[2-(2-Amino- | 59 | 4958.6 |
| | ethoxy)-ethoxy]-acetyl)-(γ-Glu)-CO—(CH₂)₁₈—CO₂H)AQ- | | |
| | Aib-AFIEYLLEGGPSSGAPPP-gE-GE-NH₂ | | |
| 54 | Y-Aib-QGTFTSDFSK-αMeL-LDKK((2-[2-(2-Amino- | 60 | 5089.7 |
| | ethoxy)-ethoxy]-acetyl)-(γ-Glu)-CO—(CH₂)₁₈—CO₂H)AQ- | | |
| | Aib-AFIEYLLEGGPSSGEPPPGEG-(2-[2-(2-Amino- | | |
| | ethoxy)-ethoxy]-acetic amide) | | |
| 55 | Y-Aib-QGTFTSDFSK-αMeL-LDKK((2-[2-(2-Amino- | 61 | 5059.7 |
| | ethoxy)-ethoxy]-acetyl)₂-(γ-Glu)-CO—(CH₂)₁₈—CO₂H))AQ- | | |
| | Aib-AFIEYLLE-Aib-GPSSGAPPPGEG-NH₂ | | |
| 56 | Y-Aib-QGTFTSDFSK-αMeL-LDKK((2-[2-(2-Amino- | 62 | 5033.6 |
| | ethoxy)-ethoxy]-acetyl)-(γ-Glu)-CO—(CH₂)₁₆—CO₂H)AQ- | | |
| | Aib-AFIEYLLEGGPSSGAPPPSEG-(2-[2-(2-Amino- | | |
| | ethoxy)-ethoxy]-acetic amide) | | |
| 57 | Y-Aib-QGTFTSDFSK-αMeL-LDKK((2-[2-(2-Amino- | 63 | 4916.5 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSEG-NH₂ | | |
| 58 | Y-Aib-QGTFTSDFSK-αMeL-LDKK((γ-Glu)₂-CO—(CH₂)₁₆- | 64 | 4872.4 |
| | CO₂H)AQ-Aib-AFIEYLLEGGPSSGAPPPSEG-NH₂ | | |
| 59 | Y-Aib-QGTFTSDFS-Orn-αMeL-LDKK((2-[2-(2-Amino- | 65 | 4960.5 |
| | ethoxy)-ethoxy]-acetyl)-(γ-Glu)-CO—(CH₂)₁₈—CO₂H)AQ- | | |
| | Aib-AFIEYLLEGGPSSGEPPPSEG-NH₂ | | |
| 60 | Y-Aib-QGTFTSDFS-Orn-αMeL-LDKK((2-[2-(2-Amino- | 66 | 4844.5 |
| | ethoxy)-ethoxy]-acetyl)-(γ-Glu)-CO—(CH₂)₁₈—CO₂H)AQ- | | |
| | Aib-AFIEYLLAGGPSSGAPPPSEG-NH₂ | | |
| 61 | Y-Aib-QGTFTSDFS-Orn-αMeL-LDKK((2-[2-(2-Amino- | 67 | 4930.6 |
| | ethoxy)-ethoxy]-acetyl)-(γ-Glu)-CO—(CH₂)₁₈—CO₂H)AQ- | | |
| | Aib-AFIEYLLE-Aib-GPSSGAPPPSEG-NH₂ | | |
| 62 | Y-Aib-QGTFTSDFS-Orn-αMeL-LDKK((2-[2-(2-Amino- | 68 | 4802.4 |
| | ethoxy)-ethoxy]-acetyl)-(γ-Glu)-CO—(CH₂)₁₆—CO₂H)AQ- | | |
| | Aib-AFIEYLLEGGPSSGAPPPGGS-NH₂ | | |
| 63 | Y-Aib-QGTFTSDFS-Orn-αMeL-LDKK((2-[2-(2-Amino- | 69 | 4844.4 |
| | ethoxy)-ethoxy]-acetyl)-(γ-Glu)-CO—(CH₂)₁₆—CO₂H)AQ- | | |
| | Aib-AFIEYLLEGGPSSGAPPPGGE-NH₂ | | |
| 64 | Y-Aib-QGTFTSDFS-Orn-αMeL-LDKK((2-[2-(2-Amino- | 70 | 4859.4 |
| | ethoxy)-ethoxy]-acetyl)-(γ-Glu)-CO—(CH₂)₁₆—CO₂H)AQ- | | |
| | Aib-AFIEYLLEGGPSSGAPPPGGSG-NH₂ | | |
| 65 | Y-Aib-QGTFTSDFS-Orn-αMeL-LDKK((2-[2-(2-Amino- | 71 | 4931.5 |
| | ethoxy)-ethoxy]-acetyl)-(γ-Glu)-CO—(CH₂)₁₆—CO₂H)AQ- | | |
| | Aib-AFIEYLLEGGPSSGAPPPGGSE-NH₂ | | |
| 66 | Y-Aib-QGTFTSDFS-Orn-αMeL-LDKK((2-[2-(2-Amino- | 72 | 4947.5 |
| | ethoxy)-ethoxy]-acetyl)-(γ-Glu)-CO—(CH₂)₁₆—CO₂H)AQ- | | |
| | Aib-AFIEYLLEGGPSSGAPPPGGS-(2-[2-(2-Amino- | | |
| | ethoxy)-ethoxy]-acetic amide) | | |
| 67 | Y-Aib-QGTFTSDFS-Dab-αMeL-LDKK((2-[2-(2-Amino- | 73 | 4788.4 |
| | ethoxy)-ethoxy]-acetyl)-(γ-Glu)-CO—(CH₂)₁₆—CO₂H)AQ- | | |
| | Aib-AFIEYLLEGGPSSGAPPPGGS-NH₂ | | |
| 68 | Y-Aib-QGTFTSDFS-Dap-αMeL-LDKK((2-[2-(2-Amino- | 74 | 4774.3 |
| | ethoxy)-ethoxy]-acetyl)-(γ-Glu)-CO—(CH₂)₁₆—CO₂H)AQ- | | |
| | Aib-AFIEYLLEGGPSSGAPPPGGS-NH₂ | | |
| 69 | Y-Aib-QGTFTSDFS-Orn-αMeL-LDKK((2-[2-(2-Amino- | 75 | 5103.7 |
| | ethoxy)-ethoxy]-acetyl)₂-(γ-Glu)-CO—(CH₂)₁₈—CO₂H))AQ- | | |
| | Aib-AFIEYLLE-Aib-GPSSGEPPPGEG-NH₂ | | |
| 70 | Y-Aib-QGTFTSDFS-Orn-αMeL-LDKK((2-[2-(2-Amino- | 76 | 5045.7 |
| | ethoxy)-ethoxy]-acetyl)₂-(γ-Glu)-CO—(CH₂)₁₈—CO₂H))AQ- | | |
| | Aib-AFIEYLLE-Aib-GPSSGAPPPGEG-NH₂ | | |
| 71 | Y-Aib-QGTFTSDFS-Orn-αMeL-LDKK((2-[2-(2-Amino- | 77 | 5075.7 |
| | ethoxy)-ethoxy]-acetyl)₂-(γ-Glu)-CO—(CH₂)₁₈—CO₂H))AQ- | | |
| | Aib-AFIEYLLE-Aib-GPSSGAPPPSEG-NH₂ | | |
| 72 | Y-Aib-QGTFTSDFS-Orn-αMeL-LDKK((2-[2-(2-Amino- | 78 | 5061.7 |
| | ethoxy)-ethoxy]-acetyl)₂-(γ-Glu)-CO—(CH₂)₁₈—CO₂H))AQ- | | |
| | Aib-AFIEYLLEaGPSSGAPPPSEG | | |
| 73 | Y-Aib-QGTFTSDFS-Orn-αMeL-LDKK((2-[2-(2-Amino- | 79 | 4888.5 |
| | ethoxy)-ethoxy]-acetyl)-(γ-Glu)-CO—(CH₂)₁₈—CO₂H)AQ- | | |
| | Aib-AFIEYLLE-Aib-GPSSGAPPPSGS-NH₂ | | |
| 74 | Y-Aib-QGTFTSDFS-Orn-αMeL-LDKK((2-[2-(2-Amino- | 80 | 4860.5 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSGS-NH₂ | | |
| 75 | Y-Aib-QGTFTSDFS-Orn-αMeL-LDKK((2-[2-(2-Amino- | 81 | 5005.6 |
| | ethoxy)-ethoxy]-acetyl)₂-(γ-Glu)-CO—(CH₂)₁₆—CO₂H))AQ- | | |
| | Aib-AFIEYLLE-Aib-GPSSGAPPPSGS-NH₂ | | |
| 76 | Y-Aib-QGTFTSDFS-Orn-αMeL-LDKK((2-[2-(2-Amino- | 82 | 4802.4 |
| | ethoxy)-ethoxy]-acetyl)-(γ-Glu)-CO—(CH₂)₁₈—CO₂H)AQ- | | |
| | Aib-AFIEYLLAGGPSSGAPPPSGS-NH₂ | | |
| 77 | Y-Aib-QGTFTSDFSR-αMeL-LDKK((2-[2-(2-Amino- | 83 | 4844.5 |
| | ethoxy)-ethoxy]-acetyl)-(γ-Glu)-CO—(CH₂)₁₈—CO₂H)AQ- | | |
| | Aib-AFIEYLLAGGPSSGAPPPSGS-NH₂ | | |
| 78 | Y-Aib-QGTFTSDFS-Orn-αMeL-LDKK((2-[2-(2-Amino- | 84 | 4883.5 |
| | ethoxy)-ethoxy]-acetyl)-(γ-Glu)-CO—(CH₂)₁₈—CO₂H)AQ- | | |
| | Aib-AFIEWLLEGGPSSGAPPPSGS-NH₂ | | |
| 79 | Y-Aib-QGTFTSDFSR-αMeL-LDKK((2-[2-(2-Amino- | 85 | 4902.5 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSGS-NH₂ | | |
| 80 | Y-Aib-QGTFTSDFSR-αMeL-LDKK((2-[2-(2-Amino- | 86 | 5047.7 |
| | ethoxy)-ethoxy]-acetyl)₂-(γ-Glu)-CO—(CH₂)₁₆—CO₂H))AQ- | | |
| | Aib-AFIEYLLE-Aib-GPSSGAPPPSGS-NH₂ | | |
| 81 | Y-Aib-QGTFTSDFS-Orn-αMeL-LDKK((2-[2-(2-Amino- | 87 | 4830.4 |
| | ethoxy)-ethoxy]-acetyl)-(γ-Glu)-CO—(CH₂)₁₈—CO₂H)AQ- | | |
| | Aib-AFIEYLLAGGPSSGAPPPSGD-NH₂ | | |
| 82 | Y-Aib-QGTFTSDFS-Orn-αMeL-LDKK((2-[2-(2-Amino- | 88 | 4844.5 |
| | ethoxy)-ethoxy]-acetyl)-(γ-Glu)-CO—(CH₂)₁₈—CO₂H)AQ- | | |
| | Aib-AFIEYLLAGGPSSGAPPPSGE-NH₂ | | |
| 83 | Y-Aib-QGTFTSDFS-Orn-αMeL-LDKK((2-[2-(2-Amino- | 89 | 4947.6 |
| | ethoxy)-ethoxy]-acetyl)-(γ-Glu)-CO—(CH₂)₁₈—CO₂H)AQ- | | |
| | Aib-AFIEYLLAGGPSSGAPPPSGS-(2-[2-(2-Amino- | | |
| | ethoxy)-ethoxy]-acetic amide) | | |
| 84 | Y-Aib-QGTFTSDFS-Orn-αMeL-LDKK((2-[2-(2-Amino- | 90 | 4931.5 |
| | ethoxy)-ethoxy]-acetyl)-(γ-Glu)-CO—(CH₂)₁₈—CO₂H)AQ- | | |
| | Aib-AFIEYLLAGGPSSGAPPPSGSE-NH₂ | | |
| 85 | Y-Aib-QGTFTSDFS-Orn-αMeL-LDKK((2-[2-(2-Amino- | 91 | 4802.4 |
| | ethoxy)-ethoxy]-acetyl)-(γ-Glu)-CO—(CH₂)₁₆—CO₂H)AQ- | | |
| | Aib-AFIEYLLAGGPSSGAPPPSGD-NH₂ | | |
| 86 | Y-Aib-QGTFTSDFS-Orn-αMeL-LDKK((2-[2-(2-Amino- | 92 | 4816.4 |
| | ethoxy)-ethoxy]-acetyl)-(γ-Glu)-CO—(CH₂)₁₆—CO₂H)AQ- | | |
| | Aib-AFIEYLLAGGPSSGAPPPSGE-NH₂ | | |
| 87 | Y-Aib-QGTFTSDFS-Orn-αMeL-LDKK((2-[2-(2-Amino- | 93 | 4788.4 |
| | ethoxy)-ethoxy]-acetyl)-(γ-Glu)-CO—(CH₂)₁₆—CO₂H)AQ- | | |
| | Aib-AFIEYLLAGGPSSGAPPPSGT-NH₂ | | |
| 88 | Y-Aib-QGTFTSDFS-Orn-αMeL-LDKK((2-[2-(2-Amino- | 94 | 4903.5 |
| | ethoxy)-ethoxy]-acetyl)-(γ-Glu)-CO—(CH₂)₁₆—CO₂H)AQ- | | |
| | Aib-AFIEYLLAGGPSSGAPPPSGS-γE-NH₂ | | |
| 89 | Y-Aib-QGTFTSDFS-Orn-αMeL-LDKK((2-[2-(2-Amino- | 95 | 4919.5 |
| | ethoxy)-ethoxy]-acetyl)-(γ-Glu)-CO—(CH₂)₁₆—CO₂H)AQ- | | |
| | Aib-AFIEYLLAGGPSSGAPPPSGS-(2-[2-(2-Amino- | | |
| | ethoxy)-ethoxy]-acetic amide) | | |
| 90 | Y-Aib-QGTFTSDFS-Orn-αMeL-LDKK((2-[2-(2-Amino- | 96 | 4903.5 |
| | ethoxy)-ethoxy]-acetyl)-(γ-Glu)-CO—(CH₂)₁₆—CO₂H)AQ- | | |
| | Aib-AFIEYLLAGGPSSGAPPPSGSE-NH₂ | | |
| 91 | Y-Aib-QGTFTSDFS-Orn-αMeL-LDKK((2-[2-(2-Amino- | 97 | 4961.6 |
| | ethoxy)-ethoxy]-acetyl)-(γ-Glu)-CO—(CH₂)₁₆—CO₂H)AQ- | | |
| | Aib-AFIEYLLAGGPSSGAPPPSGE-(2-[2-(2-Amino- | | |
| | ethoxy)-ethoxy]-acetic amide) | | |
| 92 | Y-Aib-QGTFTSDFS-Orn-αMeL-LDKK((2-[2-(2-Amino- | 98 | 4860.5 |
| | ethoxy)-ethoxy]-acetyl)-(γ-Glu)-CO—(CH₂)₁₈—CO₂H)AQ- | | |
| | Aib-AFIEYLLAGGPSSGEPPPSGS-NH₂ | | |
| 93 | Y-Aib-QGTFTSDFS-Orn-αMeL-LDKK((2-[2-(2-Amino- | 99 | 4816.4 |
| | ethoxy)-ethoxy]-acetyl)-(γ-Glu)-CO—(CH₂)₁₆—CO₂H)AQ- | | |
| | Aib-AFIEYLLAGGPSSGAPPPEGS-NH₂ | | |
| 94 | Y-Aib-QGTFTSDFS-Orn-αMeL-LDKK((2-[2-(2-Amino- | 100 | 4832.4 |
| | ethoxy)-ethoxy]-acetyl)-(γ-Glu)-CO—(CH₂)₁₆—CO₂H)AQ- | | |
| | Aib-AFIEYLLAGGPSSGEPPPSGS-NH₂ | | |
| 95 | Y-Aib-QGTFTSDFS-Orn-αMeL-LDKK((2-[2-(2-Amino- | 101 | 4774.4 |
| | ethoxy)-ethoxy]-acetyl)-(γ-Glu)-CO—(CH₂)₁₆—CO₂H)AQ- | | |
| | Aib-AFIEYLLAGGPSSGAPPPSGS-NH₂ | | |
| 96 | Y-Aib-QGTFTSDFS-Orn-αMeL-LDKK((2-[2-(2-Amino- | 102 | 4873.5 |
| | ethoxy)-ethoxy]-acetyl)-(γ-Glu)-CO—(CH₂)₁₆—CO₂H)AQ- | | |
| | Aib-QFIEYLLAGGPSSGAPPPEGS-NH₂ | | |
| 97 | Y-Aib-QGTFTSDFS-Orn-αMeL-LDKK((2-[2-(2-Amino- | 103 | 4874.5 |
| | ethoxy)-ethoxy]-acetyl)-(γ-Glu)-CO—(CH₂)₁₆—CO₂H)AQ- | | |
| | Aib-AFIEYLLAGGPSSGEPPPSGE-NH₂ | | |
| 98 | Y-Aib-QGTFTSDFS-Orn-αMeL-LDKK((2-[2-(2-Amino- | 104 | 4916.5 |
| | ethoxy)-ethoxy]-acetyl)-(γ-Glu)-CO—(CH₂)₁₆—CO₂H)AQ- | | |
| | Aib-AFIEYLLAGGPSSGEPPPEGE-NH₂ | | |
| 99 | Y-Aib-QGTFTSDFS-Orn-αMeL-LDKK((2-[2-(2-Amino- | 105 | 4858.5 |
| | ethoxy)-ethoxy]-acetyl)-(γ-Glu)-CO—(CH₂)₁₆—CO₂H)AQ- | | |
| | Aib-AFIEYLLAGGPSSGAPPPEGE-NH₂ | | |
| 100 | Y-Aib-QGTFTSDFS-Orn-αMeL-LDKK((2-[2-(2-Amino- | 106 | 4925.5 |
| | ethoxy)-ethoxy]-acetyl)-(γ-Glu)-CO—(CH₂)₁₈—CO₂H)AQ- | | |
| | Aib-AFIEWLLEGGPSSGAPPPSGE-NH₂ | | |
| 101 | Y-Aib-QGTFTSDFS-Orn-αMeL-LDKK((2-[2-(2-Amino- | 107 | 4967.6 |
| | ethoxy)-ethoxy]-acetyl)-(γ-Glu)-CO—(CH₂)₁₈—CO₂H)AQ- | | |
| | Aib-AFIEWLLEGGPSSGAPPPEGE-NH₂ | | |
| 102 | Y-Aib-QGTFTSDFS-Orn-αMeL-LDKK((2-[2-(2-Amino- | 108 | 4944.5 |
| | ethoxy)-ethoxy]-acetyl)-(γ-Glu)-CO—(CH₂)₁₈—CO₂H)AQ- | | |
| | Aib-AFIEYLLAGGPSSGEPPPEGE-NH₂ | | |
| 103 | Y-Aib-QGTFTSDFS-Orn-αMeL-LDKK((2-[2-(2-Amino- | 109 | 4925.5 |
| | ethoxy)-ethoxy]-acetyl)-(γ-Glu)-CO—(CH₂)₁₈—CO₂H)AQ- | | |
| | Aib-AFIEWLLAGGPSSGEPPPSGE-NH₂ | | |
| 104 | Y-Aib-QGTFTSDFS-Orn-αMeL-LDKK((2-[2-(2-Amino- | 110 | 4941.5 |
| | ethoxy)-ethoxy]-acetyl)-(γ-Glu)-CO—(CH₂)₁₈—CO₂H)AQ- | | |
| | Aib-AFIEWLLEGG-Hyp-SSGAPPPSGE-NH₂ | | |
| 105 | Y-Aib-QGTFTSDFS-Orn-αMeL-LDKK((2-[2-(2-Amino- | 111 | 4941.5 |
| | ethoxy)-ethoxy]-acetyl)-(γ-Glu)-CO—(CH₂)₁₈—CO₂H)AQ- | | |
| | Aib-AFIEWLLEGGPSSGAPP-Hyp-SGE-NH₂ | | |
| 106 | Y-Aib-QGTFTSDFS-Orn-αMeL-LDKK((2-[2-(2-Amino- | 112 | 4906.5 |
| | ethoxy)-ethoxy]-acetyl)-(γ-Glu)-CO—(CH₂)₁₈—CO₂H)AQ- | | |
| | Aib-AFIEYLLAGGTSSGEPPPSGE-NH₂ | | |
| 107 | Y-Aib-QGTFTSDFS-Orn-αMeL-LDKK((2-[2-(2-Amino- | 113 | 5019.6 |
| | ethoxy)-ethoxy]-acetyl)₂-(γ-Glu)-CO—(CH₂)₁₆—CO₂H))AQ- | | |
| | Aib-AFIEYLLEGGPSSGAPPPSGE-NH₂ | | |
| 108 | Y-Aib-QGTFTSDFSR-αMeL-LDKK((2-[2-(2-Amino- | 114 | 4916.5 |
| | ethoxy)-ethoxy]-acetyl)-(γ-Glu)-CO—(CH₂)₁₆—CO₂H)AQ- | | |
| | Aib-AFIEYLLEGGPSSGAPPPSGE-NH₂ | | |
| 109 | Y-Aib-QGTFTSDFSR-αMeL-LDKK((2-[2-(2-Amino- | 115 | 5061.7 |
| | ethoxy)-ethoxy]-acetyl)₂-(γ-Glu)-CO—(CH₂)₁₆—CO₂H))AQ- | | |
| | Aib-AFIEYLLEGGPSSGAPPPSGE-NH₂ | | |
| 110 | Y-Aib-QGTFTSDFS-Orn-αMeL-LDKK((2-[2-(2-Amino- | 116 | 4902.5 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSGE-NH₂ | | |
| 111 | Y-Aib-QGTFTSDFS-Orn-αMeL-LDKK((2-[2-(2-Amino- | 117 | 5047.7 |
| | ethoxy)-ethoxy]-acetyl)₂-(γ-Glu)-CO—(CH₂)₁₆—CO₂H))AQ- | | |
| | Aib-AFIEYLLE-Aib-GPSSGAPPPSGE-NH₂ | | |
| 112 | Y-Aib-QGTFTSD-4Pal-S-Orn-αMeL-LDKK((2-[2-(2- | 118 | 4817.4 |
| | Amino-ethoxy)-ethoxy]-acetyl)-(γ-Glu)-CO—(CH₂)₁₆- | | |
| | CO₂H)AQ-Aib-AFIEYLLAGGPSSGAPPPSGE-NH₂ | | |
| 113 | Y-Aib-QGTFTSD-4Pal-SR-αMeL-LDKK((2-[2-(2-Amino- | 119 | 4859.4 |
| | ethoxy)-ethoxy]-acetyl)-(γ-Glu)-CO—(CH₂)₁₆—CO₂H)AQ- | | |
| | Aib-AFIEYLLAGGPSSGAPPPSGE-NH₂ | | |
| 114 | Y-Aib-QGTFTSD-4Pal-SR-αMeL-LDKK((2-[2-(2-Amino- | 120 | 4945.5 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSGE-NH₂ | | |
| 115 | Y-Aib-QGTFTSD-4Pal-S-Orn-αMeL-LDKK((2-[2-(2- | 121 | 4956.5 |
| | Amino-ethoxy)-ethoxy]-acetyl)-(γ-Glu)-CO—(CH₂)₁₆- | | |
| | CO₂H)AQ-Aib-AFIEWLLEGGPSSGEPPPSGE-NH₂ | | |
| 116 | Y-Aib-QGTFTSDFS-Orn-αMeL-LDKK((2-[2-(2-Amino- | 122 | 5091.7 |
| | ethoxy)-ethoxy]-acetyl)₂-(γ-Glu)-CO—(CH₂)₁₆—CO₂H))AQ- | | |
| | Aib-AFIEYLLEaGPSSGEPPPSEG-NH₂ | | |
| 117 | Y-Aib-QGTFTSDFS-Orn-αMeL-LDKK((2-[2-(2-Amino- | 123 | 5091.7 |
| | ethoxy)-ethoxy]-acetyl)₂-(γ-Glu)-CO—(CH₂)₁₆—CO₂H))AQ- | | |
| | Aib-AFIEYLLEaGPSSGEPPPSGE-NH₂ | | |
| 118 | Y-Aib-QGTFTSDFS-Orn-αMeL-LDKK((2-[2-(2-Amino- | 124 | 5019.6 |
| | ethoxy)-ethoxy]-acetyl)₂-(γ-Glu)-CO—(CH₂)₁₆—CO₂H))AQ- | | |
| | Aib-AFIEYLLEGGPSSGAPPPGES-NH₂ | | |
| 119 | Y-Aib-QGTFTSDFS-Or-αMeL-LDKK((2-[2-(2-Amino- | 125 | 4897.5 |
| | ethoxy)-ethoxy]-acetyl)-(γ-Glu)-CO—(CH₂)₁₆—CO₂H)AQ- | | |
| | Aib-AFIEWLLEGGPSSGAPPPSGE-NH₂ | | |
| 120 | Y-Aib-QGTFTSDFS-Orn-αMeL-LDKK((2-[2-(2-Amino- | 126 | 4939.5 |
| | ethoxy)-ethoxy]-acetyl)-(γ-Glu)-CO—(CH₂)₁₆—CO₂H)AQ- | | |
| | Aib-AFIEWLLEGGPSSGAPPPEGE-NH₂ | | |
| 121 | Y-Aib-QGTFTSDFS-Orn-αMeL-LDKK((2-[2-(2-Amino- | 127 | 4897.5 |
| | ethoxy)-ethoxy]-acetyl)-(γ-Glu)-CO—(CH₂)₁₆—CO₂H)AQ- | | |
| | Aib-AFIEWLLEGGPSSGAPPPEGS-NH₂ | | |
| 122 | Y-Aib-QGTFTSDFS-Orn-αMeL-LDKK((2-[2-(2-Amino- | 128 | 4937.5 |
| | ethoxy)-ethoxy]-acetyl)-(γ-Glu)-CO—(CH₂)₁₆—CO₂H)AQ- | | |
| | Aib-AFIEWLLEGGHSSGAPPPSGE-NH₂ | | |
| 123 | Y-Aib-QGTFTSDFS-Orn-αMeL-LDKK((2-[2-(2-Amino- | 129 | 4987.5 |
| | ethoxy)-ethoxy]-acetyl)-(γ-Glu)-CO—(CH₂)₁₆—CO₂H)AQ- | | |
| | Aib-AFIEWLLEGGESSGEPPPSGE-NH₂ | | |
| 124 | Y-Aib-QGTFTSDFS-Orn-αMeL-LDKK((2-[2-(2-Amino- | 130 | 4995.6 |
| | ethoxy)-ethoxy]-acetyl)-(γ-Glu)-CO—(CH₂)₁₆—CO₂H)AQ- | | |
| | Aib-AFIEWLLEGGHSSGEPPPSGE-NH₂ | | |
| 125 | Y-Aib-QGTFTSDFS-Orn-αMeL-LDKK((2-[2-(2-Amino- | 131 | 4929.5 |
| | ethoxy)-ethoxy]-acetyl)-(γ-Glu)-CO—(CH₂)₁₆—CO₂H)AQ- | | |
| | Aib-AFIEWLLEGGESSGAPPPEGS-NH₂ | | |
| 126 | Y-Aib-QGTFTSDFS-Orn-αMeL-LDKK((2-[2-(2-Amino- | 132 | 4937.5 |
| | ethoxy)-ethoxy]-acetyl)-(γ-Glu)-CO—(CH₂)₁₆—CO₂H)AQ- | | |
| | Aib-AFIEWLLEGGHSSGAPPPEGS-NH₂ | | |
| 127 | Y-Aib-QGTFTSDFS-Orn-αMeL-LDKK((2-[2-(2-Amino- | 133 | 4995.6 |
| | ethoxy)-ethoxy]-acetyl)-(γ-Glu)-CO—(CH₂)₁₆—CO₂H)AQ- | | |
| | Aib-AFIEWLLEGGHSSGEPPPEGS-NH₂ | | |
| 128 | Y-Aib-QGTFTSDFS-Orn-αMeL-LDKK((2-[2-(2-Amino- | 134 | 5033.6 |
| | ethoxy)-ethoxy]-acetyl)₂-(γ-Glu)-CO—(CH₂)₁₆—CO₂H))AQ- | | |
| | Aib-AFIEYLLEaGPSSGAPPPSGE-NH₂ | | |
| 129 | Y-Aib-QGTFTSDFS-Orn-αMeL-LDKK((2-[2-(2-Amino- | 135 | 4888.5 |
| | ethoxy)-ethoxy]-acetyl)-(γ-Glu)-CO—(CH₂)₁₆—CO₂H)AQ- | | |
| | Aib-AFIEYLLEaGPSSGAPPPSGE-NH₂ | | |
| 130 | Y-Aib-QGTFTSDFS-Orn-αMeL-LDKK((2-[2-(2-Amino- | 136 | 4874.5 |
| | ethoxy)-ethoxy]-acetyl)-(γ-Glu)-CO—(CH₂)₁₆—CO₂H)AQ- | | |
| | Aib-AFIEYLLEGGPSSGAPPPEGS-NH₂ | | |
| 131 | Y-Aib-QGTFTSDFS-Or-αMeL-LDKK((2-[2-(2-Amino- | 137 | 4890.5 |
| | ethoxy)-ethoxy]-acetyl)-(γ-Glu)-CO—(CH₂)₁₆—CO₂H)AQ- | | |
| | Aib-AFIEYLLEGGPSSGEPPPSGS-NH₂ | | |
| 132 | Y-Aib-QGTFTSD-4Pal-SR-αMeL-LDKK((2-[2-(2-Amino- | 138 | 4935.5 |
| | ethoxy)-ethoxy]-acetyl)-(γ-Glu)-CO—(CH₂)₁₆—CO₂H)AQ- | | |
| | Aib-AFIEYLLE-Aib-GESSGAPPPSGS-NH₂ | | |
| 133 | Y-Aib-QGTFTSDFS-Dab-αMeL-LDKK((2-[2-(2-Amino- | 139 | 4846.4 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSGS-NH₂ | | |
| 134 | Y-Aib-QGTFTSDFS-Dap-αMeL-LDKK((2-[2-(2-Amino- | 140 | 4832.4 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSGS-NH₂ | | |
| 135 | Y-Aib-QGTFTSDFS-Orn-αMeL-LDKK((2-[2-(2-Amino- | 141 | 4946.5 |
| | ethoxy)-ethoxy]-acetyl)-(γ-Glu)-CO—(CH₂)₁₆—CO₂H)AQ- | | |
| | Aib-AFIEYLLEaGPSSGEPPPSGE-NH₂ | | |
| 136 | Y-Aib-QGTFTSDFS-Orn-αMeL-LDKK((2-[2-(2-Amino- | 142 | 4913.5 |
| | ethoxy)-ethoxy]-acetyl)-(γ-Glu)-CO—(CH₂)₁₆—CO₂H)AQ- | | |
| | Aib-AFIEWLLEGGPSSGA-Hyp-PPSGE-NH₂ | | |
| 137 | Y-Aib-QGTFTSDFS-Orn-αMeL-LDKK((2-[2-(2-Amino- | 143 | 4913.5 |
| | ethoxy)-ethoxy]-acetyl)-(γ-Glu)-CO—(CH₂)₁₆—CO₂H)AQ- | | |
| | Aib-AFIEWLLEGGPSSGAP-Hyp-PSGE-NH₂ | | |
| 138 | Y-Aib-QGTFTSDFS-Orn-αMeL-LDKK((2-[2-(2-Amino- | 144 | 4913.5 |
| | ethoxy)-ethoxy]-acetyl)-(γ-Glu)-CO—(CH₂)₁₆—CO₂H)AQ- | | |
| | Aib-AFIEWLLEGGPSSGAPP-Hyp-SGE-NH₂ | | |
| 139 | Y-Aib-QGTFTSDFS-Orn-αMeL-LDKK((2-[2-(2-Amino- | 145 | 4832.4 |
| | ethoxy)-ethoxy]-acetyl)₂-(γ-Glu)-CO—(CH₂)₁₆—CO₂H))AQ- | | |
| | Aib-AFIEYLLEGGPSSGAPPPSGS-NH₂ | | |
| 140 | Y-Aib-QGTFTSDFS-Orn-αMeL-LDKK((2-[2-(2-Amino- | 146 | 4977.6 |
| | ethoxy)-ethoxy]-acetyl)₂-(γ-Glu)-CO—(CH₂)₁₆—CO₂H))AQ- | | |
| | Aib-AFIEYLLEGGPSSGAPPPSGS-NH₂ | | |
| 141 | Y-Aib-QGTFTSDFSK-αMeL-LDKK((2-[2-(2-Amino- | 147 | 4846.4 |
| | ethoxy)-ethoxy]-acetyl)-(γ-Glu)-CO—(CH₂)₁₆—CO₂H)AQ- | | |
| | Aib-AFIEYLLEGGPSSGAPPPSGS-NH₂ | | |
| 142 | Y-Aib-QGTFTSDFSK-αMeL-LDKK((2-[2-(2-Amino- | 148 | 4991.6 |
| | ethoxy)-ethoxy]-acetyl)₂-(γ-Glu)-CO—(CH₂)₁₆—CO₂H))AQ- | | |
| | Aib-AFIEYLLEGGPSSGAPPPSGS-NH₂ | | |
| 143 | Y-Aib-QGTFTSDFSR-αMeL-LDKK((2-[2-(2-Amino- | 149 | 4874.5 |
| | ethoxy)-ethoxy]-acetyl)-(γ-Glu)-CO—(CH₂)₁₆—CO₂H)AQ- | | |
| | Aib-AFIEYLLEGGPSSGAPPPSGS-NH₂ | | |
| 144 | Y-Aib-QGTFTSDFSR-αMeL-LDKK((2-[2-(2-Amino- | 150 | 5019.6 |
| | ethoxy)-ethoxy]-acetyl)₂-(γ-Glu)-CO—(CH₂)₁₆—CO₂H))AQ- | | |
| | Aib-AFIEYLLEGGPSSGAPPPSGS-NH₂ | | |
| 145 | Y-Aib-QGTFTSDFS-Or-αMeL-LDKK((2-[2-(2-Amino- | 151 | 4864.4 |
| | ethoxy)-ethoxy]-acetyl)-(γ-Glu)-CO—(CH₂)₁₆—CO₂H)AQ- | | |
| | Aib-AFIEYLLEGGESSGAPPPSGS-NH₂ | | |
| 146 | Y-Aib-QGTFTSDFSK-αMeL-LDKK((2-[2-(2-Amino- | 152 | 4878.4 |
| | ethoxy)-ethoxy]-acetyl)-(γ-Glu)-CO—(CH₂)₁₆—CO₂H)AQ- | | |
| | Aib-AFIEYLLEGGESSGAPPPSGS-NH₂ | | |
| 147 | Y-Aib-QGTFTSDFSK-αMeL-LDKK((2-[2-(2-Amino- | 153 | 5023.6 |
| | ethoxy)-ethoxy]-acetyl)₂-(γ-Glu)-CO—(CH₂)₁₆—CO₂H))AQ- | | |
| | Aib-AFIEYLLEGGESSGAPPPSGS-NH₂ | | |
| 148 | Y-Aib-QGTFTSDFSR-αMeL-LDKK((2-[2-(2-Amino- | 154 | 4906.5 |
| | ethoxy)-ethoxy]-acetyl)-(γ-Glu)-CO—(CH₂)₁₆—CO₂H)AQ- | | |
| | Aib-AFIEYLLEGGESSGAPPPSGS-NH₂ | | |
| 149 | Y-Aib-QGTFTSDFSR-αMeL-LDKK((2-[2-(2-Amino- | 155 | 5051.6 |
| | ethoxy)-ethoxy]-acetyl)₂-(γ-Glu)-CO—(CH₂)₁₆—CO₂H))AQ- | | |
| | Aib-AFIEYLLEGGESSGAPPPSGS-NH₂ | | |
| 150 | Y-Aib-QGTFTSDFSQ-αMeL-LDKK((2-[2-(2-Amino- | 156 | 4846.4 |
| | ethoxy)-ethoxy]-acetyl)-(γ-Glu)-CO—(CH₂)₁₆—CO₂H)AQ- | | |
| | Aib-AFIEYLLEGGPSSGAPPPSGS-NH₂ | | |
| 151 | Y-Aib-QGTFTSDFSQ-αMeL-LDKK((2-[2-(2-Amino- | 157 | 4874.5 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSGS-NH₂ | | |
| 152 | Y-Aib-QGTFTSDFS-Orn-αMeL-LDKK((2-[2-(2-Amino- | 158 | 4874.5 |
| | ethoxy)-ethoxy]-acetyl)-(γ-Glu)-CO—(CH₂)₁₆—CO₂H)AQ- | | |
| | Aib-AFIEYLLAGGPSSGEPPPEGS-NH₂ | | |
| 153 | Y-Aib-QGTFTSDFS-Orn-αMeL-LDKK((2-[2-(2-Amino- | 159 | 4961.5 |
| | ethoxy)-ethoxy]-acetyl)-(γ-Glu)₂-CO—(CH₂)₁₆—CO₂H)AQ- | | |
| | Aib-AFIEYLLAGGPSSGEPPPSGS-NH₂ | | |
| 154 | Y-Aib-QGTFTSD-4Pal-S-Orn-αMeL-LDKK((2-[2-(2- | 160 | 4833.4 |
| | Amino-ethoxy)-ethoxy]-acetyl)-(γ-Glu)-CO—(CH₂)₁₆- | | |
| | CO₂H)AQ-Aib-AFIEYLLAGGPSSGEPPPSGS-NH₂ | | |
| 155 | Y-Aib-QGTFTSDFS-Orn-αMeL-LDKK((2-[2-(2-Amino- | 161 | 5049.6 |
| | ethoxy)-ethoxy]-acetyl)₂-(γ-Glu)-CO—(CH₂)₁₆—CO₂H))AQ- | | |
| | Aib-AFIEYLLEaGPSSGEPPPSGS-NH₂ | | |
| 156 | Y-Aib-QGTFTSDFS-Orn-αMeL-LDKK((2-[2-(2-Amino- | 162 | 5063.7 |
| | ethoxy)-ethoxy]-acetyl)₂-(γ-Glu)-CO—(CH₂)₁₆—CO₂H))AQ- | | |
| | Aib-AFIEYLLEaGPSSGEPPPSGT-NH₂ | | |
| 157 | Y-Aib-QGTFTSDFS-Or-αMeL-LDKK((2-[2-(2-Amino- | 163 | 5090.7 |
| | ethoxy)-ethoxy]-acetyl)₂-(γ-Glu)-CO—(CH₂)₁₆—CO₂H))AQ- | | |
| | Aib-AFIEYLLEaGPSSGEPPPSGQ-NH₂ | | |
| 158 | Y-Aib-QGTFTSDFS-Orn-αMeL-LDKK((2-[2-(2-Amino- | 164 | 4816.4 |
| | ethoxy)-ethoxy]-acetyl)-(γ-Glu)-CO—(CH₂)₁₆—CO₂H)AQ- | | |
| | Aib-AFIEYLLEGGPSSGAPPPGGT-NH₂ | | |
| 159 | Y-Aib-QGTFTSDFS-Orn-αMeL-LDKK((2-[2-(2-Amino- | 165 | 4843.4 |
| | ethoxy)-ethoxy]-acetyl)-(γ-Glu)-CO—(CH₂)₁₆—CO₂H)AQ- | | |
| | Aib-AFIEYLLEGGPSSGAPPPGGQ-NH₂ | | |
| 160 | Y-Aib-QGTFTSDFS-Orn-αMeL-LDKK((2-[2-(2-Amino- | 166 | 4888.5 |
| | ethoxy)-ethoxy]-acetyl)-(γ-Glu)-CO—(CH₂)₁₆—CO₂H)AQ- | | |
| | Aib-AFIEYLLEGGPSSGAPPPEGT-NH₂ | | |
| 161 | Y-Aib-QGTFTSDFS-Orn-αMeL-LDKK((2-[2-(2-Amino- | 167 | 4835.4 |
| | ethoxy)-ethoxy]-acetyl)-(γ-Glu)-CO—(CH₂)₁₆—CO₂H)AQ- | | |
| | Aib-AFIEYLLEGGESSGAPPPGGS-NH₂ | | |
| 162 | Y-Aib-QGTFTSDFS-Orn-αMeL-LDKK((2-[2-(2-Amino- | 168 | 4843.4 |
| | ethoxy)-ethoxy]-acetyl)-(γ-Glu)-CO—(CH₂)₁₆—CO₂H)AQ- | | |
| | Aib-AFIEYLLEGGHSSGAPPPGGS-NH₂ | | |
| 163 | Y-Aib-QGTFTSDFS-Orn-αMeL-LDKK((2-[2-(2-Amino- | 169 | 4854.4 |
| | ethoxy)-ethoxy]-acetyl)-(γ-Glu)-CO—(CH₂)₁₆—CO₂H)AQ- | | |
| | Aib-AFIEYLLEGG-4Pal-SSGAPPPGGS-NH₂ | | |
| 164 | Y-Aib-QGTFTSDFS-Orn-αMeL-LDKK((2-[2-(2-Amino- | 170 | 4901.4 |
| | ethoxy)-ethoxy]-acetyl)-(γ-Glu)-CO—(CH₂)₁₆—CO₂H)AQ- | | |
| | Aib-AFIEYLLEGGHSSGEPPPGGS-NH₂ | | |
| 165 | Y-Aib-QGTFTSDFS-Orn-αMeL-LDKK((2-[2-(2-Amino- | 171 | 5019.7 |
| | ethoxy)-ethoxy]-acetyl)₂-(γ-Glu)-CO—(CH₂)₁₆—CO₂H))AQ- | | |
| | Aib-AFIEYLLE-Aib-GPSSGAPPPSGT-NH₂ | | |
| 166 | Y-Aib-QGTFTSDFS-Orn-αMeL-LDKK((2-[2-(2-Amino- | 172 | 5063.7 |
| | ethoxy)-ethoxy]-acetyl)₂-(γ-Glu)-CO—(CH₂)₁₆—CO₂H))AQ- | | |
| | Aib-AFIEYLLE-Aib-GPSSGEPPPSGS-NH₂ | | |
| 167 | Y-Aib-QGTFTSDFS-Orn-αMeL-LDKK((2-[2-(2-Amino- | 173 | 5077.7 |
| | ethoxy)-ethoxy]-acetyl)₂-(γ-Glu)-CO—(CH₂)₁₆—CO₂H))AQ- | | |
| | Aib-AFIEYLLE-Aib-GPSSGEPPPSGT-NH₂ | | |
| 168 | Y-Aib-QGTFTSDFS-Orn-αMeL-LDKK((2-[2-(2-Amino- | 174 | 4855.5 |
| | ethoxy)-ethoxy]-acetyl)-(γ-Glu)-CO—(CH₂)₁₆—CO₂H)AQ- | | |
| | Aib-AFIEWLLEGGPSSGAPPPSGS-NH₂ | | |
| 169 | Y-Aib-QGTFTSDFS-Orn-αMeL-LDKK((2-[2-(2-Amino- | 175 | 5082.7 |
| | ethoxy)-ethoxy]-acetyl)₂-(γ-Glu)-CO—(CH₂)₁₆—CO₂H))AQ- | | |
| | Aib-AFIEWLLEGGHSSGAPPPSGE-NH₂ | | |
| 170 | Y-Aib-QGTFTSDFS-Orn-αMeL-LDKK((2-[2-(2-Amino- | 176 | 4895.5 |
| | ethoxy)-ethoxy]-acetyl)-(γ-Glu)-CO—(CH₂)₁₆—CO₂H)AQ- | | |
| | Aib-AFIEWLLEGGHSSGAPPPSGS-NH₂ | | |
| 171 | Y-Aib-QGTFTSDFS-Orn-αMeL-LDKK((2-[2-(2-Amino- | 177 | 4909.5 |
| | ethoxy)-ethoxy]-acetyl)-(γ-Glu)-CO—(CH₂)₁₆—CO₂H)AQ- | | |
| | Aib-AFIEWLLEGGHSSGAPPPSGT-NH₂ | | |
| 172 | Y-Aib-QGTFTSDFS-Orn-αMeL-LDKK((2-[2-(2-Amino- | 178 | 4887.5 |
| | ethoxy)-ethoxy]-acetyl)-(γ-Glu)-CO—(CH₂)₁₆—CO₂H)AQ- | | |
| | Aib-AFIEWLLEGGSSSGAPPPSGE-NH₂ | | |
| 173 | Y-Aib-QGTFTSDFS-Orn-αMeL-LDKK((2-[2-(2-Amino- | 179 | 4845.4 |
| | ethoxy)-ethoxy]-acetyl)-(γ-Glu)-CO—(CH₂)₁₆—CO₂H)AQ- | | |
| | Aib-AFIEWLLEGGSSSGAPPPSGS-NH₂ | | |
| 174 | Y-Aib-QGTFTSDFS-Orn-αMeL-LDKK((2-[2-(2-Amino- | 180 | 4859.4 |
| | ethoxy)-ethoxy]-acetyl)-(γ-Glu)-CO—(CH₂)₁₆—CO₂H)AQ- | | |
| | Aib-AFIEWLLEGGSSSGAPPPSGT-NH₂ | | |
| 175 | Y-Aib-QGTFTSDFS-Orn-αMeL-LDKK((2-[2-(2-Amino- | 181 | 4901.5 |
| | ethoxy)-ethoxy]-acetyl)-(γ-Glu)-CO—(CH₂)₁₆—CO₂H)AQ- | | |
| | Aib-AFIEWLLEGGTSSGAPPPSGE-NH₂ | | |
| 176 | Y-Aib-QGTFTSDFS-Orn-αMeL-LDKK((2-[2-(2-Amino- | 182 | 4860.4 |
| | ethoxy)-ethoxy]-acetyl)-(γ-Glu)-CO—(CH₂)₁₆—CO₂H)AQ- | | |
| | Aib-AFIEWLLEGGTSSGAPPPSGS-NH₂ | | |
| 177 | Y-Aib-QGTFTSDFS-Orn-αMeL-LDKK((2-[2-(2-Amino- | 183 | 4873.5 |
| | ethoxy)-ethoxy]-acetyl)-(γ-Glu)-CO—(CH₂)₁₆—CO₂H)AQ- | | |
| | Aib-AFIEWLLEGGTSSGAPPPSGT-NH₂ | | |
| 178 | Y-Aib-QGTFTSD-F(4CN)-S-Orn-αMeL-LDKK((2-[2-(2- | 184 | 5030.6 |
| | Amino-ethoxy)-ethoxy]-acetyl)₂-(γ-Glu)-CO—(CH₂)₁₆- | | |
| | CO₂H))AQ-Aib-AFIEYLLE-Aib-GPSSGAPPPSGS-NH₂ | | |
| 179 | Y-Aib-QGTFTSDFS-Orn-αMeL-LDKK((2-[2-(2-Amino- | 185 | 5037.6 |
| | ethoxy)-ethoxy]-acetyl)₂-(γ-Glu)-CO—(CH₂)₁₆—CO₂H))AQ- | | |
| | Aib-AFIEYLLE-Aib-GESSGAPPPSGS-NH₂ | | |
| 180 | Y-Aib-QGTFTSDFS-Orn-αMeL-LDKK((2-[2-(2-Amino- | 186 | 5051.7 |
| | ethoxy)-ethoxy]-acetyl)₂-(γ-Glu)-CO—(CH₂)₁₆—CO₂H))AQ- | | |
| | Aib-AFIEYLLE-Aib-GESSGAPPPSGT-NH₂ | | |
| 181 | Y-Aib-QGTFTSDFS-Orn-αMeL-LDKK((2-[2-(2-Amino- | 187 | 5045.7 |
| | ethoxy)-ethoxy]-acetyl)₂-(γ-Glu)-CO—(CH₂)₁₆—CO₂H))AQ- | | |
| | Aib-AFIEYLLE-Aib-GHSSGAPPPSGS-NH₂ | | |
| 182 | Y-Aib-QGTFTSDFS-Orn-αMeL-LDKK((2-[2-(2-Amino- | 188 | 5059.7 |
| | ethoxy)-ethoxy]-acetyl)₂-(γ-Glu)-CO—(CH₂)₁₆—CO₂H))AQ- | | |
| | Aib-AFIEYLLE-Aib-GHSSGAPPPSGT-NH₂ | | |
| 183 | Y-Aib-QGTFTSDFS-Orn-αMeL-LDKK((2-[2-(2-Amino- | 189 | 5056.7 |
| | ethoxy)-ethoxy]-acetyl)₂-(γ-Glu)-CO—(CH₂)₁₆—CO₂H))AQ- | | |
| | Aib-AFIEYLLE-Aib-G-4Pal-SSGAPPPSGS-NH₂ | | |
| 184 | Y-Aib-QGTFTSDFS-Orn-αMeL-LDKK((2-[2-(2-Amino- | 190 | 5070.7 |
| | ethoxy)-ethoxy]-acetyl)₂-(γ-Glu)-CO—(CH₂)₁₆—CO₂H))AQ- | | |
| | Aib-AFIEYLLE-Aib-G-4Pal-SSGAPPPSGT-NH₂ | | |
| 185 | Y-Aib-QGTFTSDFS-Orn-αMeL-LDKK((2-[2-(2-Amino- | 191 | 5079.7 |
| | ethoxy)-ethoxy]-acetyl)₂-(γ-Glu)-CO—(CH₂)₁₆—CO₂H))AQ- | | |
| | Aib-AFIEYLLE-Aib-GESSGAPPPSGE-NH₂ | | |
| 186 | Y-Aib-QGTFTSDFS-Orn-αMeL-LDKK((2-[2-(2-Amino- | 192 | 5087.7 |
| | ethoxy)-ethoxy]-acetyl)₂-(γ-Glu)-CO—(CH₂)₁₆—CO₂H))AQ- | | |
| | Aib-AFIEYLLE-Aib-GHSSGAPPPSGE-NH₂ | | |
| 187 | Y-Aib-QGTFTSD-4Pal-SR-αMeL-LDKK((2-[2-(2-Amino- | 193 | 4935.5 |
| | ethoxy)-ethoxy]-acetyl)-(γ-Glu)-CO—(CH₂)₁₆—CO₂H)AQ- | | |
| | Aib-AFIEYLIE-Aib-GESSGAPPPSGS-NH₂ | | |
| 188 | Y-Aib-QGTFTSD-4Pal-S-Dab-αMeL-LDKK((2-[2-(2- | 194 | 4879.4 |
| | Amino-ethoxy)-ethoxy]-acetyl)-(γ-Glu)-CO—(CH₂)₁₆- | | |
| | CO₂H)AQ-Aib-AFIEYLIE-Aib-GESSGAPPPSGS-NH₂ | | |
| 189 | Y-Aib-QGTFTSDFS-Orn-αMeL-LDKK((2-[2-(2-Amino- | 195 | 5098.7 |
| | ethoxy)-ethoxy]-acetyl)₂-(γ-Glu)-CO—(CH₂)₁₆—CO₂H))AQ- | | |
| | Aib-AFIEYLLE-Aib-G-4Pal-SSGAPPPSGE-NH₂ | | |
| 190 | Y-Aib-QGTFTSD-F(4CN)-S-Orn-αMeL-LDKK((2-[2-(2- | 196 | 4925.5 |
| | Amino-ethoxy)-ethoxy]-acetyl)-(γ-Glu)-CO—(CH₂)₁₆- | | |
| | CO₂H)AQ-Aib-AFIEYLLE-Aib-GHSSGAPPPSGS-NH₂ | | |
| 191 | Y-Aib-QGTFTSD-F(4CN)-S-Orn-αMeL-LDKK((2-[2-(2- | 197 | 4885.5 |
| | Amino-ethoxy)-ethoxy]-acetyl)-(γ-Glu)-CO—(CH₂)₁₆- | | |
| | CO₂H)AQ-Aib-AFIEYLLE-Aib-GPSSGAPPPSGS-NH₂ | | |
| 192 | Y-Aib-QGTFTSDFS-Orn-αMeL-LDKK((2-[2-(2-Amino- | 198 | 4989.6 |
| | ethoxy)-ethoxy]-acetyl)-(γ-Glu)₂-CO—(CH₂)₁₆—CO₂H)AQ- | | |
| | Aib-AFIEYLLE-Aib-GPSSGAPPPSGS-NH₂ | | |
| 193 | Y-Aib-QGTFTSDFS-Orn-αMeL-LDKK((γ-Glu)-(2-[2-(2- | 199 | 4989.6 |
| | Amino-ethoxy)-ethoxy]-acetyl)-(γ-Glu)-CO—(CH₂)₁₆- | | |
| | CO₂H)AQ-Aib-AFIEYLLE-Aib-GPSSGAPPPSGS-NH₂ | | |
| 194 | Y-Aib-QGTFTSDFS-Orn-αMeL-LDKK((2-[2-(2-Amino- | 200 | 5047.7 |
| | ethoxy)-ethoxy]-acetyl)₂-(γ-Glu)-CO—(CH₂)₁₆—CO₂H))AQ- | | |
| | Aib-AFIEYLLE-Aib-GPSSGAPPPEGS-NH₂ | | |
| 195 | Y-Aib-QGTFTSDFS-Orn-αMeL-LDKK((2-[2-(2-Amino- | 201 | 5105.7 |
| | ethoxy)-ethoxy]-acetyl)₂-(γ-Glu)-CO—(CH₂)₁₆—CO₂H))AQ- | | |
| | Aib-AFIEYLLE-Aib-GPSSGEPPPEGS-NH₂ | | |
| 196 | Y-Aib-QGTFTSDFS-Orn-αMeL-LDKK((2-[2-(2-Amino- | 202 | 4803.4 |
| | ethoxy)-ethoxy]-acetyl)-(γ-Glu)-CO—(CH₂)₁₆—CO₂H)AQ- | | |
| | Aib-AFIEYLLEGGPSSGAPPPGGS | | |
| 197 | Y-Aib-QGTFTSDFS-Orn-αMeL-LDKK((2-[2-(2-Amino- | 203 | 4817.4 |
| | ethoxy)-ethoxy]-acetyl)-(γ-Glu)-CO—(CH₂)₁₆—CO₂H)AQ- | | |
| | Aib-AFIEYLLEGGPSSGAPPPGGT | | |
| 198 | Y-Aib-QGTFTSDFS-Orn-αMeL-LDKK((γ-Glu)₂-CO— | 204 | 4786.3 |
| | (CH₂)₁₆—CO₂H)AQ-Aib-AFIEYLLEGGPSSGAPPPGGS- | | |
| | NH₂ | | |
| 199 | Y-Aib-QGTFTSDFS-Orn-αMeL-LDKK((γ-Glu)₂-CO— | 205 | 4816.4 |
| | (CH₂)₁₆—CO₂H)AQ-Aib-AFIEYLLEGGPSSGAPPPSGS- | | |
| | NH₂ | | |
| 200 | Y-Aib-QGTFTSDFS-Orn-αMeL-LDKK((2-[2-(2-Amino- | 206 | 4874.5 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSGT-NH₂ | | |
| 201 | Y-Aib-QGTFTSDFS-Orn-αMeL-LDKK((γ-Glu)₂-CO— | 207 | 4844.4 |
| | (CH₂)₁₆—CO₂H)AQ-Aib-AFIEYLLE-Aib-GPSSGAPPPSGS- | | |
| | NH₂ | | |
| 202 | Y-Aib-QGTFTSDFS-Orn-αMeL-LDKK((γ-Glu)₃-CO— | 208 | 4973.5 |
| | (CH₂)₁₆—CO₂H)AQ-Aib-AFIEYLLE-Aib-GPSSGAPPPSGS- | | |
| | NH₂ | | |
| 203 | Y-Aib-QGTFTSDFS-Dap-αMeL-LDKK((γ-Glu)₂-CO— | 209 | 4816.4 |
| | (CH₂)₁₆—CO₂H)AQ-Aib-AFIEYLLE-Aib-GPSSGAPPPSGS- | | |
| | NH₂ | | |
| 204 | Y-Aib-QGTFTSDFS-Orn-αMeL-LDKK((γ-Glu)₂-CO— | 210 | 4844.4 |
| | (CH₂)₁₆—CO₂H)AQ-Aib-AFIEYLLEaGPSSGAPPPSGT-NH₂ | | |
| 205 | Y-Aib-QGTFTSDFS-Orn-αMeL-LDKK((γ-Glu)₃-CO— | 211 | 4973.5 |
| | (CH₂)₁₆—CO₂H)AQ-Aib-AFIEYLLEaGPSSGAPPPSGT-NH₂ | | |
| 206 | Y-Aib-QGTFTSDFS-Orn-αMeL-LDKK((2-[2-(2-Amino- | 212 | 4930.5 |
| | ethoxy)-ethoxy]-acetyl)-(γ-Glu)-CO—(CH₂)₁₆—CO₂H)AQ- | | |
| | Aib-AFIEYLLEaGPSSGAPPPEGE-NH₂ | | |
| 207 | Y-Aib-QGTFTSDFS-Orn-αMeL-LDKK((2-[2-(2-Amino- | 213 | 4928.5 |
| | ethoxy)-ethoxy]-acetyl)-(γ-Glu)-CO—(CH₂)₁₆—CO₂H)AQ- | | |
| | Aib-AFIEYLLEaGHSSGAPPPSGE-NH₂ | | |
| 208 | Y-Aib-QGTFTSDFS-Orn-αMeL-LDKK((2-[2-(2-Amino- | 214 | 4961.6 |
| | ethoxy)-ethoxy]-acetyl)-(γ-Glu)-CO—(CH₂)₁₆—CO₂H)AQ- | | |
| | Aib-AFIEYLLEaG-K(Ac)-SSGAPPPSGE-NH₂ | | |
| 209 | Y-Aib-QGTFTSDFS-Orn-αMeL-LDKK((2-[2-(2-Amino- | 215 | 4846.4 |
| | ethoxy)-ethoxy]-acetyl)-(γ-Glu)-CO—(CH₂)₁₆—CO₂H)AQ- | | |
| | Aib-AFIEYLLEaGPSSGAPPPSGS-NH₂ | | |
| 210 | Y-Aib-QGTFTSDFS-Orn-αMeL-LDKK((2-[2-(2-Amino- | 216 | 4860.5 |
| | ethoxy)-ethoxy]-acetyl)-(γ-Glu)-CO—(CH₂)₁₆—CO₂H)AQ- | | |
| | Aib-AFIEYLLEaGPSSGAPPPSGT-NH₂ | | |
| 211 | Y-Aib-QGTFTSDFS-Orn-αMeL-LDKK((2-[2-(2-Amino- | 217 | 4907.5 |
| | ethoxy)-ethoxy]-acetyl)-(γ-Glu)-CO—(CH₂)₁₆—CO₂H)AQ- | | |
| | Aib-AFIEYLLEaGPSSGAPPPSG-4Pal-NH₂ | | |
| 212 | Y-Aib-QGTFTSDFS-Orn-αMeL-LDKK((2-[2-(2-Amino- | 218 | 4847.4 |
| | ethoxy)-ethoxy]-acetyl)-(γ-Glu)-CO—(CH₂)₁₆—CO₂H)AQ- | | |
| | Aib-AFIEYLLEaGPSSGAPPPSGS | | |
| 213 | Y-Aib-QGTFTSDFS-Orn-αMeL-LDKK((2-[2-(2-Amino- | 219 | 4861.5 |
| | ethoxy)-ethoxy]-acetyl)-(γ-Glu)-CO—(CH₂)₁₆—CO₂H)AQ- | | |
| | Aib-AFIEYLLEaGPSSGAPPPSGT | | |
| 214 | Y-Aib-QGTFTSDFS-Orn-αMeL-LDKK((2-[2-(2-Amino- | 220 | 4888.5 |
| | ethoxy)-ethoxy]-acetyl)-(γ-Glu)-CO—(CH₂)₁₆—CO₂H)AQ- | | |
| | Aib-AFIEYLLEaGPSS-Aib-APPPSGT-NH₂ | | |
| 215 | Y-Aib-QGTFTSDFSK-αMeL-LDKK((2-[2-(2-Amino- | 221 | 4878.4 |
| | ethoxy)-ethoxy]-acetyl)-(γ-Glu)-CO—(CH₂)₁₆—CO₂H)AQ- | | |
| | Aib-AFIEYLIEGGESSGAPPPSGS-NH₂ | | |
| 216 | Y-Aib-QGTFTSDFSK-αMeL-LDKK((2-[2-(2-Amino- | 222 | 4906.5 |
| | ethoxy)-ethoxy]-acetyl)-(γ-Glu)-CO—(CH₂)₁₆—CO₂H)AQ- | | |
| | Aib-AFIEYLIE-Aib-GESSGAPPPSGS-NH₂ | | |
| 217 | Y-Aib-QGTFTSDFSK-αMeL-LDKK((2-[2-(2-Amino- | 223 | 4948.5 |
| | ethoxy)-ethoxy]-acetyl)-(γ-Glu)-CO—(CH₂)₁₆—CO₂H)AQ- | | |
| | Aib-AFIEYLIE-Aib-GESSGAPPPSGE-NH₂ | | |
| 218 | Y-Aib-QGTFTSDFS-Orn-αMeL-LDKK((2-[2-(2-Amino- | 224 | 4892.5 |
| | ethoxy)-ethoxy]-acetyl)-(γ-Glu)-CO—(CH₂)₁₆—CO₂H)AQ- | | |
| | Aib-AFIEYLIE-Aib-GESSGAPPPSGS-NH₂ | | |
| 219 | Y-Aib-QGTFTSDFS-Orn-αMeL-LDKK((2-[2-(2-Amino- | 225 | 4934.5 |
| | ethoxy)-ethoxy]-acetyl)-(γ-Glu)-CO—(CH₂)₁₆—CO₂H)AQ- | | |
| | Aib-AFIEYLIE-Aib-GESSGAPPPSGE-NH₂ | | |
| 220 | Y-Aib-QGTFTSDFSR-αMeL-LDKK((2-[2-(2-Amino- | 226 | 4906.5 |
| | ethoxy)-ethoxy]-acetyl)-(γ-Glu)-CO—(CH₂)₁₆—CO₂H)AQ- | | |
| | Aib-AFIEYLIEGGESSGAPPPSGS-NH₂ | | |
| 221 | Y-Aib-QGTFTSDFSR-αMeL-LDKK((2-[2-(2-Amino- | 227 | 4934.5 |
| | ethoxy)-ethoxy]-acetyl)-(γ-Glu)-CO—(CH₂)₁₆—CO₂H)AQ- | | |
| | Aib-AFIEYLIE-Aib-GESSGAPPPSGS-NH₂ | | |
| 222 | Y-Aib-QGTFTSD-4Pal-S-Orn-αMeL-LDKK((2-[2-(2- | 228 | 5012.6 |
| | Amino-ethoxy)-ethoxy]-acetyl)-(γ-Glu)-CO—(CH₂)₁₆- | | |
| | CO₂H)AQ-αMe4Pal-AFIEYLIE-Aib-GESSGAPPPSGE- | | |
| | NH₂ | | |
| 223 | Y-Aib-QGTFTSD-4Pal-S-Orn-αMeL-LDKK((2-[2-(2- | 229 | 4970.5 |
| | Amino-ethoxy)-ethoxy]-acetyl)-(γ-Glu)-CO—(CH₂)₁₆- | | |
| | CO₂H)AQ-αMe4Pal-AFIEYLIE-Aib-GESSGAPPPSGS- | | |
| | NH₂ | | |
| 224 | Y-Aib-QGTFTSD-4Pal-SR-αMeL-LDKK((2-[2-(2-Amino- | 230 | 5012.6 |
| | ethoxy)-ethoxy]-acetyl)-(γ-Glu)-CO—(CH₂)₁₆—CO₂H)AQ- | | |
| | αMe4Pal-AFIEYLIE-Aib-GESSGAPPPSGS-NH₂ | | |
| 225 | Y-Aib-QGTFTSD-4Pal-SR-αMeL-LDKK((2-[2-(2-Amino- | 231 | 5054.6 |
| | ethoxy)-ethoxy]-acetyl)-(γ-Glu)-CO—(CH₂)₁₆—CO₂H)AQ- | | |
| | αMe4Pal-AFIEYLIE-Aib-GESSGAPPPSGE-NH₂ | | |
| 226 | Y-Aib-QGTFTSD-4Pal-SQ-αMeL-LDKK((2-[2-(2-Amino- | 232 | 4984.5 |
| | ethoxy)-ethoxy]-acetyl)-(γ-Glu)-CO—(CH₂)₁₆—CO₂H)AQ- | | |
| | αMe4Pal-AFIEYLIE-Aib-GESSGAPPPSGS-NH₂ | | |
| 227 | Y-Aib-QGTFTSD-4Pal-SQ-αMeL-LDKK((2-[2-(2-Amino- | 233 | 5026.6 |
| | ethoxy)-ethoxy]-acetyl)-(γ-Glu)-CO—(CH₂)₁₆—CO₂H)AQ- | | |
| | αMe4Pal-AFIEYLIE-Aib-GESSGAPPPSGE-NH₂ | | |
| 228 | Y-Aib-QGTFTSDFS-Orn-αMeL-LDKK((2-[2-(2-Amino- | 234 | 5011.6 |
| | ethoxy)-ethoxy]-acetyl)-(γ-Glu)-CO—(CH₂)₁₆—CO₂H)AQ- | | |
| | αMe4Pal-AFIEYLIE-Aib-GESSGAPPPSGE-NH₂ | | |
| 229 | Y-Aib-QGTFTSDFS-Orn-αMeL-LDKK((2-[2-(2-Amino- | 235 | 4969.6 |
| | ethoxy)-ethoxy]-acetyl)-(γ-Glu)-CO—(CH₂)₁₆—CO₂H)AQ- | | |
| | αMe4Pal-AFIEYLIE-Aib-GESSGAPPPSGS-NH₂ | | |
| 230 | Y-Aib-QGTFTSDFSR-αMeL-LDKK((2-[2-(2-Amino- | 236 | 5011.6 |
| | ethoxy)-ethoxy]-acetyl)-(γ-Glu)-CO—(CH₂)₁₆—CO₂H)AQ- | | |
| | αMe4Pal-AFIEYLIE-Aib-GESSGAPPPSGS-NH₂ | | |
| 231 | Y-Aib-QGTFTSDFSR-αMeL-LDKK((2-[2-(2-Amino- | 237 | 5053.6 |
| | ethoxy)-ethoxy]-acetyl)-(γ-Glu)-CO—(CH₂)₁₆—CO₂H)AQ- | | |
| | αMe4Pal-AFIEYLIE-Aib-GESSGAPPPSGE-NH₂ | | |
| 232 | Y-Aib-QGTFTSDFSQ-αMeL-LDKK((2-[2-(2-Amino- | 238 | 4983.5 |
| | ethoxy)-ethoxy]-acetyl)-(γ-Glu)-CO—(CH₂)₁₆—CO₂H)AQ- | | |
| | αMe4Pal-AFIEYLIE-Aib-GESSGAPPPSGS-NH₂ | | |
| 233 | Y-Aib-QGTFTSDFSQ-αMeL-LDKK((2-[2-(2-Amino- | 239 | 5025.6 |
| | ethoxy)-ethoxy]-acetyl)-(γ-Glu)-CO—(CH₂)₁₆—CO₂H)AQ- | | |
| | αMe4Pal-AFIEYLIE-Aib-GESSGAPPPSGE-NH₂ | | |
| 234 | Y-Aib-QGTFTSDFS-Orn-αMeL-LDKK((2-[2-(2-Amino- | 240 | 4818.4 |
| | ethoxy)-ethoxy]-acetyl)-(γ-Glu)-CO—(CH₂)₁₆—CO₂H)AQ- | | |
| | Aib-AFIEYLLEGG-Hyp-SSGAPPPGGS-NH₂ | | |
| 235 | Y-Aib-QGTFTSDFS-Orn-αMeL-LDKK((2-[2-(2-Amino- | 241 | 4818.4 |
| | ethoxy)-ethoxy]-acetyl)-(γ-Glu)-CO—(CH₂)₁₆—CO₂H)AQ- | | |
| | Aib-AFIEYLLEGGPSSGA-Hyp-PPGGS-NH₂ | | |
| 236 | Y-Aib-QGTFTSDFS-Orn-αMeL-LDKK((2-[2-(2-Amino- | 242 | 4818.4 |
| | ethoxy)-ethoxy]-acetyl)-(γ-Glu)-CO—(CH₂)₁₆—CO₂H)AQ- | | |
| | Aib-AFIEYLLEGGPSSGAP-Hyp-PGGS-NH₂ | | |
| 237 | Y-Aib-QGTFTSDFS-Orn-αMeL-LDKK((2-[2-(2-Amino- | 243 | 4818.4 |
| | ethoxy)-ethoxy]-acetyl)-(γ-Glu)-CO—(CH₂)₁₆—CO₂H)AQ- | | |
| | Aib-AFIEYLLEGGPSSGAPP-Hyp-GGS-NH₂ | | |
| 238 | Y-Aib-QGTFTSDFS-Orn-αMeL-LDKK((2-[2-(2-Amino- | 244 | 4888.5 |
| | ethoxy)-ethoxy]-acetyl)-(γ-Glu)-CO—(CH₂)₁₆—CO₂H)AQ- | | |
| | Aib-AFIEYLLE-Aib-GPSS-Aib-APPPSGS-NH₂ | | |
| 239 | Y-Aib-QGTFTSDFS-Orn-αMeL-LDKK((2-[2-(2-Amino- | 245 | 4874.5 |
| | ethoxy)-ethoxy]-acetyl)-(γ-Glu)-CO—(CH₂)₁₆—CO₂H)AQ- | | |
| | Aib-AFIEYLLE-Aib-GPSSG-Aib-PPPSGS-NH₂ | | |
| 240 | Y-Aib-QGTFTSDFS-Orn-αMeL-LDKK((2-[2-(2-Amino- | 246 | 4892.5 |
| | ethoxy)-ethoxy]-acetyl)-(γ-Glu)-CO—(CH₂)₁₆—CO₂H)AQ- | | |
| | Aib-AFIEYLLE-Aib-GESSGAPPPSGS-NH₂ | | |
| 241 | Y-Aib-QGTFTSDFS-Orn-αMeL-LDKK((2-[2-(2-Amino- | 247 | 4900.5 |
| | ethoxy)-ethoxy]-acetyl)-(γ-Glu)-CO—(CH₂)₁₆—CO₂H)AQ- | | |
| | Aib-AFIEYLLE-Aib-GHSSGAPPPSGS-NH₂ | | |
| 242 | Y-Aib-QGTFTSDFS-Orn-αMeL-LDKK((2-[2-(2-Amino- | 248 | 4911.5 |
| | ethoxy)-ethoxy]-acetyl)-(γ-Glu)-CO—(CH₂)₁₆—CO₂H)AQ- | | |
| | Aib-AFIEYLLE-Aib-G-4Pal-SSGAPPPSGS-NH₂ | | |
| 243 | Y-Aib-QGTFTSDFS-Orn-αMeL-LDKK((2-[2-(2-Amino- | 249 | 5029.6 |
| | ethoxy)-ethoxy]-acetyl)-(γ-Glu)₂-CO—(CH₂)₁₆—CO₂H)AQ- | | |
| | Aib-AFIEYLLE-Aib-GHSSGAPPPSGS-NH₂ | | |
| 244 | Y-Aib-QGTFTSDFS-Orn-αMeL-LDKK((γ-Glu)-(2-[2-(2- | 250 | 5021.6 |
| | Amino-ethoxy)-ethoxy]-acetyl)-(γ-Glu)-CO—(CH₂)₁₆- | | |
| | CO₂H)AQ-Aib-AFIEYLLE-Aib-GESSGAPPPSGS-NH₂ | | |
| 245 | Y-Aib-QGTFTSDFS-Orn-αMeL-LDKK((γ-Glu)-(2-[2-(2- | 251 | 5029.6 |
| | Amino-ethoxy)-ethoxy]-acetyl)-(γ-Glu)-CO—(CH₂)₁₆- | | |
| | CO₂H)AQ-Aib-AFIEYLLE-Aib-GHSSGAPPPSGS-NH₂ | | |
| 246 | Y-Aib-QGTFTSDFS-Orn-αMeL-LDKK((2-[2-(2-Amino- | 252 | 4934.5 |
| | ethoxy)-ethoxy]-acetyl)-(γ-Glu)-CO—(CH₂)₁₆—CO₂H)AQ- | | |
| | Aib-AFIEYLLE-Aib-GESSGAPPPSGE-NH₂ | | |
| 247 | Y-Aib-QGTFTSDFS-Orn-αMeL-LDKK((2-[2-(2-Amino- | 253 | 4942.5 |
| | ethoxy)-ethoxy]-acetyl)-(γ-Glu)-CO—(CH₂)₁₆—CO₂H)AQ- | | |
| | Aib-AFIEYLLE-Aib-GHSSGAPPPSGE-NH₂ | | |
| 248 | Y-Aib-QGTFTSDFS-Orn-αMeL-LDKK((2-[2-(2-Amino- | 254 | 4953.6 |
| | ethoxy)-ethoxy]-acetyl)-(γ-Glu)-CO—(CH₂)₁₆—CO₂H)AQ- | | |
| | Aib-AFIEYLLE-Aib-G-4Pal-SSGAPPPSGE-NH₂ | | |
| 249 | Y-Aib-QGTFTSDFS-Orn-αMeL-LDKK((2-[2-(2-Amino- | 255 | 4905.5 |
| | ethoxy)-ethoxy]-acetyl)-(γ-Glu)-CO—(CH₂)₁₆—CO₂H)AQ- | | |
| | Aib-AFIEWLLEGGPSSGAPPPHGS-NH₂ | | |
| 250 | Y-Aib-QGTFTSDFS-Orn-αMeL-LDKK((2-[2-(2-Amino- | 256 | 4869.5 |
| | ethoxy)-ethoxy]-acetyl)-(γ-Glu)-CO—(CH₂)₁₆—CO₂H)AQ- | | |
| | Aib-AFIEWLLEGGPSSGAPPPTGS-NH₂ | | |
| 251 | Y-Aib-QGTFTSDFS-Orn-αMeL-LDKK((2-[2-(2-Amino- | 257 | 4916.5 |
| | ethoxy)-ethoxy]-acetyl)-(γ-Glu)-CO—(CH₂)₁₆—CO₂H)AQ- | | |
| | Aib-AFIEWLLEGGPSSGAPPP-4Pal-GS-NH₂ | | |
| 252 | Y-Aib-QGTFTSDFS-Orn-αMeL-LDKK((2-[2-(2-Amino- | 258 | 4939.5 |
| | ethoxy)-ethoxy]-acetyl)-(γ-Glu)-CO—(CH₂)₁₆—CO₂H)AQ- | | |
| | Aib-AFIEYLLEaG-4Pal-SSGAPPPSGE-NH₂ | | |
| 253 | Y-Aib-QGTFTSDFS-Orn-αMeL-LDKK((2-[2-(2-Amino- | 259 | 5057.7 |
| | ethoxy)-ethoxy]-acetyl)₂-(γ-Glu)-CO—(CH₂)₁₆—CO₂H))AQ- | | |
| | Aib-AFIEYLLEaGPSSGHPPPSGS-NH₂ | | |
| 254 | Y-Aib-QGTFTSDFS-Or-αMeL-LDKK((2-[2-(2-Amino- | 260 | 5068.7 |
| | ethoxy)-ethoxy]-acetyl)₂-(γ-Glu)-CO—(CH₂)₁₆—CO₂H))AQ- | | |
| | Aib-AFIEYLLEaGPSSG-4Pal-PPPSGS-NH₂ | | |
| 255 | Y-Aib-QGTFTSDFS-Orn-αMeL-LDKK((2-[2-(2-Amino- | 261 | 5107.7 |
| | ethoxy)-ethoxy]-acetyl)₂-(γ-Glu)-CO—(CH₂)₁₆—CO₂H))AQ- | | |
| | Aib-AFIEYLLEaGPSSGHPPPSGH-NH₂ | | |
| 256 | Y-Aib-QGTFTSDFS-Or-αMeL-LDKK((2-[2-(2-Amino- | 262 | 5118.7 |
| | ethoxy)-ethoxy]-acetyl)₂-(γ-Glu)-CO—(CH₂)₁₆—CO₂H))AQ- | | |
| | Aib-AFIEYLLEaGPSSGHPPPSG-4Pal-NH₂ | | |
| 257 | Y-Aib-QGTFTSDFS-Orn-αMeL-LDKK((2-[2-(2-Amino- | 263 | 5118.7 |
| | ethoxy)-ethoxy]-acetyl)₂-(γ-Glu)-CO—(CH₂)₁₆—CO₂H))AQ- | | |
| | Aib-AFIEYLLEaGPSSG-4Pal-PPPSGH-NH₂ | | |
| 258 | Y-Aib-QGTFTSDFS-Orn-αMeL-LDKK((2-[2-(2-Amino- | 264 | 5129.8 |
| | ethoxy)-ethoxy]-acetyl)₂-(γ-Glu)-CO—(CH₂)₁₆—CO₂H))AQ- | | |
| | Aib-AFIEYLLEaGPSSG-4Pal-PPPSG-4Pal-NH₂ | | |
| 259 | Y-Aib-QGTFTSDFS-Orn-αMeL-LDKK((2-[2-(2-Amino- | 265 | 4896.5 |
| | ethoxy)-ethoxy]-acetyl)-(γ-Glu)-CO—(CH₂)₁₆—CO₂H)AQ- | | |
| | Aib-AFIEYLLEaGPSSGAPPPSGH-NH₂ | | |
| 260 | Y-Aib-QGTFTSDFS-Orn-αMeL-LDKK((γ-Glu)₂-CO— | 266 | 4830.4 |
| | (CH₂)₁₆—CO₂H)AQ-Aib-AFIEYLLEGGPSSGAPPPSGT- | | |
| | NH₂ | | |
| 261 | Y-Aib-QGTFTSDFS-Orn-αMeL-LDKK((2-[2-(2-Amino- | 267 | 4863.5 |
| | ethoxy)-ethoxy]-acetyl)-(γ-Glu)-CO—(CH₂)₁₆—CO₂H)AQ- | | |
| | Aib-AFIEYLLEGGPSSGAPPPGG-4Pal-NH₂ | | |
| 262 | Y-Aib-QGTFTSDFS-Orn-αMeL-LDKK((2-[2-(2-Amino- | 268 | 4958.6 |
| | ethoxy)-ethoxy]-acetyl)-(γ-Glu)-CO—(CH₂)₁₆—CO₂H)AQ- | | |
| | Aib-AFIEWLLEGGPSSGAPPPEG-4Pal-NH₂ | | |
| 263 | Y-Aib-QGTFTSDFS-Orn-αMeL-LDKK((2-[2-(2-Amino- | 269 | 4873.5 |
| | ethoxy)-ethoxy]-acetyl)-(γ-Glu)-CO—(CH₂)₁₆—CO₂H)AQ- | | |
| | Aib-AFIEWLLEaGSSSGAPPPSGT-NH₂ | | |
| 264 | Y-Aib-QGTFTSDFS-Orn-αMeL-LDKK((2-[2-(2-Amino- | 270 | 4887.5 |
| | ethoxy)-ethoxy]-acetyl)-(γ-Glu)-CO—(CH₂)₁₆—CO₂H)AQ- | | |
| | Aib-AFIEWLLE-Aib-GSSSGAPPPSGT-NH₂ | | |
| 265 | Y-Aib-QGTFTSDFS-Orn-αMeL-LDKK((2-[2-(2-Amino- | 271 | 4951.5 |
| | ethoxy)-ethoxy]-acetyl)₂-(γ-Glu)-CO—(CH₂)₁₆—CO₂H))AQ- | | |
| | Aib-AFIEWLLEaGHSSGAPPPSGE-NH₂ | | |
| 266 | Y-Aib-QGTFTSDFS-Orn-αMeL-LDKK((2-[2-(2-Amino- | 272 | 4965.6 |
| | ethoxy)-ethoxy]-acetyl)₂-(γ-Glu)-CO—(CH₂)₁₆—CO₂H))AQ- | | |
| | Aib-AFIEWLLE-Aib-GHSSGAPPPSGE-NH₂ | | |
| 267 | Y-Aib-QGTFTSDFS-Orn-αMeL-LDKK((2-[2-(2-Amino- | 273 | 4948.5 |
| | ethoxy)-ethoxy]-acetyl)-(γ-Glu)-CO—(CH₂)₁₆—CO₂H)AQ- | | |
| | Aib-AFIEWLLEGG-4Pal-SSGAPPPSGE-NH₂ | | |
| 268 | Y-Aib-QGTFTSDFS-Orn-αMeL-LDKK((2-[2-(2-Amino- | 274 | 4920.5 |
| | ethoxy)-ethoxy]-acetyl)-(γ-Glu)-CO—(CH₂)₁₆—CO₂H)AQ- | | |
| | Aib-AFIEWLLEGG-4Pal-SSGAPPPSGT-NH₂ | | |
| 269 | Y-Aib-QGTFTSDFS-Orn-αMeL-LDKK((2-[2-(2-Amino- | 275 | 4945.5 |
| | ethoxy)-ethoxy]-acetyl)-(γ-Glu)-CO—(CH₂)₁₆—CO₂H)AQ- | | |
| | Aib-AFIEWLLEGGHSSGAPPPSGH-NH₂ | | |
| 270 | Y-Aib-QGTFTSDFS-Orn-αMeL-LDKK((2-[2-(2-Amino- | 276 | 4895.5 |
| | ethoxy)-ethoxy]-acetyl)-(γ-Glu)-CO—(CH₂)₁₆—CO₂H)AQ- | | |
| | Aib-AFIEWLLEGGSSSGAPPPSGH-NH₂ | | |
| 271 | Y-Aib-QGTFTSDFS-Orn-αMeL-LDKK((2-[2-(2-Amino- | 277 | 5052.6 |
| | ethoxy)-ethoxy]-acetyl)₂-(γ-Glu)-CO—(CH₂)₁₆—CO₂H))AQ- | | |
| | Aib-AFIEWLLEGGHSSGAPPPGGE-NH₂ | | |
| 272 | Y-Aib-QGTFTSDFS-Orn-αMeL-LDKK((2-[2-(2-Amino- | 278 | 4829.4 |
| | ethoxy)-ethoxy]-acetyl)-(γ-Glu)-CO—(CH₂)₁₆—CO₂H)AQ- | | |
| | Aib-AFIEWLLEGGSSSGAPPPGGT-NH₂ | | |
| 273 | Y-Aib-QGTFTSDFS-Orn-αMeL-LDKK((2-[2-(2-Amino- | 279 | 4907.5 |
| | ethoxy)-ethoxy]-acetyl)-(γ-Glu)-CO—(CH₂)₁₆—CO₂H)AQ- | | |
| | Aib-AFIEWLLEGGHSSGAPPPGGE-NH₂ | | |
| 274 | Y-Aib-QGTFTSD-4Pal-S-Orn-αMeL-LDKK((2-[2-(2- | 280 | 4908.5 |
| | Amino-ethoxy)-ethoxy]-acetyl)-(γ-Glu)-CO—(CH₂)₁₆- | | |
| | CO₂H)AQ-Aib-AFIEYLLEaGPSSGAPPPSG-4Pal-NH₂ | | |
| 275 | Y-Aib-QGTFTSD-4Pal-S-Orn-αMeL-LDKK((2-[2-(2- | 281 | 4889.5 |
| | Amino-ethoxy)-ethoxy]-acetyl)-(γ-Glu)-CO—(CH₂)₁₆- | | |
| | CO₂H)AQ-Aib-AFIEYLLEaGPSS-Aib-APPPSGT-NH₂ | | |
| 276 | Y-Aib-QGTFTSDFS-Orn-αMeL-LDKK((γ-Glu)-(2-[2-(2- | 282 | 5003.6 |
| | Amino-ethoxy)-ethoxy]-acetyl)-(γ-Glu)-CO—(CH₂)₁₆- | | |
| | CO₂H)AQ-Aib-AFIEYLLE-Aib-GPSSGAPPPSGT-NH₂ | | |
| 277 | Y-Aib-QGTFTSDFS-Orn-αMeL-LDKK((γ-Glu)-(2-[2-(2- | 283 | 5050.7 |
| | Amino-ethoxy)-ethoxy]-acetyl)-(γ-Glu)-CO—(CH₂)₁₆- | | |
| | CO₂H)AQ-Aib-AFIEYLLE-Aib-GPSSGAPPPSG-4Pal-NH₂ | | |
| 278 | Y-Aib-QGTFTSDFS-Orn-αMeL-LDKK((2-[2-(2-Amino- | 284 | 5003.6 |
| | ethoxy)-ethoxy]-acetyl)-(γ-Glu)₂-CO—(CH₂)₁₆—CO₂H)AQ- | | |
| | Aib-AFIEYLLE-Aib-GPSSGAPPPSGT-NH₂ | | |
| 279 | Y-Aib-QGTFTSDFS-Orn-αMeL-LDKK((2-[2-(2-Amino- | 285 | 5050.7 |
| | ethoxy)-ethoxy]-acetyl)-(γ-Glu)₂-CO—(CH₂)₁₆—CO₂H)AQ- | | |
| | Aib-AFIEYLLE-Aib-GPSSGAPPPSG-4Pal-NH₂ | | |
| 280 | Y-Aib-QGTFTSD-4Pal-SK-αMeL-LDKK((2-[2-(2-Amino- | 286 | 4907.5 |
| | ethoxy)-ethoxy]-acetyl)-(γ-Glu)-CO—(CH₂)₁₆—CO₂H)AQ- | | |
| | Aib-AFIEYLIE-Aib-GESSGAPPPSGS-NH₂ | | |
| 281 | Y-Aib-QGTFTSD-4Pal-SK-αMeL-LDKK((2-[2-(2-Amino- | 287 | 5007.6 |
| | ethoxy)-ethoxy]-acetyl)-(γ-Glu)-CO—(CH₂)₁₆—CO₂H)AQ- | | |
| | Aib-AFIEYLIE-Aib-GESSGEPPPSGE-NH₂ | | |
| 282 | Y-Aib-QGTFTSD-4Pal-SK-αMeL-LDKK((2-[2-(2-Amino- | 288 | 4965.5 |
| | ethoxy)-ethoxy]-acetyl)-(γ-Glu)-CO—(CH₂)₁₆—CO₂H)AQ- | | |
| | Aib-AFIEYLIE-Aib-GESSGEPPPSGS-NH₂ | | |
| 283 | Y-Aib-QGTFTSD-4Pal-SK-αMeL-LDKK((2-[2-(2-Amino- | 289 | 4893.5 |
| | ethoxy)-ethoxy]-acetyl)-(γ-Glu)-CO—(CH₂)₁₆—CO₂H)AQ- | | |
| | Aib-AFIEYLIEaGESSGAPPPSGS-NH₂ | | |
| 284 | Y-Aib-QGTFTSD-4Pal-SK-αMeL-LDKK((2-[2-(2-Amino- | 290 | 4951.5 |
| | ethoxy)-ethoxy]-acetyl)-(γ-Glu)-CO—(CH₂)₁₆—CO₂H)AQ- | | |
| | Aib-AFIEYLIEaGESSGEPPPSGS-NH₂ | | |
| 285 | Y-Aib-QGTFTSD-4Pal-SK-αMeL-LDKK((2-[2-(2-Amino- | 291 | 4849.4 |
| | ethoxy)-ethoxy]-acetyl)-(γ-Glu)-CO—(CH₂)₁₆—CO₂H)AQ- | | |
| | Aib-AFIEYLIA-Aib-GESSGAPPPSGS-NH₂ | | |
| 286 | Y-Aib-QGTFTSD-4Pal-SK-αMeL-LDKK((2-[2-(2-Amino- | 292 | 4949.5 |
| | ethoxy)-ethoxy]-acetyl)-(γ-Glu)-CO—(CH₂)₁₆—CO₂H)AQ- | | |
| | Aib-AFIEYLIA-Aib-GESSGEPPPSGE-NH₂ | | |
| 287 | Y-Aib-QGTFTSD-4Pal-SK-αMeL-LDKK((2-[2-(2-Amino- | 293 | 4907.5 |
| | ethoxy)-ethoxy]-acetyl)-(γ-Glu)-CO—(CH₂)₁₆—CO₂H)AQ- | | |
| | Aib-AFIEYLIA-Aib-GESSGEPPPSGS-NH₂ | | |
| 288 | Y-Aib-QGTFTSDFSI-αMeL-LDKK((2-[2-(2-Amino- | 294 | 4959.6 |
| | ethoxy)-ethoxy]-acetyl)-(γ-Glu)-CO—(CH₂)₁₈—CO₂H)AQ- | | |
| | Aib-AFIEYLLEGGPSSGEPPPSEG-NH₂ | | |
| 289 | Y-Aib-QGTFTSDYSI-αMeL-LDKK((2-[2-(2-Amino- | 295 | 5046.6 |
| | ethoxy)-ethoxy]-acetyl)-(γ-Glu)-CO—(CH₂)₁₈— | | |
| | CO₂H)AQRAFIEYLLEGGPSSGEPPPSEG-NH₂ | | |
| 290 | H-Aib-QGT-αMeF(2F)-TSDYSILLDKK((2-[2-(2-Amino- | 296 | 4954.5 |
| | ethoxy)-ethoxy]-acetyl)-(γ-Glu)-CO—(CH₂)₁₈—CO₂H)AA- | | |
| | Aib-AFIEYLIETGPSSGEPPPSEG-NH₂ | | |
| 291 | H-Aib-QGT-αMeF(2F)-TSDYSI-αMeL-LDKK((2-[2-(2- | 297 | 4926.5 |
| | Amino-ethoxy)-ethoxy]-acetyl)-(γ-Glu)-CO—(CH₂)₁₆- | | |
| | CO₂H)AA-Aib-AFIEYLIETGPSSGEPPPSEG-NH₂ | | |
| 292 | Y-Aib-QGT-αMeF(2F)-TSDYSK-αMeL-LDKK((2-[2-(2- | 298 | 5053.5 |
| | Amino-ethoxy)-ethoxy]-acetyl)-(γ-Glu)-CO—(CH₂)₁₆- | | |
| | CO₂H)AQ-Aib-QFIEYLEEGGPSSGEPPPSEG-NH₂ | | |
| 293 | Y-Aib-QGT-αMeF(2F)-TSDYSILLDKIAQK((2-[2-(2- | 299 | 5021.6 |
| | Amino-ethoxy)-ethoxy]-acetyl)-(γ-Glu)-CO—(CH₂)₁₈— | | |
| | CO₂H)AFIEYLLEGGPSSGEPPPSEG-NH₂ | | |
| 294 | Y-Aib-QGTFTSDYSI-αMeL-LDKK((2-[2-(2-Amino- | 300 | 5063.7 |
| | ethoxy)-ethoxy]-acetyl)₂-(γ-Glu)-CO—(CH₂)₁₈—CO₂H)AA- | | |
| | Aib-AFIEYLLEGGPSSGEPPPSEG-NH₂ | | |
| 295 | Y-Aib-QGT-αMeF(2F)-TSDYSKLLDKIAQK((2-[2-(2- | 301 | 5036.6 |
| | Amino-ethoxy)-ethoxy]-acetyl)-(γ-Glu)-CO—(CH₂)₁₈— | | |
| | CO₂H)AFIEYLLEGGPSSGEPPPSEG-NH₂ | | |
| 296 | Y-Aib-QGT-αMeF(2F)-TSDYSILLDKIAAK((2-[2-(2- | 302 | 4964.5 |
| | Amino-ethoxy)-ethoxy]-acetyl)-(γ-Glu)-CO—(CH₂)₁₈— | | |
| | CO₂H)AFIEYLIEGGPSSGEPPPSEG-NH₂ | | |
| 297 | NMeY-Aib-QGT-αMeF-TSDYSSLLDKIAAK((2-[2-(2- | 303 | 5079.7 |
| | Amino-ethoxy)-ethoxy]-acetyl)₂-(γ-Glu)-CO—(CH₂)₁₈— | | |
| | CO₂H)AFIEYLLEGGPSSGEPPPSEG-NH₂ | | |
| 298 | H-Aib-QGT-αMeF(2F)-TSDYSILLDKK((2-[2-(2-Amino- | 304 | 4924.5 |
| | ethoxy)-ethoxy]-acetyl)-(γ-Glu)-CO—(CH₂)₁₈—CO₂H)AA- | | |
| | Aib-AFIEYLIEAGPSSGEPPPSEG-NH₂ | | |
| 299 | NMeY-Aib-QGT-αMeF-TSDYSILLDKIAAK((2-[2-(2- | 305 | 5105.7 |
| | Amino-ethoxy)-ethoxy]-acetyl)₂-(γ-Glu)-CO—(CH₂)₁₈— | | |
| | CO₂H)AFIEYLIEGGPSSGEPPPSEG-NH₂ | | |
| 300 | NMeY-Aib-QGT-αMeF-TSDYSILLDKIAAK((2-[2-(2- | 306 | 5055.7 |
| | Amino-ethoxy)-ethoxy]-acetyl)₂-(γ-Glu)-CO—(CH₂)₁₈— | | |
| | CO₂H)AFVQWLIAGGPSSGEPPPSEG-NH₂ | | |
| 301 | NMeY-Aib-QGT-αMeF-TSDYSILLDKQAAK((2-[2-(2- | 307 | 5070.7 |
| | Amino-ethoxy)-ethoxy]-acetyl)₂-(γ-Glu)-CO—(CH₂)₁₈— | | |
| | CO₂H)AFVQWLIAGGPSSGEPPPSEG-NH₂ | | |
| 302 | NMeY-Aib-QGT-αMeF-TSDYSILLDKQAAK((2-[2-(2- | 308 | 5128.7 |
| | Amino-ethoxy)-ethoxy]-acetyl)₂-(γ-Glu)-CO—(CH₂)₁₈— | | |
| | CO₂H)AFVQWLIEGGPSSGEPPPSEG-NH₂ | | |
| 303 | Y-Aib-QGT-αMeF(2F)-TSDYSKLLDEIAQK((2-[2-(2- | 309 | 5037.6 |
| | Amino-ethoxy)-ethoxy]-acetyl)-(γ-Glu)-CO—(CH₂)₁₈— | | |
| | CO₂H)AFIEYLLEGGPSSGEPPPSEG-NH₂ | | |
| 304 | Y-Aib-QGT-αMeF(2F)-TSDYSKLLDKIAQK((2-[2-(2- | 310 | 5036.6 |
| | Amino-ethoxy)-ethoxy]-acetyl)-(γ-Glu)-CO—(CH₂)₁₈— | | |
| | CO₂H)AFIEYLIEGGPSSGEPPPSEG-NH₂ | | |
| 305 | Y-Aib-QGTFTSDYSI-αMeL-LDKQAAK((2-[2-(2-Amino- | 311 | 5057.7 |
| | ethoxy)-ethoxy]-acetyl)₂-(γ-Glu)-CO—(CH₂)₁₈— | | |
| | CO₂H)AFVEWLLAGGPSSGEPPPSEG-NH₂ | | |
| 306 | Y-Aib-QGT-αMeF(2F)-TSDYSILLDKAAQK((2-[2-(2- | 312 | 5022.6 |
| | Amino-ethoxy)-ethoxy]-acetyl)-(γ-Glu)-CO—(CH₂)₁₈—CO₂H)- | | |
| | Orn-FIEYLIEGGPSSGEPPPSEG-NH₂ | | |
| 307 | Y-Aib-QGT-αMeF-TSDYSILLDKIAAK((2-[2-(2-Amino- | 313 | 5092.7 |
| | ethoxy)-ethoxy]-acetyl)₂-(γ-Glu)-CO—(CH₂)₁₆- | | |
| | CO₂H)NFIEYLVEGGPSSGEPPPSEG-NH₂ | | |
| 308 | Y-Aib-QGTFTSDYSI-αMeL-LDKIAAK((2-[2-(2-Amino- | 314 | 5061.7 |
| | ethoxy)-ethoxy]-acetyl)₂-(γ-Glu)-CO—(CH₂)₁₈—CO₂H)-Orn- | | |
| | FVQYLIAGGPSSGEPPPSEG-NH₂ | | |
| 309 | Y-Aib-QGT-αMeF(2F)-TSDYSKLLDKQAQK((2-[2-(2- | 315 | 5108.6 |
| | Amino-ethoxy)-ethoxy]-acetyl)-(γ-Glu)-CO—(CH₂)₁₈— | | |
| | CO₂H)QFIEYLIEGGPSSGEPPPSEG-NH₂ | | |
| 310 | Y-Aib-QGT-αMeF(2F)-TSDYSKLLDKIAQK((2-[2-(2- | 316 | 5102.7 |
| | Amino-ethoxy)-ethoxy]-acetyl)-(γ-Glu)-CO—(CH₂)₁₈—CO₂H)- | | |
| | Orn-FIEWLIEGGPSSGEPPPSEG-NH₂ | | |
| 311 | Y-Aib-QGT-αMeF(2F)-TSDYSKILDKIAQK((2-[2-(2- | 317 | 5036.6 |
| | Amino-ethoxy)-ethoxy]-acetyl)-(γ-Glu)-CO—(CH₂)₁₈— | | |
| | CO₂H)AFIEYLLEGGPSSGEPPPSEG-NH₂ | | |
| 312 | Y-Aib-QGTFTSDYSI-αMeL-LDKIAAK((2-[2-(2-Amino- | 318 | 5076.7 |
| | ethoxy)-ethoxy]-acetyl)₂-(γ-Glu)-CO—(CH₂)₁₈— | | |
| | CO₂H)QFVEYLIAGGPSSGEPPPSEG-NH₂ | | |
| 313 | Y-Aib-QGTFTSDYSI-αMeL-LDKK((2-[2-(2-Amino- | 319 | 4917.5 |
| | ethoxy)-ethoxy]-acetyl)-(γ-Glu)-CO—(CH₂)₁₈— | | |
| | CO₂H)AQRAFIEYLLEGGPSSGEPPPSG-NH₂ | | |
| 314 | Y-Aib-QGTFTSDYSK-αMeL-LDKK((2-[2-(2-Amino- | 320 | 5055.6 |
| | ethoxy)-ethoxy]-acetyl)-(γ-Glu)-CO—(CH₂)₁₈— | | |
| | CO₂H)AQQAFIQWLIEGGPSSGEPPPSEG-NH₂ | | |
| 315 | Y-Aib-QGTFTSDYSK-αMeL-LDKK((2-[2-(2-Amino- | 321 | 5041.6 |
| | ethoxy)-ethoxy]-acetyl)-(γ-Glu)-CO—(CH₂)₁₈— | | |
| | CO₂H)AQQAFVQWLIEGGPSSGEPPPSEG-NH₂ | | |
| 316 | Y-Aib-QGTFTSDYSKLLDKK((2-[2-(2-Amino-ethoxy)- | 322 | 5012.6 |
| | ethoxy]-acetyl)-(γ-Glu)-CO—(CH₂)₁₈—CO₂H)AQ-Aib- | | |
| | AFVQWLIEGGPSSGEPPPSEG-NH₂ | | |
| 317 | Y-Aib-QGTFTSDYSI-αMeL-LDKQAAK((2-[2-(2-Amino- | 323 | 5085.7 |
| | ethoxy)-ethoxy]-acetyl)₂-(γ-Glu)-CO—(CH₂)₁₈— | | |
| | CO₂H)AFVEWLLA-Aib-GPSSGEPPPSEG-NH₂ | | |
| 318 | Y-Aib-QGTFTSDYSI-αMeL-LDKQAAK((2-[2-(2-Amino- | 324 | 5156.7 |
| | ethoxy)-ethoxy]-acetyl)₂-(γ-Glu)-CO—(CH₂)₁₈— | | |
| | CO₂H)QFIEWLIEGGPSSGEPPPGEG-NH₂ | | |
| 319 | Y-Aib-QGTFTSDYSI-αMeL-LDKQAAK((2-[2-(2-Amino- | 325 | 5170.8 |
| | ethoxy)-ethoxy]-acetyl)₂-(γ-Glu)-CO—(CH₂)₁₈— | | |
| | CO₂H)QFIEWLIEGGPSSGEPPPGAE-NH₂ | | |
| 320 | Y-Aib-QGTFTSDYSI-αMeL-LDKQAAK((2-[2-(2-Amino- | 326 | 5084.7 |
| | ethoxy)-ethoxy]-acetyl)₂-(γ-Glu)-CO—(CH₂)₁₈— | | |
| | CO₂H)QFVEWLLAGGPSSGEPPPGEG-NH₂ | | |
| 321 | Y-Aib-QGT-αMeF(2F)-TSD-F(4CN)-SILLDKQAQK((2-[2- | 327 | 5190.7 |
| | (2-Amino-ethoxy)-ethoxy]-acetyl)₂-(γ-Glu)-CO—(CH₂)₁₈— | | |
| | CO₂H)AFIEYLLEGGPSSGEPPPSEG-NH₂ | | |
| 322 | Y-Aib-QGTFTSDYSK-αMeL-LDKK((2-[2-(2-Amino- | 328 | 4947.5 |
| | ethoxy)-ethoxy]-acetyl)-(γ-Glu)-CO—(CH₂)₁₈— | | |
| | CO₂H)AQQAFIEYLIAGGPSSGAPPPSSE-NH₂ | | |
| 323 | Y-Aib-QGTFTSDYSI-αMeL-LDKIAAK((2-[2-(2-Amino- | 329 | 5184.8 |
| | ethoxy)-ethoxy]-acetyl)₂-(γ-Glu)-CO—(CH₂)₁₈— | | |
| | CO₂H)QFVQWLIE-Aib-GPSSGEPPPSEG-NH₂ | | |
| 324 | Y-Aib-QGTFTSDYSI-αMeL-LDKIAAK((2-[2-(2-Amino- | 330 | 5127.8 |
| | ethoxy)-ethoxy]-acetyl)₂-(γ-Glu)-CO—(CH₂)₁₈— | | |
| | CO₂H)QFVEWLLA-Aib-GPSSGEPPPSEG-NH₂ | | |
| 325 | Y-Aib-QGTFTSDYSI-αMeL-LDKQAAK((2-[2-(2-Amino- | 331 | 5127.8 |
| | ethoxy)-ethoxy]-acetyl)₂-(γ-Glu)-CO—(CH₂)₁₈— | | |
| | CO₂H)QFVNWLLA-Aib-GPSSGEPPPSEG-NH₂ | | |
| 326 | Y-Aib-QGTFTSDYSI-αMeL-LDKQAAK((2-[2-(2-Amino- | 332 | 5142.8 |
| | ethoxy)-ethoxy]-acetyl)₂-(γ-Glu)-CO—(CH₂)₁₈— | | |
| | CO₂H)QFVEWLIA-Aib-GPSSGEPPPSEG-NH₂ | | |
| 327 | Y-Aib-QGTFTSDYSI-αMeL-LDKQAAK((2-[2-(2-Amino- | 333 | 5084.7 |
| | ethoxy)-ethoxy]-acetyl)₂-(γ-Glu)-CO—(CH₂)₁₈— | | |
| | CO₂H)QFVEWLLA-Aib-GPSSGAPPPSEG-NH₂ | | |
| 328 | Y-Aib-QGTFTSDYSI-αMeL-LD-Orn-QAAK((2-[2-(2- | 334 | 5128.7 |
| | Amino-ethoxy)-ethoxy]-acetyl)₂-(γ-Glu)-CO—(CH₂)₁₈— | | |
| | CO₂H)QFVEWLLA-Aib-GPSSGEPPPSEG-NH₂ | | |
| 329 | Y-Aib-QGTFTSDYSI-αMeL-LDKQAAK((ε-K)₂-(γ-Glu)- | 335 | 5108.8 |
| | CO—(CH₂)₁₈—CO₂H)QFVEWLLA-Aib-GPSSGEPPPSEG | | |
| 330 | Y-Aib-QGT-αMeF(2F)-TSDYSKLLDKIAQK((2-[2-(2- | 336 | 5008.6 |
| | Amino-ethoxy)-ethoxy]-acetyl)-(γ-Glu)-CO—(CH₂)₁₆- | | |
| | CO₂H)AFIEYLIEGGPSSGEPPPSEG-NH₂ | | |
| 331 | Y-Aib-QGT-αMeF(2F)-TSDYSILLDKQAAK((2-[2-(2- | 337 | 5009.5 |
| | Amino-ethoxy)-ethoxy]-acetyl)-(γ-Glu)-CO—(CH₂)₁₆- | | |
| | CO₂H)AFIEYLIEGGPSSGEPPPSEG-NH₂ | | |
| 332 | Y-Aib-QGTFTSDYSI-αMeL-LDKQAAK((2-[2-(2-Amino- | 338 | 5098.7 |
| | ethoxy)-ethoxy]-acetyl)₂-(γ-Glu)-CO—(CH₂)₁₈— | | |
| | CO₂H)QFVEWLIAGGPSSGEPPPAEG-NH₂ | | |
| 333 | Y-Aib-QGTFTSDYSI-αMeL-LDKQAAK((2-[2-(2-Amino- | 339 | 5112.7 |
| | ethoxy)-ethoxy]-acetyl)₂-(γ-Glu)-CO—(CH₂)₁₈— | | |
| | CO₂H)QFVEWLIA-Aib-GPSSGEPPPGEG-NH₂ | | |
| 334 | Y-Aib-QGTFTSDYSI-αMeL-LDKQAAK((2-[2-(2-Amino- | 340 | 5126.8 |
| | ethoxy)-ethoxy]-acetyl)₂-(γ-Glu)-CO—(CH₂)₁₈— | | |
| | CO₂H)QFVEWLIA-Aib-GPSSGEPPPAEG-NH₂ | | |
| 335 | Y-Aib-QGTFTSDYSI-αMeL-LDKQAAK((2-[2-(2-Amino- | 341 | 5070.7 |
| | ethoxy)-ethoxy]-acetyl)₂-(γ-Glu)-CO—(CH₂)₁₈— | | |
| | CO₂H)QFVEWLIA-Aib-GPSSGEPPPGSG-NH₂ | | |
| 336 | Y-Aib-QGTFTSDYSI-αMeL-LDKQAAK((2-[2-(2-Amino- | 342 | 5084.7 |
| | ethoxy)-ethoxy]-acetyl)₂-(γ-Glu)-CO—(CH₂)₁₈— | | |
| | CO₂H)QFVEWLIA-Aib-GPSSGEPPPGTG-NH₂ | | |
| 337 | Y-Aib-QGTFTSDYSK-αMeL-LDKK((2-[2-(2-Amino- | 343 | 4961.5 |
| | ethoxy)-ethoxy]-acetyl)-(γ-Glu)-CO—(CH₂)₁₈— | | |
| | CO₂H)AQQAFIEYLIAGGPSSGEPPPSGD-NH₂ | | |
| 338 | Y-Aib-QGT-αMeF(2F)-TSDYSKLLDKIAQK((2-[2-(2- | 344 | 5035.6 |
| | Amino-ethoxy)-ethoxy]-acetyl)-(γ-Glu)-CO—(CH₂)₁₈— | | |
| | CO₂H)AFIQYLIEGGPSSGEPPPSEG-NH₂ | | |
| 339 | Y-Aib-QGT-αMeF(2F)-TSDYSKLLDKIAQK((2-[2-(2- | 345 | 4978.6 |
| | Amino-ethoxy)-ethoxy]-acetyl)-(γ-Glu)-CO—(CH₂)₁₈— | | |
| | CO₂H)AFIEYLIEGGPSSGAPPPSEG-NH₂ | | |
| 340 | Y-Aib-QGT-αMeF(2F)-TSDYSILLDKQAAK((2-[2-(2- | 346 | 4979.5 |
| | Amino-ethoxy)-ethoxy]-acetyl)-(γ-Glu)-CO—(CH₂)₁₈— | | |
| | CO₂H)EFIEYLIEGGPSSGAPPPSEG-NH₂ | | |
| 341 | Y-Aib-QGT-αMeF(2F)-TSD-F(4CN)-SILLDKQAQK((2-[2- | 347 | 5044.6 |
| | (2-Amino-ethoxy)-ethoxy]-acetyl)-(γ-Glu)-CO—(CH₂)₁₈— | | |
| | CO₂H)AFIQYLLEGGPSSGEPPPSEG-NH₂ | | |
| 342 | Y-Aib-QGTFTSDYSK-αMeL-LDKK((2-[2-(2-Amino- | 348 | 5055.6 |
| | ethoxy)-ethoxy]-acetyl)-(γ-Glu)-CO—(CH₂)₁₈— | | |
| | CO₂H)AQQAFVQWLIEaGPSSGEPPPSEG-NH₂ | | |
| 343 | Y-Aib-QGTFTSDYSK-αMeL-LDKK((2-[2-(2-Amino- | 349 | 4998.6 |
| | ethoxy)-ethoxy]-acetyl)-(γ-Glu)-CO—(CH₂)₁₈—CO₂H)AQ- | | |
| | Aib-AFVQWLLEGGPSSGEPPPSEG-NH₂ | | |
| 344 | Y-Aib-QGTFTSDYSK-αMeL-LDKK((2-[2-(2-Amino- | 350 | 4975.6 |
| | ethoxy)-ethoxy]-acetyl)-(γ-Glu)-CO—(CH₂)₁₈— | | |
| | CO₂H)AQQAFIEYLLAGGPSSGEPPPSGE-NH₂ | | |
| 345 | Y-Aib-QGT-αMeF(2F)-TSDYSILLDKQAQK((2-[2-(2- | 351 | 5181.7 |
| | Amino-ethoxy)-ethoxy]-acetyl)₂-(γ-Glu)-CO—(CH₂)₁₈— | | |
| | CO₂H)AFIEYLLEGGPSSGEPPPSEG-NH₂ | | |
| 346 | Y-Aib-QGT-αMeF(2F)-TSDFSILLDKQAQK((2-[2-(2- | 352 | 5165.7 |
| | Amino-ethoxy)-ethoxy]-acetyl)₂-(γ-Glu)-CO—(CH₂)₁₈— | | |
| | CO₂H)AFIEYLLEGGPSSGEPPPSEG-NH₂ | | |
| 347 | Y-Aib-QGT-αMeF(2F)-TSD-4Pal-SILLDKQAQK((2-[2-(2- | 353 | 5166.7 |
| | Amino-ethoxy)-ethoxy]-acetyl)₂-(γ-Glu)-CO—(CH₂)₁₈— | | |
| | CO₂H)AFIEYLLEGGPSSGEPPPSEG-NH₂ | | |
| 348 | Y-Aib-QGT-αMeF(2F)-TSDYSILLDKQAAK((2-[2-(2- | 354 | 5182.7 |
| | Amino-ethoxy)-ethoxy]-acetyl)₂-(γ-Glu)-CO—(CH₂)₁₈— | | |
| | CO₂H)EFIEYLIEGGPSSGEPPPSEG-NH₂ | | |
| 349 | Y-Aib-QGTFTSDYSI-αMeL-LDKQAAK((2-[2-(2-Amino- | 355 | 5133.8 |
| | ethoxy)-ethoxy]-acetyl)₂-(γ-Glu)-CO—(CH₂)₁₈— | | |
| | CO₂H)QFIEYLIA-Aib-GPSSGEPPPSEG-NH₂ | | |
| 350 | Y-Aib-QGT-αMeF(2F)-TSD-F(4CN)-SILLDKQAQK((2-[2- | 356 | 5132.7 |
| | (2-Amino-ethoxy)-ethoxy]-acetyl)₂-(γ-Glu)-CO—(CH₂)₁₈— | | |
| | CO₂H)AFIEYLLEGGPSSGEPPPSAG-NH₂ | | |
| 351 | Y-Aib-QGT-αMeF(2F)-TSDYSILLDAQAAK((2-[2-(2- | 357 | 4980.5 |
| | Amino-ethoxy)-ethoxy]-acetyl)-(γ-Glu)-CO—(CH₂)₁₈— | | |
| | CO₂H)EFIEYLIEGGPSSGEPPPSEG-NH₂ | | |
| 352 | Y-Aib-QGT-αMeF(2F)-TSD-F(4CN)-SILLDKQAQK((2-[2- | 358 | 5174.7 |
| | (2-Amino-ethoxy)-ethoxy]-acetyl)-(γ-Glu)₂-CO—(CH₂)₁₈— | | |
| | CO₂H)AFIEYLLEGGPSSGEPPPSEG-NH₂ | | |
| 353 | Y-Aib-QGT-αMeF(2F)-TSD-F(4CN)-SILLDKQAQK((2-[2- | 359 | 5189.8 |
| | (2-Amino-ethoxy)-ethoxy]-acetyl)₂-(γ-Glu)-CO—(CH₂)₁₈— | | |
| | CO₂H)AFIQYLLEGGPSSGEPPPSEG-NH₂ | | |
| 354 | Y-Aib-QGT-αMeF(2F)-TSDYSKILDKIAQK((2-[2-(2- | 360 | 5035.6 |
| | Amino-ethoxy)-ethoxy]-acetyl)-(γ-Glu)-CO—(CH₂)₁₈— | | |
| | CO₂H)AFIQYLIEGGPSSGEPPPSEG-NH₂ | | |
| 355 | Y-Aib-QGT-αMeF(2F)-TSDYSKILDKIAQK((2-[2-(2- | 361 | 5036.6 |
| | Amino-ethoxy)-ethoxy]-acetyl)-(γ-Glu)-CO—(CH₂)₁₈— | | |
| | CO₂H)AFIEYLIEGGPSSGAPPPSEG-NH₂ | | |
| 356 | Y-Aib-QGT-αMeF(2F)-TSDYSKLLDKIAQK((2-[2-(2- | 362 | 4978.6 |
| | Amino-ethoxy)-ethoxy]-acetyl)-(γ-Glu)-CO—(CH₂)₁₈— | | |
| | CO₂H)AFIEYLIEGGPSSGAPPPSGE-NH₂ | | |
| 357 | Y-Aib-QGT-αMeF(2F)-TSDYSKLLDKIAQK((2-[2-(2- | 363 | 5020.6 |
| | Amino-ethoxy)-ethoxy]-acetyl)-(γ-Glu)-CO—(CH₂)₁₈— | | |
| | CO₂H)AFIEYLIEGGPSSGAPPPEGE-NH₂ | | |
| 358 | Y-Aib-QGTFTSDYSK-αMeL-LDKK((2-[2-(2-Amino- | 364 | 4894.4 |
| | ethoxy)-ethoxy]-acetyl)-(γ-Glu)-CO—(CH₂)₁₆—CO₂H)AQ- | | |
| | Aib-AFIEYLLEGGESSGAPPPSGS-NH₂ | | |
| 359 | Y-Aib-QGTFTSDYS-Orn-αMeL-LDKK((2-[2-(2-Amino- | 365 | 4880.4 |
| | ethoxy)-ethoxy]-acetyl)-(γ-Glu)-CO—(CH₂)₁₆—CO₂H)AQ- | | |
| | Aib-AFIEYLLEGGESSGAPPPSGS-NH₂ | | |
| 360 | Y-Aib-QGTFTSDYS-Dap-αMeL-LDKK((2-[2-(2-Amino- | 366 | 4852.4 |
| | ethoxy)-ethoxy]-acetyl)-(γ-Glu)-CO—(CH₂)₁₆—CO₂H)AQ- | | |
| | Aib-AFIEYLLEGGESSGAPPPSGS-NH₂ | | |
| 361 | Y-Aib-QGTFTSDYSR-αMeL-LDKK((2-[2-(2-Amino- | 367 | 4922.5 |
| | ethoxy)-ethoxy]-acetyl)-(γ-Glu)-CO—(CH₂)₁₆—CO₂H)AQ- | | |
| | Aib-AFIEYLLEGGESSGAPPPSGS-NH₂ | | |
| 362 | Y-Aib-QGTFTSDYSQ-αMeL-LDKK((2-[2-(2-Amino- | 368 | 4894.4 |
| | ethoxy)-ethoxy]-acetyl)-(γ-Glu)-CO—(CH₂)₁₆—CO₂H)AQ- | | |
| | Aib-AFIEYLLEGGESSGAPPPSGS-NH₂ | | |
| 363 | Y-Aib-QGT-αMeF(2F)-TSD-F(4CN)-SKLLDKIAQK((2-[2- | 369 | 4945.5 |
| | (2-Amino-ethoxy)-ethoxy]-acetyl)-(γ-Glu)-CO—(CH₂)₁₈— | | |
| | CO₂H)AFIEYLIEGGPSSGAPPPSGS-NH₂ | | |
| 364 | Y-Aib-QGT-αMeF(2F)-TSD-F(4CN)-SKLLDKIAQK((2-[2- | 370 | 5003.6 |
| | (2-Amino-ethoxy)-ethoxy]-acetyl)-(γ-Glu)-CO—(CH₂)₁₈— | | |
| | CO₂H)AFIEYLIEGGPSSGEPPPSGS-NH₂ | | |
| 365 | Y-Aib-QGT-αMeF(2F)-TSD-4Pal-SI-αMeL-LD-Orn-K((2- | 371 | 4849.5 |
| | [2-(2-Amino-ethoxy)-ethoxy]-acetyl)-(γ-Glu)-CO—(CH₂)₁₈— | | |
| | CO₂H)AQ-Aib-Orn-FIe-αMeY-LLEGGPSSGEPPPS | | |
| 366 | Y-Aib-QGT-αMeF(2F)-TSD-4Pal-SI-αMeL-LD-Orn-K((2- | 372 | 4820.4 |
| | [2-(2-Amino-ethoxy)-ethoxy]-acetyl)-(γ-Glu)-CO—(CH₂)₁₈— | | |
| | CO₂H)AQ-Aib-Aib-FIe-αMeY-LLEGGPSSGEPPPS | | |
| 367 | Y-Aib-QGT-αMeF(2F)-TSD-4Pal-SI-αMeL-LD-Orn-K((2- | 373 | 4821.4 |
| | [2-(2-Amino-ethoxy)-ethoxy]-acetyl)-(γ-Glu)-CO—(CH₂)₁₆- | | |
| | CO₂H)AQ-Aib-Orn-FIe-αMeY-LLEGGPSSGEPPPS | | |
| 368 | Y-Aib-QGT-αMeF(2F)-TSDY-αMeS-I-αMeL-LD-Orn- | 374 | 4850.4 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LLEGGPSSGEPPPS-NH₂ | | |
| 369 | Y-Aib-QGT-αMeF(2F)-TSD-4Pal-αMeS-I-αMeL-LD-Orn- | 375 | 4835.4 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LLEGGPSSGEPPPS-NH₂ | | |
| 370 | Y-Aib-QGT-αMeF(2F)-TSD-4Pal-SI-αMeL-LD-Orn-K((2- | 376 | 4863.4 |
| | [2-(2-Amino-ethoxy)-ethoxy]-acetyl)-(γ-Glu)-CO—(CH₂)₁₆- | | |
| | CO₂H)AQ-Aib-Orn-FIe-αMeY-LLEGGPSSGEPPPE-NH₂ | | |
| 371 | Y-Aib-QGT-αMeF(2F)-TSD-4Pal-SI-αMeL-LD-Orn-K((γ- | 377 | 4805.4 |
| | Glu)₂-CO—(CH₂)₁₆—CO₂H)AQ-Aib-Orn-FIe-αMeY- | | |
| | LLEGGPSSGEPPPS-NH₂ | | |
| 372 | Y-Aib-QGT-αMeF(2F)-TSD-4Pal-SI-αMeL-LD-Orn-K((γ- | 378 | 4847.4 |
| | Glu)₂-CO—(CH₂)₁₆—CO₂H)AQ-Aib-Orn-FIe-αMeY- | | |
| | LLEGGPSSGEPPPE-NH₂ | | |
| 373 | Y-Aib-QGT-αMeF(2F)-TSD-4Pal-SI-αMeL-LD-Orn-K((γ- | 379 | 4875.5 |
| | Glu)₂-CO—(CH₂)₁₆—CO₂H)AQ-Aib-Orn-FIe-αMeY- | | |
| | LLEGGPSS-Aib-EPPPE-NH₂ | | |
| 374 | Y-Aib-QGT-αMeF(2F)-TSD-4Pal-αMeS-I-αMeL-LD-Orn- | 380 | 4819.4 |
| | K((γ-Glu)₂-CO—(CH₂)₁₆—CO₂H)AQ-Aib-Orn-FIe-αMeY- | | |
| | LLEGGPSSGEPPPS-NH₂ | | |
| 375 | Y-Aib-QGT-αMeF(2F)-TSD-4Pal-αMeS-I-αMeL-LD-Orn- | 381 | 4819.4 |
| | K((γ-Glu)₂-CO—(CH₂)₁₆—CO₂H)AQ-Aib-Orn-FIe-αMeY- | | |
| | LIEGGPSSGEPPPS-NH₂ | | |
| 376 | Y-Aib-QGT-αMeF(2F)-TSD-4Pal-αMeS-I-αMeL-LD-Orn- | 382 | 4964.5 |
| | K((2-[2-(2-Amino-ethoxy)-ethoxy]-acetyl)-(γ-Glu)₂-CO— | | |
| | (CH₂)₁₆—CO₂H)AQ-Aib-Orn-FIe-αMeY- | | |
| | LLEGGPSSGEPPPS-NH₂ | | |
| 377 | Y-Aib-QGT-αMeF(2F)-TSD-4Pal-αMeS-I-αMeL-LD-Orn- | 383 | 4877.5 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LLEGGPSSGEPPPE-NH₂ | | |
| 378 | Y-Aib-QGT-αMeF(2F)-TSD-4Pal-αMeS-I-αMeL-LD-Orn- | 384 | 4877.5 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LIEGGPSSGEPPPE-NH₂ | | |
| 379 | H-Aib-QGT-αMeF(2F)-TSD-4Pal-αMeS-I-αMeL-LD-Orn- | 385 | 4905.5 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LLEGGPSSGEPPPE-NH₂ | | |
| 380 | Y-Aib-QGT-αMeF(2F)-TSD-4Pal-αMeS-I-αMeL-LD-Orn- | 386 | 4851.4 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY-LLEGGPSS-Aib- | | |
| | EPPPE-NH₂ | | |
| 381 | H-Aib-QGT-αMeF(2F)-TSDY-αMeS-I-αMeL-LD-Orn- | 387 | 4809.4 |
| | K((2-[2-(2-Amino-ethoxy)-ethoxy]-acetyl)-(γ-Glu)-CO— | | |
| | (CH₂)₁₆—CO₂H)AQ-Aib-AFIeYLLEGGPSSGEPPPE-NH₂ | | |
| 382 | Y-Aib-QGTFTSDY-αMeS-I-αMeL-LDKK((2-[2-(2-Amino- | 388 | 4890.5 |
| | ethoxy)-ethoxy]-acetyl)₂-(γ-Glu)-CO—(CH₂)₁₈—CO₂H)AQ- | | |
| | Aib-AFIEYLIEGGPSSGAPPPS-NH₂ | | |
| 383 | Y-Aib-QGT-αMeF(2F)-TSDYSI-αMeL-LDKK((2-[2-(2- | 389 | 5067.7 |
| | Amino-ethoxy)-ethoxy]-acetyl)₂-(γ-Glu)-CO—(CH₂)₁₈— | | |
| | CO₂H)AA-Aib-AFIEYLIETGPSSGAPPPSEG-NH₂ | | |
| 384 | Y-Aib-QGT-αMeF(2F)-TSDY-αMeS-I-αMeL-LD-Orn- | 390 | 4906.5 |
| | K((2-[2-(2-Amino-ethoxy)-ethoxy]-acetyl)-(γ-Glu)-CO— | | |
| | (CH₂)₁₆—CO₂H)AQ-Aib-Orn-FIEYLLEGGPSSGAPPPS | | |
| 385 | Y-Aib-QGT-αMeF(2F)-TSDYSILLDKK((2-[2-(2-Amino- | 391 | 4894.5 |
| | ethoxy)-ethoxy]-acetyl)₂-(γ-Glu)-CO—(CH₂)₁₈—CO₂H)AQ- | | |
| | Aib-AFIEYLLEGGPSSGAPPPS-NH₂ | | |
| 386 | Y-Aib-QGT-αMeF(2F)-TSDYSILLDKK((2-[2-(2-Amino- | 392 | 4936.5 |
| | ethoxy)-ethoxy]-acetyl)₂-(γ-Glu)-CO—(CH₂)₁₈—CO₂H)AQ- | | |
| | Aib-AFIEYLLEGGPSSGAPPPE-NH₂ | | |
| 387 | Y-Aib-QGT-αMeF(2F)-TSDYSILLDKK((2-[2-(2-Amino- | 393 | 4737.2 |
| | ethoxy)-ethoxy]-acetyl)-(γ-Glu)-CO—(CH₂)₁₆—CO₂H)AQ- | | |
| | Aib-AFIEYLEEGGPSSGAPPPS-NH₂ | | |
| 388 | Y-Aib-QGT-αMeF(2F)-TSDYSILLDKK((2-[2-(2-Amino- | 394 | 4779.3 |
| | ethoxy)-ethoxy]-acetyl)-(γ-Glu)-CO—(CH₂)₁₆—CO₂H)AQ- | | |
| | Aib-AFIEYLEEGGPSSGAPPPE-NH₂ | | |
| 389 | Y-Aib-QGT-αMeF-TSDY-αMeS-S-αMeL-LD-Orn-K((2-[2- | 395 | 4848.4 |
| | (2-Amino-ethoxy)-ethoxy]-acetyl)-(γ-Glu)-CO—(CH₂)₁₆- | | |
| | CO₂H)AQ-Aib-Orn-FIe-αMeY-LLEGGPSSGEPPPE-NH₂ | | |
| 390 | Y-Aib-QGT-αMeF-TSDY-αMeS-SLLD-Orn-K((2-[2-(2- | 396 | 4834.4 |
| | Amino-ethoxy)-ethoxy]-acetyl)-(γ-Glu)-CO—(CH₂)₁₆- | | |
| | CO₂H)AQ-Aib-Orn-FIe-αMeY-LLEGGPSSGEPPPE-NH₂ | | |
| 391 | Y-Aib-QGT-αMeF-TSDY-αMeS-I-αMeL-LD-Orn-K((2-[2- | 397 | 4860.5 |
| | (2-Amino-ethoxy)-ethoxy]-acetyl)-(γ-Glu)-CO—(CH₂)₁₆- | | |
| | CO₂H)AQ-Aib-Orn-FIeYLLEGGPSSGEPPPE-NH₂ | | |
| 392 | Y-Aib-QGT-αMeF-TSDYSILLDKK((2-[2-(2-Amino- | 398 | 4719.3 |
| | ethoxy)-ethoxy]-acetyl)-(γ-Glu)-CO—(CH₂)₁₆—CO₂H)AQ- | | |
| | Aib-AFIEYLEEGGPSSGAPPPS-NH₂ | | |
| 393 | Y-Aib-QGT-αMeF-TSDY-αMeS-ILLDKK((2-[2-(2-Amino- | 399 | 4733.3 |
| | ethoxy)-ethoxy]-acetyl)-(γ-Glu)-CO—(CH₂)₁₆—CO₂H)AQ- | | |
| | Aib-AFIEYLEEGGPSSGAPPPS-NH₂ | | |
| 394 | Y-Aib-QGT-αMeF-TSDYSI-αMeL-LDKK((2-[2-(2-Amino- | 400 | 4733.3 |
| | ethoxy)-ethoxy]-acetyl)-(γ-Glu)-CO—(CH₂)₁₆—CO₂H)AQ- | | |
| | Aib-AFIEYLEEGGPSSGAPPPS-NH₂ | | |
| 395 | Y-Aib-QGT-αMeF-TSDYSI-αMeL-LDKK((2-[2-(2-Amino- | 401 | 4919.4 |
| | ethoxy)-ethoxy]-acetyl)-(γ-Glu)-CO—(CH₂)₁₆—CO₂H)AQ- | | |
| | Aib-AFIEYLEEGGPSSGAPPPSEG-NH₂ | | |
| 396 | Y-Aib-QGT-αMeF-TSDYSI-αMeL-LDKK((2-[2-(2-Amino- | 402 | 4775.3 |
| | ethoxy)-ethoxy]-acetyl)-(γ-Glu)-CO—(CH₂)₁₆—CO₂H)AQ- | | |
| | Aib-AFIEYLEEGGPSSGAPPPE-NH₂ | | |
| 397 | Y-Aib-QGT-αMeF-TSDYSI-αMeL-LD-Orn-K((2-[2-(2- | 403 | 4761.3 |
| | Amino-ethoxy)-ethoxy]-acetyl)-(γ-Glu)-CO—(CH₂)₁₆- | | |
| | CO₂H)AQ-Aib-AFIEYLEEGGPSSGAPPPE | | |
| 398 | Y-Aib-QGT-αMeF(2F)-TSDYSILLDKK((2-[2-(2-Amino- | 404 | 4923.4 |
| | ethoxy)-ethoxy]-acetyl)-(γ-Glu)-CO—(CH₂)₁₆—CO₂H)AQ- | | |
| | Aib-AFIEYLEEGGPSSGAPPPSEG-NH₂ | | |
| 399 | Y-Aib-QGT-αMeF(2F)-TSDYSILLDKK((2-[2-(2-Amino- | 405 | 4965.4 |
| | ethoxy)-ethoxy]-acetyl)-(γ-Glu)-CO—(CH₂)₁₆—CO₂H)AQ- | | |
| | Aib-AFIEYLEEGGPSSGAPPPEEG-NH₂ | | |
| 400 | Y-Aib-QGT-αMeF(2F)-TSDYSI-αMeL-LDKK((2-[2-(2- | 406 | 4793.3 |
| | Amino-ethoxy)-ethoxy]-acetyl)-(γ-Glu)-CO—(CH₂)₁₆- | | |
| | CO₂H)AQ-Aib-AFIEYLEEGGPSSGAPPPE | | |
| 401 | Y-Aib-QGT-αMeF(2F)-TSD-4Pal-αMeS-I-αMeL-LD-Orn- | 407 | 5021.6 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LIEGGPSSGEPPPSEG-NH₂ | | |
| 402 | Y-Aib-QGT-αMeF(2F)-TSDY-αMeS-I-αMeL-LD-Orn- | 408 | 5053.6 |
| | K((2-[2-(2-Amino-ethoxy)-ethoxy]-acetyl)₂-(γ-Glu)-CO— | | |
| | (CH₂)₁₆—CO₂H)AQ-Aib-Orn-FIe-αMeY- | | |
| | LEEGGPSSGEPPPE-NH₂ | | |
| 403 | Y-Aib-QGT-αMeF(2F)-TSDY-αMeS-I-αMeL-LD-Orn- | 409 | 5094.6 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LEEGGPSSGEPPPEEG-NH₂ | | |
| 404 | Y-Aib-QGT-αMeF(2F)-TSDYSI-αMeL-LD-Orn-K((2-[2-(2- | 410 | 4907.5 |
| | Amino-ethoxy)-ethoxy]-acetyl)-(γ-Glu)-CO—(CH₂)₁₆- | | |
| | CO₂H)AQ-Aib-AFIeYLLEGGPSSGAPPPSEG-NH₂ | | |
| 405 | Y-Aib-QGTFTSDFSK-αMeL-LDKK((ε-K)₂-(γ-Glu)-CO— | 411 | 5027.7 |
| | (CH₂)₁₈—CO₂H)AQ-Aib-AFIEYLAAGGPSSGEPPPSGE- | | |
| | NH₂ | | |
| 406 | Y-Aib-QGT-αMeF(2F)-TSDYSILLDKK((γ-Glu)₂-CO— | 412 | 4721.2 |
| | (CH₂)₁₆—CO₂H)AQ-Aib-AFIEYLEEGGPSSGAPPPS-NH₂ | | |
| 407 | Y-Aib-QGT-αMeF(2F)-TSDYSILLDKK((2-[2-(2-Amino- | 413 | 4795.3 |
| | ethoxy)-ethoxy]-acetyl)-(γ-Glu)-CO—(CH₂)₁₆—CO₂H)AQ- | | |
| | Aib-AFIEYLEEGGPSSGEPPPS-NH₂ | | |
| 408 | Y-Aib-QGT-αMeF(2F)-TSDYSILLDKK((2-[2-(2-Amino- | 414 | 4866.4 |
| | ethoxy)-ethoxy]-acetyl)-(γ-Glu)-CO—(CH₂)₁₆—CO₂H)AQ- | | |
| | Aib-AFIEYLEEGGPSSGAPPPE-NH₂ | | |
| 409 | Y-Aib-QGT-αMeF(2F)-TSDYSSLLDKK((2-[2-(2-Amino- | 415 | 4711.2 |
| | ethoxy)-ethoxy]-acetyl)-(γ-Glu)-CO—(CH₂)₁₆—CO₂H)AQ- | | |
| | Aib-AFIEYLEEGGPSSGAPPPS-NH₂ | | |
| 410 | Y-Aib-QGT-αMeF(2F)-TSDYSILLDAK((2-[2-(2-Amino- | 416 | 4866.3 |
| | ethoxy)-ethoxy]-acetyl)-(γ-Glu)-CO—(CH₂)₁₆- | | |
| | CO₂H)AFIEYLEEGGPSSGAPPPSEG-NH₂ | | |
| 411 | Y-Aib-QGT-αMeF(2F)-TSDYSILLDKK((2-[2-(2-Amino- | 417 | 4908.4 |
| | ethoxy)-ethoxy]-acetyl)-(γ-Glu)-CO—(CH₂)₁₆—CO₂H)AQ- | | |
| | Aib-AFIE-4Pal-LEEGGPSSGAPPPSEG-NH₂ | | |
| 412 | Y-Aib-QGT-αMeF(2F)-TSDYSILLDKK((2-[2-(2-Amino- | 418 | 4907.4 |
| | ethoxy)-ethoxy]-acetyl)-(γ-Glu)-CO—(CH₂)₁₆—CO₂H)AQ- | | |
| | Aib-AFIEFLEEGGPSSGAPPPSEG-NH₂ | | |
| 413 | Y-Aib-QGT-αMeF(2F)-TSD-4Pal-SILLDKK((γ-Glu)₂-CO— | 419 | 4706.2 |
| | (CH₂)₁₆—CO₂H)AQ-Aib-AFIEYLEEGGPSSGAPPPS-NH₂ | | |
| 414 | Y-Aib-QGT-αMeF(2F)-TSDYSILLD-Orn-K((γ-Glu)₂-CO— | 420 | 4707.2 |
| | (CH₂)₁₆—CO₂H)AQ-Aib-AFIEYLEEGGPSSGAPPPS-NH₂ | | |
| 415 | Y-Aib-QGT-αMeF(2F)-TSDYSILLDKK((γ-Glu)₂-CO— | 421 | 4793.3 |
| | (CH₂)₁₆—CO₂H)AQ-Aib-Aad-FIEYLEEGGPSSGAPPPS- | | |
| | NH₂ | | |
| 416 | Y-Aib-QGT-αMeF(2F)-TSDYSILLDKK((γ-Glu)₂-CO— | 422 | 4764.2 |
| | (CH₂)₁₆—CO₂H)AQ-Aib-NFIEYLEEGGPSSGAPPPS-NH₂ | | |
| 417 | Y-Aib-QGT-αMeF(2F)-TSDYSI-αMeL-LDKK((2-[2-(2- | 423 | 4951.5 |
| | Amino-ethoxy)-ethoxy]-acetyl)-(γ-Glu)-CO—(CH₂)₁₆- | | |
| | CO₂H)AQ-Aib-AFIEYLLETGPSSGAPPPSEG-NH₂ | | |
| 418 | Y-Aib-QGT-αMeF-TSD-4Pal-SILLDKK((2-[2-(2-Amino- | 424 | 4833.4 |
| | ethoxy)-ethoxy]-acetyl)₂-(γ-Glu)-CO—(CH₂)₁₆—CO₂H)AQ- | | |
| | Aib-AFIEYLLEGGPSSGAPPPS-NH₂ | | |
| 419 | Y-Aib-QGT-αMeF-TSDYSILLDKK((2-[2-(2-Amino- | 425 | 4848.5 |
| | ethoxy)-ethoxy]-acetyl)₂-(γ-Glu)-CO—(CH₂)₁₆—CO₂H)AQ- | | |
| | Aib-AFIeYLLEGGPSSGAPPPS-NH₂ | | |
| 420 | Y-Aib-QGT-αMeF-TSDY-αMeS-I-αMeL-LD-Orn-K((2-[2- | 426 | 4817.4 |
| | (2-Amino-ethoxy)-ethoxy]-acetyl)-(γ-Glu)-CO—(CH₂)₁₆- | | |
| | CO₂H)AQ-Aib-AFIeYLLEGGPSSGEPPPE-NH₂ | | |
| 421 | Y-Aib-QGT-αMeF(2F)-TSDYSI-αMeL-LDKK((2-[2-(2- | 427 | 4751.3 |
| | Amino-ethoxy)-ethoxy]-acetyl)-(γ-Glu)-CO—(CH₂)₁₆- | | |
| | CO₂H)AQ-Aib-AFIEYLEEGGPSSGAPPPS-NH₂ | | |
| 422 | Y-Aib-QGT-αMeF(2F)-TSD-4Pal-SI-αMeL-LDKK((2-[2- | 428 | 4736.3 |
| | (2-Amino-ethoxy)-ethoxy]-acetyl)-(γ-Glu)-CO—(CH₂)₁₆- | | |
| | CO₂H)AQ-Aib-AFIEYLEEGGPSSGAPPPS-NH₂ | | |
| 423 | Y-Aib-QGT-αMeF(2F)-TSDYSI-αMeL-LDKK((2-[2-(2- | 429 | 4783.3 |
| | Amino-ethoxy)-ethoxy]-acetyl)-(γ-Glu)-CO—(CH₂)₁₆- | | |
| | CO₂H)AQ-Aib-AFIEYLEEGGPSSGAPEPS-NH₂ | | |
| 424 | Y-Aib-QGT-αMeF(2F)-TSDYSI-αMeL-LDKK((2-[2-(2- | 430 | 4880.4 |
| | Amino-ethoxy)-ethoxy]-acetyl)-(γ-Glu)-CO—(CH₂)₁₆- | | |
| | CO₂H)AQ-Aib-AFIEYLEEGGPSSGAPPPSE-NH₂ | | |
| 425 | H-Aib-QGT-αMeF(2F)-TSDYSILLDKK((2-[2-(2-Amino- | 431 | 4711.2 |
| | ethoxy)-ethoxy]-acetyl)-(γ-Glu)-CO—(CH₂)₁₆—CO₂H)AQ- | | |
| | Aib-AFIEYLEEGGPSSGAPPPS-NH₂ | | |
| 426 | H-Aib-QGT-αMeF(2F)-TSDYSILLDKK((γ-Glu)₂-CO— | 432 | 4695.2 |
| | (CH₂)₁₆—CO₂H)AQ-Aib-AFIEYLEEGGPSSGAPPPS-NH₂ | | |
| 427 | Y-Aib-QGT-αMeF(2F)-TSDYSILLD-Orn-K((2-[2-(2- | 433 | 4852.4 |
| | Amino-ethoxy)-ethoxy]-acetyl)₂-(γ-Glu)-CO—(CH₂)₁₆- | | |
| | CO₂H)AQ-Aib-AFIeYLLEGGPSSGAPPPS-NH₂ | | |
| 428 | Y-Aib-QGT-αMeF(2F)-TSDYSILLD-Orn-K((γ-Glu)₂-CO— | 434 | 4691.2 |
| | (CH₂)₁₆—CO₂H)AQ-Aib-AFIeYLLEGGPSSGAPPPS-NH₂ | | |
| 429 | Y-Aib-QGT-αMeF(2F)-TSD-4Pal-αMeS-I-αMeL-LD-Orn- | 435 | 4995.6 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LIEGGPSSGAPEPSEG-NH₂ | | |
| 430 | Y-Aib-QGT-αMeF(2F)-TSDYSILLDKIAQ-Aib-AFIK((2- | 436 | 4721.3 |
| | [2-(2-Amino-ethoxy)-ethoxy]-acetyl)-(γ-Glu)-CO—(CH₂)₁₆- | | |
| | CO₂H)YLEEGGPSSGAPPPS-NH₂ | | |
| 431 | Y-Aib-QGT-αMeF(2F)-TSDYSI-αMeL-LDKK((2-[2-(2- | 437 | 4937.4 |
| | Amino-ethoxy)-ethoxy]-acetyl)-(γ-Glu)-CO—(CH₂)₁₆- | | |
| | CO₂H)AQ-Aib-AFIEYLEEGGPSSGAPPPSEG-NH₂ | | |
| 432 | Y-Aib-QGT-αMeF(2F)-TSDYSILLDKK((γ-Glu)₂-CO— | 438 | 4779.2 |
| | (CH₂)₁₆—CO₂H)AQ-Aib-AFIEYLEEGGPSSGEPPPS-NH₂ | | |
| 433 | Y-Aib-QGT-αMeF(2F)-TSDYSILLDKK((γ-Glu)₂-CO— | 439 | 4850.3 |
| | (CH₂)₁₆—CO₂H)AQ-Aib-AFIEYLEEGGPSSGAPPPSE-NH₂ | | |
| 434 | Y-Aib-QGT-αMeF(2F)-TSDYSILLDKK((γ-Glu)₂-CO— | 440 | 4907.4 |
| | (CH₂)₁₆—CO₂H)AQ-Aib-AFIEYLEEGGPSSGAPPPSEG- | | |
| | NH₂ | | |
| 435 | Y-Aib-QGT-αMeF(2F)-TSDYSILLDKK((γ-Glu)₂-CO— | 441 | 4907.4 |
| | (CH₂)₁₆—CO₂H)AQ-Aib-AFIEYLEEGGPSSGAPPPSGE- | | |
| | NH₂ | | |
| 436 | Y-Aib-QGT-αMeF(2F)-TSD-4Pal-αMeS-I-αMeL-LD-Orn- | 442 | 4834.4 |
| | K((γ-Glu)₂-CO—(CH₂)₁₆—CO₂H)AQ-Aib-SFIe-αMeY- | | |
| | LIEGGPSSGEPPPE-NH₂ | | |
| 437 | Y-Aib-QGT-αMeF(2F)-TSD-4Pal-αMeS-I-αMeL-LD-Orn- | 443 | 4819.4 |
| | K((γ-Glu)₂-CO—(CH₂)₁₆—CO₂H)AQ-Aib--Orn-FIe-αMeY- | | |
| | LAEGGPSSGEPPPE-NH₂ | | |
| 438 | Y-Aib-QGT-αMeF(2F)-TSD-4Pal-αMeS-I-αMeL-LD-Orn- | 444 | 4835.3 |
| | K((γ-Glu)₂-CO—(CH₂)₁₆—CO₂H)AQ-Aib-Orn-FIe-αMeY- | | |
| | LSEGGPSSGEPPPE-NH₂ | | |
| 439 | Y-Aib-QGT-αMeF(2F)-TSD-4Pal-αMeS-I-αMeL-LD-Orn- | 445 | 4876.4 |
| | K((γ-Glu)₂-CO—(CH₂)₁₆—CO₂H)AQ-Aib-Orn-FIe-αMeY- | | |
| | LQEGGPSSGEPPPE-NH₂ | | |
| 440 | Y-Aib-QGT-αMeF(2F)-TSD-4Pal-SI-αMeL-LDKK((γ- | 446 | 4720.2 |
| | Glu)₂-CO—(CH₂)₁₆—CO₂H)AQ-Aib- | | |
| | AFIEYLEEGGPSSGAPPPS-NH₂ | | |
| 441 | Y-Aib-QGT-αMeF(2F)-TSD-4Pal-SI-αMeL-LD-Orn-K((γ- | 447 | 4706.2 |
| | Glu)₂-CO—(CH₂)₁₆—CO₂H)AQ-Aib- | | |
| | AFIEYLEEGGPSSGAPPPS-NH₂ | | |
| 442 | Y-Aib-QGT-αMeF(2F)-TSD-4Pal-SI-αMeL-LDKK((2-[2- | 448 | 4794.3 |
| | (2-Amino-ethoxy)-ethoxy]-acetyl)-(γ-Glu)-CO—(CH₂)₁₆- | | |
| | CO₂H)AQ-Aib-AFIEYLEEGGPSSGEPPPS-NH₂ | | |
| 443 | Y-Aib-QGT-αMeF(2F)-TSD-4Pal-SI-αMeL-LDKK((2-[2- | 449 | 4768.3 |
| | (2-Amino-ethoxy)-ethoxy]-acetyl)-(γ-Glu)-CO—(CH₂)₁₆- | | |
| | CO₂H)AQ-Aib-AFIEYLEEGGPSSGAPEPS-NH₂ | | |
| 444 | Y-Aib-QGT-αMeF(2F)-TSD-4Pal-αMeS-I-αMeL-LD-Orn- | 450 | 4861.5 |
| | IAQ-Aib-Orn-FIe-αMeY-LIK((2-[2-(2-Amino-ethoxy)- | | |
| | ethoxy]-acetyl)-(γ-Glu)-CO—(CH₂)₁₆—CO₂H)GGPSSGEPPPE | | |
| 445 | Y-Aib-QGT-αMeF(2F)-TSD-4Pal-SI-αMeL-LDKK((2-[2- | 451 | 4778.3 |
| | (2-Amino-ethoxy)-ethoxy]-acetyl)-(γ-Glu)-CO—(CH₂)₁₆- | | |
| | CO₂H)AQ-Aib-AFIEYLEEGGPSSGAPPPE-NH₂ | | |
| 446 | Y-Aib-QGT-αMeF(2F)-TSD-4Pal-SI-αMeL-LDKK((γ- | 452 | 4678.2 |
| | Glu)₂-CO—(CH₂)₁₆—CO₂H)AQ-Aib- | | |
| | AFIEYLSEGGPSSGAPPPS-NH₂ | | |
| 447 | Y-Aib-QGT-αMeF(2F)-TSD-4Pal-αMeS-E-αMeL-LD-Orn- | 453 | 4877.4 |
| | K((γ-Glu)₂-CO—(CH₂)₁₆—CO₂H)AQ-Aib-Orn-FIe-αMeY- | | |
| | LIEGGPSSGEPPPE-NH₂ | | |
| 448 | Y-Aib-QGT-αMeF(2F)-TSD-4Pal-αMeS-I-αMeL-LD-Orn- | 454 | 4847.4 |
| | K((γ-Glu)₂-CO—(CH₂)₁₆—CO₂H)AQ-Aib--Orn- | | |
| | FIeYLIEGGPSSGEPPPE-NH₂ | | |
| 449 | Y-Aib-QGT-αMeF(2F)-TSD-4Pal-αMeS-I-αMeL-LD-Orn- | 455 | 4847.4 |
| | K((γ-Glu)₂-CO—(CH₂)₁₆—CO₂H)AQ-Aib- | | |
| | NFIeYLIEGGPSSGEPPPE-NH₂ | | |
| 450 | Y-Aib-QGT-αMeF(2F)-TSD-4Pal-αMeS-I-αMeL-LD-Orn- | 456 | 4876.4 |
| | K((γ-Glu)₂-CO—(CH₂)₁₆—CO₂H)AQ-Aib-NFIe-αMeY- | | |
| | LQEGGPSSGEPPPE-NH₂ | | |
| 451 | Y-Aib-QGT-αMeF(2F)-TSD-4Pal-αMeS-I-αMeL-LD-Orn- | 457 | 5006.5 |
| | K((2-[2-(2-Amino-ethoxy)-ethoxy]-acetyl)-(γ-Glu)₂-CO— | | |
| | (CH₂)₁₆—CO₂H)AQ-Aib-NFIe-αMeY-LIEGGPSSGEPPPE- | | |
| | NH₂ | | |
| 452 | Y-Aib-QGT-αMeF(2F)-TSD-4Pal-αMeS-I-αMeL-LD-Orn- | 458 | 4893.4 |
| | K((γ-Glu)₂-CO—(CH₂)₁₆—CO₂H)AQ-Aib-NFIe-αMeY- | | |
| | LIEGGPSSGEPEPE-NH₂ | | |
| 453 | Y-Aib-QGT-αMeF(2F)-TSD-4Pal-αMeS-I-αMeL-LD-Orn- | 459 | 4862.5 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMe4Pal- | | |
| | LIEGGPSSGEPPPE-NH₂ | | |
| 454 | Y-Aib-QGT-αMeF(2F)-TSD-4Pal-SILLD-Orn-IAQ-Aib- | 460 | 4692.3 |
| | AFIK((2-[2-(2-Amino-ethoxy)-ethoxy]-acetyl)-(γ-Glu)-CO— | | |
| | (CH₂)₁₆—CO₂H)YLEEGGPSSGAPPPS-NH₂ | | |
| 455 | Y-Aib-QGT-αMeF(2F)-TSDYSI-αMeL-LDKIAQ-Aib- | 461 | 4735.3 |
| | AFIK((2-[2-(2-Amino-ethoxy)-ethoxy]-acetyl)-(γ-Glu)-CO— | | |
| | (CH₂)₁₆—CO₂H)YLEEGGPSSGAPPPS-NH₂ | | |
| 456 | Y-Aib-QGT-αMeF(2F)-TSD-4Pal-αMeS-I-αMeL-LD-Orn- | 462 | 4877.4 |
| | K((2-[2-(2-Amino-ethoxy)-ethoxy]-acetyl)-(γ-Glu)-CO— | | |
| | (CH₂)₁₆—CO₂H)AQ-Aib-NFIe-αMeY-LIEGGPSSGEPPPE- | | |
| | NH₂ | | |
| 457 | Y-Aib-QGT-αMeF(2F)-TSD-4Pal-αMeS-I-αMeL-LD-Orn- | 463 | 4864.4 |
| | K((2-[2-(2-Amino-ethoxy)-ethoxy]-acetyl)-(γ-Glu)-CO— | | |
| | (CH₂)₁₆—CO₂H)AQ-Aib-TFIe-αMeY-LIEGGPSSGEPPPE- | | |
| | NH₂ | | |
| 458 | Y-Aib-QGT-αMeF(2F)-TSD-4Pal-αMeS-I-αMeL-LD-Orn- | 464 | 4863.4 |
| | K((2-[2-(2-Amino-ethoxy)-ethoxy]-acetyl)-(γ-Glu)-CO— | | |
| | (CH₂)₁₆—CO₂H)AQ-Aib-Orn-FIeYLIEGGPSSGEPPPE | | |
| 459 | Y-Aib-QGT-αMeF(2F)-TSD-4Pal-αMeS-I-αMeL-LD-Orn- | 465 | 4863.4 |
| | K((2-[2-(2-Amino-ethoxy)-ethoxy]-acetyl)-(γ-Glu)-CO— | | |
| | (CH₂)₁₆—CO₂H)AQ-Aib-Orn-FIEYLIEGGPSSGEPPPE-NH₂ | | |
| 460 | Y-Aib-QGT-αMeF(2F)-TSD-4Pal-αMeS-I-αMeL-LD-Orn- | 466 | 4863.4 |
| | K((2-[2-(2-Amino-ethoxy)-ethoxy]-acetyl)-(γ-Glu)-CO— | | |
| | (CH₂)₁₆—CO₂H)AQ-Aib-Orn-FIeYLLEGGPSSGEPPPE-NH₂ | | |
| 461 | Y-Aib-QGT-αMeF(2F)-TSD-4Pal-αMeS-I-αMeL-LD-Orn- | 467 | 4909.5 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LIEGGPSSGEPEPE-NH₂ | | |
| 462 | Y-Aib-QGT-αMeF(2F)-TSD-4Pal-αMeS-I-αMeL-LD-Orn- | 468 | 5063.6 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LIEGGPSSGEPPPEEG-NH₂ | | |
| 463 | Y-Aib-QGT-αMeF(2F)-TSD-4Pal-αMeS-I-αMeL-LD-Orn- | 469 | 5007.6 |
| | K((2-[2-(2-Amino-ethoxy)-ethoxy]-acetyl)-(γ-Glu)-CO— | | |
| | (CH₂)₁₆—CO₂H)AQ-Aib-Orn-FIeYLIEGGPSSGEPPPSEG- | | |
| | NH₂ | | |
| 464 | Y-Aib-QGT-αMeF(2F)-TSD-4Pal-αMeS-ILLDKK((2-[2-(2- | 470 | 4778.3 |
| | Amino-ethoxy)-ethoxy]-acetyl)-(γ-Glu)-CO—(CH₂)₁₆- | | |
| | CO₂H)AQ-Aib-AFIEYLEEGGPSSGAPPPE-NH₂ | | |
| 465 | Y-Aib-QGT-αMeF(2F)-TSD-3Pal-αMeS-ILLDKK((2-[2-(2- | 471 | 4778.3 |
| | Amino-ethoxy)-ethoxy]-acetyl)-(γ-Glu)-CO—(CH₂)₁₆- | | |
| | CO₂H)AQ-Aib-AFIEYLEEGGPSSGAPPPE-NH₂ | | |
| 466 | H-Aib-QGT-αMeF(2F)-TSD-4Pal-αMeS-I-αMeL-LD-Orn- | 472 | 4794.3 |
| | K((2-[2-(2-Amino-ethoxy)-ethoxy]-acetyl)-(γ-Glu)-CO— | | |
| | (CH₂)₁₆—CO₂H)AQ-Aib-AFIeYLLEGGPSSGEPPPE-NH₂ | | |
| 467 | Y-Aib-QGT-αMeF(2F)-TSDY-αMeS-ELLDKK((2-[2-(2- | 473 | 4809.3 |
| | Amino-ethoxy)-ethoxy]-acetyl)-(γ-Glu)-CO—(CH₂)₁₆- | | |
| | CO₂H)AQ-Aib-AFIEYLEEGGPSSGAPPPE-NH₂ | | |
| 468 | Y-Aib-QGT-αMeF(2F)-TSDY-αMeS-ILLDKK((2-[2-(2- | 474 | 4938.5 |
| | Amino-ethoxy)-ethoxy]-acetyl)₂-(γ-Glu)-CO—(CH₂)₁₆- | | |
| | CO₂H)AQ-Aib-AFIEYLEEGGPSSGAPPPE-NH₂ | | |
| 469 | Y-Aib-QGT-αMeF(2F)-TSDY-αMeS-ILLDKK((2-[2-(2- | 475 | 4996.5 |
| | Amino-ethoxy)-ethoxy]-acetyl)₂-(γ-Glu)-CO—(CH₂)₁₆- | | |
| | CO₂H)AQ-Aib-AFIEYLEEGGPSSGEPPPE-NH₂ | | |
| 470 | Y-Aib-QGT-αMeF(2F)-TSDYSILLDKK((γ-Glu)₂-CO— | 476 | 4979.4 |
| | (CH₂)₁₆—CO₂H)AQ-Aib-AFIEYLEEGGPSSGAPPPSEG- | | |
| | NH₂ | | |
| 471 | Y-Aib-QGT-αMeF(2F)-TSDYSILLDKK((γ-Glu)₂-CO— | 477 | 4949.4 |
| | (CH₂)₁₆—CO₂H)AQ-Aib-AFIEYLEEGGPSSGAPPPEEG- | | |
| | NH₂ | | |
| 472 | Y-Aib-QGT-αMeF(2F)-TSD-4Pal-αMeS-I-αMeL-LD-Orn- | 478 | 5140.7 |
| | K((2-[2-(2-Amino-ethoxy)-ethoxy]-acetyl)₂-(γ-Glu)-CO— | | |
| | (CH₂)₁₆—CO₂H)AQ-Aib-Orn-FIe-αMeY- | | |
| | LIEGGPSSGAPEPSEG-NH₂ | | |
| 473 | Y-Aib-QGT-αMeF(2F)-TSD-4Pal-αMeS-I-αMeL-LD-Orn- | 479 | 5037.5 |
| | K((γ-Glu)₂-CO—(CH₂)₁₆—CO₂H)AQ-Aib-NFIe-αMeY- | | |
| | LIEGGPSSGEPEPSEG-NH₂ | | |
| 474 | Y-Aib-QGT-αMeF(2F)-TSD-4Pal-αMeS-I-αMeL-LD-Orn- | 480 | 5079.6 |
| | K((γ-Glu)₂-CO—(CH₂)₁₆—CO₂H)AQ-Aib-NFIe-αMeY- | | |
| | LIEGGPSSGEPEPEEG-NH₂ | | |
| 475 | Y-Aib-QGT-αMeF(2F)-TSD-4Pal-SI-αMeL-LD-Orn-K((2- | 481 | 5126.7 |
| | [2-(2-Amino-ethoxy)-ethoxy]-acetyl)-(γ-Glu)-CO—(CH₂)₁₆- | | |
| | CO₂H)AQ-αMe4Pal-Orn-FIe-αMeY- | | |
| | LIEGGPSSGEPPPEEG-NH₂ | | |
| 476 | Y-Aib-QGT-αMeF(2F)-TSD-4Pal-αMeS-I-αMeL-LD-Orn- | 482 | 5049.6 |
| | K((2-[2-(2-Amino-ethoxy)-ethoxy]-acetyl)-(γ-Glu)-CO— | | |
| | (CH₂)₁₆—CO₂H)AQ-Aib-Orn-FIEYLIEGGPSSGEPPPEEG- | | |
| | NH₂ | | |
| 477 | Y-Aib-QGT-αMeF(2F)-TSD-4Pal-SI-αMeL-LD-Orn-K((2- | 483 | 5035.6 |
| | [2-(2-Amino-ethoxy)-ethoxy]-acetyl)-(γ-Glu)-CO—(CH₂)₁₆- | | |
| | CO₂H)AQ-Aib-Orn-FIEYLIEGGPSSGEPPPEEG-NH₂ | | |
| 478 | H-Aib-QGT-αMeF(2F)-TSD-4Pal-αMeS-I-αMeL-LD-Orn- | 484 | 4900.4 |
| | K((2-[2-(2-Amino-ethoxy)-ethoxy]-acetyl)-(γ-Glu)-CO— | | |
| | (CH₂)₁₆—CO₂H)AQ-αMe4Pal-NFIeYLLEGGPSSGEPPPE- | | |
| | NH₂ | | |
| 479 | H-Aib-QGT-αMeF(2F)-TSD-4Pal-αMeS-I-αMeL-LD-Orn- | 485 | 4826.3 |
| | K((2-[2-(2-Amino-ethoxy)-ethoxy]-acetyl)-(γ-Glu)-CO— | | |
| | (CH₂)₁₆—CO₂H)AQ-Aib-AFIeYLLEGGPSSGEPEPE-NH₂ | | |
| 480 | Y-Aib-QGT-αMeF(2F)-TSD-4Pal-αMeS-I-αMeL-LD-Orn- | 486 | 5005.6 |
| | K((γ-Glu)₂-CO—(CH₂)₁₆—CO₂H)AQ-Aib-Orn-FIe-αMeY- | | |
| | LIEGGPSSGEPPPSEG-NH₂ | | |
| 481 | Y-Aib-QGT-αMeF(2F)-TSD-4Pal-αMeS-I-αMeL-LD-Orn- | 487 | 5053.6 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LIEGGPSSGEPEPSEG-NH₂ | | |
| 482 | Y-Aib-QGT-αMeF(2F)-TSD-4Pal-αMeS-I-αMeL-LD-Orn- | 488 | 5049.6 |
| | K((2-[2-(2-Amino-ethoxy)-ethoxy]-acetyl)-(γ-Glu)-CO— | | |
| | (CH₂)₁₆—CO₂H)AQ-Aib-NFIEYLIEGGPSSGEPPPEEG-NH₂ | | |
| 483 | Y-Aib-QGT-αMeF(2F)-TSD-4Pal-αMeS-I-αMeL-LD-Orn- | 489 | 5049.6 |
| | K((2-[2-(2-Amino-ethoxy)-ethoxy]-acetyl)-(γ-Glu)-CO— | | |
| | (CH₂)₁₆—CO₂H)AQ-Aib-NFIEYLIEGGPSSGEPPPEGE-NH₂ | | |
| 484 | Y-Aib-QGT-αMeF(2F)-TSD-4Pal-αMeS-I-αMeL-LD-Orn- | 490 | 5063.6 |
| | K((2-[2-(2-Amino-ethoxy)-ethoxy]-acetyl)-(γ-Glu)-CO— | | |
| | (CH₂)₁₆—CO₂H)AQ-Aib-Orn-FIEYLIEAGPSSGEPPPEEG- | | |
| | NH₂ | | |
| 485 | Y-Aib-QGT-αMeF(2F)-TSD-4Pal-αMeS-I-αMeL-LD-Orn- | 491 | 5192.8 |
| | K((2-[2-(2-Amino-ethoxy)-ethoxy]-acetyl)-(γ-Glu)₂-CO— | | |
| | (CH₂)₁₆—CO₂H)AQ-Aib-Orn-FIEYLIEAGPSSGEPPPEEG- | | |
| | NH₂ | | |
| 486 | Y-Aib-QGT-αMeF(2F)-TSD-4Pal-SI-αMeL-LD-Orn-K((2- | 492 | 4780.3 |
| | [2-(2-Amino-ethoxy)-ethoxy]-acetyl)-(γ-Glu)-CO—(CH₂)₁₆- | | |
| | CO₂H)AQ-Aib-AFIEYLEEGGPSSGEPPPS-NH₂ | | |
| 487 | Y-Aib-QGT-αMeF(2F)-TSD-4Pal-SI-αMeL-LDKK((2-[2- | 493 | 4836.3 |
| | (2-Amino-ethoxy)-ethoxy]-acetyl)-(γ-Glu)-CO—(CH₂)₁₆- | | |
| | CO₂H)AQ-Aib-AFIEYLEEGGPSSGEPPPE-NH₂ | | |
| 488 | Y-Aib-QGT-αMeF(2F)-TSD-4Pal-SI-αMeL-LDKK((2-[2- | 494 | 4980.5 |
| | (2-Amino-ethoxy)-ethoxy]-acetyl)-(γ-Glu)-CO—(CH₂)₁₆- | | |
| | CO₂H)AQ-Aib-AFIEYLEEGGPSSGEPPPSEG-NH₂ | | |
| 489 | Y-Aib-QGT-αMeF(2F)-TSD-4Pal-SI-αMeL-LD-Orn-K((2- | 495 | 4993.5 |
| | [2-(2-Amino-ethoxy)-ethoxy]-acetyl)-(γ-Glu)-CO—(CH₂)₁₆- | | |
| | CO₂H)AQ-Aib-Orn-FIEYLIEGGPSSGEPPPSEG-NH₂ | | |
| 490 | Y-Aib-QGT-αMeF(2F)-TSD-4Pal-αMeS-I-αMeL-LD-Orn- | 496 | 5166.8 |
| | K((2-[2-(2-Amino-ethoxy)-ethoxy]-acetyl)₂-(γ-Glu)-CO— | | |
| | (CH₂)₁₆—CO₂H)AQ-Aib-Orn-FIe-αMeY- | | |
| | LIEGGPSSGEPPPSEG-NH₂ | | |
| 491 | Y-Aib-QGT-αMeF(2F)-TSD-4Pal-αMeS-I-αMeL-LD-Orn- | 497 | 5152.7 |
| | K((2-[2-(2-Amino-ethoxy)-ethoxy]-acetyl)₂-(γ-Glu)-CO— | | |
| | (CH₂)₁₆—CO₂H)AQ-Aib-Orn-FIEYLIEGGPSSGEPPPSEG- | | |
| | NH₂ | | |
| 492 | Y-Aib-QGT-αMeF(2F)-TSD-4Pal-αMeS-I-αMeL-LD-Orn- | 498 | 5126.7 |
| | K((2-[2-(2-Amino-ethoxy)-ethoxy]-acetyl)₂-(γ-Glu)-CO— | | |
| | (CH₂)₁₆—CO₂H)AQ-Aib-Orn-FIEYLIEGGPSSGAPEPSEG- | | |
| | NH₂ | | |
| 493 | Y-Aib-QGT-αMeF(2F)-TSD-4Pal-SI-αMeL-LDKK((2-[2- | 499 | 4980.5 |
| | (2-Amino-ethoxy)-ethoxy]-acetyl)-(γ-Glu)-CO—(CH₂)₁₆- | | |
| | CO₂H)AQ-Aib-AFIEYLEEGGPSSGEPPPSEG-NH₂ | | |
| 494 | Y-Aib-QGT-αMeF(2F)-TSD-4Pal-SI-αMeL-LD-Orn-K((2- | 500 | 5035.6 |
| | [2-(2-Amino-ethoxy)-ethoxy]-acetyl)-(γ-Glu)-CO—(CH₂)₁₆- | | |
| | CO₂H)AQ-Aib--Orn-FIEYLLEGGPSSGEPPPEEG-NH₂ | | |
| 495 | Y-Aib-QGT-αMeF(2F)-TSD-F(4NO2)-SI-αMeL-LDKK((2- | 501 | 4822.3 |
| | [2-(2-Amino-ethoxy)-ethoxy]-acetyl)-(γ-Glu)-CO—(CH₂)₁₆- | | |
| | CO₂H)AQ-Aib-AFIEYLEEGGPSSGAPPPE-NH₂ | | |
| 496 | Y-Aib-QGTFTSDFS-Orn-αMeL-LDKK((2-[2-(2-Amino- | 502 | 4787.4 |
| | ethoxy)-ethoxy]-acetyl)-(γ-Glu)-CO—(CH₂)₁₆—CO₂H)AQ- | | |
| | Aib-AFIE-4Pal-LLEGGPSSGAPPPGGS-NH₂ | | |
| 497 | Y-Aib-QGTFTSD-4Pal-S-Orn-αMeL-LDKK((2-[2-(2- | 503 | 4924.5 |
| | Amino-ethoxy)-ethoxy]-acetyl)-(γ-Glu)-CO—(CH₂)₁₆- | | |
| | CO₂H)AQ-αMe4Pal-AFIE-αMeY-LIEGGPSSGAPPPSGS- | | |
| | NH₂ | | |
| 498 | Y-Aib-QGTFTSD-4Pal-SQ-αMeL-LDKK((2-[2-(2-Amino- | 504 | 4951.5 |
| | ethoxy)-ethoxy]-acetyl)-(γ-Glu)-CO—(CH₂)₁₆—CO₂H)AQ- | | |
| | αMe4Pal-AFIE-αMe4Pal-LIE-Aib-GPSSGAPPPSGS-NH₂ | | |
| 499 | Y-Aib-QGT-αMeF(2F)-TSD-4Pal-SILLD-Orn-K((2-[2-(2- | 505 | 5084.7 |
| | Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGEPPPEGT | | |
| 500 | Y-Aib-QGT-αMeF(2F)-TSD-4Pal-SI-αMeL-LD-Orn-K((2- | 506 | 5084.7 |
| | [2-(2-Amino-ethoxy)-ethoxy]-acetyl)-(γ-Glu)-CO—(CH₂)₁₆- | | |
| | CO₂H)AQ-Me4Pal-Orn-FIeYLIEGGPSSGEPPPEGT-NH₂ | | |
| 501 | Y-Aib-QGT-αMeF(2F)-TSD-4Pal-αMeS-I-αMeL-LD-Orn- | 507 | 5108.7 |
| | K((2-[2-(2-Amino-ethoxy)-ethoxy]-acetyl)-(γ-Glu)-CO— | | |
| | (CH₂)₁₆—CO₂H)AQ-Aib-Orn-FIeYLIEGGPSSGEPPPSGTE- | | |
| | NH₂ | | |
| 502 | Y-Aib-QGT-αMeF(2F)-TSD-4Pal-αMeS-I-αMeL-LD-Orn- | 508 | 4980.5 |
| | K((2-[2-(2-Amino-ethoxy)-ethoxy]-acetyl)-(γ-Glu)-CO— | | |
| | (CH₂)₁₆—CO₂H)AQ-Aib-Orn-FIeYLIEGGPSSGEPPPSGT | | |
| 503 | Y-Aib-QGT-αMeF(2F)-TSD-4Pal-αMeS-I-αMeL-LD-Orn- | 509 | 5163.7 |
| | K((γ-Glu)₂-CO—(CH₂)₁₆—CO₂H)AQ-Aib-Orn-FIe-αMeY- | | |
| | LQEGGPSSGEPPPEGTE-NH₂ | | |
| 504 | Y-Aib-QGT-αMeF(2F)-TSD-4Pal-αMeS-I-αMeL-LD-Orn- | 510 | 5121.6 |
| | K((γ-Glu)₂-CO—(CH₂)₁₆—CO₂H)AQ-Aib-Orn-FIe-αMeY- | | |
| | LQEGGPSSGEPPPSGTE-NH₂ | | |
| 505 | Y-Aib-QGT-αMeF(2F)-TSD-4Pal-SI-αMeL-LD-Orn-K((2- | 511 | 5098.7 |
| | [2-(2-Amino-ethoxy)-ethoxy]-acetyl)-(γ-Glu)-CO—(CH₂)₁₆- | | |
| | CO₂H)AQ-Me4Pal-Orn-FIE-αMeY- | | |
| | LIEGGPSSGEPPPEGT-NH₂ | | |
| 506 | Y-Aib-QGT-αMeF(2F)-TSD-4Pal-SI-αMeL-LD-Orn-K((2- | 512 | 5114.6 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LEEGGPSSGEPPPEGT-NH₂ | | |
| 507 | Y-Aib-QGT-αMeFTSD-4Pal-SI-αMeL-LD-Orn-K((2-[2-(2- | 513 | 5098.7 |
| | Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGEPPPEGT- | | |
| | NH₂ | | |
| 508 | Y-Aib-QGT-αMeFTSD-4Pal-SI-αMeL-LD-Orn-K((2-[2-(2- | 514 | 5040.6 |
| | Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGAPPPEGT- | | |
| | NH₂ | | |
| 509 | Y-Aib-QGT-αMeF(2F)-TSD-4Pal-SILLDKK((γ-Glu)₂-CO— | 515 | 4993.5 |
| | (CH₂)₁₆—CO₂H)AQ-Aib-AFIEYLEEGGPSSGAPPPSGTE- | | |
| | NH₂ | | |
| 510 | Y-Aib-QGT-αMeF(2F)-TSD-4Pal-SILLDKK((γ-Glu)₂-CO— | 516 | 5050.5 |
| | (CH₂)₁₆—CO₂H)AQ-Aib-AFIEYLEEGGPSSGAPPPSGTGE- | | |
| | NH₂ | | |
| 511 | Y-Aib-QGT-αMeF(2F)-TSD-4Pal-SILLDKK((γ-Glu)₂-CO— | 517 | 4994.4 |
| | (CH₂)₁₆—CO₂H)AQ-Aib-AFIEYLEEGGPSSGAPPPSGTE | | |
| 512 | Y-Aib-QGT-αMeF(2F)-TSD-4Pal-SILLDKK((γ-Glu)₂-CO— | 518 | 5051.5 |
| | (CH₂)₁₆—CO₂H)AQ-Aib-AFIEYLEEGGPSSGAPPPSGTGE | | |
| 513 | Y-Aib-QGT-αMeF(2F)-TSD-4Pal-SILLD-Orn-K((γ-Glu)₂- | 519 | 4979.4 |
| | CO—(CH₂)₁₆—CO₂H)AQ-Aib- | | |
| | AFIEYLEEGGPSSGAPPPSGTE-NH₂ | | |
| 514 | Y-Aib-QGT-αMeF(2F)-TSD-4Pal-SILLD-Orn-K((γ-Glu)₂- | 520 | 5036.5 |
| | CO—(CH₂)₁₆—CO₂H)AQ-Aib- | | |
| | AFIEYLEEGGPSSGAPPPSGTGE-NH₂ | | |
| 515 | Y-Aib-QGT-αMeF(2F)-TSD-4Pal-SILLD-Orn-K((γ-Glu)₂- | 521 | 4980.4 |
| | CO—(CH₂)₁₆—CO₂H)AQ-Aib- | | |
| | AFIEYLEEGGPSSGAPPPSGTE | | |
| 516 | Y-Aib-QGT-αMeF(2F)-TSD-4Pal-SILLD-Orn-K((γ-Glu)₂- | 522 | 5037.5 |
| | CO—(CH₂)₁₆—CO₂H)AQ-Aib- | | |
| | AFIEYLEEGGPSSGAPPPSGTGE | | |
| 517 | Y-Aib-QGT-αMeF(2F)-TSD-4Pal-αMeS-I-αMeL-LD-Orn- | 523 | 5080.6 |
| | K((γ-Glu)₂-CO—(CH₂)₁₆—CO₂H)AQ-Aib-Orn-FIe-αMeY- | | |
| | LSEGGPSSGEPPPSGTE-NH₂ | | |
| 518 | Y-Aib-QGT-αMeF(2F)-TSD-4Pal-αMeS-I-αMeL-LD-Orn- | 524 | 5137.6 |
| | K((γ-Glu)₂-CO—(CH₂)₁₆—CO₂H)AQ-Aib-Orn-FIe-αMeY- | | |
| | LSEGGPSSGEPPPSGTGE-NH₂ | | |
| 519 | Y-Aib-QGT-αMeF(2F)-TSD-4Pal-αMeS-I-αMeL-LD-Orn- | 525 | 5081.6 |
| | K((γ-Glu)₂-CO—(CH₂)₁₆—CO₂H)AQ-Aib-Orn-FIe-αMeY- | | |
| | LSEGGPSSGEPPPSGTE | | |
| 520 | Y-Aib-QGT-αMeF(2F)-TSD-4Pal-αMeS-I-αMeL-LD-Orn- | 526 | 5138.6 |
| | K((γ-Glu)₂-CO—(CH₂)₁₆—CO₂H)AQ-Aib-Orn-FIe-αMeY- | | |
| | LSEGGPSSGEPPPSGTGE | | |
| 521 | Y-Aib-QGT-αMeF(2F)-TSD-4Pal-αMeS-I-αMeL-LD-Orn- | 527 | 5092.6 |
| | K((γ-Glu)₂-CO—(CH₂)₁₆—CO₂H)AQ-Aib-Orn- | | |
| | FIeYLIEGGPSSGEPPPSGTE-NH₂ | | |
| 522 | Y-Aib-QGT-αMeF(2F)-TSD-4Pal-αMeS-I-αMeL-LD-Orn- | 528 | 5093.6 |
| | K((γ-Glu)₂-CO—(CH₂)₁₆—CO₂H)AQ-Aib-Orn- | | |
| | FIeYLIEGGPSSGEPPPSGTE | | |
| 523 | Y-Aib-QGT-αMeF(2F)-TSD-4Pal-αMeS-I-αMeL-LD-Orn- | 529 | 5149.7 |
| | K((γ-Glu)₂-CO—(CH₂)₁₆—CO₂H)AQ-Aib-Orn- | | |
| | FIeYLIEGGPSSGEPPPSGTGE-NH₂ | | |
| 524 | Y-Aib-QGT-αMeF(2F)-TSD-4Pal-αMeS-I-αMeL-LD-Orn- | 530 | 5150.7 |
| | K((γ-Glu)₂-CO—(CH₂)₁₆—CO₂H)AQ-Aib-Orn- | | |
| | FIeYLIEGGPSSGEPPPSGTGE | | |
| 525 | Y-Aib-QGT-αMeF(2F)-TSD-4Pal-αMeS-I-αMeL-LD-Orn- | 531 | 5034.5 |
| | K((γ-Glu)₂-CO—(CH₂)₁₆—CO₂H)AQ-Aib-NFIe-αMeY- | | |
| | LQEGGPSSGEPPPEGT-NH₂ | | |
| 526 | Y-Aib-QGT-αMeF(2F)-TSD-4Pal-αMeS-I-αMeL-LD-Orn- | 532 | 5163.6 |
| | K((γ-Glu)₂-CO—(CH₂)₁₆—CO₂H)AQ-Aib-NFIe-αMeY- | | |
| | LQEGGPSSGEPPPEGTE-NH₂ | | |
| 527 | Y-Aib-QGT-αMeF(2F)-TSD-4Pal-αMeS-I-αMeL-LD-Orn- | 533 | 5220.7 |
| | K((γ-Glu)₂-CO—(CH₂)₁₆—CO₂H)AQ-Aib-NFIe-αMeY- | | |
| | LQEGGPSSGEPPPEGTGE-NH₂ | | |
| 528 | Y-Aib-QGT-αMeF(2F)-TSD-4Pal-αMeS-I-αMeL-LD-Orn- | 534 | 5177.7 |
| | K((γ-Glu)₂-CO—(CH₂)₁₆—CO₂H)AQ-Aib-QFIe-αMeY- | | |
| | LQEGGPSSGEPPPEGTE-NH₂ | | |
| 529 | Y-Aib-QGT-αMeF(2F)-TSD-4Pal-αMeS-I-αMeL-LD-Orn- | 535 | 5234.7 |
| | K((γ-Glu)₂-CO—(CH₂)₁₆—CO₂H)AQ-Aib-QFIe-αMeY- | | |
| | LQEGGPSSGEPPPEGTGE-NH₂ | | |
| 530 | Y-Aib-QGT-αMeF(2F)-TSD-4Pal-αMeS-I-αMeL-LD-Orn- | 536 | 5066.6 |
| | K((γ-Glu)₂-CO—(CH₂)₁₆—CO₂H)AQ-Aib-Orn- | | |
| | FIeYLSEGGPSSGEPPPSGTE-NH₂ | | |
| 531 | Y-Aib-QGT-αMeF(2F)-TSD-4Pal-αMeS-ILLD-Orn-K((γ- | 537 | 5078.6 |
| | Glu)₂-CO—(CH₂)₁₆—CO₂H)AQ-Aib-Orn- | | |
| | FIeYLIEGGPSSGEPPPSGTE-NH₂ | | |
| 532 | Y-Aib-QGT-αMeF(2F)-TSD-4Pal-αMeS-I-αMeL-LD-Orn- | 538 | 5092.6 |
| | K((γ-Glu)₂-CO—(CH₂)₁₆—CO₂H)AQ-Aib-Orn- | | |
| | FIeYLLEGGPSSGEPPPSGTE-NH₂ | | |
| 533 | Y-Aib-QGT-αMeF(2F)-TSD-4Pal-αMeS-I-αMeL-LD-Orn- | 539 | 5237.8 |
| | K((2-[2-(2-Amino-ethoxy)-ethoxy]-acetyl)-(γ-Glu)-CO— | | |
| | (CH₂)₁₆—CO₂H)AQ-Aib-Orn- | | |
| | FIeYLIEGGPSSGEPPPSGTEE-NH₂ | | |
| 534 | Y-Aib-QGT-αMeF(2F)-TSD-4Pal-αMeS-I-αMeL-LD-Orn- | 540 | 5366.9 |
| | K((2-[2-(2-Amino-ethoxy)-ethoxy]-acetyl)-(γ-Glu)-CO— | | |
| | (CH₂)₁₆—CO₂H)AQ-Aib-Orn- | | |
| | FIeYLIEGGPSSGEPPPSGTEEE-NH₂ | | |
| 535 | Y-Aib-QGT-αMeF(2F)-TSD-4Pal-αMeS-I-αMeL-LD-Orn- | 541 | 5221.8 |
| | K((γ-Glu)₂-CO—(CH₂)₁₆—CO₂H)AQ-Aib-Orn- | | |
| | FIeYLIEGGPSSGEPPPSGTEE-NH₂ | | |
| 536 | Y-Aib-QGT-αMeF(2F)-TSD-4Pal-αMeS-I-αMeL-LD-Orn- | 542 | 5350.9 |
| | K((γ-Glu)₂-CO—(CH₂)₁₆—CO₂H)AQ-Aib-Orn- | | |
| | FIeYLIEGGPSSGEPPPSGTEEE-NH₂ | | |
| 537 | Y-Aib-QGT-αMeF(2F)-TSD-4Pal-αMeS-I-αMeL-LD-Orn- | 543 | 5238.8 |
| | K((2-[2-(2-Amino-ethoxy)-ethoxy]-acetyl)-(γ-Glu)-CO— | | |
| | (CH₂)₁₆—CO₂H)AQ-Aib-Orn-FIeYLIEGGPSSGEPPPSGTEE | | |
| 538 | Y-Aib-QGT-αMeF(2F)-TSD-4Pal-αMeS-I-αMeL-LD-Orn- | 544 | 5367.9 |
| | K((2-[2-(2-Amino-ethoxy)-ethoxy]-acetyl)-(γ-Glu)-CO— | | |
| | (CH₂)₁₆—CO₂H)AQ-Aib-Orn- | | |
| | FIeYLIEGGPSSGEPPPSGTEEE-NH₂ | | |
| 539 | Y-Aib-QGT-αMeF(2F)-TSD-4Pal-αMeS-I-αMeL-LD-Orn- | 545 | 5222.7 |
| | K((γ-Glu)₂-CO—(CH₂)₁₆—CO₂H)AQ-Aib-Orn- | | |
| | FIeYLIEGGPSSGEPPPSGTEE | | |
| 540 | Y-Aib-QGT-αMeF(2F)-TSD-4Pal-αMeS-I-αMeL-LD-Orn- | 546 | 5351.8 |
| | K((γ-Glu)₂-CO—(CH₂)₁₆—CO₂H)AQ-Aib-Orn- | | |
| | FIeYLIEGGPSSGEPPPSGTEEE-NH₂ | | |
| 541 | Y-Aib-QGT-αMeF(2F)-TSD-4Pal-SI-αMeL-LD-Orn-K((γ- | 547 | 5068.6 |
| | Glu)₂-CO—(CH₂)₁₆—CO₂H)AQ-Me4Pal-Orn- | | |
| | FIeYLIEGGPSSGEPPPEGT | | |
| 542 | Y-Aib-QGT-αMeF(2F)-TSD-4Pal-αMeS-I-αMeL-LD-Orn- | 548 | 5094.6 |
| | K((γ-Glu)₂-CO—(CH₂)₁₆—CO₂H)AQ-Aib-Orn-FIe-αMeY- | | |
| | LTEGGPSSGEPPPSGTE-NH₂ | | |
| 543 | Y-Aib-QGT-αMeF(2F)-TSD-4Pal-αMeS-I-αMeL-LD-Orn- | 549 | 5151.7 |
| | K((γ-Glu)₂-CO—(CH₂)₁₆—CO₂H)AQ-Aib-Orn-FIe-αMeY- | | |
| | LTEGGPSSGEPPPSGTGE-NH₂ | | |
| 544 | Y-Aib-QGT-αMeF(2F)-TSD-4Pal-αMeS-I-αMeL-LD-Orn- | 550 | 5064.6 |
| | K((γ-Glu)₂-CO—(CH₂)₁₆—CO₂H)AQ-Aib-Orn-FIe-αMeY- | | |
| | LAEGGPSSGEPPPSGTE-NH₂ | | |
| 545 | Y-Aib-QGT-αMeF(2F)-TSD-4Pal-αMeS-I-αMeL-LD-Orn- | 551 | 5121.6 |
| | K((γ-Glu)₂-CO—(CH₂)₁₆—CO₂H)AQ-Aib-Orn-FIe-αMeY- | | |
| | LAEGGPSSGEPPPSGTGE-NH₂ | | |
| 546 | Y-Aib-QGT-αMeF(2F)-TSD-4Pal-αMeS-ILLD-Orn-K((γ- | 552 | 5119.6 |
| | Glu)₂-CO—(CH₂)₁₆—CO₂H)AQ-Aib-Orn-FIe-αMeY- | | |
| | LQEGGPSSGEPPPEGAE-NH₂ | | |
| 547 | Y-Aib-QGT-αMeF(2F)-TSD-4Pal-αMeS-ILLD-Orn-K((γ- | 553 | 5077.6 |
| | Glu)₂-CO—(CH₂)₁₆—CO₂H)AQ-Aib-Orn-FIe-αMeY- | | |
| | LQEGGPSSGEPPPSGAE-NH₂ | | |
| 548 | Y-Aib-QGT-αMeF(2F)-TSD-4Pal-αMeS-ILLD-Orn-K((γ- | 554 | 5135.7 |
| | Glu)₂-CO—(CH₂)₁₆—CO₂H)AQ-Aib-Orn- | | |
| | FIeYLIEGGPSSGEPPPSGTGE-NH₂ | | |
| 549 | Y-Aib-QGT-αMeF(2F)-TSD-4Pal-SILLDK-K((γ-Glu)₂-CO— | 555 | 5016.5 |
| | (CH₂)₁₆—CO₂H)AQ-Aib-AFIEWLEEGGPSSGAPPPSGTE- | | |
| | NH₂ | | |
| 550 | Y-Aib-QGT-αMeF(2F)-TSD-4Pal-SILLDK-K((γ-Glu)₂-CO— | 556 | 4978.5 |
| | (CH₂)₁₆—CO₂H)AQ-Aib-AFIE-4Pal- | | |
| | LEEGGPSSGAPPPSGTE-NH₂ | | |
| 551 | Y-Aib-QGT-αMeF(2F)-TSD-4Pal-SILLDK-K((γ-Glu)₂-CO— | 557 | 5007.5 |
| | (CH₂)₁₆—CO₂H)AQ-Aib-AFIE-αMeY- | | |
| | LEEGGPSSGAPPPSGTE-NH₂ | | |
| 552 | Y-Aib-QGT-αMeF(2F)-TSD-4Pal-SILLDK-K((γ-Glu)₂-CO— | 558 | 4992.5 |
| | (CH₂)₁₆—CO₂H)AQ-Aib-AFIE-Me4Pal- | | |
| | LEEGGPSSGAPPPSGTE-NH₂ | | |
| 553 | Y-Aib-QGT-αMeF(2F)-TSD-4Pal-SILLDK-K((γ-Glu)₂-CO— | 559 | 4992.5 |
| | (CH₂)₁₆—CO₂H)AQ-Aib-AFIEYLQEGGPSSGAPPPSGTE- | | |
| | NH₂ | | |
| 554 | Y-Aib-QGT-αMeF(2F)-TSD-4Pal-SILLDK-K((γ-Glu)₂-CO— | 560 | 4965.5 |
| | (CH₂)₁₆—CO₂H)AQ-Aib-AFIEYLTEGGPSSGAPPPSGTE- | | |
| | NH₂ | | |
| 555 | Y-Aib-QGT-αMeF(2F)-TSD-4Pal-αMeS-ILLD-Orn-K((γ- | 561 | 5120.7 |
| | Glu)₂-CO—(CH₂)₁₆—CO₂H)AQ-Aib-Orn- | | |
| | FIeYLIEGGPSSGEPPPSETE-NH₂ | | |
| 556 | Y-Aib-QGT-αMeF(2F)-TSD-4Pal-αMeS-ILLD-Orn-K((γ- | 562 | 5249.8 |
| | Glu)₂-CO—(CH₂)₁₆—CO₂H)AQ-Aib-Orn- | | |
| | FIeYLIEGGPSSGEPPPEGTEE-NH₂ | | |
| 557 | Y-Aib-QGT-αMeF(2F)-TSD-4Pal-αMeS-I-αMeL-LD-Orn- | 563 | 5292.9 |
| | K((γ-Glu)₂-CO—(CH₂)₁₆—CO₂H)AQ-Aib-Orn-FIe-αMeY- | | |
| | LQEGGPSSGEPPPEGTEE-NH₂ | | |
| 558 | Y-Aib-QGT-αMeF(2F)-TSD-4Pal-αMeS-I-αMeL-LD-Orn- | 564 | 5422.0 |
| | K((γ-Glu)₂-CO—(CH₂)₁₆—CO₂H)AQ-Aib-Orn-FIe-αMeY- | | |
| | LQEGGPSSGEPPPEGTEEE-NH₂ | | |
| 559 | Y-Aib-QGT-αMeF(2F)-TSD-4Pal-αMeS-I-αMeL-LD-Orn- | 565 | 5036.6 |
| | K((γ-Glu)₂-CO—(CH₂)₁₆—CO₂H)AQ-Aib-Orn-FIe-αMeY- | | |
| | LSEGGPSSGEPPPSGGE-NH₂ | | |
| 560 | Y-Aib-QGT-αMeF(2F)-TSD-4Pal-αMeS-I-αMeL-LD-Orn- | 566 | 5066.6 |
| | K((γ-Glu)₂-CO—(CH₂)₁₆—CO₂H)AQ-Aib-Orn-FIe-αMeY- | | |
| | LSEGGPSSGEPPPSGSE-NH₂ | | |
| 561 | Y-Aib-QGT-αMeF(2F)-TSD-4Pal-αMeS-I-αMeL-LD-Orn- | 567 | 5209.8 |
| | K((γ-Glu)₂-CO—(CH₂)₁₆—CO₂H)AQ-Aib-Orn-FIe-αMeY- | | |
| | LSEGGPSSGEPPPSGTEE-NH₂ | | |
| 562 | Y-Aib-QGT-αMeF(2F)-TSD-4Pal-αMeS-I-αMeL-LD-Orn- | 568 | 5338.9 |
| | K((γ-Glu)₂-CO—(CH₂)₁₆—CO₂H)AQ-Aib-Orn-FIe-αMeY- | | |
| | LSEGGPSSGEPPPSGTEEE-NH₂ | | |
| 563 | Y-Aib-QGT-αMeF(2F)-TSD-4Pal-αMeS-I-αMeL-LD-Orn- | 569 | 5080.7 |
| | K((Glu)-(γ-Glu)-CO—(CH₂)₁₆—CO₂H)AQ-Aib-Orn-FIe- | | |
| | αMeY-LSEGGPSSGEPPPSGTE-NH₂ | | |
| 564 | Y-Aib-QGT-αMeF(2F)-TSD-4Pal-αMeS-I-αMeL-LD-Orn- | 570 | 5209.8 |
| | K((Glu)-(γ-Glu)-CO—(CH₂)₁₆—CO₂H)AQ-Aib-Orn-FIe- | | |
| | αMeY-LSEGGPSSGEPPPSGTEE-NH₂ | | |
| 565 | Y-Aib-QGT-αMeF(2F)-TSD-4Pal-αMeS-I-αMeL-LD-Orn- | 571 | 5080.7 |
| | K((γ-Glu)-(Glu)-CO—(CH₂)₁₆—CO₂H)AQ-Aib-Orn-FIe- | | |
| | αMeY-LSEGGPSSGEPPPSGTE-NH₂ | | |
| 566 | Y-Aib-QGT-αMeF(2F)-TSD-4Pal-αMeS-I-αMeL-LD-Orn- | 572 | 5209.8 |
| | K((γ-Glu)-(Glu)-CO—(CH₂)₁₆—CO₂H)AQ-Aib-Orn-FIe- | | |
| | αMeY-LSEGGPSSGEPPPSGTEE-NH₂ | | |
| 567 | Y-Aib-QGT-αMeF(2F)-TSD-4Pal-αMeS-I-αMeL-LD-Orn- | 573 | 5179.8 |
| | K((γ-Glu)₂-CO—(CH₂)₁₆—CO₂H)AQ-Aib-Orn-FIe-αMeY- | | |
| | LSEGGPSSGEPPPSGAEE-NH₂ | | |
| 568 | Y-Aib-QGT-αMeF(2F)-TSD-4Pal-αMeS-I-αMeL-LD-Orn- | 574 | 5165.7 |
| | K((γ-Glu)₂-CO—(CH₂)₁₆—CO₂H)AQ-Aib-Orn-FIe-αMeY- | | |
| | LSEGGPSSGEPPPSGGEE-NH₂ | | |
| 569 | Y-Aib-QGT-αMeF(2F)-TSD-4Pal-αMeS-I-αMeL-LD-Orn- | 575 | 5195.7 |
| | K((γ-Glu)₂-CO—(CH₂)₁₆—CO₂H)AQ-Aib-Orn-FIe-αMeY- | | |
| | LSEGGPSSGEPPPSGSEE-NH₂-NH₂ | | |
| 570 | Y-Aib-QGT-αMeF(2F)-TSD-4Pal-αMeS-I-αMeL-LD-Orn- | 576 | 5236.8 |
| | K((γ-Glu)₂-CO—(CH₂)₁₆—CO₂H)AQ-Aib-Orn-FIe-αMeY- | | |
| | LSEGGPSSGEPPPSGQEE-NH₂ | | |
| 571 | Y-Aib-QGT-αMeF(2F)-TSD-4Pal-αMeS-I-αMeL-LD-Orn- | 577 | 5193.8 |
| | K((γ-Glu)₂-CO—(CH₂)₁₆—CO₂H)AQ-Aib-Orn-FIe-αMeY- | | |
| | LAEGGPSSGEPPPSGTEE-NH₂ | | |
| 572 | Y-Aib-QGT-αMeF(2F)-TSD-4Pal-αMeS-I-αMeL-LD-Orn- | 578 | 5250.8 |
| | K((γ-Glu)₂-CO—(CH₂)₁₆—CO₂H)AQ-Aib-Orn-FIe-αMeY- | | |
| | LQEGGPSSGEPPPSGTEE-NH₂ | | |
| 573 | Y-Aib-QGT-αMeF(2F)-TSD-4Pal-αMeS-I-αMeL-LD-Orn- | 579 | 5379.9 |
| | K((γ-Glu)₂-CO—(CH₂)₁₆—CO₂H)AQ-Aib-Orn-FIe-αMeY- | | |
| | LQEGGPSSGEPPPSGTEEE-NH₂ | | |
| 574 | Y-Aib-QGT-αMeF(2F)-TSD-4Pal-αMeS-ILLD-Orn-K((γ- | 580 | 5248.8 |
| | Glu)₂-CO—(CH₂)₁₆—CO₂H)AQ-Aib-Orn-FIe-αMeY- | | |
| | LQEGGPSSGEPPPEGAEE-NH₂ | | |
| 575 | Y-Aib-QGT-αMeF(2F)-TSD-4Pal-αMeS-ILLD-Orn-K((γ- | 581 | 5206.8 |
| | Glu)₂-CO—(CH₂)₁₆—CO₂H)AQ-Aib-Orn-FIe-αMeY- | | |
| | LQEGGPSSGEPPPSGAEE-NH₂ | | |
| 576 | Y-Aib-QGT-αMeF(2F)-TSD-4Pal-αMeS-ILLD-Orn-K((γ- | 582 | 5207.8 |
| | Glu)₂-CO—(CH₂)₁₆—CO₂H)AQ-Aib-Orn- | | |
| | FIeYLIEGGPSSGEPPPSGTEE-NH₂ | | |
| 577 | Y-Aib-QGT-αMeF(2F)-TSD-4Pal-αMeS-ILLD-Orn-K((γ- | 583 | 5379.0 |
| | Glu)₂-CO—(CH₂)₁₆—CO₂H)AQ-Aib-Orn- | | |
| | FIeYLIEGGPSSGEPPPEGTEEE-NH₂ | | |
| 578 | Y-Aib-QGT-αMeF(2F)-TSD-4Pal-SILLDKK((γ-Glu)₂-CO— | 584 | 5144.7 |
| | (CH₂)₁₆—CO₂H)AQ-Aib- | | |
| | AFIEWLQEGGPSSGAPPPSGTEE-NH₂ | | |
| 579 | Y-Aib-QGT-αMeF(2F)-TSD-4Pal-SILLDKK((γ-Glu)₂-CO— | 585 | 5202.7 |
| | (CH₂)₁₆—CO₂H)AQ-Aib-AFIEWLQEGGPSSGEPPPSGTEE- | | |
| | NH₂ | | |
| 580 | Y-Aib-QGT-αMeF(2F)-TSD-4Pal-SILLDKK((γ-Glu)₂-CO— | 586 | 5121.7 |
| | (CH₂)₁₆—CO₂H)AQ-Aib-AFIEYLQEGGPSSGAPPPSGTEE- | | |
| | NH₂ | | |
| 581 | Y-Aib-QGT-αMeF(2F)-TSD-4Pal-SILLD-Orn-K((γ-Glu)₂- | 587 | 5107.6 |
| | CO—(CH₂)₁₆—CO₂H)AQ-Aib- | | |
| | AFIEYLQEGGPSSGAPPPSGTEE-NH₂ | | |
| 582 | Y-Aib-QGT-αMeF(2F)-TSD-4Pal-SILLD-Orn-K((γ-Glu)₂- | 588 | 5150.7 |
| | CO—(CH₂)₁₆—CO₂H)AQ-Aib-Orn- | | |
| | FIEYLQEGGPSSGAPPPSGTEE-NH₂ | | |
| 583 | Y-Aib-QGT-αMeF(2F)-TSD-4Pal-SILLD-Orn-K((γ-Glu)₂- | 589 | 5165.7 |
| | CO—(CH₂)₁₆—CO₂H)AQ-Aib- | | |
| | AFIEYLQEGGPSSGEPPPSGTEE-NH₂ | | |
| 584 | Y-Aib-QGT-αMeF(2F)-TSD-4Pal-SILLD-Orn-K((γ-Glu)₂- | 590 | 5270.9 |
| | CO—(CH₂)₁₆—CO₂H)AQ-Me4Pal-Orn- | | |
| | FIeYLIEGGPSSGEPPPSGTEE-NH₂ | | |
| 585 | Y-Aib-QGT-αMeF(2F)-TSD-4Pal-αMeS-I-αMeL-LD-Orn- | 591 | 5278.8 |
| | K((γ-Glu)₂-CO—(CH₂)₁₆—CO₂H)AQ-Aib-Orn-FIe-αMeY- | | |
| | LQEGGPSSGEPPPEGSEE-NH₂ | | |
| 586 | Y-Aib-QGT-αMeF(2F)-TSD-4Pal-αMeS-I-αMeL-LD-Orn- | 592 | 5248.8 |
| | K((γ-Glu)₂-CO—(CH₂)₁₆—CO₂H)AQ-Aib-Orn-FIe-αMeY- | | |
| | LQEGGPSSGEPPPEGGEE-NH₂ | | |
| 587 | Y-Aib-QGT-αMeF(2F)-TSD-4Pal-αMeS-I-αMeL-LD-Orn- | 593 | 5262.8 |
| | K((γ-Glu)₂-CO—(CH₂)₁₆—CO₂H)AQ-Aib-Orn-FIe-αMeY- | | |
| | LQEGGPSSGEPPPEGAEE-NH₂ | | |
| 588 | Y-Aib-QGT-αMeF(2F)-TSD-4Pal-αMeS-I-αMeL-LD-Orn- | 594 | 5320.9 |
| | K((γ-Glu)₂-CO—(CH₂)₁₆—CO₂H)AQ-Aib-Orn-FIe-αMeY- | | |
| | LQEGGPSSGEPPPEGEEE-NH₂ | | |
| 589 | Y-Aib-QGT-αMeF(2F)-TSD-4Pal-αMeS-I-αMeL-LD-Orn- | 595 | 5179.7 |
| | K((γ-Glu)₂-CO—(CH₂)₁₆—CO₂H)AQ-Aib-Orn-FIe-αMeY- | | |
| | LAEGGPSSGEPPPSGSEE-NH₂ -NH₂ | | |
| 590 | Y-Aib-QGT-αMeF(2F)-TSD-4Pal-αMeS-I-αMeL-LD-Orn- | 596 | 5149.7 |
| | K((γ-Glu)₂-CO—(CH₂)₁₆—CO₂H)AQ-Aib-Orn-FIe-αMeY- | | |
| | LAEGGPSSGEPPPSGGEE-NH₂ | | |
| 591 | Y-Aib-QGT-αMeF(2F)-TSD-4Pal-αMeS-I-αMeL-LD-Orn- | 597 | 5163.7 |
| | K((γ-Glu)₂-CO—(CH₂)₁₆—CO₂H)AQ-Aib-Orn-FIe-αMeY- | | |
| | LAEGGPSSGEPPPSGAEE-NH₂ | | |
| 592 | Y-Aib-QGT-αMeF(2F)-TSD-4Pal-αMeS-I-αMeL-LD-Orn- | 598 | 5221.8 |
| | K((γ-Glu)₂-CO—(CH₂)₁₆—CO₂H)AQ-Aib-Orn-FIe-αMeY- | | |
| | LAEGGPSSGEPPPSGEEE-NH₂ | | |
| 593 | Y-Aib-QGT-αMeF(2F)-TSD-4Pal-αMeS-I-αMeL-LD-Orn- | 599 | 5292.9 |
| | K((Glu)-(γ-Glu)-CO—(CH₂)₁₆—CO₂H)AQ-Aib-Orn-FIe- | | |
| | αMeY-LQEGGPSSGEPPPEGTEE-NH₂ | | |
| 594 | Y-Aib-QGT-αMeF(2F)-TSD-4Pal-αMeS-I-αMeL-LD-Orn- | 600 | 5422.0 |
| | K((Glu)-(γ-Glu)-CO—(CH₂)₁₆—CO₂H)AQ-Aib-Orn-FIe- | | |
| | αMeY-LQEGGPSSGEPPPEGTEEE-NH₂ | | |
| 595 | Y-Aib-QGT-αMeF(2F)-TSD-4Pal-αMeS-I-αMeL-LD-Orn- | 601 | 5292.9 |
| | K(e-(γ-Glu)-CO—(CH₂)₁₆—CO₂H)AQ-Aib-Orn-FIe-αMeY- | | |
| | LQEGGPSSGEPPPEGTEE-NH₂ | | |
| 596 | Y-Aib-QGT-αMeF(2F)-TSD-4Pal-αMeS-I-αMeL-LD-Orn- | 602 | 5422.0 |
| | K(e-(γ-Glu)-CO—(CH₂)₁₆—CO₂H)AQ-Aib-Orn-FIe-αMeY- | | |
| | LQEGGPSSGEPPPEGTEEE-NH₂ | | |
| 597 | Y-Aib-QGT-αMeF(2F)-TSD-4Pal-αMeS-I-αMeL-LD-Orn- | 603 | 5265.8 |
| | K((γ-Glu)₂-CO—(CH₂)₁₆—CO₂H)AQ-Aib-Orn-FIe-αMeY- | | |
| | LTEGGPSSGEPPPEGTEE-NH₂ | | |
| 598 | Y-Aib-QGT-αMeF(2F)-TSD-4Pal-αMeS-I-αMeL-LD-Orn- | 604 | 5395.0 |
| | K((γ-Glu)₂-CO—(CH₂)₁₆—CO₂H)AQ-Aib-Orn-FIe-αMeY- | | |
| | LTEGGPSSGEPPPEGTEEE-NH₂ | | |
| 599 | Y-Aib-QGT-αMeF(2F)-TSD-4Pal-αMeS-I-αMeL-LD-Orn- | 605 | 5320.9 |
| | K((γ-Glu)₂-CO—(CH₂)₁₆—CO₂H)AQ-Aib-Orn-FIe-αMeY- | | |
| | LQEGGPSSGEPPPEGTEE-NH₂ | | |
| 600 | Y-Aib-QGT-αMeF(2F)-TSD-4Pal-αMeS-I-αMeL-LD-Orn- | 606 | 5450.0 |
| | K((γ-Glu)₂-CO—(CH₂)₁₆—CO₂H)AQ-Aib-Orn-FIe-αMeY- | | |
| | LQEGGPSSGEPPPEGTEEE-NH₂ | | |
| 601 | Y-Aib-QGT-αMeF(2F)-TSD-4Pal-αMeS-I-αMeL-LD-Orn- | 607 | 5293.8 |
| | K((γ-Glu)₂-CO—(CH₂)₁₆—CO₂H)AQ-Aib-Orn-FIe-αMeY- | | |
| | LQEGGPSSGEPPPEGTEE | | |
| 602 | Y-Aib-QGT-αMeF(2F)-TSD-4Pal-αMeS-I-αMeL-LD-Orn- | 608 | 5423.0 |
| | K((γ-Glu)₂-CO—(CH₂)₁₆—CO₂H)AQ-Aib-Orn-FIe-αMeY- | | |
| | LQEGGPSSGEPPPEGTEE | | |
| 603 | Y-Aib-QGT-αMeF(2F)-TSD-4Pal-αMeS-ILLD-Orn-K((γ- | 609 | 5235.8 |
| | Glu)₂-CO—(CH₂)₁₆—CO₂H)AQ-Aib-Orn- | | |
| | FIeYLIEGGPSSGEPPPEGSEE-NH₂ | | |
| 604 | Y-Aib-QGT-αMeF(2F)-TSD-4Pal-αMeS-ILLD-Orn-K((γ- | 610 | 5219.8 |
| | Glu)₂-CO—(CH₂)₁₆—CO₂H)AQ-Aib-Orn- | | |
| | FIeYLIEGGPSSGEPPPEGAEE-NH₂ | | |
| 605 | Y-Aib-QGT-αMeF(2F)-TSD-4Pal-αMeS-ILLD-Orn-K((γ- | 611 | 5205.8 |
| | Glu)₂-CO—(CH₂)₁₆—CO₂H)AQ-Aib-Orn- | | |
| | FIeYLIEGGPSSGEPPPEGGEE-NH₂ | | |
| 606 | Y-Aib-QGT-αMeF(2F)-TSD-4Pal-αMeS-ILLD-Orn-K((γ- | 612 | 5277.8 |
| | Glu)₂-CO—(CH₂)₁₆—CO₂H)AQ-Aib-Orn- | | |
| | FIeYLIEGGPSSGEPPPEGEEE-NH₂ | | |
| 607 | Y-Aib-QGT-αMeF(2F)-TSD-4Pal-SILLDKK((γ-Glu)₂-CO— | 613 | 5108.6 |
| | (CH₂)₁₆—CO₂H)AQ-Aib-AFIEYLEEGGPSSGEPPPSGTGE- | | |
| | NH₂ | | |
| 608 | Y-Aib-QGT-αMeF(2F)-TSD-4Pal-SILLDKK((γ-Glu)₂-CO— | 614 | 5180.7 |
| | (CH₂)₁₆—CO₂H)AQ-Aib-AFIEYLEEGGPSSGEPPPSGTEE- | | |
| | NH₂ | | |
| 609 | Y-Aib-QGT-αMeF(2F)-TSD-4Pal-SILLDKK((γ-Glu)₂-CO— | 615 | 5122.7 |
| | (CH₂)₁₆—CO₂H)AQ-Aib-AFIEYLEEGGPSSGAPPPSGTEE- | | |
| | NH₂ | | |
| 610 | Y-Aib-QGT-αMeF(2F)-TSD-4Pal-SILLDKK((γ-Glu)₂-CO— | 616 | 5108.6 |
| | (CH₂)₁₆—CO₂H)AQ-Aib-AFIEYLEEGGPSSGAPPPSGSEE- | | |
| | NH₂ | | |
| 611 | Y-Aib-QGT-αMeF(2F)-TSD-4Pal-SILLDKK((γ-Glu)₂-CO— | 617 | 5150.7 |
| | (CH₂)₁₆—CO₂H)AQ-Aib-AFIEYLEEGGPSSGAPPPSGEEE- | | |
| | NH₂ | | |
| 612 | Y-Aib-QGT-αMeF(2F)-TSD-4Pal-SILLDKK((γ-Glu)₂-CO— | 618 | 5078.6 |
| | (CH₂)₁₆—CO₂H)AQ-Aib-AFIEYLEEGGPSSGAPPPSGGEE- | | |
| | NH₂ | | |
| 613 | Y-Aib-QGT-αMeF(2F)-TSD-4Pal-SI-αMeL-LD-Orn-K((2- | 619 | 5186.8 |
| | [2-(2-Amino-ethoxy)-ethoxy]-acetyl)₂-(γ-Glu)-CO—(CH₂)₁₆- | | |
| | CO₂H)AQ-Me4Pal-EFIe-αMeY-LLEGGPSSGAPPPSGE- | | |
| | NH₂ | | |
| 614 | Y-Aib-QGT-αMeF(2F)-TSD-4Pal-SI-αMeL-LD-Orn-K((2- | 620 | 5041.7 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LIEGGPSSGAPPPSGE- | | |
| | NH₂ | | |
| 615 | Y-Aib-QGT-αMeF(2F)-TSD-4Pal-SI-αMeL-LD-Orn-K((2- | 621 | 5041.7 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LLEGGPSSGAPPPSGE- | | |
| | NH₂ | | |
| 616 | Y-Aib-QGT-αMeF(2F)-TSD-4Pal-SI-αMeL-LD-Orn-K((γ- | 622 | 5025.6 |
| | Glu)₂-CO—(CH₂)₁₆—CO₂H)AQ-Me4Pal-EFIe-αMeY- | | |
| | LIEGGPSSGAPPPSGE-NH₂ | | |
| 617 | Y-Aib-QGT-αMeF(2F)-TSD-4Pal-SI-αMeL-LD-Orn-K((γ- | 623 | 5025.6 |
| | Glu)₂-CO—(CH₂)₁₆—CO₂H)AQ-Me4Pal-EFIe-αMeY- | | |
| | LLEGGPSSGAPPPSGE-NH₂ | | |
| 618 | Y-Aib-QGT-αMeF(2F)-TSD-4Pal-SILLDKK((γ-Glu)₂-CO— | 624 | 5036.6 |
| | (CH₂)₁₆—CO₂H)AQ-Aib-AFIEYLEEGGPSSGAPPPSGSGE- | | |
| | NH₂ | | |
| 619 | Y-Aib-QGT-αMeF(2F)-TSD-4Pal-SILLDKK((γ-Glu)₂-CO— | 625 | 5006.5 |
| | (CH₂)₁₆—CO₂H)AQ-Aib-AFIEYLEEGGPSSGAPPPSGGGE- | | |
| | NH₂ | | |
| 620 | Y-Aib-QGT-αMeF(2F)-TSD-4Pal-SILLDKK((γ-Glu)₂-CO— | 626 | 5020.6 |
| | (CH₂)₁₆—CO₂H)AQ-Aib-AFIEYLEEGGPSSGAPPPSGAGE- | | |
| | NH₂ | | |
| 621 | Y-Aib-QGT-αMeF(2F)-TSD-4Pal-SILLDKK((γ-Glu)₂-CO— | 627 | 5078.6 |
| | (CH₂)₁₆—CO₂H)AQ-Aib-AFIEYLEEGGPSSGAPPPSGEGE- | | |
| | NH₂ | | |
| 622 | Y-Aib-QGT-αMeF(2F)-TSD-4Pal-SI-αMeL-LDKK((γ- | 628 | 5064.6 |
| | Glu)₂-CO—(CH₂)₁₆—CO₂H)AQ-Aib- | | |
| | AFIEYLEEGGPSSGAPPPSGTGE-NH₂ | | |
| 623 | Y-Aib-QGT-αMeF(2F)-TSD-4Pal-SI-αMeL-LDKK((γ- | 629 | 5092.6 |
| | Glu)₂-CO—(CH₂)₁₆—CO₂H)AQ-Aib- | | |
| | AFIEYLEEGGPSSGAPPPSGEGE-NH₂ | | |
| 624 | Y-Aib-QGT-αMeF(2F)-TSD-4Pal-SI-αMeL-LDKK((γ- | 630 | 5122.7 |
| | Glu)₂-CO—(CH₂)₁₆—CO₂H)AQ-Aib- | | |
| | AFIEYLEEGGPSSGEPPPSGTGE-NH₂ | | |
| 625 | Y-Aib-QGT-αMeF(2F)-TSD-4Pal-SI-αMeL-LDKK((γ- | 631 | 5150.7 |
| | Glu)₂-CO—(CH₂)₁₆—CO₂H)AQ-Aib- | | |
| | AFIEYLEEGGPSSGEPPPSGEGE-NH₂ | | |
| 626 | Y-Aib-QGT-αMeF(2F)-TSD-4Pal-SI-αMeL-LDKK((γ- | 632 | 5106.7 |
| | Glu)₂-CO—(CH₂)₁₆—CO₂H)AQ-Aib- | | |
| | AFIEYLEEGGPSSGAPPPEGTGE-NH₂ | | |
| 627 | Y-Aib-QGT-αMeF(2F)-TSD-4Pal-SI-αMeL-LDKK((γ- | 633 | 5134.7 |
| | Glu)₂-CO—(CH₂)₁₆—CO₂H)AQ-Aib- | | |
| | AFIEYLEEGGPSSGAPPPEGTGE-NH₂ | | |
| 628 | Y-Aib-QGT-αMeF(2F)-TSD-4Pal-SI-αMeL-LDKK((γ- | 634 | 5164.7 |
| | Glu)₂-CO—(CH₂)₁₆—CO₂H)AQ-Aib- | | |
| | AFIEYLEEGGPSSGEPPPEGTGE-NH₂ | | |
| 629 | Y-Aib-QGT-αMeF(2F)-TSD-4Pal-SI-αMeL-LDKK((γ- | 635 | 5192.7 |
| | Glu)₂-CO—(CH₂)₁₆—CO₂H)AQ-Aib- | | |
| | AFIEYLEEGGPSSGEPPPEGTGE-NH₂ | | |
| 630 | Y-Aib-QGT-αMeF(2F)-TSD-4Pal-SI-αMeL-LDKK((γ- | 636 | 5166.7 |
| | Glu)₂-CO—(CH₂)₁₆—CO₂H)AQ-Aib- | | |
| | AFIEYLEEGGPSSGEPPPSGSEE-NH₂ | | |
| 631 | Y-Aib-QGT-αMeF(2F)-TSD-4Pal-SI-αMeL-LDKK((γ- | 637 | 5108.6 |
| | Glu)₂-CO—(CH₂)₁₆—CO₂H)AQ-Aib- | | |
| | AFIEYLAEGGPSSGEPPPSGSEE-NH₂ | | |
| 632 | Y-Aib-QGT-αMeF(2F)-TSD-4Pal-SI-αMeL-LDKK((γ- | 638 | 5124.6 |
| | Glu)₂-CO—(CH₂)₁₆—CO₂H)AQ-Aib- | | |
| | AFIEYLSEGGPSSGEPPPSGSEE-NH₂ | | |
| 633 | Y-Aib-QGT-αMeF(2F)-TSD-4Pal-SI-αMeL-LDKK((γ- | 639 | 5138.6 |
| | Glu)₂-CO—(CH₂)₁₆—CO₂H)AQ-Aib- | | |
| | AFIEYLTEGGPSSGEPPPSGSEE-NH₂ | | |
| 634 | Y-Aib-QGT-αMeF(2F)-TSD-4Pal-SI-αMeL-LDKK((γ- | 640 | 5165.7 |
| | Glu)₂-CO—(CH₂)₁₆—CO₂H)AQ-Aib- | | |
| | AFIEYLQEGGPSSGEPPPSGSEE-NH₂ | | |
| 635 | Y-Aib-QGT-αMeF(2F)-TSD-4Pal-SI-αMeL-LDKK((γ- | 641 | 5209.7 |
| | Glu)₂-CO—(CH₂)₁₆—CO₂H)AQ-Aib-Orn- | | |
| | FIEYLEEGGPSSGEPPPSGSEE-NH₂ | | |
| 636 | Y-Aib-QGT-αMeF(2F)-TSD-4Pal-SI-αMeL-LDKK((γ- | 642 | 5151.7 |
| | Glu)₂-CO—(CH₂)₁₆—CO₂H)AQ-Aib-Orn- | | |
| | FIEYLAEGGPSSGEPPPSGSEE-NH₂ | | |
| 637 | Y-Aib-QGT-αMeF(2F)-TSD-4Pal-SI-αMeL-LDKK((γ- | 643 | 5167.7 |
| | Glu)₂-CO—(CH₂)₁₆—CO₂H)AQ-Aib-Orn- | | |
| | FIEYLSEGGPSSGEPPPSGSEE-NH₂ | | |
| 638 | Y-Aib-QGT-αMeF(2F)-TSD-4Pal-SI-αMeL-LDKK((γ- | 644 | 5181.7 |
| | Glu)₂-CO—(CH₂)₁₆—CO₂H)AQ-Aib-Orn- | | |
| | FIEYLTEGGPSSGEPPPSGSEE-NH₂ | | |
| 639 | Y-Aib-QGT-αMeF(2F)-TSD-4Pal-SI-αMeL-LDKK((γ- | 645 | 5208.7 |
| | Glu)₂-CO—(CH₂)₁₆—CO₂H)AQ-Aib-Orn- | | |
| | FIEYLQEGGPSSGEPPPSGSEE-NH₂ | | |
| 640 | Y-Aib-QGT-αMeF(2F)-TSD-4Pal-SI-αMeL-LD-Orn-K((2- | 646 | 4999.6 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LIEGGPSSGAPPPSGS- | | |
| | NH₂ | | |
| 641 | Y-Aib-QGT-αMeF(2F)-TSD-4Pal-SI-αMeL-LD-Orn-K((2- | 647 | 4999.6 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LLEGGPSSGAPPPSGS- | | |
| | NH₂ | | |
| 642 | Y-Aib-QGT-αMeF(2F)-TSD-4Pal-SI-αMeL-LD-Orn-K((2- | 648 | 4957.5 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LAEGGPSSGAPPPSGS- | | |
| | NH₂ | | |
| 643 | Y-Aib-QGT-αMeF(2F)-TSD-4Pal-SI-αMeL-LD-Orn-K((2- | 649 | 5014.6 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LQEGGPSSGAPPPSGS- | | |
| | NH₂ | | |
| 644 | Y-Aib-QGT-αMeF(2F)-TSD-4Pal-SI-αMeL-LD-Orn-K((2- | 650 | 5057.7 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LIEGGPSSGEPPPSGS- | | |
| | NH₂ | | |
| 645 | Y-Aib-QGT-αMeF(2F)-TSD-4Pal-SI-αMeL-LD-Orn-K((2- | 651 | 5057.7 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LLEGGPSSGEPPPSGS- | | |
| | NH₂ | | |
| 646 | Y-Aib-QGT-αMeF(2F)-TSD-4Pal-SI-αMeL-LD-Orn-K((γ- | 652 | 4941.5 |
| | Glu)₂-CO—(CH₂)₁₆—CO₂H)AQ-Me4Pal-EFIe-αMeY- | | |
| | LAEGGPSSGAPPPSGS-NH₂ | | |
| 647 | Y-Aib-QGT-αMeF(2F)-TSD-4Pal-SI-αMeL-LD-Orn-K((γ- | 653 | 4998.6 |
| | Glu)₂-CO—(CH₂)₁₆—CO₂H)AQ-Me4Pal-EFIe-αMeY- | | |
| | LQEGGPSSGAPPPSGS-NH₂ | | |
| 648 | Y-Aib-QGT-αMeF(2F)-TSD-4Pal-SI-αMeL-LD-Orn-K((2- | 654 | 5072.6 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LQEGGPSSGEPPPSGS- | | |
| | NH₂ | | |
| 649 | Y-Aib-QGT-αMeF(2F)-TSD-4Pal-SI-αMeL-LD-Orn-K((γ- | 655 | 5056.6 |
| | Glu)₂-CO—(CH₂)₁₆—CO₂H)AQ-Me4Pal-EFIe-αMeY- | | |
| | LQEGGPSSGEPPPSGS-NH₂ | | |
| 650 | Y-Aib-QGT-αMeF(2F)-TSD-4Pal-SI-αMeL-LDKK((γ- | 656 | 5193.7 |
| | Glu)₂-CO—(CH₂)₁₆—CO₂H)AQ-Aib-Orn- | | |
| | FIEYLAEGGPSSGEPPPEGSEE-NH₂ | | |
| 651 | Y-Aib-QGT-αMeF(2F)-TSD-4Pal-SI-αMeL-LDKK((γ- | 657 | 5209.7 |
| | Glu)₂-CO—(CH₂)₁₆—CO₂H)AQ-Aib-Orn- | | |
| | FIEYLSEGGPSSGEPPPEGSEE-NH₂ | | |
| 652 | Y-Aib-QGT-αMeF(2F)-TSD-4Pal-SI-αMeL-LDKK((γ- | 658 | 5223.8 |
| | Glu)₂-CO—(CH₂)₁₆—CO₂H)AQ-Aib-Orn- | | |
| | FIEYLTEGGPSSGEPPPEGSEE-NH₂ | | |
| 653 | Y-Aib-QGT-αMeF(2F)-TSD-4Pal-SI-αMeL-LDKK((γ- | 659 | 5250.8 |
| | Glu)₂-CO—(CH₂)₁₆—CO₂H)AQ-Aib-Orn- | | |
| | FIEYLQEGGPSSGEPPPEGSEE-NH₂ | | |
| 654 | Y-Aib-QGT-αMeF(2F)-TSD-4Pal-αMeS-I-αMeL-LD-Orn- | 660 | 5305.0 |
| | K((γ-Glu)-(6-aminohexanoic acid)-CO—(CH₂)₁₆—CO₂H)AQ- | | |
| | Aib-Orn-FIe-αMeY-LQEGGPSSGEPPPEGTEE-NH₂ | | |
| 655 | Y-Aib-QGT-αMeF(2F)-TSD-4Pal-αMeS-I-αMeL-LD-Orn- | 661 | 5337.0 |
| | K((γ-Glu)-(2-[2-(2-Amino-ethoxy)-ethoxy]-acetyl)-CO— | | |
| | (CH₂)₁₆—CO₂H)AQ-Aib-Orn-FIe-αMeY- | | |
| | LQEGGPSSGEPPPEGTEE-NH₂ | | |
| 656 | Y-Aib-QGT-αMeF(2F)-TSD-4Pal-αMeS-I-αMeL-LD-Orn- | 662 | 5320.0 |
| | K((γ-Glu)-(8-Lys)-CO—(CH₂)₁₆—CO₂H)AQ-Aib-Orn-FIe- | | |
| | αMeY-LQEGGPSSGEPPPEGTEE-NH₂ | | |
| 657 | Y-Aib-QGT-αMeF(2F)-TSD-4Pal-αMeS-I-αMeL-LD-Orn- | 663 | 5320.9 |
| | K((γ-Glu)-e-CO—(CH₂)₁₆—CO₂H)AQ-Aib-Orn-FIe-αMeY- | | |
| | LQEGGPSSGEPPPEGTEE-NH₂ | | |
| 658 | Y-Aib-QGT-αMeF(2F)-TSD-4Pal-αMeS-I-αMeL-LD-Orn- | 664 | 5248.9 |
| | K((γ-Glu)-(Gly)-CO—(CH₂)₁₆—CO₂H)AQ-Aib-Orn-FIe- | | |
| | αMeY-LQEGGPSSGEPPPEGTEE-NH₂ | | |
| 659 | Y-Aib-QGT-αMeF(2F)-TSD-4Pal-SILLDKK((γ-Glu)₂-CO— | 665 | 5035.6 |
| | (CH₂)₁₆—CO₂H)AQ-Aib-AFIEYLEEGGPSSGAPPPEGTE- | | |
| | NH₂ | | |
| 660 | Y-Aib-QGT-αMeF(2F)-TSD-4Pal-SILLDKK((γ-Glu)₂-CO— | 666 | 5092.6 |
| | (CH₂)₁₆—CO₂H)AQ-Aib-AFIEYLEEGGPSSGAPPPEGTGE- | | |
| | NH₂ | | |
| 661 | Y-Aib-QGT-αMeF(2F)-TSD-4Pal-SILLDKK((γ-Glu)₂-CO— | 667 | 5064.6 |
| | (CH₂)₁₆—CO₂H)AQ-Aib- | | |
| | AFIEYLAadEGGPSSGAPPPSGTGE-NH₂ | | |
| 662 | Y-Aib-QGT-αMeF(2F)-TSD-4Pal-SILLDKK((2-[2-(2- | 668 | 5066.6 |
| | Amino-ethoxy)-ethoxy]-acetyl)-(γ-Glu)-CO—(CH₂)₁₆- | | |
| | CO₂H)AQ-Aib-AFIEYLEEGGPSSGAPPPSGTGE-NH₂ | | |
| 663 | Y-Aib-QGT-αMeF(2F)-TSD-4Pal-SILLDKK((2-[2-(2- | 669 | 5080.7 |
| | Amino-ethoxy)-ethoxy]-acetyl)-(γ-Glu)-CO—(CH₂)₁₆- | | |
| | CO₂H)AQ-Aib-AFIEYLAadEGGPSSGAPPPSGTGE-NH₂ | | |
| 664 | Y-Aib-QGT-αMeF(2F)-TSD-4Pal-SILLDKK((γ-Glu)₂-CO— | 670 | 5064.6 |
| | (CH₂)₁₆—CO₂H)AQ-Aib-AFIE-αMeY- | | |
| | LEEGGPSSGAPPPSGTGE-NH₂ | | |
| 665 | Y-Aib-QGT-αMeF(2F)-TSD-4Pal-SILLD-Orn-K((γ-Glu)₂- | 671 | 5050.6 |
| | CO—(CH₂)₁₆—CO₂H)AQ-Aib-AFIE-αMeY- | | |
| | LEEGGPSSGAPPPSGTGE-NH₂ | | |
| 666 | Y-Aib-QGT-αMeF(2F)-TSD-4Pal-SILLDKK((γ-Glu)₂-CO— | 672 | 5050.6 |
| | (CH₂)₁₆—CO₂H)AQ-Aib-AFIE-αMeY- | | |
| | LEEGGPSSGAPPPSGSGE-NH₂ | | |
| 667 | Y-Aib-QGT-αMeF(2F)-TSD-4Pal-SILLDKK((γ-Glu)₂-CO— | 673 | 5092.6 |
| | (CH₂)₁₆—CO₂H)AQ-Aib-AFIE-αMeY- | | |
| | LEEGGPSSGAPPPSGEGE-NH₂ | | |
| 668 | Y-Aib-QGT-αMeF(2F)-TSD-4Pal-SILLDKK((γ-Glu)₂-CO— | 674 | 5020.6 |
| | (CH₂)₁₆—CO₂H)AQ-Aib-AFIE-αMeY- | | |
| | LEEGGPSSGAPPPSGGGE-NH₂ | | |
| 669 | Y-Aib-QGT-αMeF(2F)-TSD-4Pal-SILLDKK((2-[2-(2- | 675 | 5080.7 |
| | Amino-ethoxy)-ethoxy]-acetyl)-(γ-Glu)-CO—(CH₂)₁₆- | | |
| | CO₂H)AQ-Aib-AFIE-αMeY-LEEGGPSSGAPPPSGTGE- | | |
| | NH₂ | | |
| 670 | Y-Aib-QGT-αMeF(2F)-TSD-4Pal-SILLD-Orn-K((2-[2-(2- | 676 | 4985.6 |
| | Amino-ethoxy)-ethoxy]-acetyl)-(γ-Glu)-CO—(CH₂)₁₆- | | |
| | CO₂H)AQ-Me4Pal-EFIe-αMeY-LLEGGPSSGAPPPSGE | | |
| 671 | Y-Aib-QGT-αMeF(2F)-TSD-4Pal-αMeS-I-αMeL-LD-Orn- | 677 | 4879.5 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGS-NH₂ | | |
| 672 | Y-Aib-QGT-αMeF(2F)-TSD-4Pal-αMeS-I-αMeL-LD-Orn- | 678 | 4879.5 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LLEGGPSSGAPPPSGS-NH₂ | | |
| 673 | Y-Aib-QGT-αMeF(2F)-TSD-4Pal-αMeS-I-αMeL-LD-Orn- | 679 | 4863.5 |
| | K((γ-Glu)₂-CO—(CH₂)₁₆—CO₂H)AA-Aib-EFIe-αMeY- | | |
| | LIEGGPSSGAPPPSGS-NH₂ | | |
| 674 | Y-Aib-QGT-αMeF(2F)-TSD-4Pal-SILLD-Orn-K((γ-Glu)₂- | 680 | 5093.7 |
| | CO—(CH₂)₁₆—CO₂H)AQ-Aib-Orn-FIE-αMeY- | | |
| | LEEGGPSSGAPPPSGTGE-NH₂ | | |
| 675 | Y-Aib-QGT-αMeF(2F)-TSD-4Pal-SI-αMeL-LD-Orn-K((2- | 681 | 5126.8 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGEPPPEGE- | | |
| | NH₂ | | |
| 676 | Y-Aib-QGT-αMeF(2F)-TSD-4Pal-SI-αMeL-LD-Orn-K((2- | 682 | 5140.8 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGEPPPEAE- | | |
| | NH₂ | | |
| 677 | Y-Aib-QGT-αMeF(2F)-TSD-4Pal-SI-αMeL-LD-Orn-K((2- | 683 | 5156.8 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGEPPPESE- | | |
| | NH₂ | | |
| 678 | Y-Aib-QGT-αMeF(2F)-TSD-4Pal-SI-αMeL-LD-Orn-K((2- | 684 | 5170.8 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGEPPPETE- | | |
| | NH₂ | | |
| 679 | Y-Aib-QGT-αMeF(2F)-TSD-4Pal-SI-αMeL-LD-Orn-K((2- | 685 | 5227.9 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LIEGGPSSGEPPPEGTE-NH₂ | | |
| 680 | Y-Aib-QGT-αMeF(2F)-TSD-4Pal-SI-αMeL-LD-Orn-K((2- | 686 | 5284.9 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LIEGGPSSGEPPPEGTGE-NH₂ | | |
| 681 | Y-Aib-QGT-αMeF(2F)-TSD-4Pal-SI-αMeL-LD-Orn-K((γ- | 687 | 5110.7 |
| | Glu)₂-CO—(CH₂)₁₆—CO₂H)AQ-Me4Pal-Orn-FIe-αMeY- | | |
| | LIEGGPSSGEPPPEEG-NH₂ | | |
| 682 | Y-Aib-QGT-αMeF(2F)-TSD-4Pal-SI-αMeL-LD-Orn-K((γ- | 688 | 5068.7 |
| | Glu)₂-CO—(CH₂)₁₆—CO₂H)AQ-Me4Pal-Orn-FIe-αMeY- | | |
| | LIEGGPSSGEPPPSGE-NH₂ | | |
| 683 | Y-Aib-QGT-αMeF(2F)-TSD-4Pal-SI-αMeL-LD-Orn-K((γ- | 689 | 5110.7 |
| | Glu)₂-CO—(CH₂)₁₆—CO₂H)AQ-Me4Pal-Orn-FIe-αMeY- | | |
| | LIEGGPSSGEPPPEGE-NH₂ | | |
| 684 | Y-Aib-QGT-αMeF(2F)-TSD-4Pal-SI-αMeL-LD-Orn-K((γ- | 690 | 5124.8 |
| | Glu)₂-CO—(CH₂)₁₆—CO₂H)AQ-Me4Pal-Orn-FIe-αMeY- | | |
| | LIEGGPSSGEPPPEAE-NH₂ | | |
| 685 | Y-Aib-QGT-αMeF(2F)-TSD-4Pal-SI-αMeL-LD-Orn-K((γ- | 691 | 5140.8 |
| | Glu)₂-CO—(CH₂)₁₆—CO₂H)AQ-Me4Pal-Orn-FIe-αMeY- | | |
| | LIEGGPSSGEPPPESE-NH₂ | | |
| 686 | Y-Aib-QGT-αMeF(2F)-TSD-4Pal-SI-αMeL-LD-Orn-K((γ- | 692 | 5154.8 |
| | Glu)₂-CO—(CH₂)₁₆—CO₂H)AQ-Me4Pal-Orn-FIe-αMeY- | | |
| | LIEGGPSSGEPPPETE-NH₂ | | |
| 687 | Y-Aib-QGT-αMeF(2F)-TSD-4Pal-SI-αMeL-LD-Orn-K((γ- | 693 | 5211.8 |
| | Glu)₂-CO—(CH₂)₁₆—CO₂H)AQ-Me4Pal-Orn-FIe-αMeY- | | |
| | LIEGGPSSGEPPPEGTE | | |
| 688 | Y-Aib-QGT-αMeF(2F)-TSD-4Pal-SI-αMeL-LD-Orn-K((γ- | 694 | 5268.9 |
| | Glu)₂-CO—(CH₂)₁₆—CO₂H)AQ-Me4Pal-Orn-FIe-αMeY- | | |
| | LIEGGPSSGEPPPEGTGE-NH₂ | | |
| 689 | Y-Aib-QGT-αMeF(2F)-TSD-4Pal-αMeS-I-αMeL-LD-Orn- | 695 | 5079.7 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGTGE-NH₂ | | |
| 690 | Y-Aib-QGT-αMeF(2F)-TSD-4Pal-αMeS-I-αMeL-LD-Orn- | 696 | 5063.7 |
| | K((γ-Glu)₂-CO—(CH₂)₁₆—CO₂H)AA-Aib-EFIe-αMeY- | | |
| | LIEGGPSSGAPPPSGTGE-NH₂ | | |
| 691 | Y-Aib-QGT-αMeF(2F)-TSD-4Pal-SI-αMeL-LD-Orn-K((2- | 697 | 5227.9 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LIEGGPSSGEPPPEGTe-NH₂ | | |
| 692 | Y-Aib-QGT-αMeF(2F)-TSD-4Pal-SI-αMeL-LD-Orn-K((2- | 698 | 5284.9 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LIEGGPSSGEPPPEGEGe-NH₂ | | |
| 693 | Y-Aib-QGT-αMeF(2F)-TSD-4Pal-αMeS-I-αMeL-LD-Orn- | 699 | 4865.5 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LVEGGPSSGAPPPSGS-NH₂ | | |
| 694 | Y-Aib-QGT-αMeF(2F)-TSD-4Pal-αMeS-I-αMeL-LD-Orn- | 700 | 4999.6 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGAPPPSGS-NH₂ | | |
| 695 | Y-Aib-QGT-αMeF(2F)-TSD-4Pal-αMeS-I-αMeL-LD-Orn- | 701 | 4907.5 |
| | K((γ-Glu)₂-CO—(CH₂)₁₆—CO₂H)AA-Aib-EFIe-αMeY- | | |
| | LVEGGPSSGEPPPSGS-NH₂ | | |
| 696 | Y-Aib-QGT-αMeF(2F)-TSD-4Pal-αMeS-I-αMeL-LD-Orn- | 702 | 4983.6 |
| | K((γ-Glu)₂-CO—(CH₂)₁₆—CO₂H)AQ-Me4Pal-EFIe-αMeY- | | |
| | LVEGGPSSGAPPPSGS-NH₂ | | |
| 697 | Y-Aib-QGT-αMeF(2F)-TSD-4Pal-αMeS-I-αMeL-LD-Orn- | 703 | 5221.9 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LIEGGPSSGEPPPEGTGE-NH₂ | | |
| 698 | Y-Aib-QGT-αMeF(2F)-TSD-4Pal-αMeS-I-αMeL-LD-Orn- | 704 | 5063.7 |
| | K((2-[2-(2-Amino-ethoxy)-ethoxy]-acetyl)-(γ-Glu)-CO— | | |
| | (CH₂)₁₆—CO₂H)AQ-Aib-Orn-FIe-αMeY- | | |
| | LIEGGPSSGEPPPEGE-NH₂ | | |
| 699 | Y-Aib-QGT-αMeF(2F)-TSD-4Pal-αMeS-I-αMeL-LD-Orn- | 705 | 5013.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPSGS-NH₂ | | |
| 700 | Y-Aib-QGT-αMeF(2F)-TSD-4Pal-αMeS-I-αMeL-LD-Orn- | 706 | 4997.6 |
| | K((γ-Glu)₂-CO—(CH₂)₁₆—CO₂H)AQ-Me4Pal-EFIe-αMeY- | | |
| | LIEGGPSSGAPPPSGS-NH₂ | | |
| 701 | Y-Aib-QGT-αMeF(2F)-TSD-4Pal-αMeS-I-αMeL-LD-Orn- | 707 | 4921.6 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGE-NH₂ | | |
| 702 | Y-Aib-QGT-αMeF(2F)-TSD-4Pal-αMeS-I-αMeL-LD-Orn- | 708 | 5008.6 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGSE-NH₂ | | |
| 703 | Y-Aib-QGT-αMeF(2F)-TSD-4Pal-αMeS-I-αMeL-LD-Orn- | 709 | 5022.7 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGEE-NH₂ | | |
| 704 | Y-Aib-QGT-αMeF(2F)-TSD-4Pal-αMeS-I-αMeL-LD-Orn- | 710 | 5137.7 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGSEE-NH₂ | | |
| 705 | Y-Aib-QGT-αMeF(2F)-TSD-4Pal-αMeS-I-αMeL-LD-Orn- | 711 | 5151.8 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGEEE-NH₂ | | |
| 706 | Y-Aib-QGT-αMeF(2F)-TSD-4Pal-SI-αMeL-LD-Orn-K((2- | 712 | 5042.7 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGEPPPSGS- | | |
| | NH₂ | | |
| 707 | Y-Aib-QGT-αMeF(2F)-TSD-4Pal-αMeS-I-αMeL-LD-Orn- | 713 | 4937.6 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGEPPPSGS-NH₂ | | |
| 708 | Y-Aib-QGT-αMeF(2F)-TSD-4Pal-αMeS-I-αMeL-LD-Orn- | 714 | 4921.5 |
| | K((γ-Glu)₂-CO—(CH₂)₁₆—CO₂H)AA-Aib-EFIe-αMeY- | | |
| | LIEGGPSSGAPPPSGS-NH₂ | | |
| 709 | Y-Aib-QGT-αMeF(2F)-TSD-4Pal-αMeS-I-αMeL-LD-Orn- | 715 | 5121.7 |
| | K((γ-Glu)₂-CO—(CH₂)₁₆—CO₂H)AA-Aib-EFIe-αMeY- | | |
| | LIEGGPSSGAPPPSGTGE-NH₂ | | |
| 710 | Y-Aib-QGT-αMeF(2F)-TSD-4Pal-αMeS-I-αMeL-LD-Orn- | 716 | 5071.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGEPPPSGS-NH₂ | | |
| 711 | Y-Aib-QGT-αMeF(2F)-TSD-4Pal-αMeS-I-αMeL-LD-Orn- | 717 | 5271.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGEPPPSGTGE-NH₂ | | |
| 712 | Y-Aib-QGT-αMeF(2F)-TSD-4Pal-αMeS-I-αMeL-LD-Orn- | 718 | 5142.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPSGSE-NH₂ | | |
| 713 | Y-Aib-QGT-αMeF(2F)-TSD-4Pal-αMeS-I-αMeL-LD-Orn- | 719 | 5271.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPSGSEE-NH₂ | | |
| 714 | Y-Aib-QGT-αMeF(2F)-TSD-4Pal-αMeS-I-αMeL-LD-Orn- | 720 | 5213.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPSGTGE-NH₂ | | |
| 715 | Y-Aib-QGT-αMeF(2F)-TSD-4Pal-αMeS-I-αMeL-LD-Orn- | 721 | 5055.7 |
| | K((γ-Glu)₂-CO—(CH₂)₁₆—CO₂H)AQ-Me4Pal-EFIe-αMeY- | | |
| | LIEGGPSSGEPPPSGS-NH₂ | | |
| 716 | Y-Aib-QGT-αMeF(2F)-TSD-4Pal-αMeS-I-αMeL-LD-Orn- | 722 | 5184.8 |
| | K((γ-Glu)₂-CO—(CH₂)₁₆—CO₂H)AQ-Me4Pal-EFIe-αMeY- | | |
| | LIEGGPSSGEPPPSGSE-NH₂ | | |
| 717 | Y-Aib-QGT-αMeF(2F)-TSD-4Pal-αMeS-I-αMeL-LD-Orn- | 723 | 5126.7 |
| | K((γ-Glu)₂-CO—(CH₂)₁₆—CO₂H)AQ-Me4Pal-EFIe-αMeY- | | |
| | LIEGGPSSGAPPPSGSE-NH₂ | | |
| 718 | Y-Aib-QGT-αMeF(2F)-TSD-4Pal-αMeS-I-αMeL-LD-Orn- | 724 | 5197.8 |
| | K((γ-Glu)₂-CO—(CH₂)₁₆—CO₂H)AQ-Me4Pal-EFIe-αMeY- | | |
| | LIEGGPSSGAPPPSGTGE-NH₂ | | |
| 719 | Y-Aib-QGT-αMeF(2F)-TSD-4Pal-αMeS-I-αMeL-LD-Orn- | 725 | 5022.7 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGEPPPSGTE-NH₂ | | |
| 720 | Y-Aib-QGT-αMeF(2F)-TSD-4Pal-αMeS-I-αMeL-LD-Orn- | 726 | 5022.7 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGEPPPSGSe-NH₂ | | |
| 721 | Y-Aib-QGT-αMeF(2F)-TSD-4Pal-αMeS-I-αMeL-LD-Orn- | 727 | 4921.6 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGe-NH₂ | | |
| 722 | Y-Aib-QGT-αMeF(2F)-TSD-4Pal-αMeS-I-αMeL-LD-Orn- | 728 | 4921.6 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY-LIEGGPSSGAPPPSG- | | |
| | YE-NH₂ | | |
| 723 | Y-Aib-QGT-αMeF(2F)-TSD-4Pal-αMeS-I-αMeL-LD-Orn- | 729 | 5079.7 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGTGe-NH₂ | | |
| 724 | Y-Aib-QGT-αMeF(2F)-TSD-4Pal-αMeS-I-αMeL-LD-Orn- | 730 | 5079.7 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGTG-yE-NH₂ | | |
| 725 | Y-Aib-QGT-αMeF(2F)-TSD-4Pal-αMeS-I-αMeL-LD-Orn- | 731 | 5151.8 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGTEE-NH₂ | | |
| 726 | Y-Aib-QGT-αMeF(2F)-TSD-4Pal-αMeS-I-αMeL-LD-Orn- | 732 | 5151.8 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGTE-γE-NH₂ | | |
| 727 | Y-Aib-QGT-αMeF(2F)-TSD-4Pal-SI-αMeL-LD-Orn-K((2- | 733 | 5112.7 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGDPPPEGE- | | |
| | NH₂ | | |
| 728 | Y-Aib-QGT-αMeF(2F)-TSD-4Pal-SI-αMeL-LD-Orn-K((2- | 734 | 5126.8 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGDPPPEAE- | | |
| | NH₂ | | |
| 729 | Y-Aib-QGT-αMeF(2F)-TSD-4Pal-SI-αMeL-LD-Orn-K((2- | 735 | 5142.8 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGDPPPESE- | | |
| | NH₂ | | |
| 730 | Y-Aib-QGT-αMeF(2F)-TSD-4Pal-SI-αMeL-LD-Orn-K((2- | 736 | 5156.8 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGDPPPETE- | | |
| | NH₂ | | |
| 731 | Y-Aib-QGT-αMeF(2F)-TSD-4Pal-SI-αMeL-LD-Orn-K((2- | 737 | 5213.9 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LIEGGPSSGDPPPEGTE-NH₂ | | |
| 732 | Y-Aib-QGT-αMeF(2F)-TSD-4Pal-SI-αMeL-LD-Orn-K((2- | 738 | 5270.9 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LIEGGPSSGDPPPEGTGE-NH₂ | | |
| 733 | Y-Aib-QGT-αMeF(2F)-TSD-4Pal-αMeS-I-αMeL-LD-Orn- | 739 | 5057.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGEPPPSGS-NH₂ | | |
| 734 | Y-Aib-QGT-αMeF(2F)-TSD-4Pal-αMeS-I-αMeL-LD-Orn- | 740 | 5257.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGEPPPSGTGE-NH₂ | | |
| 735 | Y-Aib-QGT-αMeF(2F)-TSD-4Pal-αMeS-I-αMeL-LD-Orn- | 741 | 5213.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGEPPPSGGGE-NH₂ | | |
| 736 | Y-Aib-QGT-αMeF(2F)-TSD-4Pal-αMeS-I-αMeL-LD-Orn- | 742 | 5315.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGEPPPSGSEE-NH₂ | | |
| 737 | Y-Aib-QGT-αMeF(2F)-TSD-4Pal-αMeS-I-αMeL-LD-Orn- | 743 | 5041.6 |
| | K((γ-Glu)₂-CO—(CH₂)₁₆—CO₂H)AQ-Me4Pal-EFIe-αMeY- | | |
| | LVEGGPSSGEPPPSGS-NH₂ | | |
| 738 | Y-Aib-QGT-αMeF(2F)-TSD-4Pal-αMeS-I-αMeL-LD-Orn- | 744 | 5241.8 |
| | K((γ-Glu)₂-CO—(CH₂)₁₆—CO₂H)AQ-Me4Pal-EFIe-αMeY- | | |
| | LVEGGPSSGEPPPSGTGE-NH₂ | | |
| 739 | Y-Aib-QGT-αMeF(2F)-TSD-4Pal-αMeS-I-αMeL-LD-Orn- | 745 | 5197.8 |
| | K((γ-Glu)₂-CO—(CH₂)₁₆—CO₂H)AQ-Me4Pal-EFIe-αMeY- | | |
| | LVEGGPSSGEPPPSGGGE-NH₂ | | |
| 740 | Y-Aib-QGT-αMeF(2F)-TSD-4Pal-αMeS-I-αMeL-LD-Orn- | 746 | 5299.9 |
| | K((γ-Glu)₂-CO—(CH₂)₁₆—CO₂H)AQ-Me4Pal-EFIe-αMeY- | | |
| | LVEGGPSSGEPPPSGSEE-NH₂ | | |
| 741 | Y-Aib-QGT-αMeF(2F)-TSD-4Pal-αMeS-I-αMeL-LD-Orn- | 747 | 5169.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPSGGGE-NH₂ | | |
| 742 | Y-Aib-QGT-αMeF(2F)-TSD-4Pal-αMeS-I-αMeL-LD-Orn- | 748 | 5227.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGEPPPSGGGE-NH₂ | | |
| 743 | Y-Aib-QGT-αMeF(2F)-TSD-4Pal-αMeS-I-αMeL-LD-Orn- | 749 | 5153.8 |
| | K((γ-Glu)₂-CO—(CH₂)₁₆—CO₂H)AQ-Me4Pal-EFIe-αMeY- | | |
| | LIEGGPSSGAPPPSGGGE-NH₂ | | |
| 744 | Y-Aib-QGT-αMeF(2F)-TSD-4Pal-αMeS-I-αMeL-LD-Orn- | 750 | 5211.8 |
| | K((γ-Glu)₂-CO—(CH₂)₁₆—CO₂H)AQ-Me4Pal-EFIe-αMeY- | | |
| | LIEGGPSSGEPPPSGGGE-NH₂ | | |
| 745 | Y-Aib-QGT-αMeF(2F)-TSD-4Pal-αMeS-I-αMeL-LD-Orn- | 751 | 5035.7 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGAPPPSGGGE-NH₂ | | |
| 746 | Y-Aib-QGT-αMeF(2F)-TSD-4Pal-αMeS-I-αMeL-LD-Orn- | 752 | 5093.7 |
| | K((2-[2-(2-Amino-ethoxy)-ethoxy]-acetyl)-(γ-Glu)-CO— | | |
| | (CH₂)₁₆—CO₂H)AA-Aib-EFIe-αMeY- | | |
| | LIEGGPSSGEPPPSGGGE-NH₂ | | |
| 747 | Y-Aib-QGT-αMeF(2F)-TSD-4Pal-αMeS-I-αMeL-LD-Orn- | 753 | 5019.6 |
| | K((γ-Glu)₂-CO—(CH₂)₁₆—CO₂H)AA-Aib-EFIe-αMeY- | | |
| | LIEGGPSSGAPPPSGGGE-NH₂ | | |
| 748 | Y-Aib-QGT-αMeF(2F)-TSD-4Pal-αMeS-I-αMeL-LD-Orn- | 754 | 5077.7 |
| | K((γ-Glu)₂-CO—(CH₂)₁₆—CO₂H)AA-Aib-EFIe-αMeY- | | |
| | LIEGGPSSGEPPPSGGGE-NH₂ | | |
| 749 | Y-Aib-QGT-αMeF(2F)-TSD-4Pal-αMeS-I-αMeL-LD-Orn- | 755 | 5041.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGAPPPSGE-NH₂ | | |
| 750 | Y-Aib-QGT-αMeF(2F)-TSD-4Pal-αMeS-I-αMeL-LD-Orn- | 756 | 5099.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGEPPPSGE-NH₂ | | |
| 751 | Y-Aib-QGT-αMeF(2F)-TSD-4Pal-αMeS-I-αMeL-LD-Orn- | 757 | 5128.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGAPPPSGSE-NH₂ | | |
| 752 | Y-Aib-QGT-αMeF(2F)-TSD-4Pal-αMeS-I-αMeL-LD-Orn- | 758 | 5186.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGEPPPSGSE-NH₂ | | |
| 753 | Y-Aib-QGT-αMeF(2F)-TSD-4Pal-SI-αMeL-LD-Orn-K((2- | 759 | 5285.9 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LIEGGPSSGEPPPESEE-NH₂ | | |
| 754 | Y-Aib-QGT-αMeF(2F)-TSD-4Pal-SI-αMeL-LD-Orn-K((2- | 760 | 5343.0 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LIEGGPSSGEPPPESEEE-NH₂ | | |
| 755 | Y-Aib-QGT-αMeF(2F)-TSD-4Pal-SI-αMeL-LD-Orn-K((2- | 761 | 5098.8 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGAPPPESE- | | |
| | NH₂ | | |
| 756 | Y-Aib-QGT-αMeF(2F)-TSD-4Pal-SI-αMeL-LD-Orn-K((2- | 762 | 5227.9 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LIEGGPSSGAPPPESEE-NH₂ | | |
| 757 | Y-Aib-QGT-αMeF(2F)-TSD-4Pal-SI-αMeL-LD-Orn-K((2- | 763 | 5240.9 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LIEGGPSSGEPPPEGGGE-NH₂ | | |
| 758 | Y-Aib-QGT-αMeF(2F)-TSD-4Pal-SI-αMeL-LD-Orn-K((2- | 764 | 5182.8 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY- | | |
| | LIEGGPSSGAPPPEGGGE-NH₂ | | |
| 759 | Y-Aib-QGT-αMeF(2F)-TSD-4Pal-αMeS-I-αMeL-LD-Orn- | 765 | 5211.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPEGGGE-NH₂ | | |
| 760 | Y-Aib-QGT-αMeF(2F)-TSD-4Pal-αMeS-I-αMeL-LD-Orn- | 766 | 5269.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGEPPPEGGGE-NH₂ | | |
| 761 | Y-Aib-QGT-αMeF(2F)-TSD-4Pal-αMeS-I-αMeL-LD-Orn- | 767 | 5195.8 |
| | K((γ-Glu)₂-CO—(CH₂)₁₆—CO₂H)AQ-Me4Pal-EFIe-αMeY- | | |
| | LIEGGPSSGAPPPEGGGE-NH₂ | | |
| 762 | Y-Aib-QGT-αMeF(2F)-TSD-4Pal-αMeS-I-αMeL-LD-Orn- | 768 | 5253.8 |
| | K((γ-Glu)₂-CO—(CH₂)₁₆—CO₂H)AQ-Me4Pal-EFIe-αMeY- | | |
| | LIEGGPSSGEPPPEGGGE-NH₂ | | |
| 763 | Y-Aib-QGT-αMeF(2F)-TSD-4Pal-αMeS-I-αMeL-LD-Orn- | 769 | 5313.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGEPPPEGTGE-NH₂ | | |
| 764 | Y-Aib-QGT-αMeF(2F)-TSD-4Pal-αMeS-I-αMeL-LD-Orn- | 770 | 5239.8 |
| | K((γ-Glu)₂-CO—(CH₂)₁₆—CO₂H)AQ-Me4Pal-EFIe-αMeY- | | |
| | LIEGGPSSGAPPPEGTGE-NH₂ | | |
| 765 | Y-Aib-QaT-αMeF(2F)-TSD-4Pal-SI-αMeL-LD-Orn-K((2- | 771 | 5170.8 |
| | [2-(2-Amino-ethoxy)-ethoxy]-acetyl)-(γ-Glu)-CO—(CH₂)₁₆- | | |
| | CO₂H)AQ-Me4Pal-Orn-FIe-αMeY-LIEGGPSSGEPPPESE- | | |
| | NH₂ | | |
| 766 | Y-Aib-QaT-αMeF(2F)-TSD-4Pal-αMeS-I-αMeL-LD-Orn- | 772 | 5285.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGEPPPSGTGE-NH₂ | | |
| 767 | Y-Aib-QGT-αMeF(2F)-TSD-4Pal-αMeS-I-αMeL-LD-Orn- | 773 | 5091.7 |
| | K((2-[2-(2-Amino-ethoxy)-ethoxy]-acetyl)-(γ-Glu)-CO— | | |
| | (CH₂)₁₆—CO₂H)YQ-Me4Pal-EFIe-αMeY- | | |
| | LVEGGPSSGAPPPSGS-NH₂ | | |
| 768 | Y-Aib-QGT-αMeF(2F)-TSD-4Pal-αMeS-I-αMeL-LE-Orn- | 774 | 5013.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGAPPPSGS-NH₂ | | |
| 769 | Y-Aib-QGT-αMeF(2F)-TSD-4Pal-αMeS-I-αMeL-LD-Orn- | 775 | 5155.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGAPPPSGGGE-NH₂ | | |
| 770 | Y-Aib-QGTFTSDFS-Orn-αMeL-LDKK((γ-Glu)₂-CO— | 776 | 4814.4 |
| | (CH₂)₁₆—CO₂H)AQ-Aib-AFIEYLLEGGPSS-Aib- | | |
| | APPPGGS-NH₂ | | |
| 771 | Y-Aib-QGTFTSDFS-Orn-αMeL-LDKK((γ-Glu)₂-CO— | 777 | 4800.37 |
| | (CH₂)₁₆—CO₂H)AQ-Aib-AFIEYLLEGGPSSG-Aib- | | |
| | PPPGGS-NH₂ | | |
| 772 | Y-Aib-QGTFTSDFSQ-αMeL-LDKK((2-[2-(2-Amino- | 778 | 4830.4 |
| | ethoxy)-ethoxy]-acetyl)-(γ-Glu)-CO—(CH₂)₁₆—CO₂H)AQ- | | |
| | Aib-AFIEYLLAGGPSSGAPPPEGS-NH₂ | | |
| 773 | Y-Aib-QGTFTSDFSQ-αMeL-LDKK((2-[2-(2-Amino- | 779 | 4846.4 |
| | ethoxy)-ethoxy]-acetyl)-(γ-Glu)-CO—(CH₂)₁₆—CO₂H)AQ- | | |
| | Aib-AFIEYLLAGGPSSGEPPPSGS-NH₂ | | |
| 774 | Y-Aib-QGTFTSDFSQ-αMeL-LDKK((2-[2-(2-Amino- | 780 | 4911.5 |
| | ethoxy)-ethoxy]-acetyl)-(γ-Glu)-CO—(CH₂)₁₆—CO₂H)AQ- | | |
| | Aib-AFIEWLLEGGPSSGAPPPEGS-NH₂ | | |
| 775 | Y-Aib-QGTFTSDFSQ-αMeL-LDKK((2-[2-(2-Amino- | 781 | 4860.4 |
| | ethoxy)-ethoxy]-acetyl)-(γ-Glu)-CO—(CH₂)₁₆—CO₂H)AQ- | | |
| | Aib-AFIEYLLEaGPSSGAPPPSGS-NH₂ | | |
| 776 | Y-Aib-QGTFTSDFSQ-αMeL-LDKK((2-[2-(2-Amino- | 782 | 4874.5 |
| | ethoxy)-ethoxy]-acetyl)-(γ-Glu)-CO—(CH₂)₁₆—CO₂H)AQ- | | |
| | Aib-AFIEYLLEaGPSSGAPPPSGT-NH₂ | | |
| 777 | Y-Aib-QGTFTSDFSQ-αMeL-LDKK((2-[2-(2-Amino- | 783 | 4921.5 |
| | ethoxy)-ethoxy]-acetyl)-(γ-Glu)-CO—(CH₂)₁₆—CO₂H)AQ- | | |
| | Aib-AFIEYLLEaGPSSGAPPPSG-4Pal-NH₂ | | |
| 778 | Y-Aib-QGTFTSDFSQ-αMeL-LDKK((2-[2-(2-Amino- | 784 | 4875.4 |
| | ethoxy)-ethoxy]-acetyl)-(γ-Glu)-CO—(CH₂)₁₆—CO₂H)AQ- | | |
| | Aib-AFIEYLLEaGPSSGAPPPSGT | | |
| 779 | Y-Aib-QGTFTSDFSQ-αMeL-LDKK((2-[2-(2-Amino- | 785 | 4902.5 |
| | ethoxy)-ethoxy]-acetyl)-(γ-Glu)-CO—(CH₂)₁₆—CO₂H)AQ- | | |
| | Aib-AFIEYLLEaGPSS-Aib-APPPSGT-NH₂ | | |
| 780 | Y-Aib-QGTFTSDFSQ-αMeL-LDKK((2-[2-(2-Amino- | 786 | 4903.5 |
| | ethoxy)-ethoxy]-acetyl)-(γ-Glu)-CO—(CH₂)₁₆—CO₂H)AQ- | | |
| | Aib-AFIEYLLEaGPSS-Aib-APPPSGT | | |
| 781 | Y-Aib-QGTFTSDFSQ-αMeL-LDKK((2-[2-(2-Amino- | 787 | 4888.5 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSGT-NH₂ | | |
| 782 | Y-Aib-QGTFTSDFSQ-αMeL-LDKK((2-[2-(2-Amino- | 788 | 4935.5 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSG-4Pal-NH₂ | | |
| 783 | Y-Aib-QGTFTSDFSQ-αMeL-LDKK((2-[2-(2-Amino- | 789 | 4889.5 |
| | ethoxy)-ethoxy]-acetyl)-(γ-Glu)-CO—(CH₂)₁₆—CO₂H)AQ- | | |
| | Aib-AFIEYLLE-Aib-GPSSGAPPPSGT | | |
| 784 | Y-Aib-QGTFTSDFSQ-αMeL-LDKK((γ-Glu)₂-CO—(CH₂)₁₆- | 790 | 4858.4 |
| | CO₂H)AQ-Aib-AFIEYLLE-Aib-GPSSGAPPPSGS-NH₂ | | |
| 785 | Y-Aib-QGTFTSDFSQ-αMeL-LDKK((2-[2-(2-Amino- | 791 | 4920.5 |
| | ethoxy)-ethoxy]-acetyl)-(γ-Glu)-CO—(CH₂)₁₆—CO₂H)AQ- | | |
| | Aib-AFIEYLLE-Aib-GESSGAPPPSGT-NH₂ | | |
| 786 | Y-Aib-QGTFTSDFSQ-αMeL-LDKK((2-[2-(2-Amino- | 792 | 4921.5 |
| | ethoxy)-ethoxy]-acetyl)-(γ-Glu)-CO—(CH₂)₁₆—CO₂H)AQ- | | |
| | Aib-AFIEYLLE-Aib-GESSGAPPPSGT | | |
| 787 | Y-Aib-QGTFTSDFSQ-αMeL-LDKK((2-[2-(2-Amino- | 793 | 5051.6 |
| | ethoxy)-ethoxy]-acetyl)₂-(γ-Glu)-CO—(CH₂)₁₆—CO₂H)AQ- | | |
| | Aib-AFIEYLLE-Aib-GESSGAPPPSGS-NH₂ | | |
| 788 | Y-Aib-QGTFTSDFSQ-αMeL-LDKK((2-[2-(2-Amino- | 794 | 4964.5 |
| | ethoxy)-ethoxy]-acetyl)-(γ-Glu)-CO—(CH₂)₁₆—CO₂H)AQ- | | |
| | Aib-AFIEYLLE-Aib-GESSGEPPPSGS-NH₂ | | |
| 789 | Y-Aib-QGTFTSDFSQ-αMeL-LDKK((2-[2-(2-Amino- | 795 | 4948.5 |
| | ethoxy)-ethoxy]-acetyl)-(γ-Glu)-CO—(CH₂)₁₆—CO₂H)AQ- | | |
| | Aib-AFIEYLLE-Aib-GESSGAPPPSGE-NH₂ | | |
| 790 | Y-Aib-QGTFTSDFSQ-αMeL-LDKK((2-[2-(2-Amino- | 796 | 5006.5 |
| | ethoxy)-ethoxy]-acetyl)-(γ-Glu)-CO—(CH₂)₁₆—CO₂H)AQ- | | |
| | Aib-AFIEYLLE-Aib-GESSGEPPPSGE-NH₂ | | |
| 791 | Y-Aib-QGTFTSD-4Pal-S-Orn-αMeL-LDKK((2-[2-(2- | 797 | 4942.5 |
| | Amino-ethoxy)-ethoxy]-acetyl)-(γ-Glu)-CO—(CH₂)₁₆- | | |
| | CO₂H)AQ-Me4Pal-AFIEYLIEGGESSGAPPPSGS-NH₂ | | |
| 792 | Y-Aib-QGTFTSD-4Pal-SR-αMeL-LDKK((2-[2-(2-Amino- | 798 | 4984.5 |
| | ethoxy)-ethoxy]-acetyl)-(γ-Glu)-CO—(CH₂)₁₆—CO₂H)AQ- | | |
| | Me4Pal-AFIEYLIEGGESSGAPPPSGS-NH₂ | | |
| 793 | Y-Aib-QGTFTSD-4Pal-SQ-αMeL-LDKK((2-[2-(2-Amino- | 799 | 4956.5 |
| | ethoxy)-ethoxy]-acetyl)-(γ-Glu)-CO—(CH₂)₁₆—CO₂H)AQ- | | |
| | Me4Pal-AFIEYLIEGGESSGAPPPSGS-NH₂ | | |
| 794 | Y-Aib-QGTFTSDFSR-αMeL-LDKK((2-[2-(2-Amino- | 800 | 4983.5 |
| | ethoxy)-ethoxy]-acetyl)-(γ-Glu)-CO—(CH₂)₁₆—CO₂H)AQ- | | |
| | Me4Pal-AFIEYLIEGGESSGAPPPSGS-NH₂ | | |
| 795 | Y-Aib-QGTFTSDFSQ-αMeL-LDKK((2-[2-(2-Amino- | 801 | 4955.5 |
| | ethoxy)-ethoxy]-acetyl)-(γ-Glu)-CO—(CH₂)₁₆—CO₂H)AQ- | | |
| | Me4Pal-AFIEYLIEGGESSGAPPPSGS-NH₂ | | |
| 796 | Y-Aib-QGTFTSDFS-Orn-αMeL-LDKK((2-[2-(2-Amino- | 802 | 4860.5 |
| | ethoxy)-ethoxy]-acetyl)-(γ-Glu)-CO—(CH₂)₁₆—CO₂H)AQ- | | |
| | Aib-AFIEYLIE-Aib-GPSSGAPPPSGS-NH₂ | | |
| 797 | Y-Aib-QGTFTSDFSK-αMeL-LDKK((2-[2-(2-Amino- | 803 | 4874.5 |
| | ethoxy)-ethoxy]-acetyl)-(γ-Glu)-CO—(CH₂)₁₆—CO₂H)AQ- | | |
| | Aib-AFIEYLIE-Aib-GPSSGAPPPSGS-NH₂ | | |
| 798 | Y-Aib-QGTFTSDFSR-αMeL-LDKK((2-[2-(2-Amino- | 804 | 4902.5 |
| | ethoxy)-ethoxy]-acetyl)-(γ-Glu)-CO—(CH₂)₁₆—CO₂H)AQ- | | |
| | Aib-AFIEYLIE-Aib-GPSSGAPPPSGS-NH₂ | | |
| 799 | Y-Aib-QGTFTSDFSDab-αMeL-LDKK((2-[2-(2-Amino- | 805 | 4846.4 |
| | ethoxy)-ethoxy]-acetyl)-(γ-Glu)-CO—(CH₂)₁₆—CO₂H)AQ- | | |
| | Aib-AFIEYLIE-Aib-GPSSGAPPPSGS-NH₂ | | |
| 800 | Y-Aib-QGTFTSDFSDap-αMeL-LDKK((2-[2-(2-Amino- | 806 | 4832.4 |
| | ethoxy)-ethoxy]-acetyl)-(γ-Glu)-CO—(CH₂)₁₆—CO₂H)AQ- | | |
| | Aib-AFIEYLIE-Aib-GPSSGAPPPSGS-NH₂ | | |
| 801 | Y-Aib-QGTFTSD-4Pal-SK-αMeL-LDKK((2-[2-(2-Amino- | 807 | 4875.5 |
| | ethoxy)-ethoxy]-acetyl)-(γ-Glu)-CO—(CH₂)₁₆—CO₂H)AQ- | | |
| | Aib-AFIEYLIE-Aib-GPSSGAPPPSGS-NH₂ | | |
| 802 | Y-Aib-QGTFTSD-4Pal-SR-αMeL-LDKK((2-[2-(2-Amino- | 808 | 4903.5 |
| | ethoxy)-ethoxy]-acetyl)-(γ-Glu)-CO—(CH₂)₁₆—CO₂H)AQ- | | |
| | Aib-AFIEYLIE-Aib-GPSSGAPPPSGS-NH₂ | | |
| 803 | Y-Aib-QGTFTSD-4Pal-SDab-αMeL-LDKK((2-[2-(2- | 809 | 4847.4 |
| | Amino-ethoxy)-ethoxy]-acetyl)-(γ-Glu)-CO—(CH₂)₁₆- | | |
| | CO₂H)AQ-Aib-AFIEYLIE-Aib-GPSSGAPPPSGS-NH₂ | | |
| 804 | Y-Aib-QGTFTSDFS-Orn-αMeL-LDKK((2-[2-(2-Amino- | 810 | 4890.5 |
| | ethoxy)-ethoxy]-acetyl)-(γ-Glu)-CO—(CH₂)₁₆—CO₂H)AQ- | | |
| | Aib-AFIEYLIE-Aib-SPSSGAPPPSGS-NH₂ | | |
| 805 | Y-Aib-QGTFTSDFSK-αMeL-LDKK((2-[2-(2-Amino- | 811 | 4904.5 |
| | ethoxy)-ethoxy]-acetyl)-(γ-Glu)-CO—(CH₂)₁₆—CO₂H)AQ- | | |
| | Aib-AFIEYLIE-Aib-SPSSGAPPPSGS-NH₂ | | |
| 806 | Y-Aib-QGTFTSDFSR-αMeL-LDKK((2-[2-(2-Amino- | 812 | 4932.5 |
| | ethoxy)-ethoxy]-acetyl)-(γ-Glu)-CO—(CH₂)₁₆—CO₂H)AQ- | | |
| | Aib-AFIEYLIE-Aib-SPSSGAPPPSGS-NH₂ | | |
| 807 | Y-Aib-QGTFTSD-4Pal-SR-αMeL-LDKK((2-[2-(2-Amino- | 813 | 4933.5 |
| | ethoxy)-ethoxy]-acetyl)-(γ-Glu)-CO—(CH₂)₁₆—CO₂H)AQ- | | |
| | Aib-AFIEYLIE-Aib-SPSSGAPPPSGS-NH₂ | | |
| 808 | Y-Aib-QGTFTSD-4Pal-S-Orn-αMeL-LDKK((2-[2-(2- | 814 | 4938.5 |
| | Amino-ethoxy)-ethoxy]-acetyl)-(γ-Glu)-CO—(CH₂)₁₆- | | |
| | CO₂H)AQ-Me4Pal-AFIEYLIE-Aib-GPSSGAPPPSGS-NH₂ | | |
| 809 | Y-Aib-QGTFTSD-4Pal-SR-αMeL-LDKK((2-[2-(2-Amino- | 815 | 4980.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGS-NH₂ | | |
| 810 | Y-Aib-QGTFTSD-4Pal-SQ-αMeL-LDKK((2-[2-(2-Amino- | 816 | 4952.5 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGS-NH₂ | | |
| 811 | Y-Aib-QGTFTSDFS-Orn-αMeL-LDKK((2-[2-(2-Amino- | 817 | 4937.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGS-NH₂ | | |
| 812 | Y-Aib-QGTFTSDFSR-αMeL-LDKK((2-[2-(2-Amino- | 818 | 4979.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGS-NH₂ | | |
| 813 | Y-Aib-QGTFTSDFSQ-αMeL-LDKK((2-[2-(2-Amino- | 819 | 4951.5 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGS-NH₂ | | |
| 814 | Y-Aib-QGTFTSD-4Pal-S-Orn-αMeL-LDKK((2-[2-(2- | 820 | 5083.7 |
| | Amino-ethoxy)-ethoxy]-acetyl)₂-(γ-Glu)-CO—(CH₂)₁₆- | | |
| | CO₂H)AQ-Me4Pal-AFIEYLIE-Aib-GPSSGAPPPSGS-NH₂ | | |
| 815 | Y-Aib-QGTFTSD-4Pal-SR-αMeL-LDKK((2-[2-(2-Amino- | 821 | 5125.7 |
| | ethoxy)-ethoxy]-acetyl)₂-(γ-Glu)-CO—(CH₂)₁₆—CO₂H)AQ- | | |
| | Me4Pal-AFIEYLIE-Aib-GPSSGAPPPSGS-NH₂ | | |
| 816 | Y-Aib-QGTFTSD-4Pal-SQ-αMeL-LDKK((2-[2-(2-Amino- | 822 | 5097.7 |
| | ethoxy)-ethoxy]-acetyl)₂-(γ-Glu)-CO—(CH₂)₁₆—CO₂H)AQ- | | |
| | Me4Pal-AFIEYLIE-Aib-GPSSGAPPPSGS-NH₂ | | |
| 817 | Y-Aib-QGTFTSDFSQ-αMeL-LDKK((2-[2-(2-Amino- | 823 | 5096.7 |
| | ethoxy)-ethoxy]-acetyl)₂-(γ-Glu)-CO—(CH₂)₁₆—CO₂H)AQ- | | |
| | Me4Pal-AFIEYLIE-Aib-GPSSGAPPPSGS-NH₂ | | |
| 818 | Y-Aib-QGTFTSDFSQ-αMeL-LD-Orn-K((2-[2-(2-Amino- | 824 | 4937.5 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGS-NH₂ | | |
| 819 | Y-Aib-QGTFTSDFSQ-αMeL-LDKK((2-[2-(2-Amino- | 825 | 4935.5 |
| | ethoxy)-ethoxy]-acetyl)-(γ-Glu)₂-CO—(CH₂)₁₆—CO₂H)AQ- | | |
| | Me4Pal-AFIEYLIE-Aib-GPSSGAPPPSGS-NH₂ | | |
| 820 | Y-Aib-QGTFTSD-4Pal-SQ-αMeL-LDKK((2-[2-(2-Amino- | 826 | 4861.4 |
| | ethoxy)-ethoxy]-acetyl)-(γ-Glu)-CO—(CH₂)₁₆—CO₂H)AQ- | | |
| | Aib-AFIEYLLEaGPSSGAPPPSGS-NH₂ | | |
| 821 | Y-Aib-QGTFTSD-4Pal-SQ-αMeL-LDK-((2-[2-(2-Amino- | 827 | 4875.4 |
| | ethoxy)-ethoxy]-acetyl)-(γ-Glu)-CO—(CH₂)₁₆—CO₂H)AQ- | | |
| | Aib-AFIEYLLEaGPSSGAPPPSGT-NH₂ | | |
| 822 | Y-Aib-QGTFTSD-4Pal-SQ-αMeL-LDKK((2-[2-(2-Amino- | 828 | 4922.5 |
| | ethoxy)-ethoxy]-acetyl)-(γ-Glu)-CO—(CH₂)₁₆—CO₂H)AQ- | | |
| | Aib-AFIEYLLEaGPSSGAPPPSG-4Pal-NH₂ | | |
| 823 | Y-Aib-QGTFTSD-4Pal-SQ-αMeL-LDKK((2-[2-(2-Amino- | 829 | 4876.4 |
| | ethoxy)-ethoxy]-acetyl)-(γ-Glu)-CO—(CH₂)₁₆—CO₂H)AQ- | | |
| | Aib-AFIEYLLEaGPSSGAPPPSGT | | |
| 824 | Y-Aib-QGTFTSD-4Pal-SQ-αMeL-LDKK((2-[2-(2-Amino- | 830 | 4903.5 |
| | ethoxy)-ethoxy]-acetyl)-(γ-Glu)-CO—(CH₂)₁₆—CO₂H)AQ- | | |
| | Aib-AFIEYLLEaGPSS-Aib-APPPSGT | | |
| 825 | Y-Aib-QGTFTSD-4Pal-SQ-αMeL-LDKK((2-[2-(2-Amino- | 831 | 4904.5 |
| | ethoxy)-ethoxy]-acetyl)-(γ-Glu)-CO—(CH₂)₁₆—CO₂H)AQ- | | |
| | Aib-AFIEYLLEaGPSS-Aib-APPPSGT | | |
| 826 | Y-Aib-QGTFTSD-4Pal-SQ-αMeL-LDKK((2-[2-(2-Amino- | 832 | 4924.5 |
| | ethoxy)-ethoxy]-acetyl)-(γ-Glu)-CO—(CH₂)₁₆—CO₂H)AQ- | | |
| | Me4Pal-AFIEYLIEGGPSSGAPPPSGS-NH₂ | | |
| 827 | Y-Aib-QGTFTSD-4Pal-SQ-αMeL-LDKK((2-[2-(2-Amino- | 833 | 4866.4 |
| | ethoxy)-ethoxy]-acetyl)-(γ-Glu)-CO—(CH₂)₁₆—CO₂H)AQ- | | |
| | Me4Pal-AFIEYLIAGGPSSGAPPPSGS-NH₂ | | |
| 828 | Y-Aib-QGTFTSD-4Pal-SQ-αMeL-LDKK((2-[2-(2-Amino- | 834 | 4924.5 |
| | ethoxy)-ethoxy]-acetyl)-(γ-Glu)-CO—(CH₂)₁₆—CO₂H)AQ- | | |
| | Me4Pal-AFIEYLIAGGPSSGEPPPSGS-NH₂ | | |
| 829 | Y-Aib-QGTFTSD-4Pal-SQ-αMeL-LDKK((2-[2-(2-Amino- | 835 | 4938.5 |
| | ethoxy)-ethoxy]-acetyl)-(γ-Glu)-CO—(CH₂)₁₆—CO₂H)AQ- | | |
| | Me4Pal-AFIEYLIEaGPSSGAPPPSGS-NH₂ | | |
| 830 | Y-Aib-QGTFTSD-4Pal-SQ-αMeL-LDKK((2-[2-(2-Amino- | 836 | 4947.5 |
| | ethoxy)-ethoxy]-acetyl)-(γ-Glu)-CO—(CH₂)₁₆—CO₂H)AQ- | | |
| | Me4Pal-AFIEWLIEGGPSSGAPPPSGS-NH₂ | | |
| 831 | Y-Aib-QGTFTSD-4Pal-SQ-αMeL-LDKK((2-[2-(2-Amino- | 837 | 4961.5 |
| | ethoxy)-ethoxy]-acetyl)-(γ-Glu)-CO—(CH₂)₁₆—CO₂H)AQ- | | |
| | Me4Pal-AFIEWLIEGGPSSGAPPPSGT-NH₂ | | |
| 832 | Y-Aib-QGTFTSD-4Pal-SQ-αMeL-LDKK((2-[2-(2-Amino- | 838 | 5008.6 |
| | ethoxy)-ethoxy]-acetyl)-(γ-Glu)-CO—(CH₂)₁₆—CO₂H)AQ- | | |
| | Me4Pal-AFIEWLIEGGPSSGAPPPSG-4Pal-NH₂ | | |
| 833 | Y-Aib-QGTFTSD-4Pal-SQ-αMeL-LD-Orn-K((2-[2-(2- | 839 | 4938.5 |
| | Amino-ethoxy)-ethoxy]-acetyl)-(γ-Glu)₂-CO—(CH₂)₁₆- | | |
| | CO₂H)AQ-Me4Pal-AFIEYLIE-Aib-GPSSGAPPPSGS-NH₂ | | |
| 834 | Y-Aib-QGTFTSD-4Pal-SQ-αMeL-LDKK((2-[2-(2-Amino- | 840 | 4966.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGT-NH₂ | | |
| 835 | Y-Aib-QGTFTSD-4Pal-SQ-αMeL-LDKK((2-[2-(2-Amino- | 841 | 5013.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSG-4Pal-NH₂ | | |
| 836 | Y-Aib-QGTFTSD-4Pal-S-Orn-αMeL-LDKK((2-[2-(2- | 842 | 4847.4 |
| | Amino-ethoxy)-ethoxy]-acetyl)-(γ-Glu)-CO—(CH₂)₁₆- | | |
| | CO₂H)AQ-Aib-AFIE-αMeY-LIEGGPSSGAPPPSGS-NH₂ | | |
| 837 | Y-Aib-QGTFTSD-4Pal-SR-αMeL-LDKK((2-[2-(2-Amino- | 843 | 4889.5 |
| | ethoxy)-ethoxy]-acetyl)-(γ-Glu)-CO—(CH₂)₁₆—CO₂H)AQ- | | |
| | Aib-AFIE-αMeY-LIEGGPSSGAPPPSGS-NH₂ | | |
| 838 | Y-Aib-QGTFTSD-4Pal-SQ-αMeL-LDKK((2-[2-(2-Amino- | 844 | 4861.4 |
| | ethoxy)-ethoxy]-acetyl)-(γ-Glu)-CO—(CH₂)₁₆—CO₂H)AQ- | | |
| | Aib-AFIE-αMeY-LIEGGPSSGAPPPSGS-NH₂ | | |
| 839 | Y-Aib-QGTFTSDFS-Orn-αMeL-LDKK((2-[2-(2-Amino- | 845 | 4846.4 |
| | ethoxy)-ethoxy]-acetyl)-(γ-Glu)-CO—(CH₂)₁₆—CO₂H)AQ- | | |
| | Aib-AFIE-αMeY-LIEGGPSSGAPPPSGS-NH₂ | | |
| 840 | Y-Aib-QGTFTSDFSR-αMeL-LDKK((2-[2-(2-Amino- | 846 | 4888.5 |
| | ethoxy)-ethoxy]-acetyl)-(γ-Glu)-CO—(CH₂)₁₆—CO₂H)AQ- | | |
| | Aib-AFIE-αMeY-LIEGGPSSGAPPPSGS-NH₂ | | |
| 841 | Y-Aib-QGTFTSDFSQ-αMeL-LDKK((2-[2-(2-Amino- | 847 | 4860.4 |
| | ethoxy)-ethoxy]-acetyl)-(γ-Glu)-CO—(CH₂)₁₆—CO₂H)AQ- | | |
| | Aib-AFIE-αMeY-LIEGGPSSGAPPPSGS-NH₂ | | |
| 842 | Y-Aib-QGTFTSD-4Pal-S-Orn-αMeL-LDKK((2-[2-(2- | 848 | 4924.5 |
| | Amino-ethoxy)-ethoxy]-acetyl)-(γ-Glu)-CO—(CH₂)₁₆- | | |
| | CO₂H)AQ-Me4Pal-AFIE-αMeY-LIEGGPSSGAPPPSGS- | | |
| | NH₂ | | |
| 843 | Y-Aib-QGTFTSD-4Pal-SR-αMeL-LDKK((2-[2-(2-Amino- | 849 | 4966.6 |
| | ethoxy)-ethoxy]-acetyl)-(γ-Glu)-CO—(CH₂)₁₆—CO₂H)AQ- | | |
| | Me4Pal-AFIE-αMeY-LIEGGPSSGAPPPSGS-NH₂ | | |
| 844 | Y-Aib-QGTFTSD-4Pal-SQ-αMeL-LDKK((2-[2-(2-Amino- | 850 | 4938.5 |
| | ethoxy)-ethoxy]-acetyl)-(γ-Glu)-CO—(CH₂)₁₆—CO₂H)AQ- | | |
| | Me4Pal-AFIE-αMeY-LIEGGPSSGAPPPSGS-NH₂ | | |
| 845 | Y-Aib-QGTFTSDFS-Orn-αMeL-LDKK((2-[2-(2-Amino- | 851 | 4923.5 |
| | ethoxy)-ethoxy]-acetyl)-(γ-Glu)-CO—(CH₂)₁₆—CO₂H)AQ- | | |
| | Me4Pal-AFIE-αMeY-LIEGGPSSGAPPPSGS-NH₂ | | |
| 846 | Y-Aib-QGTFTSDFSR-αMeL-LDKK((2-[2-(2-Amino- | 852 | 4965.6 |
| | ethoxy)-ethoxy]-acetyl)-(γ-Glu)-CO—(CH₂)₁₆—CO₂H)AQ- | | |
| | Me4Pal-AFIE-αMeY-LIEGGPSSGAPPPSGS-NH₂ | | |
| 847 | Y-Aib-QGTFTSDFSQ-αMeL-LDKK((2-[2-(2-Amino- | 853 | 4937.5 |
| | ethoxy)-ethoxy]-acetyl)-(γ-Glu)-CO—(CH₂)₁₆—CO₂H)AQ- | | |
| | Me4Pal-AFIE-αMeY-LIEGGPSSGAPPPSGS-NH₂ | | |
| 848 | Y-Aib-QGTFTSD-4Pal-SQ-αMeL-LDKK((2-[2-(2-Amino- | 854 | 4982.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSSS-NH₂ | | |
| 849 | Y-Aib-QGTFTSD-4Pal-SQ-αMeL-LD-Orn-K((2-[2-(2- | 855 | 4924.5 |
| | Amino-ethoxy)-ethoxy]-acetyl)-(γ-Glu)-CO—(CH₂)₁₆- | | |
| | CO₂H)AQ-Me4Pal-AFIE-αMeY-LIEGGPSSGAPPPSGS- | | |
| | NH₂ | | |
| 850 | Y-Aib-QGTFTSD-4Pal-SQ-αMeL-LD-Orn-K((2-[2-(2- | 856 | 4847.4 |
| | Amino-ethoxy)-ethoxy]-acetyl)-(γ-Glu)-CO—(CH₂)₁₆- | | |
| | CO₂H)AQ-Aib-AFIE-αMeY-LIEGGPSSGAPPPSGS-NH₂ | | |
| 851 | Y-Aib-QGTFTSD-4Pal-SQ-αMeL-LDKK((2-[2-(2-Amino- | 857 | 4994.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPEGS-NH₂ | | |
| 852 | Y-Aib-QGTFTSD-4Pal-SQ-αMeL-LDKK((2-[2-(2-Amino- | 858 | 4980.5 |
| | ethoxy)-ethoxy]-acetyl)-(γ-Glu)-CO—(CH₂)₁₆—CO₂H)AQ- | | |
| | Me4Pal-AFIE-αMeY-LIEGGPSSGAPPPEGS-NH₂ | | |
| 853 | Y-Aib-QGTFTSD-4Pal-SQ-αMeL-LDKK((2-[2-(2-Amino- | 859 | 4903.5 |
| | ethoxy)-ethoxy]-acetyl)-(γ-Glu)-CO—(CH₂)₁₆—CO₂H)AQ- | | |
| | Aib-AFIE-αMeY-LIEGGPSSGAPPPEGS-NH₂ | | |
| 854 | Y-Aib-QGTFTSD-4Pal-SQ-αMeL-LDKK((2-[2-(2-Amino- | 860 | 5009.6 |
| | ethoxy)-ethoxy]-acetyl)-(γ-Glu)-CO—(CH₂)₁₆—CO₂H)AQ- | | |
| | Me4Pal-QFIEYLIE-Aib-GPSSGAPPPSGS-NH₂ | | |
| 855 | Y-Aib-QGTFTSD-4Pal-SQ-αMeL-LDKK((2-[2-(2-Amino- | 861 | 4995.6 |
| | ethoxy)-ethoxy]-acetyl)-(γ-Glu)-CO—(CH₂)₁₆—CO₂H)AQ- | | |
| | Me4Pal-QFIE-αMeY-LIEGGPSSGAPPPSGS-NH₂ | | |
| 856 | Y-Aib-QGTFTSD-4Pal-SQ-αMeL-LD-Orn-K((2-[2-(2- | 862 | 4995.6 |
| | Amino-ethoxy)-ethoxy]-acetyl)-(γ-Glu)-CO—(CH₂)₁₆- | | |
| | CO₂H)AQ-Me4Pal-QFIEYLIE-Aib-GPSSGAPPPSGS-NH₂ | | |
| 857 | Y-Aib-QGTFTSD-4Pal-SQ-αMeL-LD-Orn-K((2-[2-(2- | 863 | 4981.5 |
| | Amino-ethoxy)-ethoxy]-acetyl)-(γ-Glu)-CO—(CH₂)₁₆- | | |
| | CO₂H)AQ-Me4Pal-QFIE-αMeY-LIEGGPSSGAPPPSGS- | | |
| | NH₂ | | |
| 858 | Y-Aib-QGTFTSD-4Pal-SQ-αMeL-LDKK((γ-Glu)₂-CO— | 864 | 4936.5 |
| | (CH₂)₁₆—CO₂H)AQ-Me4Pal-AFIEYLIE-Aib- | | |
| | GPSSGAPPPSGS-NH₂ | | |
| 859 | Y-Aib-QGTFTSD-4Pal-SQ-αMeL-LDKK((γ-Glu)₃-CO— | 865 | 5065.6 |
| | (CH₂)₁₆—CO₂H)AQ-Me4Pal-AFIEYLIE-Aib- | | |
| | GPSSGAPPPSGS-NH₂ | | |
| 860 | Y-Aib-QGTFTSD-4Pal-SQ-αMeL-LDKK((2-[2-(2-Amino- | 866 | 5081.6 |
| | ethoxy)-ethoxy]-acetyl)-(γ-Glu)₂-CO—(CH₂)₁₆—CO₂H)AQ- | | |
| | Me4Pal-AFIEYLIE-Aib-GPSSGAPPPSGS-NH₂ | | |
| 861 | Y-Aib-QGTFTSD-4Pal-SQ-αMeL-LDKK((γ-Glu)-(2-[2-(2- | 867 | 5081.6 |
| | Amino-ethoxy)-ethoxy]-acetyl)-(γ-Glu)-CO—(CH₂)₁₆- | | |
| | CO₂H)AQ-Me4Pal-AFIEYLIE-Aib-GPSSGAPPPSGS-NH₂ | | |
| 862 | Y-Aib-QGTFTSDFSQ-αMeL-LDKK((γ-Glu)₃-CO—(CH₂)₁₆- | 868 | 5064.6 |
| | CO₂H)AQ-Me4Pal-AFIEYLIE-Aib-GPSSGAPPPSGS-NH₂ | | |
| 863 | Y-Aib-QGTFTSDFSQ-αMeL-LDKK((2-[2-(2-Amino- | 869 | 5080.7 |
| | ethoxy)-ethoxy]-acetyl)-(γ-Glu)₂-CO—(CH₂)₁₆—CO₂H)AQ- | | |
| | Me4Pal-AFIEYLIE-Aib-GPSSGAPPPSGS-NH₂ | | |
| 864 | Y-Aib-QGTFTSDFSQ-αMeL-LDKK((γ-Glu)-(2-[2-(2- | 870 | 5080.7 |
| | Amino-ethoxy)-ethoxy]-acetyl)-(γ-Glu)-CO—(CH₂)₁₆- | | |
| | CO₂H)AQ-Me4Pal-AFIEYLIE-Aib-GPSSGAPPPSGS-NH₂ | | |
| 865 | Y-Aib-QGTFTSD-4Pal-S-Orn-αMeL-LDKK((γ-Glu)₂-CO— | 871 | 4922.5 |
| | (CH₂)₁₆—CO₂H)AQ-Me4Pal-AFIEYLIE-Aib- | | |
| | GPSSGAPPPSGS-NH₂ | | |
| 866 | Y-Aib-QGTFTSD-4Pal-S-Orn-αMeL-LDKK((γ-Glu)₃-CO— | 872 | 5051.6 |
| | (CH₂)₁₆—CO₂H)AQ-Me4Pal-AFIEYLIE-Aib- | | |
| | GPSSGAPPPSGS-NH₂ | | |
| 867 | Y-Aib-QGTFTSD-4Pal-S-Orn -- αMeL-LDKK((2-[2-(2- | 873 | 5067.7 |
| | Amino-ethoxy)-ethoxy]-acetyl)-(γ-Glu)₂-CO—(CH₂)₁₆- | | |
| | CO₂H)AQ-Me4Pal-AFIEYLIE-Aib-GPSSGAPPPSGS-NH₂ | | |
| 868 | Y-Aib-QGTFTSD-4Pal-S-Orn -- αMeL-LDKK((γ-Glu)-(2-[2- | 874 | 5067.7 |
| | (2-Amino-ethoxy)-ethoxy]-acetyl)-(γ-Glu)-CO—(CH₂)₁₆- | | |
| | CO₂H)AQ-Me4Pal-AFIEYLIE-Aib-GPSSGAPPPSGS-NH₂ | | |
| 869 | Y-Aib-QGTFTSD-4Pal-SQ-αMeL-LDKK((2-[2-(2-Amino- | 875 | 4995.6 |
| | ethoxy)-ethoxy]-acetyl)-(γ-Glu)₂-CO—(CH₂)₁₆—CO₂H)AQ- | | |
| | Me4Pal-AFIEYLIAGGPSSGAPPPSGS-NH₂ | | |
| 870 | Y-Aib-QGTFTSD-4Pal-SQ-αMeL-LDKK((γ-Glu)₂-CO— | 876 | 4850.4 |
| | (CH₂)₁₆—CO₂H)AQ-Me4Pal-AFIEYLIAGGPSSGAPPPSGS- | | |
| | NH₂ | | |
| 871 | Y-Aib-QGTFTSD-4Pal-SQ-αMeL-LDKK((2-[2-(2-Amino- | 877 | 5076.6 |
| | ethoxy)-ethoxy]-acetyl)-(γ-Glu)₂-CO—(CH₂)₁₆—CO₂H)AQ- | | |
| | Me4Pal-AFIEWLIEGGPSSGAPPPSGS-NH₂ | | |
| 872 | Y-Aib-QGTFTSD-4Pal-SQ-αMeL-LDKK((γ-Glu)₂-CO— | 878 | 4931.5 |
| | (CH₂)₁₆—CO₂H)AQ-Me4Pal-AFIEWLIEGGPSSGAPPPSGS- | | |
| | NH₂ | | |
| 873 | Y-Aib-QGTFTSDFSQ-αMeL-LDKK((2-[2-(2-Amino- | 879 | 4923.5 |
| | ethoxy)-ethoxy]-acetyl)-(γ-Glu)-CO—(CH₂)₁₆—CO₂H)AQ- | | |
| | Me4Pal-AFIEYLIEGGPSSGAPPPSGS-NH₂ | | |
| 874 | Y-Aib-QGTFTSDFSQ-αMeL-LDKK((γ-Glu)₂-CO—(CH₂)₁₆- | 880 | 4907.4 |
| | CO₂H)AQ-Me4Pal-AFIEYLIEGGPSSGAPPPSGS-NH₂ | | |
| 875 | Y-Aib-QGTFTSDFSQ-αMeL-LDKK((2-[2-(2-Amino- | 881 | 4865.4 |
| | ethoxy)-ethoxy]-acetyl)-(γ-Glu)-CO—(CH₂)₁₆—CO₂H)AQ- | | |
| | Me4Pal-AFIEYLIAGGPSSGAPPPSGS-NH₂ | | |
| 876 | Y-Aib-QGTFTSDFSQ-αMeL-LDKK((γ-Glu)₂-CO—(CH₂)₁₆- | 882 | 4849.4 |
| | CO₂H)AQ-Me4Pal-AFIEYLIAGGPSSGAPPPSGS-NH₂ | | |
| 877 | Y-Aib-QGTFTSDFSQ-αMeL-LDKK((2-[2-(2-Amino- | 883 | 4946.5 |
| | ethoxy)-ethoxy]-acetyl)-(γ-Glu)-CO—(CH₂)₁₆—CO₂H)AQ- | | |
| | Me4Pal-AFIEWLIEGGPSSGAPPPSGS-NH₂ | | |
| 878 | Y-Aib-QGTFTSDFSQ-αMeL-LDKK((2-[2-(2-Amino- | 884 | 4930.5 |
| | ethoxy)-ethoxy]-acetyl)-(γ-Glu)-CO—(CH₂)₁₆—CO₂H)AQ- | | |
| | Me4Pal-AFIEWLIEGGPSSGAPPPSGS-NH₂ | | |
| 879 | Y-Aib-QGTFTSDFS-Orn-αMeL-LDKK((2-[2-(2-Amino- | 885 | 4995.6 |
| | ethoxy)-ethoxy]-acetyl)-(γ-Glu)-CO—(CH₂)₁₆—CO₂H)AQ- | | |
| | Me4Pal-EFIEYLIE-Aib-GPSSGAPPPSGS-NH₂ | | |
| 880 | Y-Aib-QGTFTSDFS-Orn-αMeL-LDKK((2-[2-(2-Amino- | 886 | 4981.6 |
| | ethoxy)-ethoxy]-acetyl)-(γ-Glu)-CO—(CH₂)₁₆—CO₂H)AQ- | | |
| | Me4Pal-DFIEYLIE-Aib-GPSSGAPPPSGS-NH₂ | | |
| 881 | Y-Aib-QGTFTSD-4Pal-SQ-αMeL-LDKK((2-[2-(2-Amino- | 887 | 4994.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGE-NH₂ | | |
| 882 | Y-Aib-QGTFTSD-4Pal-SQ-αMeL-LDKK((γ-Glu)₂-CO— | 888 | 4978.5 |
| | (CH₂)₁₆—CO₂H)AQ-Me4Pal-AFIEYLIE-Aib- | | |
| | GPSSGAPPPSGE-NH₂ | | |
| 883 | Y-Aib-QGTFTSD-4Pal-SQ-αMeL-LDKK((γ-Glu)₂-CO— | 889 | 4892.4 |
| | (CH₂)₁₆—CO₂H)AQ-Me4Pal-AFIEYLIAGGPSSGAPPPSGE- | | |
| | NH₂ | | |
| 884 | Y-Aib-QGTFTSD-4Pal-SQ-αMeL-LDKK((γ-Glu)₂-CO— | 890 | 5020.6 |
| | (CH₂)₁₆—CO₂H)AQ-Me4Pal-AFIEYLIE-Aib- | | |
| | GPSSGAPPPEGE-NH₂ | | |
| 885 | Y-Aib-QGTFTSD-4Pal-SQ-αMeL-LDKK((2-[2-(2-Amino- | 891 | 4953.5 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGS | | |
| 886 | Y-Aib-QGTFTSD-4Pal-SQ-αMeL-LDKK((2-[2-(2-Amino- | 892 | 5081.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGSE-NH₂ | | |
| 887 | Y-Aib-QGTFTSD-4Pal-SQ-αMeL-LDKK((2-[2-(2-Amino- | 893 | 5082.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGSE | | |
| 888 | Y-Aib-QGTFTSD-4Pal-SQ-αMeL-LDKK((2-[2-(2-Amino- | 894 | 5138.7 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGSGE-NH₂ | | |
| 889 | Y-Aib-QGTFTSD-4Pal-SQ-αMeL-LDKK((2-[2-(2-Amino- | 895 | 5139.7 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGSGE | | |
| 890 | Y-Aib-QGTFTSD-4Pal-SQ-αMeL-LDKK((γ-Glu)₂-CO— | 896 | 5065.6 |
| | (CH₂)₁₆—CO₂H)AQ-Me4Pal-AFIEYLIE-Aib- | | |
| | GPSSGAPPPSGSE-NH₂ | | |
| 891 | Y-Aib-QGTFTSD-4Pal-SQ-αMeL-LDKK((γ-Glu)₂-CO— | 897 | 5122.6 |
| | (CH₂)₁₆—CO₂H)AQ-Me4Pal-AFIEYLIE-Aib- | | |
| | GPSSGAPPPSGSGE | | |
| 892 | Y-Aib-QGTFTSD-4Pal-SQ-αMeL-LDKK((γ-Glu)₂-CO— | 898 | 5066.6 |
| | (CH₂)₁₆—CO₂H)AQ-Me4Pal-AFIEYLIE-Aib- | | |
| | GPSSGAPPPSGSE | | |
| 893 | Y-Aib-QGTFTSD-4Pal-SQ-αMeL-LDKK((γ-Glu)₂-CO— | 899 | 5123.6 |
| | (CH₂)₁₆—CO₂H)AQ-Me4Pal-AFIEYLIE-Aib- | | |
| | GPSSGAPPPSGSGE | | |
| 894 | Y-Aib-QGTFTSD-4Pal-SQ-αMeL-LDKK((Glu)-(γ-Glu)- | 900 | 4936.5 |
| | CO—(CH₂)₁₆—CO₂H)AQ-Me4Pal-AFIEYLIE-Aib- | | |
| | GPSSGAPPPSGS-NH₂ | | |
| 895 | Y-Aib-QGTFTSD-4Pal-SQ-αMeL-LDKK((Glu)₂-(γ-Glu)- | 901 | 5065.6 |
| | CO—(CH₂)₁₆—CO₂H)AQ-Me4Pal-AFIEYLIE-Aib- | | |
| | GPSSGAPPPSGS-NH₂ | | |
| 896 | Y-Aib-QGTFTSD-4Pal-SQ-αMeL-LDKK((Glu)-(γ-Glu)- | 902 | 5065.6 |
| | (Glu)-CO—(CH₂)₁₆—CO₂H)AQ-Me4Pal-AFIEYLIE-Aib- | | |
| | GPSSGAPPPSGS-NH₂ | | |
| 897 | Y-Aib-QGTFTSD-4Pal-SQ-αMeL-LDKK((2-[2-(2-Amino- | 903 | 5210.8 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGSEE-NH₂ | | |
| 898 | Y-Aib-QGTFTSD-4Pal-SQ-αMeL-LDKK((2-[2-(2-Amino- | 904 | 5339.9 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGSEEE-NH₂ | | |
| 899 | Y-Aib-QGTFTSD-4Pal-SQ-αMeL-LDKK((γ-Glu)₂-CO— | 905 | 5194.7 |
| | (CH₂)₁₆—CO₂H)AQ-Me4Pal-AFIEYLIE-Aib- | | |
| | GPSSGAPPPSGSEE-NH₂ | | |
| 900 | Y-Aib-QGTFTSD-4Pal-SQ-αMeL-LDKK((γ-Glu)₂-CO— | 906 | 5323.8 |
| | (CH₂)₁₆—CO₂H)AQ-Me4Pal-AFIEYLIE-Aib- | | |
| | GPSSGAPPPSGSEEE-NH₂ | | |
| 901 | Y-Aib-QGTFTSD-4Pal-SK-αMeL-LDKK((2-[2-(2-Amino- | 907 | 5005.6 |
| | ethoxy)-ethoxy]-acetyl)-(γ-Glu)-CO—(CH₂)₁₆—CO₂H)AQ- | | |
| | Aib-AFIEYLIE-Aib-GPSSGAPPPSGSE-NH₂ | | |
| 902 | Y-Aib-QGTFTSD-4Pal-SK-αMeL-LDKK((2-[2-(2-Amino- | 908 | 5134.7 |
| | ethoxy)-ethoxy]-acetyl)-(γ-Glu)-CO—(CH₂)₁₆—CO₂H)AQ- | | |
| | Aib-AFIEYLIE-Aib-GPSSGAPPPSGSEE-NH₂ | | |
| 903 | Y-Aib-QGTFTSD-4Pal-SK-αMeL-LDKK((2-[2-(2-Amino- | 909 | 5263.8 |
| | ethoxy)-ethoxy]-acetyl)-(γ-Glu)-CO—(CH₂)₁₆—CO₂H)AQ- | | |
| | Aib-AFIEYLIE-Aib-GPSSGAPPPSGSEEE-NH₂ | | |
| 904 | Y-Aib-QGTFTSD-4Pal-SK-αMeL-LDKK((γ-Glu)₂-CO— | 910 | 4988.6 |
| | (CH₂)₁₆—CO₂H)AQ-Aib-AFIEYLIE-Aib- | | |
| | GPSSGAPPPSGSE-NH₂ | | |
| 905 | Y-Aib-QGTFTSD-4Pal-SK-αMeL-LDKK((γ-Glu)₂-CO— | 911 | 5117.7 |
| | (CH₂)₁₆—CO₂H)AQ-Aib-AFIEYLIE-Aib- | | |
| | GPSSGAPPPSGSEE-NH₂ | | |
| 906 | Y-Aib-QGTFTSD-4Pal-SK-αMeL-LDKK((γ-Glu)₂-CO— | 912 | 5246.8 |
| | (CH₂)₁₆—CO₂H)AQ-Aib-AFIEYLIE-Aib- | | |
| | GPSSGAPPPSGSEEE-NH₂ | | |
| 907 | Y-Aib-QGTFTSD-4Pal-SK-αMeL-LDKK((γ-Glu)₂-CO— | 913 | 4859.4 |
| | (CH₂)₁₆—CO₂H)AQ-Aib-AFIEYLIE-Aib-GPSSGAPPPSGS- | | |
| | NH₂ | | |
| 908 | Y-Aib-QGTFTSD-4Pal-SK-αMeL-LDKK((2-[2-(2-Amino- | 914 | 4847.4 |
| | ethoxy)-ethoxy]-acetyl)-(γ-Glu)-CO—(CH₂)₁₆—CO₂H)AQ- | | |
| | Aib-AFIEYLIEGGPSSGAPPPSGS-NH₂ | | |
| 909 | Y-Aib-QGTFTSD-4Pal-SR-αMeL-LDKK((2-[2-(2-Amino- | 915 | 4889.5 |
| | ethoxy)-ethoxy]-acetyl)-(γ-Glu)-CO—(CH₂)₁₆—CO₂H)AQ- | | |
| | Aib-AFIE-αMeY-LLEGGPSSGAPPPSGS-NH₂ | | |
| 910 | Y-Aib-QGTFTSD-4Pal-SQ-αMeL-LDKK((2-[2-(2-Amino- | 916 | 4861.4 |
| | ethoxy)-ethoxy]-acetyl)-(γ-Glu)-CO—(CH₂)₁₆—CO₂H)AQ- | | |
| | Aib-AFIE-αMeY-LLEGGPSSGAPPPSGS-NH₂ | | |
| 911 | Y-Aib-QGTFTSD-4Pal-SQ-αMeL-LD-Orn-K((2-[2-(2- | 917 | 4847.4 |
| | Amino-ethoxy)-ethoxy]-acetyl)-(γ-Glu)-CO—(CH₂)₁₆- | | |
| | CO₂H)AQ-Aib-AFIE-αMeY-LLEGGPSSGAPPPSGS-NH₂ | | |
| 912 | Y-Aib-QGTFTSDFSK-αMeL-LDKK((2-[2-(2-Amino- | 918 | 4873.5 |
| | ethoxy)-ethoxy]-acetyl)-(γ-Glu)-CO—(CH₂)₁₆—CO₂H)AQ- | | |
| | Aib-AFIE-Me4Pal-LIE-Aib-GPSSGAPPPSGS-NH₂ | | |
| 913 | Y-Aib-QGTFTSDFSQ-αMeL-LDKK((2-[2-(2-Amino- | 919 | 4873.5 |
| | ethoxy)-ethoxy]-acetyl)-(γ-Glu)-CO—(CH₂)₁₆—CO₂H)AQ- | | |
| | Aib-AFIE-Me4Pal-LIE-Aib-GPSSGAPPPSGS-NH₂ | | |
| 914 | Y-Aib-QGTFTSD-4Pal-SK-αMeL-LDKK((2-[2-(2-Amino- | 920 | 4874.5 |
| | ethoxy)-ethoxy]-acetyl)-(γ-Glu)-CO—(CH₂)₁₆—CO₂H)AQ- | | |
| | Aib-AFIE-Me4Pal-LIE-Aib-GPSSGAPPPSGS-NH₂ | | |
| 915 | Y-Aib-QGTFTSD-4Pal-SK-αMeL-LDKK((2-[2-(2-Amino- | 921 | 4952.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSGS-NH₂ | | |
| 916 | Y-Aib-QGTFTSD-4Pal-SK-αMeL-LDKK((2-[2-(2-Amino- | 922 | 4951.6 |
| | ethoxy)-ethoxy]-acetyl)-(γ-Glu)-CO—(CH₂)₁₆—CO₂H)AQ- | | |
| | Me4Pal-AFIE-Me4Pal-LIE-Aib-GPSSGAPPPSGS-NH₂ | | |
| 917 | Y-Aib-QGTFTSD-4Pal-SQ-αMeL-LDKK((2-[2-(2-Amino- | 923 | 4951.5 |
| | ethoxy)-ethoxy]-acetyl)-(γ-Glu)-CO—(CH₂)₁₆—CO₂H)AQ- | | |
| | Me4Pal-AFIE-Me4Pal-LIE-Aib-GPSSGAPPPSGS-NH₂ | | |
| 918 | Y-Aib-QGTFTSD-4Pal-SQ-αMeL-LDKK((2-[2-(2-Amino- | 924 | 4995.5 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSeG | | |
| 919 | Y-Aib-QGTFTSD-4Pal-SQ-αMeL-LDKK((2-[2-(2-Amino- | 925 | 5067.6 |
| | ethoxy)-ethoxy]-acetyl)-(γ-Glu)-CO—(CH₂)₁₆—CO₂H)AQ- | | |
| | Me4Pal-AFIEYLIE-Aib-GPSSGAPPPSeE | | |
| 920 | Y-Aib-QGTFTSD-4Pal-SQ-αMeL-LDKK(e-(γ-Glu)-CO— | 926 | 4936.5 |
| | (CH₂)₁₆—CO₂H)AQ-Me4Pal-AFIEYLIE-Aib- | | |
| | GPSSGAPPPSGS-NH₂ | | |
| 921 | Y-Aib-QGTFTSD-4Pal-SQ-αMeL-LDKK((e)₂-(γ-Glu)-CO— | 927 | 5065.6 |
| | (CH₂)₁₆—CO₂H)AQ-Me4Pal-AFIEYLIE-Aib- | | |
| | GPSSGAPPPSGS-NH₂ | | |
| 922 | Y-Aib-QGTFTSD-4Pal-SQ-αMeL-LDKK((γ-Glu)-(e)₂-CO— | 928 | 5065.6 |
| | (CH₂)₁₆—CO₂H)AQ-Me4Pal-AFIEYLIE-Aib- | | |
| | GPSSGAPPPSGS-NH₂ | | |
| 923 | Y-Aib-QGTFTSD-4Pal-SQ-αMeL-LDKK(e-(γ-Glu)-e-CO— | 929 | 5065.6 |
| | (CH₂)₁₆—CO₂H)AQ-Me4Pal-AFIEYLIE-Aib- | | |
| | GPSSGAPPPSGS-NH₂ | | |
| 924 | Y-Aib-QGTFTSD-4Pal-SQ-αMeL-LDKK(e-(γ-Glu)-CO— | 930 | 5066.6 |
| | (CH₂)₁₆—CO₂H)AQ-Me4Pal-AFIEYLIE-Aib- | | |
| | GPSSGAPPPSGSE-NH₂ | | |
| 925 | Y-Aib-QGTFTSD-4Pal-SQ-αMeL-LDKK((e)₂-(γ-Glu)-CO— | 931 | 5195.7 |
| | (CH₂)₁₆—CO₂H)AQ-Me4Pal-AFIEYLIE-Aib- | | |
| | GPSSGAPPPSGSE-NH₂ | | |
| 926 | Y-Aib-QGTFTSD-4Pal-SQ-αMeL-LDKK((γ-Glu)-(e)₂-CO— | 932 | 5195.7 |
| | (CH₂)₁₆—CO₂H)AQ-Me4Pal-AFIEYLIE-Aib- | | |
| | GPSSGAPPPSGSE-NH₂ | | |
| 927 | Y-Aib-QGTFTSD-4Pal-SQ-αMeL-LDKK(e-(γ-Glu)-e-CO— | 933 | 5195.7 |
| | (CH₂)₁₆—CO₂H)AQ-Me4Pal-AFIEYLIE-Aib- | | |
| | GPSSGAPPPSGSE-NH₂ | | |
| 928 | Y-Aib-QGTFTSD-4Pal-SQ-αMeL-LDKK(e-(γ-Glu)-CO— | 934 | 5195.7 |
| | (CH₂)₁₆—CO₂H)AQ-Me4Pal-AFIEYLIE-Aib- | | |
| | GPSSGAPPPSGSEE-NH₂ | | |
| 929 | Y-Aib-QGTFTSD-4Pal-SQ-αMeL-LDKK((e)₂-(γ-Glu)-CO— | 935 | 5324.8 |
| | (CH₂)₁₆—CO₂H)AQ-Me4Pal-AFIEYLIE-Aib- | | |
| | GPSSGAPPPSGSEE-NH₂ | | |
| 930 | Y-Aib-QGTFTSD-4Pal-SQ-αMeL-LDKK((γ-Glu)-(e)₂-CO— | 936 | 5324.8 |
| | (CH₂)₁₆—CO₂H)AQ-Me4Pal-AFIEYLIE-Aib- | | |
| | GPSSGAPPPSGSEE-NH₂ | | |
| 931 | Y-Aib-QGTFTSD-4Pal-SQ-αMeL-LDKK(e-(γ-Glu)-e-CO— | 937 | 5324.8 |
| | (CH₂)₁₆—CO₂H)AQ-Me4Pal-AFIEYLIE-Aib- | | |
| | GPSSGAPPPSGSEE-NH₂ | | |
| 932 | Y-Aib-QGTFTSD-4Pal-SQ-αMeL-LD-Orn-K((2-[2-(2- | 938 | 4924.5 |
| | Amino-ethoxy)-ethoxy]-acetyl)-(γ-Glu)-CO—(CH₂)₁₆- | | |
| | CO₂H)AQ-Me4Pal-AFIE-αMeY-LLEGGPSSGAPPPSGS- | | |
| | NH₂ | | |
| 933 | Y-Aib-QGTFTSD-4Pal-S-Orn -- αMeL-LD-Orn-K((2-[2-(2- | 939 | 4833.4 |
| | Amino-ethoxy)-ethoxy]-acetyl)-(γ-Glu)-CO—(CH₂)₁₆- | | |
| | CO₂H)AQ-Aib-AFIE-αMeY-LLEGGPSSGAPPPSGS-NH₂ | | |
| 934 | Y-Aib-QGTFTSD-4Pal-SQ-αMeL-LDKK((γ-Glu)₂-CO— | 940 | 4979.5 |
| | (CH₂)₁₆—CO₂H)AQ-Me4Pal-QFIE-αMeY- | | |
| | LIEGGPSSGAPPPSGS-NH₂ | | |
| 935 | Y-Aib-QGTFTSD-4Pal-SQ-αMeL-LD-Orn-K((γ-Glu)₂-CO— | 941 | 4922.5 |
| | (CH₂)₁₆—CO₂H)AQ-Me4Pal-AFIEYLIE-Aib- | | |
| | GPSSGAPPPSGS-NH₂ | | |
| 936 | Y-Aib-QGTFTSD-4Pal-SQ-αMeL-LD-Orn-K((2-[2-(2- | 942 | 5051.6 |
| | Amino-ethoxy)-ethoxy]-acetyl)-(γ-Glu)₂-CO—(CH₂)₁₆- | | |
| | CO₂H)AQ-Me4Pal-AFIEYLIE-Aib-GPSSGAPPPSGSE- | | |
| | NH₂ | | |
| 937 | Y-Aib-QGTFTSD-4Pal-SQ-αMeL-LD-Orn-K((γ-Glu)₂-CO— | 943 | 5180.7 |
| | (CH₂)₁₆—CO₂H)AQ-Me4Pal-AFIEYLIE-Aib- | | |
| | GPSSGAPPPSGSEE-NH₂ | | |
| 938 | Y-Aib-QGTFTSD-4Pal-SQ-αMeL-LDK-K((γ-Glu)₂-CO— | 944 | 4845.4 |
| | (CH₂)₁₆—CO₂H)AQ-Aib-AFIE-αMeY- | | |
| | LLEGGPSSGAPPPSGS-NH₂ | | |
| 939 | Y-Aib-QGTFTSD-4Pal-S-Orn-αMeL-LD-Orn-K((γ-Glu)₂- | 945 | 4908.5 |
| | CO—(CH₂)₁₆—CO₂H)AQ-Me4Pal-AFIEYLIE-Aib- | | |
| | GPSSGAPPPSGS-NH₂ | | |
| 940 | Y-Aib-QGTFTSD-4Pal-S-Orn-αMeL-LD-Orn-K((γ-Glu)₂- | 946 | 4965.5 |
| | CO—(CH₂)₁₆—CO₂H)AQ-Me4Pal-QFIEYLIE-Aib- | | |
| | GPSSGAPPPSGS-NH₂ | | |
| 941 | Y-Aib-QGTFTSD-4Pal-S-Orn-αMeL-LDKK((γ-Glu)₂-CO— | 947 | 4831.4 |
| | (CH₂)₁₆—CO₂H)AQ-Aib-AFIE-αMeY- | | |
| | LIEGGPSSGAPPPSGS-NH₂ | | |
| 942 | Y-Aib-QGTFTSD-4Pal-S-Orn-αMeL-LDKK((γ-Glu)₂-CO— | 948 | 4831.4 |
| | (CH₂)₁₆—CO₂H)AQ-Aib-AFIE-αMeY- | | |
| | LLEGGPSSGAPPPSGS-NH₂ | | |
| 943 | Y-Aib-QGTFTSD-4Pal-S-Orn-αMeL-LD-Orn-K((γ-Glu)₃- | 949 | 5166.7 |
| | CO—(CH₂)₁₆—CO₂H)AQ-Me4Pal-AFIEYLIE-Aib- | | |
| | GPSSGAPPPSGSE-NH₂ | | |
| 944 | Y-Aib-QGTFTSD-4Pal-S-Orn-αMeL-LDKK((γ-Glu)₂-CO— | 950 | 5089.6 |
| | (CH₂)₁₆—CO₂H)AQ-Aib-AFIE-αMeY- | | |
| | LLEGGPSSGAPPPSGSEE-NH₂ | | |
| 945 | Y-Aib-QGTFTSD-4Pal-SQ-αMeL-LDKK((γ-Glu)₂-CO— | 951 | 5103.6 |
| | (CH₂)₁₆—CO₂H)AQ-Aib-AFIE-αMeY- | | |
| | LLEGGPSSGAPPPSGSEE-NH₂ | | |
| 946 | Y-Aib-QGTFTSD-4Pal-SQ-αMeL-LD-Orn-K((γ-Glu)₂-CO— | 952 | 5089.6 |
| | (CH₂)₁₆—CO₂H)AQ-Aib-AFIE-αMeY- | | |
| | LLEGGPSSGAPPPSGSEE-NH₂ | | |
| 947 | Y-Aib-QGTFTSD-4Pal-SQ-αMeL-LDKK((2-[2-(2-Amino- | 953 | 5053.6 |
| | ethoxy)-ethoxy]-acetyl)-(γ-Glu)₂-CO—(CH₂)₁₆—CO₂H)AQ- | | |
| | Me4Pal-AFIEYLIAGGPSSGEPPPSGS-NH₂ | | |
| 948 | Y-Aib-QGTFTSD-4Pal-SQ-αMeL-LDKK((2-[2-(2-Amino- | 954 | 4923.5 |
| | ethoxy)-ethoxy]-acetyl)-(γ-Glu)-CO—(CH₂)₁₆—CO₂H)AQ- | | |
| | Me4Pal-AFIE-Me4Pal-LIEGGPSSGAPPPSGS-NH₂ | | |
| 949 | Y-Aib-QGTFTSD-4Pal-S-Orn-αMeL-LDKK((2-[2-(2- | 955 | 4909.5 |
| | Amino-ethoxy)-ethoxy]-acetyl)-(γ-Glu)-CO—(CH₂)₁₆- | | |
| | CO₂H)AQ-Me4Pal-AFIE-Me4Pal-LIEGGPSSGAPPPSGS- | | |
| | NH₂ | | |
| 950 | Y-Aib-QGTFTSD-4Pal-SR-αMeL-LDKK((2-[2-(2-Amino- | 956 | 4951.5 |
| | ethoxy)-ethoxy]-acetyl)-(γ-Glu)-CO—(CH₂)₁₆—CO₂H)AQ- | | |
| | Me4Pal-AFIE-Me4Pal-LIEGGPSSGAPPPSGS-NH₂ | | |
| 951 | Y-Aib-QGTFTSD-4Pal-SQ-αMeL-LDKK((γ-Glu)₂-CO— | 957 | 4907.4 |
| | (CH₂)₁₆—CO₂H)AQ-Me4Pal-AFIE-Me4Pal- | | |
| | LIEGGPSSGAPPPSGS-NH₂ | | |
| 952 | Y-Aib-QGTFTSD-4Pal-S-Orn-αMeL-LDKK((γ-Glu)₂-CO— | 958 | 4893.5 |
| | (CH₂)₁₆—CO₂H)AQ-Me4Pal-AFIE-Me4Pal- | | |
| | LIEGGPSSGAPPPSGS-NH₂ | | |
| 953 | Y-Aib-QGTFTSD-4Pal-SR-αMeL-LDKK((γ-Glu)₂-CO— | 959 | 4935.5 |
| | (CH₂)₁₆—CO₂H)AQ-Me4Pal-AFIE-Me4Pal- | | |
| | LIEGGPSSGAPPPSGS-NH₂ | | |
| 954 | Y-Aib-QGTFTSD-4Pal-SQ-αMeL-LDKK((2-[2-(2-Amino- | 960 | 5181.7 |
| | ethoxy)-ethoxy]-acetyl)-(γ-Glu)-CO—(CH₂)₁₆—CO₂H)AQ- | | |
| | Me4Pal-AFIE-Me4Pal-LIEGGPSSGAPPPSGSEE-NH₂ | | |
| 955 | Y-Aib-QGTFTSD-4Pal-S-Orn-αMeL-LDKK((2-[2-(2- | 961 | 5167.7 |
| | Amino-ethoxy)-ethoxy]-acetyl)-(γ-Glu)-CO—(CH₂)₁₆- | | |
| | CO₂H)AQ-Me4Pal-AFIE-Me4Pal- | | |
| | LIEGGPSSGAPPPSGSEE-NH₂ | | |
| 956 | Y-Aib-QGTFTSD-4Pal-SR-αMeL-LDKK((2-[2-(2-Amino- | 962 | 5209.8 |
| | ethoxy)-ethoxy]-acetyl)-(γ-Glu)-CO—(CH₂)₁₆—CO₂H)AQ- | | |
| | Me4Pal-AFIE-Me4Pal-LIEGGPSSGAPPPSGSEE-NH₂ | | |
| 957 | Y-Aib-QGTFTSD-4Pal-SQ-αMeL-LDKK((γ-Glu)₂-CO— | 963 | 5165.7 |
| | (CH₂)₁₆—CO₂H)AQ-Me4Pal-AFIE-Me4Pal- | | |
| | LIEGGPSSGAPPPSGSEE-NH₂ | | |
| 958 | Y-Aib-QGTFTSD-4Pal-S-Orn-αMeL-LDKK((γ-Glu)₂-CO— | 964 | 5151.7 |
| | (CH₂)₁₆—CO₂H)AQ-Me4Pal-AFIE-Me4Pal- | | |
| | LIEGGPSSGAPPPSGSEE-NH₂ | | |
| 959 | Y-Aib-QGTFTSD-4Pal-SR-αMeL-LDKK((γ-Glu)₂-CO— | 965 | 5193.7 |
| | (CH₂)₁₆—CO₂H)AQ-Me4Pal-AFIE-Me4Pal- | | |
| | LIEGGPSSGAPPPSGSEE-NH₂ | | |
| 960 | Y-Aib-QGTFTSD-4Pal-SQ-αMeL-LDKK((γ-Glu)₂-CO— | 966 | 5109.6 |
| | (CH₂)₁₆—CO₂H)AQ-Me4Pal- | | |
| | AFIEYLIAGGPSSGAPPPSGSEE-NH₂ | | |
| 961 | Y-Aib-QGTFTSD-4Pal-S-Orn-αMeL-LDKK((γ-Glu)₂-CO— | 967 | 5095.6 |
| | (CH₂)₁₆—CO₂H)AQ-Me4Pal- | | |
| | AFIEYLIAGGPSSGAPPPSGSEE-NH₂ | | |
| 962 | Y-Aib-QGTFTSD-4Pal-SR-αMeL-LDKK((γ-Glu)₂-CO— | 968 | 5137.7 |
| | (CH₂)₁₆—CO₂H)AQ-Me4Pal- | | |
| | AFIEYLIAGGPSSGAPPPSGSEE-NH₂ | | |
| 963 | Y-Aib-QGTFTSD-4Pal-SQ-αMeL-LDKK((γ-Glu)₂-CO— | 969 | 5141.6 |
| | (CH₂)₁₆—CO₂H)AQ-Me4Pal- | | |
| | AFIEYLIAGGESSGAPPPSGSEE-NH₂ | | |
| 964 | Y-Aib-QGTFTSD-4Pal-S-Orn-αMeL-LDKK((γ-Glu)₂-CO— | 970 | 5127.6 |
| | (CH₂)₁₆—CO₂H)AQ-Me4Pal- | | |
| | AFIEYLIAGGESSGAPPPSGSEE-NH₂ | | |
| 965 | Y-Aib-QGTFTSD-4Pal-SR-αMeL-LDKK((γ-Glu)₂-CO— | 971 | 5169.7 |
| | (CH₂)₁₆—CO₂H)AQ-Me4Pal- | | |
| | AFIEYLIAGGESSGAPPPSGSEE-NH₂ | | |
| 966 | Y-Aib-QGTFTSD-4Pal-S-Orn-αMeL-LDKK((γ-Glu)₂-CO— | 972 | 5038.6 |
| | (CH₂)₁₆—CO₂H)AQ-Me4Pal-AFIE-αMeY- | | |
| | LIEGGPSSGAPPPSGSE-NH₂ | | |
| 967 | Y-Aib-QGTFTSD-4Pal-S-Orn-αMeL-LDKK((γ-Glu)₂-CO— | 973 | 5167.7 |
| | (CH₂)₁₆—CO₂H)AQ-Me4Pal-AFIE-αMeY- | | |
| | LIEGGPSSGAPPPSGSEE-NH₂ | | |
| 968 | Y-Aib-QGTFTSD-4Pal-SQ-αMeL-LDKK((γ-Glu)₂-CO— | 974 | 4908.4 |
| | (CH₂)₁₆—CO₂H)AQ-Me4Pal-AFIEYLIAGGPSSGEPPPSGS- | | |
| | NH₂ | | |
| 969 | Y-Aib-QGTFTSD-4Pal-SQ-αMeL-LDKK((γ-Glu)₂-CO— | 975 | 4892.4 |
| | (CH₂)₁₆—CO₂H)AQ-Me4Pal-AFIEYLIAGGPSSGAPPPEGS | | |
| 970 | Y-Aib-QGTFTSD-4Pal-SQ-αMeL-LDKK((γ-Glu)₂-CO— | 976 | 4951.5 |
| | (CH₂)₁₆—CO₂H)AQ-Me4Pal-AFIEYLIAGGPSSGEPPPEGS | | |
| 971 | Y-Aib-QGTFTSD-4Pal-SQ-αMeL-LDKK((γ-Glu)₂-CO— | 977 | 5095.6 |
| | (CH₂)₁₆—CO₂H)AQ-Me4Pal- | | |
| | AFIEYLIAGGPSSGEPPPSGSGE | | |
| 972 | Y-Aib-QGTFTSD-4Pal-S-Orn-αMeL-LD-Or-K((γ-Glu)₂- | 978 | 5209.8 |
| | CO—(CH₂)₁₆—CO₂H)AQ-Me4Pal-QFIE-αMeY- | | |
| | LIEGGPSSGAPPPSGSEE-NH₂ | | |
| 973 | Y-Aib-QGTFTSD-4Pal-S-Orn-αMeL-LD-Orn-K((γ-Glu)₂- | 979 | 5210.8 |
| | CO—(CH₂)₁₆—CO₂H)AQ-Me4Pal-EFIE-αMeY- | | |
| | LIEGGPSSGAPPPSGSEE-NH₂ | | |
| 974 | Y-Aib-QGTFTSD-4Pal-S-Orn-αMeL-LD-Orn-K((γ-Glu)₂- | 980 | 5195.8 |
| | CO—(CH₂)₁₆—CO₂H)AQ-Me4Pal-Orn-FIE-αMeY- | | |
| | LIEGGPSSGAPPPSGSEE-NH₂ | | |
| 975 | Y-Aib-QGTFTSD-4Pal-S-Orn-αMeL-LD-Orn-K((γ-Glu)₂- | 981 | 5209.8 |
| | CO—(CH₂)₁₆—CO₂H)AQ-Me4Pal-QFIE-αMeY- | | |
| | LLEGGPSSGAPPPSGSEE-NH₂ | | |
| 976 | Y-Aib-QGTFTSD-4Pal-S-Orn-αMeL-LD-Orn-K((γ-Glu)₂- | 982 | 5210.8 |
| | CO—(CH₂)₁₆—CO₂H)AQ-Me4Pal-EFIE-αMeY- | | |
| | LLEGGPSSGAPPPSGSEE-NH₂ | | |
| 977 | Y-Aib-QGTFTSD-4Pal-SQ-αMeL-LD-Orn-K((γ-Glu)₂-CO— | 983 | 5024.5 |
| | (CH₂)₁₆—CO₂H)AQ-Me4Pal- | | |
| | AFIEYLIAGGPSSGEPPPSGSE | | |
| 978 | Y-Aib-QGTFTSD-4Pal-SQ-αMeL-LD-Orn-K((γ-Glu)₂-CO— | 984 | 5081.6 |
| | (CH₂)₁₆—CO₂H)AQ-Me4Pal- | | |
| | AFIEYLIAGGPSSGEPPPSGSGE | | |
| 979 | Y-Aib-QGTFTSD-4Pal-S-Orn-αMeL-LDKK((γ-Glu)₂-CO— | 985 | 4936.5 |
| | (CH₂)₁₆—CO₂H)AQ-Me4Pal-AFIEYLIAGGPSSGEPPPSGE- | | |
| | NH₂ | | |
| 980 | Y-Aib-QGTFTSD-4Pal-S-Orn-αMeL-LDKK((γ-Glu)₂-CO— | 986 | 5037.6 |
| | (CH₂)₁₆—CO₂H)AQ-Me4Pal- | | |
| | AFIEYLIAGGPSSGEPPPSGTE-NH₂ | | |
| 981 | Y-Aib-QGTFTSD-4Pal-S-Orn-αMeL-LDKK((γ-Glu)₂-CO— | 987 | 5166.7 |
| | (CH₂)₁₆—CO₂H)AQ-Me4Pal- | | |
| | AFIEYLIAGGPSSGEPPPSGTEE-NH₂ | | |
| 982 | Y-Aib-QGTFTSD-4Pal-S-Orn-αMeL-LDKK((γ-Glu)₂-CO— | 988 | 5295.8 |
| | (CH₂)₁₆—CO₂H)AQ-Me4Pal- | | |
| | AFIEYLIAGGPSSGEPPPSGTEEE-NH₂ | | |
| 983 | Y-Aib-QGTFTSD-4Pal-S-Orn-αMeL-LD-Orn-K((γ-Glu)₂- | 989 | 4922.5 |
| | CO—(CH₂)₁₆—CO₂H)AQ-Me4Pal- | | |
| | AFIEYLIAGGPSSGEPPPSGE-NH₂ | | |
| 984 | Y-Aib-QGTFTSD-4Pal-S-Orn-αMeL-LD-Orn-K((γ-Glu)₂- | 990 | 5023.6 |
| | CO—(CH₂)₁₆—CO₂H)AQ-Me4Pal- | | |
| | AFIEYLIAGGPSSGEPPPSGTE-NH₂ | | |
| 985 | Y-Aib-QGTFTSD-4Pal-S-Orn-αMeL-LD-Orn-K((γ-Glu)₂- | 991 | 5152.7 |
| | CO—(CH₂)₁₆—CO₂H)AQ-Me4Pal- | | |
| | AFIEYLIAGGPSSGEPPPSGTEE-NH₂ | | |
| 986 | Y-Aib-QGTFTSD-4Pal-SQ-αMeL-LD-Orn-K((γ-Glu)₂-CO— | 992 | 5281.8 |
| | (CH₂)₁₆—CO₂H)AQ-Me4Pal- | | |
| | AFIEYLIAGGPSSGEPPPSGTEEE-NH₂ | | |
| 987 | Y-Aib-QGTFTSD-4Pal-S-Orn-αMeL-LDKK((2-[2-(2- | 993 | 5124.7 |
| | Amino-ethoxy)-ethoxy]-acetyl)-(γ-Glu)₂-CO—(CH₂)₁₆- | | |
| | CO₂H)AQ-Me4Pal-AFIE-Me4Pal-LIE-Aib- | | |
| | GPSSGEPPPSGS-NH₂ | | |
| 988 | Y-Aib-QGTFTSD-4Pal-S-Orn-αMeL-LDKK((2-[2-(2- | 994 | 5253.8 |
| | Amino-ethoxy)-ethoxy]-acetyl)-(γ-Glu)₂-CO—(CH₂)₁₆- | | |
| | CO₂H)AQ-Me4Pal-AFIE-Me4Pal-LIE-Aib- | | |
| | GPSSGEPPPSGSE-NH₂ | | |
| 989 | Y-Aib-QGTFTSD-4Pal-S-Orn-αMeL-LDKK((γ-Glu)₂-CO— | 995 | 4979.6 |
| | (CH₂)₁₆—CO₂H)AQ-Me4Pal-AFIE-Me4Pal-LIE-Aib- | | |
| | GPSSGEPPPSGS-NH₂ | | |
| 990 | Y-Aib-QGTFTSD-4Pal-S-Orn-αMeL-LDKK((γ-Glu)₂-CO— | 996 | 5108.7 |
| | (CH₂)₁₆—CO₂H)AQ-Me4Pal-AFIE-Me4Pal-LIE-Aib- | | |
| | GPSSGEPPPSGSE-NH₂ | | |
| 991 | Y-Aib-QGTFTSD-4Pal-S-Orn-αMeL-LD-Or-K((γ-Glu)₂- | 997 | 4965.5 |
| | CO—(CH₂)₁₆—CO₂H)AQ-Me4Pal-AFIE-Me4Pal-LIE-Aib- | | |
| | GPSSGEPPPSGS-NH₂ | | |
| 992 | Y-Aib-QGTFTSD-4Pal-S-Orn-αMeL-LD-Orn-K((γ-Glu)₂- | 998 | 5136.8 |
| | CO—(CH₂)₁₆—CO₂H)AQ-Me4Pal-QFIE-αMeY- | | |
| | LIAGGPSSGAPPPSGSEE-NH₂ | | |
| 993 | Y-Aib-QGTFTSD-4Pal-S-Orn-αMeL-LD-Orn-K((γ-Glu)₂- | 999 | 5137.7 |
| | CO—(CH₂)₁₆—CO₂H)AQ-Me4Pal-EFIE-αMeY- | | |
| | LIAGGPSSGAPPPSGSEE-NH₂ | | |
| 994 | Y-Aib-QGTFTSD-4Pal-S-Orn-αMeL-LD-Orn-K((γ-Glu)₂- | 1000 | 5122.8 |
| | CO—(CH₂)₁₆—CO₂H)AQ-Me4Pal-Orn-FIE-αMeY- | | |
| | LIAGGPSSGAPPPSGSEE-NH₂ | | |
| 995 | Y-Aib-QGTFTSD-4Pal-S-Orn-αMeL-LD-Orn-K((γ-Glu)₂- | 1001 | 5136.8 |
| | CO—(CH₂)₁₆—CO₂H)AQ-Me4Pal-QFIE-αMeY- | | |
| | LLAGGPSSGAPPPSGSEE-NH₂ | | |
| 996 | Y-Aib-QGTFTSD-4Pal-S-Orn-αMeL-LD-Orn-K((γ-Glu)₂- | 1002 | 5137.7 |
| | CO—(CH₂)₁₆—CO₂H)AQ-Me4Pal-EFIE-αMeY- | | |
| | LLAGGPSSGAPPPSGSEE-NH₂ | | |
| 997 | Y-Aib-QGTFTSD-4Pal-S-Orn-αMeL-LD-Orn-K((γ-Glu)₂- | 1003 | 5122.8 |
| | CO—(CH₂)₁₆—CO₂H)AQ-Me4Pal-Orn-FIE-αMeY- | | |
| | LLAGGPSSGAPPPSGSEE-NH₂ | | |
| 998 | Y-Aib-QGTFTSD-4Pal-SQ-αMeL-LD-Orn-K((γ-Glu)₂-CO— | 1004 | 5150.7 |
| | (CH₂)₁₆—CO₂H)AQ-Me4Pal-QFIE-αMeY- | | |
| | LLAGGPSSGAPPPSGSEE-NH₂ | | |
| 999 | Y-Aib-QGTFTSD-4Pal-SQ-αMeL-LD-Orn-K((γ-Glu)₂-CO— | 1005 | 5151.7 |
| | (CH₂)₁₆—CO₂H)AQ-Me4Pal-EFIE-αMeY- | | |
| | LLAGGPSSGAPPPSGSEE-NH₂ | | |
| 1000 | Y-Aib-QGTFTSD-4Pal-S-Orn-αMeL-LD-Orn-K((γ-Glu)₂- | 1006 | 5137.7 |
| | CO—(CH₂)₁₆—CO₂H)AQ-Me4Pal- | | |
| | QFIEYLIAGGPSSGAPPPSGSEE-NH₂ | | |
| 1001 | Y-Aib-QGTFTSD-4Pal-S-Orn-αMeL-LD-Orn-K((γ-Glu)₂- | 1007 | 5138.7 |
| | CO—(CH₂)₁₆—CO₂H)AQ-Me4Pal- | | |
| | EFIEYLIAGGPSSGAPPPSGSEE-NH₂ | | |
| 1002 | Y-Aib-QGTFTSD-4Pal-S-Orn-αMeL-LD-Orn-K((γ-Glu)₂- | 1008 | 5137.7 |
| | CO—(CH₂)₁₆—CO₂H)AQ-Me4Pal- | | |
| | QFIEYLLAGGPSSGAPPPSGSEE-NH₂ | | |
| 1003 | Y-Aib-QGTFTSD-4Pal-S-Orn-αMeL-LD-Orn-K((γ-Glu)₂- | 1009 | 5138.7 |
| | CO—(CH₂)₁₆—CO₂H)AQ-Me4Pal- | | |
| | EFIEYLLAGGPSSGAPPPSGSEE-NH₂ | | |
| 1004 | Y-Aib-QGTFTSD-4Pal-S-Orn-αMeL-LD-Orn-K((γ-Glu)₂- | 1010 | 5123.8 |
| | CO—(CH₂)₁₆—CO₂H)AQ-Me4Pal-Orn- | | |
| | FIEYLLAGGPSSGAPPPSGSEE-NH₂ | | |
| 1005 | Y-Aib-QGTFTSD-4Pal-SQ-αMeL-LD-Orn-K((γ-Glu)₂-CO— | 1011 | 5151.7 |
| | (CH₂)₁₆—CO₂H)AQ-Me4Pal- | | |
| | QFIEYLIAGGPSSGAPPPSGSEE-NH₂ | | |
| 1006 | Y-Aib-QGTFTSD-4Pal-SQ-αMeL-LD-Orn-K((γ-Glu)₂-CO— | 1012 | 5151.7 |
| | (CH₂)₁₆—CO₂H)AQ-Me4Pal- | | |
| | QFIEYLLAGGPSSGAPPPSGSEE-NH₂ | | |
| 1007 | Y-Aib-QGTFTSD-4Pal-SQ-αMeL-LD-Orn-K((γ-Glu)₂-CO— | 1013 | 4894.4 |
| | (CH₂)₁₆—CO₂H)AQ-Me4Pal-AFIEYLIAGGPSSGEPPPSGS- | | |
| | NH₂ | | |
| 1008 | Y-Aib-QGTFTSD-4Pal-SQ-αMeL-LD-Orn-K((γ-Glu)₂-CO— | 1014 | 5023.5 |
| | (CH₂)₁₆—CO₂H)AQ-Me4Pal- | | |
| | AFIEYLIAGGPSSGEPPPSGSE-NH₂ | | |
| 1009 | Y-Aib-QGTFTSD-4Pal-S-Orn-αMeL-LDK-K((γ-Glu)₂-CO— | 1015 | 4965.5 |
| | (CH₂)₁₆—CO₂H)AQ-Me4Pal- | | |
| | AFIEYLIAGGPSSGAPPPSGSE-NH₂ | | |
| 1010 | Y-Aib-QGTFTSD-4Pal-S-Orn-αMeL-LDK-K((γ-Glu)₂-CO— | 1016 | 5007.6 |
| | (CH₂)₁₆—CO₂H)AQ-Me4Pal- | | |
| | AFIEYLIAGGPSSGAPPPSESE-NH₂ | | |
| 1011 | Y-Aib-QGTFTSD-4Pal-S-Orn-αMeL-LD-Orn-K((γ-Glu)₂- | 1017 | 4951.5 |
| | CO—(CH₂)₁₆—CO₂H)AQ-Me4Pal- | | |
| | AFIEYLIAGGPSSGAPPPSGSE-NH₂ | | |
| 1012 | Y-Aib-QGTFTSD-4Pal-S-Orn-αMeL-LD-Orn-K((γ-Glu)₂- | 1018 | 4993.5 |
| | CO—(CH₂)₁₆—CO₂H)AQ-Me4Pal- | | |
| | AFIEYLIAGGPSSGAPPPSESE-NH₂ | | |
| 1013 | Y-Aib-QGTFTSD-4Pal-S-Orn-αMeL-LD-Or-K((γ-Glu)₂- | 1019 | 4894.5 |
| | CO—(CH₂)₁₆—CO₂H)AQ-Me4Pal-AFIE-αMeY- | | |
| | LIEGGPSSGAPPPSGS-NH₂ | | |
| 1014 | Y-Aib-QGTFTSD-4Pal-S-Orn-αMeL-LDKK((γ-Glu)₂-CO— | 1020 | 4908.5 |
| | (CH₂)₁₆—CO₂H)AQ-Me4Pal-AFIE-αMeY- | | |
| | LIAGGPSSGEPPPSGS-NH₂ | | |
| 1015 | Y-Aib-QGTFTSD-4Pal-S-Orn-αMeL-LDKK((γ-Glu)₂-CO— | 1021 | 4892.6 |
| | (CH₂)₁₆—CO₂H)AQ-Me4Pal-AFIE-αMeY- | | |
| | LIAGGPSSGAPPPEGS-NH₂ | | |
| 1016 | Y-Aib-QGTFTSD-4Pal-SQ-αMeL-LD-Orn-K((2-[2-(2- | 1022 | 5053.7 |
| | Amino-ethoxy)-ethoxy]-acetyl)-(γ-Glu)₂-CO—(CH₂)₁₆- | | |
| | CO₂H)AQ-Me4Pal-AFIEYLIEaGPSSGAPPPSGS-NH₂ | | |
| 1017 | Y-Aib-QGTFTSD-4Pal-SQ-αMeL-LD-Orn-K((γ-Glu)₂-CO— | 1023 | 4908.5 |
| | (CH₂)₁₆—CO₂H)AQ-Me4Pal-AFIEYLIEaGPSSGAPPPSGS- | | |
| | NH₂ | | |
| 1018 | Y-Aib-QGTFTSD-4Pal-SQ-αMeL-LD-Orn-K((2-[2-(2- | 1024 | 5111.7 |
| | Amino-ethoxy)-ethoxy]-acetyl)-(γ-Glu)₂-CO—(CH₂)₁₆- | | |
| | CO₂H)AQ-Me4Pal-AFIEYLIEaGPSSGEPPPSGS-NH₂ | | |
| 1019 | Y-Aib-QGTFTSD-4Pal-SQ-αMeL-LD-Orn-K((γ-Glu)₂-CO— | 1025 | 4966.5 |
| | (CH₂)₁₆—CO₂H)AQ-Me4Pal-AFIEYLIEaGPSSGEPPPSGS- | | |
| | NH₂ | | |
| 1020 | Y-Aib-QGTFTSD-4Pal-SQ-αMeL-LD-Orn-K((2-[2-(2- | 1026 | 5095.7 |
| | Amino-ethoxy)-ethoxy]-acetyl)-(γ-Glu)₂-CO—(CH₂)₁₆- | | |
| | CO₂H)AQ-Me4Pal-AFIEYLIEaGPSSGAPPPEGS-NH₂ | | |
| 1021 | Y-Aib-QGTFTSD-4Pal-SQ-αMeL-LD-Orn-K((γ-Glu)₂-CO— | 1027 | 4950.5 |
| | (CH₂)₁₆—CO₂H)AQ-Me4Pal-AFIEYLIEaGPSSGAPPPEGS- | | |
| | NH₂ | | |
| 1022 | Y-Aib-QGTFTSD-4Pal-SQ-αMeL-LD-Orn-K((2-[2-(2- | 1028 | 5182.8 |
| | Amino-ethoxy)-ethoxy]-acetyl)-(γ-Glu)₂-CO—(CH₂)₁₆- | | |
| | CO₂H)AQ-Me4Pal-AFIEYLIEaGPSSGAPPPSGSE-NH₂ | | |
| 1023 | Y-Aib-QGTFTSD-4Pal-SQ-αMeL-LD-Orn-K((γ-Glu)₂-CO— | 1029 | 5037.6 |
| | (CH₂)₁₆—CO₂H)AQ-Me4Pal- | | |
| | AFIEYLIEaGPSSGAPPPSGSE-NH₂ | | |
| 1024 | Y-Aib-QGTFTSD-4Pal-SQ-αMeL-LDKK((2-[2-(2-Amino- | 1030 | 5196.8 |
| | ethoxy)-ethoxy]-acetyl)-(γ-Glu)₂-CO—(CH₂)₁₆—CO₂H)AQ- | | |
| | Me4Pal-AFIEYLIEaGPSSGAPPPSGSE-NH₂ | | |
| 1025 | Y-Aib-QGTFTSD-4Pal-SQ-αMeL-LDKK((γ-Glu)₂-CO— | 1031 | 5051.6 |
| | (CH₂)₁₆—CO₂H)AQ-Me4Pal- | | |
| | AFIEYLIEaGPSSGAPPPSGSE-NH₂ | | |
| 1026 | Y-Aib-QGTFTSD-4Pal-SQ-αMeL-LD-Orn-K((γ-Glu)₂-CO— | 1032 | 5281.8 |
| | (CH₂)₁₆—CO₂H)AQ-Me4Pal-QFIE-αMeY- | | |
| | LIEGGPSSGEPPPSGSEE-NH₂ | | |
| 1027 | Y-Aib-QGTFTSD-4Pal-SQ-αMeL-LD-Orn-K((γ-Glu)₂-CO— | 1033 | 5224.8 |
| | (CH₂)₁₆—CO₂H)AQ-Me4Pal-AFIE-αMeY- | | |
| | LIEGGPSSGEPPPSGSEE-NH₂ | | |
| 1028 | Y-Aib-QGTFTSD-4Pal-SQ-αMeL-LD-Orn-K((γ-Glu)₂- | 1034 | 5224.8 |
| | (Glu)-CO—(CH₂)₁₆—CO₂H)AQ-Me4Pal-AFIE-αMeY- | | |
| | LIEGGPSSGEPPPSGSEE-NH₂ | | |
| 1029 | Y-Aib-QGTFTSD-4Pal-SQ-αMeL-LDKK((2-[2-(2-Amino- | 1035 | 5124.7 |
| | ethoxy)-ethoxy]-acetyl)-(γ-Glu)-CO—(CH₂)₁₆—CO₂H)AQ- | | |
| | Me4Pal-AFIEYLIAGGPSSGAPPPSGSEE-NH₂ | | |
| 1030 | Y-Aib-QGTFTSD-4Pal-SQ-αMeL-LDKK((Glu)-(γ-Glu)- | 1036 | 5108.7 |
| | CO—(CH₂)₁₆—CO₂H)AQ-Me4Pal- | | |
| | AFIEYLIAGGPSSGAPPPSGSEE-NH₂ | | |
| 1031 | Y-Aib-QGTFTSD-4Pal-SQ-αMeL-LD-Orn-K((γ-Glu)₂-CO— | 1037 | 5094.7 |
| | (CH₂)₁₆—CO₂H)AQ-Me4Pal- | | |
| | AFIEYLIAGGPSSGAPPPSGSEE-NH₂ | | |
| 1032 | Y-Aib-QGTFTSD-4Pal-SQ-αMeL-LD-Orn-KK((Glu)-(γ- | 1038 | 5110.7 |
| | Glu)-CO—(CH₂)₁₆—CO₂H)AQ-Me4Pal- | | |
| | AFIEYLIAGGPSSGAPPPSGSEE-NH₂ | | |
| 1033 | Y-Aib-QGTFTSD-4Pal-SQ-αMeL-LD-Orn-K((Glu)₂-(γ- | 1039 | 5094.7 |
| | Glu)-CO—(CH₂)₁₆—CO₂H)AQ-Me4Pal- | | |
| | AFIEYLIAGGPSSGAPPPSGSEE-NH₂ | | |
| 1034 | Y-Aib-QGTFTSD-4Pal-SQ-αMeL-LD-Orn-K(e-(Glu)₂-CO— | 1040 | 5223.8 |
| | (CH₂)₁₆—CO₂H)AQ-Me4Pal- | | |
| | AFIEYLIAGGPSSGAPPPSGSEE-NH₂ | | |
| 1035 | Y-Aib-QGTFTSD-4Pal-SQ-αMeL-LD-Orn-K((γ-Glu)₂-CO— | 1041 | 5094.7 |
| | (CH₂)₁₆—CO₂H)AQ-Me4Pal- | | |
| | AFIEYLIAGGPSSGAPPPSGSEE-NH₂ | | |
| 1036 | Y-Aib-QGTFTSD-4Pal-SQ-αMeL-LDKK((γ-Glu)₂-CO— | 1042 | 5108.7 |
| | (CH₂)₁₆—CO₂H)AQ-Me4Pal- | | |
| | AFIEYLLAGGPSSGAPPPSGSEE-NH₂ | | |
| 1037 | Y-Aib-QGTFTSD-4Pal-SQ-αMeL-LDKK((2-[2-(2-Amino- | 1043 | 5124.7 |
| | ethoxy)-ethoxy]-acetyl)-(γ-Glu)-CO—(CH₂)₁₆—CO₂H)AQ- | | |
| | Me4Pal-AFIEYLLAGGPSSGAPPPSGSEE-NH₂ | | |
| 1038 | Y-Aib-QGTFTSDFSQ-αMeL-LD-Orn-K((2-[2-(2-Amino- | 1044 | 5233.8 |
| | ethoxy)-ethoxy]-acetyl)-(γ-Glu)-CO—(CH₂)₁₆—CO₂H)AQ- | | |
| | Aib-AFIEYLLEaGPSSGAPPPSGSEEE-NH₂ | | |
| 1039 | Y-Aib-QGTFTSDFSQ-αMeL-LD-Orn-K((γ-Glu)₂-CO— | 1045 | 5217.8 |
| | (CH₂)₁₆—CO₂H)AQ-Aib-AFIEYLLEaGPSSGAPPPSGSEEE- | | |
| | NH₂ | | |
| 1040 | Y-Aib-QGTFTSDFSQ-αMeL-LD-Orn-K((γ-Glu)₂-CO— | 1046 | 5216.8 |
| | (CH₂)₁₆—CO₂H)AQ-Aib-AFIE-Me4Pal- | | |
| | LLEaGPSSGAPPPSGSEEE-NH₂ | | |
| 1041 | Y-Aib-QGTFTSD-4Pal-SQ-αMeL-LD-Orn-K((2-[2-(2- | 1047 | 5182.8 |
| | Amino-ethoxy)-ethoxy]-acetyl)-(γ-Glu)-CO—(CH₂)₁₆- | | |
| | CO₂H)AQ-Me4Pal-AFIE-αMeY- | | |
| | LIEGGPSSGAPPPSGSEE-NH₂ | | |
| 1042 | Y-Aib-QGTFTSD-4Pal-SQ-αMeL-LD-Orn-K((2-[2-(2- | 1048 | 5182.8 |
| | Amino-ethoxy)-ethoxy]-acetyl)-(γ-Glu)-CO—(CH₂)₁₆- | | |
| | CO₂H)AQ-Me4Pal-AFIe-αMeY-LIEGGPSSGAPPPSGSEE- | | |
| | NH₂ | | |
| 1043 | Y-Aib-QGTFTSD-4Pal-SQ-αMeL-LD-Orn-K((γ-Glu)₂-CO— | 1049 | 5166.7 |
| | (CH₂)₁₆—CO₂H)AQ-Me4Pal-AFIE-αMeY- | | |
| | LIEGGPSSGAPPPSGSEE-NH₂ | | |
| 1044 | Y-Aib-QGTFTSD-4Pal-SQ-αMeL-LD-Orn-K((γ-Glu)₂-CO— | 1050 | 5166.7 |
| | (CH₂)₁₆—CO₂H)AQ-Me4Pal-AFIe-αMeY- | | |
| | LIEGGPSSGAPPPSGSEE-NH₂ | | |
| 1045 | Y-Aib-QGTFTSD-4Pal-SQ-αMeL-LD-Orn-K((2-[2-(2- | 1051 | 5239.8 |
| | Amino-ethoxy)-ethoxy]-acetyl)-(γ-Glu)-CO—(CH₂)₁₆- | | |
| | CO₂H)AQ-Me4Pal-QFIE-αMeY- | | |
| | LIEGGPSSGAPPPSGSEE-NH₂ | | |
| 1046 | Y-Aib-QGTFTSD-4Pal-SQ-αMeL-LD-Orn-K((γ-Glu)₂-CO— | 1052 | 5223.8 |
| | (CH₂)₁₆—CO₂H)AQ-Me4Pal-QFIE-αMeY- | | |
| | LIEGGPSSGAPPPSGSEE-NH₂ | | |
| 1047 | Y-Aib-QGTFTSDFS-Orn-αMeL-LDKK((γ-Glu)₂-CO— | 1053 | 5074.7 |
| | (CH₂)₁₆—CO₂H)AQ-Aib-AFIEYLLEGGPSSGAPPPGGSEE- | | |
| | NH₂ | | |
| 1048 | Y-Aib-QGTFTSDFS-Orn-αMeL-LD-Orn-K((γ-Glu)₂-CO— | 1054 | 5060.7 |
| | (CH₂)₁₆—CO₂H)AQ-Aib-AFIEYLLEGGPSSGAPPPGGSEE- | | |
| | NH₂ | | |
| 1049 | Y-Aib-QGTFTSDFS-Orn-αMeL-LD-Orn-K((γ-Glu)₂-CO— | 1055 | 5074.7 |
| | (CH₂)₁₆—CO₂H)AQ-Aib-AFIE-αMeY- | | |
| | LLEGGPSSGAPPPGGSEE-NH₂ | | |
| 1050 | Y-Aib-QGTFTSDFS-Orn-αMeL-LD-Orn-K((γ-Glu)₂-CO— | 1056 | 5059.7 |
| | (CH₂)₁₆—CO₂H)AQ-Aib-AFIE-Me4Pal- | | |
| | LLEGGPSSGAPPPGGSEE-NH₂ | | |
| 1051 | Y-Aib-QGTFTSDFS-Orn-αMeL-LDKK((γ-Glu)₂-CO— | 1057 | 5016.6 |
| | (CH₂)₁₆—CO₂H)AQ-Aib-AFIEYLLAGGPSSGAPPPEGSEE- | | |
| | NH₂ | | |
| 1052 | Y-Aib-QGTFTSDFS-Orn-αMeL-LD-Orn-K((γ-Glu)₂-CO— | 1058 | 5002.6 |
| | (CH₂)₁₆—CO₂H)AQ-Aib-AFIEYLLAGGPSSGAPPPEGSEE- | | |
| | NH₂ | | |
| 1053 | Y-Aib-QGTFTSDFS-Orn-αMeL-LD-Orn-K((γ-Glu)₂-CO— | 1059 | 5001.6 |
| | (CH₂)₁₆—CO₂H)AQ-Aib-AFIE-Me4Pal- | | |
| | LLAGGPSSGAPPPEGSEE-NH₂ | | |
| 1054 | Y-Aib-QGTFTSD-4Pal-S-Dab-αMeL-LD-Orn-K((γ-Glu)₂- | 1060 | 5061.6 |
| | CO—(CH₂)₁₆—CO₂H)AQ-Aib-AFIE-Me4Pal- | | |
| | LLAGGPSSGAPPPEGSEE-NH₂ | | |
| 1055 | Y-Aib-QGTFTSD-4Pal-S-Dab-αMeL-LD-Orn-K((γ-Glu)₂- | 1061 | 5138.7 |
| | CO—(CH₂)₁₆—CO₂H)AQ-Me4Pal-AFIE-Me4Pal- | | |
| | LLAGGPSSGAPPPEGSEE-NH₂ | | |
| 1056 | Y-Aib-QGTFTSD-4Pal-SQ-αMeL-LD-Orn-K((γ-Glu)₂-CO— | 1062 | 5094.7 |
| | (CH₂)₁₆—CO₂H)AQ-Me4Pal- | | |
| | AFIEYLLAGGPSSGAPPPSGSEE-NH₂ | | |
| 1057 | Y-Aib-QGTFTSD-4Pal-SQ-αMeL-LD-Orn-K((Glu)-(γ- | 1063 | 5094.7 |
| | Glu)-CO—(CH₂)₁₆—CO₂H)AQ-Me4Pal- | | |
| | AFIEYLLAGGPSSGAPPPSGSEE-NH₂ | | |
| 1058 | Y-Aib-QGTFTSD-4Pal-SQ-αMeL-LD-Orn-K(e-(γ-Glu)- | 1064 | 5166.7 |
| | CO—(CH₂)₁₆—CO₂H)AQ-Me4Pal-AFIE-αMeY- | | |
| | LLEGGPSSGAPPPSGSEE-NH₂ | | |
| 1059 | Y-Aib-QGTFTSD-4Pal-S-Orn-αMeL-LD-Orn-K((γ-Glu)₂- | 1065 | 5080.7 |
| | CO—(CH₂)₁₆—CO₂H)AQ-Me4Pal- | | |
| | AFIEYLIAGGPSSGAPPPSGSEE-NH₂ | | |
| 1060 | Y-Aib-QGTFTSD-4Pal-S-Orn-αMeL-LDKK((Glu)-(γ-Glu)- | 1066 | 5094.7 |
| | CO—(CH₂)₁₆—CO₂H)AQ-Me4Pal- | | |
| | AFIEYLIAGGPSSGAPPPSGSEE-NH₂ | | |
| 1061 | Y-Aib-QGTFTSD-4Pal-S-Orn-αMeL-LD-Orn-K((Glu)-(γ- | 1067 | 5080.7 |
| | Glu)-CO—(CH₂)₁₆—CO₂H)AQ-Me4Pal- | | |
| | AFIEYLIAGGPSSGAPPPSGSEE-NH₂ | | |
| 1062 | Y-Aib-QGTFTSD-4Pal-S-Orn-αMeL-LD-Orn-K((Glu)₂-(γ- | 1068 | 5209.8 |
| | Glu)-CO—(CH₂)₁₆—CO₂H)AQ-Me4Pal- | | |
| | AFIEYLIAGGPSSGAPPPSGSEE-NH₂ | | |
| 1063 | Y-Aib-QGTFTSD-4Pal-S-Orn-αMeL-LD-Orn-K(e-(γ-Glu)- | 1069 | 5080.7 |
| | CO—(CH₂)₁₆—CO₂H)AQ-Me4Pal- | | |
| | AFIEYLIAGGPSSGAPPPSGSEE-NH₂ | | |
| 1064 | Y-Aib-QGTFTSD-4Pal-S-Orn-αMeL-LD-Orn-K((γ-Glu)₂- | 1070 | 5075.7 |
| | CO—(CH₂)₁₆—CO₂H)AQ-Aib-AFIE-αMeY- | | |
| | LLEGGPSSGAPPPSGSEE-NH₂ | | |
| 1065 | Y-Aib-QGTFTSD-4Pal-S-Orn-αMeL-LD-Orn-K(e-(γ-Glu)- | 1071 | 5075.7 |
| | CO—(CH₂)₁₆—CO₂H)AQ-Aib-AFIE-αMeY- | | |
| | LLEGGPSSGAPPPSGSEE-NH₂ | | |
| 1066 | Y-Aib-QGTFTSD-4Pal-S-Orn-αMeL-LD-Orn-K((Glu)-(γ- | 1072 | 5075.7 |
| | Glu)-CO—(CH₂)₁₆—CO₂H)AQ-Aib-AFIE-αMeY- | | |
| | LLEGGPSSGAPPPSGSEE-NH₂ | | |
| 1067 | Y-Aib-QGTFTSD-4Pal-S-Orn-αMeL-LD-Orn-K((Glu)₂-(γ- | 1073 | 5204.8 |
| | Glu)-CO—(CH₂)₁₆—CO₂H)AQ-Aib-AFIE-αMeY- | | |
| | LLEGGPSSGAPPPSGSEE-NH₂ | | |
| 1068 | Y-Aib-QGTFTSD-4Pal-S-Orn-αMeL-LD-Orn-K((γ-Glu)₂- | 1074 | 5080.7 |
| | CO—(CH₂)₁₆—CO₂H)AQ-Me4Pal- | | |
| | AFIEYLLAGGPSSGAPPPSGSEE-NH₂ | | |
| 1069 | Y-Aib-QGTFTSD-4Pal-S-Orn-αMeL-LD-Or-K((γ-Glu)₂- | 1075 | 5003.6 |
| | CO—(CH₂)₁₆—CO₂H)AQ-Aib- | | |
| | AFIEYLLAGGPSSGAPPPSGSEE-NH₂ | | |
| 1070 | Y-Aib-QGTFTSD-4Pal-SQ-αMeL-LD-Orn-K(e-(γ-Glu)- | 1076 | 5166.7 |
| | CO—(CH₂)₁₆—CO₂H)AQ-Me4Pal-AFIE-αMeY- | | |
| | LIEGGPSSGAPPPSGSEE-NH₂ | | |
| 1071 | Y-Aib-QGTFTSD-4Pal-SQ-αMeL-LD-Orn-K(e-(γ-Glu)- | 1077 | 5295.9 |
| | CO—(CH₂)₁₆—CO₂H)AQ-Me4Pal-AFIE-αMeY- | | |
| | LIEGGPSSGAPPPSGSEEE-NH₂ | | |
| 1072 | Y-Aib-QGTFTSD-4Pal-SQ-αMeL-LD-Orn-K(e-(γ-Glu)- | 1078 | 5165.8 |
| | CO—(CH₂)₁₆—CO₂H)AQ-Me4Pal-AFIQ-αMeY- | | |
| | LIEGGPSSGAPPPSGSEE-NH₂ | | |
| 1073 | Y-Aib-QGTFTSD-4Pal-SQ-αMeL-LD-Orn-K(e-(γ-Glu)- | 1079 | 5294.9 |
| | CO—(CH₂)₁₆—CO₂H)AQ-Me4Pal-AFIQ-αMeY- | | |
| | LIEGGPSSGAPPPSGSEEE-NH₂ | | |
| 1074 | Y-Aib-QGTFTSD-4Pal-SQ-αMeL-LD-Orn-K(e-(γ-Glu)- | 1080 | 5108.7 |
| | CO—(CH₂)₁₆—CO₂H)AQ-Me4Pal-AFIE-αMeY- | | |
| | LIAGGPSSGAPPPSGSEE-NH₂ | | |
| 1075 | Y-Aib-QGTFTSD-4Pal-SQ-αMeL-LD-Orn-K(e-(γ-Glu)- | 1081 | 5237.8 |
| | CO—(CH₂)₁₆—CO₂H)AQ-Me4Pal-AFIE-αMeY- | | |
| | LIAGGPSSGAPPPSGSEEE-NH₂ | | |
| 1076 | Y-Aib-QGTFTSD-4Pal-SQ-αMeL-LD-Orn-K(e-(γ-Glu)- | 1082 | 5294.9 |
| | CO—(CH₂)₁₆—CO₂H)AQ-Me4Pal-AFIQ-αMeY- | | |
| | LIEGGPSSGAPPPSGSEEE-NH₂ | | |
| 1077 | Y-Aib-QGTFTSD-4Pal-SQ-αMeL-LD-Orn-K((Glu)-(γ- | 1083 | 5294.9 |
| | Glu)-CO—(CH₂)₁₆—CO₂H)AQ-Me4Pal-AFIQ-αMeY- | | |
| | LIEGGPSSGAPPPSGSEEE-NH₂ | | |
| 1078 | Y-Aib-QGTFTSD-4Pal-SQ-αMeL-LD-Orn-KK((Glu)₂-(γ- | 1084 | 5424.0 |
| | Glu)-CO—(CH₂)₁₆—CO₂H)AQ-Me4Pal-AFIQ-αMeY- | | |
| | LIEGGPSSGAPPPSGSEEE-NH₂ | | |
| 1079 | Y-Aib-QGTFTSD-4Pal-SQ-αMeL-LD-Orn-K((Glu)-(γ- | 1085 | 5237.8 |
| | Glu)₂-CO—(CH₂)₁₆—CO₂H)AQ-Me4Pal-AFIE-αMeY- | | |
| | LIAGGPSSGAPPPSGSEEE-NH₂ | | |
| 1080 | Y-Aib-QGTFTSD-4Pal-SQ-αMeL-LD-Orn-K((Glu)₂-(γ- | 1086 | 5366.9 |
| | Glu)₂-CO—(CH₂)₁₆—CO₂H)AQ-Me4Pal-AFIE-αMeY- | | |
| | LIAGGPSSGAPPPSGSEEE-NH₂ | | |
| 1081 | Y-Aib-QGTFTSD-4Pal-SQ-αMeL-LD-Orn-K(e-(γ-Glu)- | 1087 | 5294.9 |
| | CO—(CH₂)₁₆—CO₂H)AQ-Me4Pal-QFIE-αMeY- | | |
| | LIAGGPSSGAPPPSGSEEE-NH₂ | | |
| 1082 | Y-Aib-QGTFTSD-4Pal-SQ-αMeL-LD-Orn-K(e-(γ-Glu)- | 1088 | 5237.8 |
| | CO—(CH₂)₁₆—CO₂H)AQ-Me4Pal-AFIE-αMeY- | | |
| | LLAGGPSSGAPPPSGSEEE-NH₂ | | |
| 1083 | Y-Aib-QGTFTSD-4Pal-SQ-αMeL-LD-Orn-K(e-(γ-Glu)- | 1089 | 5294.9 |
| | CO—(CH₂)₁₆—CO₂H)AQ-Me4Pal-AFIQ-αMeY- | | |
| | LLEGGPSSGAPPPSGSEEE-NH₂ | | |
| 1084 | Y-Aib-QGTFTSD-4Pal-SQ-αMeL-LD-Orn-K(e-(γ-Glu)- | 1090 | 5294.9 |
| | CO—(CH₂)₁₆—CO₂H)AQ-Me4Pal-QFIE-αMeY- | | |
| | LLAGGPSSGAPPPSGSEEE-NH₂ | | |
| 1085 | Y-Aib-QGTFTSD-4Pal-SQ-αMeL-LDKK(e-(γ-Glu)-CO— | 1091 | 4892.5 |
| | (CH₂)₁₆—CO₂H)AQ-Me4Pal-AFIEYLIAGGPSSGAPPPSGE- | | |
| | NH₂ | | |
| 1086 | Y-Aib-QGTFTSD-4Pal-SQ-αMeL-LDKK((Glu)-(γ-Glu)- | 1092 | 4892.5 |
| | CO—(CH₂)₁₆—CO₂H)AQ-Me4Pal- | | |
| | AFIEYLIAGGPSSGAPPPSGE-NH₂ | | |
| 1087 | Y-Aib-QGTFTSD-4Pal-SQ-αMeL-LDKK((Glu)₂-(γ-Glu)- | 1093 | 5021.6 |
| | CO—(CH₂)₁₆—CO₂H)AQ-Me4Pal- | | |
| | AFIEYLIAGGPSSGAPPPSGE-NH₂ | | |
| 1088 | Y-Aib-QGTFTSD-4Pal-SQ-αMeL-LDKK(e-(γ-Glu)-CO— | 1094 | 5150.7 |
| | (CH₂)₁₆—CO₂H)AQ-Me4Pal- | | |
| | AFIEYLIAGGPSSGAPPPSGEEE-NH₂ | | |
| 1089 | Y-Aib-QGTFTSD-4Pal-SQ-αMeL-LDKK((Glu)-(γ-Glu)- | 1095 | 5150.7 |
| | CO—(CH₂)₁₆—CO₂H)AQ-Me4Pal- | | |
| | AFIEYLIAGGPSSGAPPPSGEEE-NH₂ | | |
| 1090 | Y-Aib-QGTFTSD-4Pal-SQ-αMeL-LDKK((Glu)₂-(γ-Glu)- | 1096 | 5279.9 |
| | CO—(CH₂)₁₆—CO₂H)AQ-Me4Pal- | | |
| | AFIEYLIAGGPSSGAPPPSGEEE-NH₂ | | |
| 1091 | Y-Aib-QGTFTSD-4Pal-SQ-αMeL-LD-Orn-K((γ-Glu)₂-CO— | 1097 | 5122.7 |
| | (CH₂)₁₆—CO₂H)AQ-Me4Pal-AFIEYLIA-Aib- | | |
| | GPSSGAPPPSGSEE-NH₂ | | |
| 1092 | Y-Aib-QGTFTSD-4Pal-SQ-αMeL-LD-Orn-K((γ-Glu)₂-CO— | 1098 | 5180.8 |
| | (CH₂)₁₆—CO₂H)AQ-Me4Pal-AFIEYLIA-Aib- | | |
| | GPSSGEPPPSGSEE-NH₂ | | |
| 1093 | Y-Aib-QGTFTSD-4Pal-SQ-αMeL-LD-Orn-K((γ-Glu)₂-CO— | 1099 | 5252.8 |
| | (CH₂)₁₆—CO₂H)AQ-Me4Pal-AFIE-αMeY-LIE-Aib- | | |
| | GPSSGEPPPSGSEE-NH₂ | | |
| 1094 | Y-Aib-QGTFTSD-4Pal-SQ-αMeL-LD-Orn-K((γ-Glu)₂-CO— | 1100 | 5194.8 |
| | (CH₂)₁₆—CO₂H)AQ-Me4Pal-AFIE-αMeY-LIA-Aib- | | |
| | GPSSGEPPPSGSEE-NH₂ | | |
| 1095 | Y-Aib-QGTFTSD-4Pal-SQ-αMeL-LD-Orn-K((2-[2-(2- | 1101 | 5196.8 |
| | Amino-ethoxy)-ethoxy]-acetyl)-(γ-Glu)-CO—(CH₂)₁₆- | | |
| | CO₂H)AQ-Me4Pal-AFIEYLIA-Aib-GPSSGEPPPSGSEE- | | |
| | NH₂ | | |
| 1096 | Y-Aib-QGTFTSD-4Pal-SQ-αMeL-LD-Orn-K((2-[2-(2- | 1102 | 5253.9 |
| | Amino-ethoxy)-ethoxy]-acetyl)-(γ-Glu)-CO—(CH₂)₁₆- | | |
| | CO₂H)AQ-Me4Pal-AFIEYLIQ-Aib-GPSSGEPPPSGSEE- | | |
| | NH₂ | | |
| 1097 | Y-Aib-QGTFTSD-4Pal-SQ-αMeL-LD-Orn-K((2-[2-(2- | 1103 | 5268.9 |
| | Amino-ethoxy)-ethoxy]-acetyl)-(γ-Glu)-CO—(CH₂)₁₆- | | |
| | CO₂H)AQ-Me4Pal-AFIE-αMeY-LIE-Aib- | | |
| | GPSSGEPPPSGSEE-NH₂ | | |
| 1098 | Y-Aib-QGTFTSD-4Pal-SQ-αMeL-LD-Orn-K((2-[2-(2- | 1104 | 5325.9 |
| | Amino-ethoxy)-ethoxy]-acetyl)-(γ-Glu)-CO—(CH₂)₁₆- | | |
| | CO₂H)AQ-Me4Pal-QFIE-αMeY-LIE-Aib- | | |
| | GPSSGEPPPSGSEE-NH₂ | | |
| 1099 | Y-Aib-QGTFTSD-4Pal-SQ-αMeL-LD-Orn-K((2-[2-(2- | 1105 | 5210.8 |
| | Amino-ethoxy)-ethoxy]-acetyl)-(γ-Glu)-CO—(CH₂)₁₆- | | |
| | CO₂H)AQ-Me4Pal-AFIE-αMeY-LIA-Aib- | | |
| | GPSSGEPPPSGSEE-NH₂ | | |
| 1100 | Y-Aib-QGTFTSD-4Pal-SQ-αMeL-LD-Orn-K((2-[2-(2- | 1106 | 5267.9 |
| | Amino-ethoxy)-ethoxy]-acetyl)-(γ-Glu)-CO—(CH₂)₁₆- | | |
| | CO₂H)AQ-Me4Pal-QFIE-αMeY-LIA-Aib- | | |
| | GPSSGEPPPSGSEE-NH₂ | | |
| 1101 | Y-Aib-QGTFTSD-4Pal-SQ-αMeL-LD-Orn-K(e-(γ-Glu)- | 1107 | 5180.8 |
| | CO—(CH₂)₁₆—CO₂H)AQ-Me4Pal-AFIEYLIA-Aib- | | |
| | GPSSGEPPPSGSEE-NH₂ | | |
| 1102 | Y-Aib-QGTFTSD-4Pal-SQ-αMeL-LD-Orn-K(e-(γ-Glu)- | 1108 | 5237.8 |
| | CO—(CH₂)₁₆—CO₂H)AQ-Me4Pal-AFIEYLIQ-Aib- | | |
| | GPSSGEPPPSGSEE-NH₂ | | |
| 1103 | Y-Aib-QGTFTSD-4Pal-SQ-αMeL-LD-Orn-K(e-(γ-Glu)- | 1109 | 5252.8 |
| | CO—(CH₂)₁₆—CO₂H)AQ-Me4Pal-AFIE-αMeY-LIE-Aib- | | |
| | GPSSGEPPPSGSEE-NH₂ | | |
| 1104 | Y-Aib-QGTFTSD-4Pal-SQ-αMeL-LD-Orn-K(e-(γ-Glu)- | 1110 | 5309.9 |
| | CO—(CH₂)₁₆—CO₂H)AQ-Me4Pal-QFIE-αMeY-LIE-Aib- | | |
| | GPSSGEPPPSGSEE-NH₂ | | |
| 1105 | Y-Aib-QGTFTSD-4Pal-SQ-αMeL-LD-Orn-K(e-(γ-Glu)- | 1111 | 5194.8 |
| | CO—(CH₂)₁₆—CO₂H)AQ-Me4Pal-AFIE-αMeY-LIA-Aib- | | |
| | GPSSGEPPPSGSEE-NH₂ | | |
| 1106 | Y-Aib-QGTFTSD-4Pal-SQ-αMeL-LD-Orn-K(e-(γ-Glu)- | 1112 | 5251.8 |
| | CO—(CH₂)₁₆—CO₂H)AQ-Me4Pal-QFIE-αMeY-LIA-Aib- | | |
| | GPSSGEPPPSGSEE-NH₂ | | |
| 1107 | Y-Aib-QGTFTSD-4Pal-SQ-αMeL-LD-Orn-K((γ-Glu)₂-CO— | 1113 | 5136.8 |
| | (CH₂)₁₆—CO₂H)AQ-Me4Pal-AFIE-αMeY-LIA-Aib- | | |
| | GPSSGAPPPSGSEE-NH₂ | | |
| 1108 | Y-Aib-QGTFTSD-4Pal-SQ-αMeL-LD-Orn-K((2-[2-(2- | 1114 | 5210.8 |
| | Amino-ethoxy)-ethoxy]-acetyl)-(γ-Glu)-CO—(CH₂)₁₆- | | |
| | CO₂H)AQ-Me4Pal-AFIE-αMeY-LIE-Aib- | | |
| | GPSSGAPPPSGSEE-NH₂ | | |
| 1109 | Y-Aib-QGTFTSD-4Pal-SQ-αMeL-LD-Orn-K((2-[2-(2- | 1115 | 5267.9 |
| | Amino-ethoxy)-ethoxy]-acetyl)-(γ-Glu)-CO—(CH₂)₁₆- | | |
| | CO₂H)AQ-Me4Pal-QFIE-αMeY-LIE-Aib- | | |
| | GPSSGAPPPSGSEE-NH₂ | | |
| 1110 | Y-Aib-QGTFTSD-4Pal-SQ-αMeL-LD-Orn-K((2-[2-(2- | 1116 | 5152.8 |
| | Amino-ethoxy)-ethoxy]-acetyl)-(γ-Glu)-CO—(CH₂)₁₆- | | |
| | CO₂H)AQ-Me4Pal-AFIE-αMeY-LIA-Aib- | | |
| | GPSSGAPPPSGSEE-NH₂ | | |
| 1111 | Y-Aib-QGTFTSD-4Pal-SQ-αMeL-LD-Orn-K((2-[2-(2- | 1117 | 5209.8 |
| | Amino-ethoxy)-ethoxy]-acetyl)-(γ-Glu)-CO—(CH₂)₁₆- | | |
| | CO₂H)AQ-Me4Pal-QFIE-αMeY-LIA-Aib- | | |
| | GPSSGAPPPSGSEE-NH₂ | | |
| 1112 | Y-Aib-QGTFTSD-4Pal-SQ-αMeL-LD-Orn-K(e-(γ-Glu)- | 1118 | 5194.8 |
| | CO—(CH₂)₁₆—CO₂H)AQ-Me4Pal-AFIE-αMeY-LIE-Aib- | | |
| | GPSSGAPPPSGSEE-NH₂ | | |
| 1113 | Y-Aib-QGTFTSD-4Pal-SQ-αMeL-LD-Orn-K(e-(γ-Glu)- | 1119 | 5251.8 |
| | CO—(CH₂)₁₆—CO₂H)AQ-Me4Pal-QFIE-αMeY-LIE-Aib- | | |
| | GPSSGAPPPSGSEE-NH₂ | | |
| 1114 | Y-Aib-QGTFTSD-4Pal-SQ-αMeL-LD-Orn-K(e-(γ-Glu)- | 1120 | 5136.8 |
| | CO—(CH₂)₁₆—CO₂H)AQ-Me4Pal-AFIE-αMeY-LIA-Aib- | | |
| | GPSSGAPPPSGSEE-NH₂ | | |
| 1115 | Y-Aib-QGTFTSD-4Pal-SQ-αMeL-LD-Orn-K(e-(γ-Glu)- | 1121 | 5193.8 |
| | CO—(CH₂)₁₆—CO₂H)AQ-Me4Pal-QFIE-αMeY-LIA-Aib- | | |
| | GPSSGAPPPSGSEE-NH₂ | | |
| 1116 | Y-Aib-QGTFTSD-4Pal-SQ-αMeL-LDKK((2-[2-(2-Amino- | 1122 | 5094.8 |
| | ethoxy)-ethoxy]-acetyl)-(γ-Glu)-CO—(CH₂)₁₆—CO₂H)AQ- | | |
| | Me4Pal-AFIE-Me4Pal-LIE-Aib-GPSSGAPPPSGTE-NH₂ | | |
| 1117 | Y-Aib-QGTFTSD-4Pal-SQ-αMeL-LDKK((2-[2-(2-Amino- | 1123 | 5151.8 |
| | ethoxy)-ethoxy]-acetyl)-(γ-Glu)-CO—(CH₂)₁₆—CO₂H)AQ- | | |
| | Me4Pal-AFIE-Me4Pal-LIE-Aib-GPSSGAPPPSGTGE-NH₂ | | |
| 1118 | Y-Aib-QGTFTSD-4Pal-SQ-αMeL-LDKK((γ-Glu)₂-CO— | 1124 | 5078.7 |
| | (CH₂)₁₆—CO₂H)AQ-Me4Pal-AFIE-Me4Pal-LIE-Aib- | | |
| | GPSSGAPPPSGTE-NH₂ | | |
| 1119 | Y-Aib-QGTFTSD-4Pal-SQ-αMeL-LDKK((γ-Glu)₂-CO— | 1125 | 5078.7 |
| | (CH₂)₁₆—CO₂H)AQ-Me4Pal-AFIE-Me4Pal-LIE-Aib- | | |
| | GPSSGAPPPSGTGE-NH₂ | | |
| 1120 | Y-Aib-QGTFTSD-4Pal-SQ-αMeL-LDKK((γ-Glu)₂-CO— | 1126 | 5135.8 |
| | (CH₂)₁₆—CO₂H)AQ-Me4Pal-AFIE-Me4Pal-LIE-Aib- | | |
| | GPSSGAPPPSGE-NH₂ | | |
| 1121 | Y-Aib-QGTFTSD-4Pal-SQ-αMeL-LDKK((γ-Glu)₂-CO— | 1127 | 4977.6 |
| | (CH₂)₁₆—CO₂H)AQ-Me4Pal-AFIE-Me4Pal-LIE-Aib- | | |
| | GPSSGAPPPSGEE-NH₂ | | |
| 1122 | Y-Aib-QGTFTSD-4Pal-SQ-αMeL-LDKK((γ-Glu)₂-CO— | 1128 | 5106.7 |
| | (CH₂)₁₆—CO₂H)AQ-Me4Pal-AFIE-Me4Pal-LIE-Aib- | | |
| | GPSSGAPPPSGSEE-NH₂ | | |
| 1123 | Y-Aib-QGTFTSD-4Pal-SQ-αMeL-LDKK((γ-Glu)₂-CO— | 1129 | 5193.8 |
| | (CH₂)₁₆—CO₂H)AQ-Me4Pal-AFIE-Me4Pal-LIE-Aib- | | |
| | GPSSGEPPPSGE-NH₂ | | |
| 1124 | Y-Aib-QGTFTSD-4Pal-SQ-αMeL-LDKK((γ-Glu)₂-CO— | 1130 | 5035.6 |
| | (CH₂)₁₆—CO₂H)AQ-Me4Pal-AFIE-Me4Pal-LIE-Aib- | | |
| | GPSSGEPPPSGEEE-NH₂ | | |
| 1125 | Y-Aib-QGTFTSD-4Pal-SQ-αMeL-LDKK((γ-Glu)₂-CO— | 1131 | 5293.9 |
| | (CH₂)₁₆—CO₂H)AQ-Me4Pal-AFIE-Me4Pal-LIE-Aib- | | |
| | GPSSGEPPPSGSEE-NH₂ | | |
| 1126 | Y-Aib-QGTFTSD-4Pal-SQ-αMeL-LDKK((γ-Glu)₂-CO— | 1132 | 5251.8 |
| | (CH₂)₁₆—CO₂H)AQ-Me4Pal-AFIEYLIE-Aib- | | |
| | GPSSGAPPPSGTE-NH₂ | | |
| 1127 | Y-Aib-QGTFTSD-4Pal-SQ-αMeL-LDKK((γ-Glu)₂-CO— | 1133 | 5079.7 |
| | (CH₂)₁₆—CO₂H)AQ-Me4Pal-AFIEYLIE-Aib- | | |
| | GPSSGAPPPSGTGE-NH₂ | | |
| 1128 | Y-Aib-QGTFTSD-4Pal-SQ-αMeL-LD-Orn-K(e-(γ-Glu)- | 1134 | 5136.8 |
| | CO—(CH₂)₁₆—CO₂H)AQ-Me4Pal-QFIQ-αMeY-LIA-Aib- | | |
| | GPSSGEPPPSGGGE-NH₂ | | |
| 1129 | Y-Aib-QGTFTSD-4Pal-SQ-αMeL-LD-Orn-K(e-(γ-Glu)- | 1135 | 5206.8 |
| | CO—(CH₂)₁₆—CO₂H)AQ-Me4Pal-QFIQ-αMeY-LIE-Aib- | | |
| | GPSSGEPPPSGGGE-NH₂ | | |
| 1130 | Y-Aib-QGTFTSD-4Pal-SQ-αMeL-LD-Orn-K(e-(γ-Glu)- | 1136 | 5091.7 |
| | CO—(CH₂)₁₆—CO₂H)AQ-Me4Pal-AFIQ-αMeY-LIA-Aib- | | |
| | GPSSGEPPPSGGGE-NH₂ | | |
| 1131 | Y-Aib-QGTFTSD-4Pal-SQ-αMeL-LD-Orn-K(e-(γ-Glu)- | 1137 | 5149.8 |
| | CO—(CH₂)₁₆—CO₂H)AQ-Me4Pal-AFIQ-αMeY-LIE-Aib- | | |
| | GPSSGEPPPSGGGE-NH₂ | | |
| 1132 | Y-Aib-QGTFTSD-4Pal-αMeS-Q-αMeL-LD-Orn-K(e-(γ- | 1138 | 5104.8 |
| | Glu)-CO—(CH₂)₁₆—CO₂H)AQ-Me4Pal-QFIQ-αMeY-LIA- | | |
| | Aib-GPSSGAPPPSGGGE-NH₂ | | |
| 1133 | Y-Aib-QGTFTSD-4Pal-αMeS-Q-αMeL-LD-Orn-K(e-(γ- | 1139 | 5134.7 |
| | Glu)-CO—(CH₂)₁₆—CO₂H)AQ-Me4Pal-QFIQ-αMeY- | | |
| | LIAGGPSSGEPPPSGGGE-NH₂ | | |
| 1134 | NMeY-Aib-QGTFTSD-4Pal-αMeS-Q-αMeL-LD-Orn-K(e- | 1140 | 5176.8 |
| | (γ-Glu)-CO—(CH₂)₁₆—CO₂H)AQ-Me4Pal-QFIQ-αMeY-LIE- | | |
| | Aib-GPSSGAPPPSGGGE-NH₂ | | |
| 1135 | NMeY-Aib-QGTFTSD-4Pal-αMeS-I-αMeL-LD-Orn-K(e- | 1141 | 5161.9 |
| | (γ-Glu)-CO—(CH₂)₁₆—CO₂H)AQ-Me4Pal-QFIQ-αMeY-LIE- | | |
| | Aib-GPSSGAPPPSGGGE-NH₂ | | |
| 1136 | Y-Aib-QGTFTSD-4Pal-αMeS-I-αMeL-LD-Orn-K(e-(γ- | 1142 | 5147.8 |
| | Glu)-CO—(CH₂)₁₆—CO₂H)AQ-Me4Pal-QFIQ-αMeY-LIE- | | |
| | Aib-GPSSGAPPPSGGGE-NH₂ | | |
| 1137 | Y-Aib-QGTFTSD-4Pal-αMeS-Q-αMeL-LD-Orn-K(e-(γ- | 1143 | 5099.7 |
| | Glu)-CO—(CH₂)₁₆—CO₂H)AQIvaQFIQ-αMeY-LIE-Aib- | | |
| | GPSSGAPPPSGGGE-NH₂ | | |
| 1138 | Y-Aib-QGTFTSD-4Pal-αMeS-Q-αMeL-LD-Orn-K(e-(γ- | 1144 | 5162.8 |
| | Glu)-CO—(CH₂)₁₆—CO₂H)AQ-Me4Pal-QFIQ-αMeY-LLE- | | |
| | Aib-GPSSGAPPPSGGGE-NH₂ | | |
| 1139 | Y-Aib-QGTFTSD-4Pal-αMeS-Q-αMeL-LD-Orn-K(e-(γ- | 1145 | 5099.7 |
| | Glu)-CO—(CH₂)₁₆—CO₂H)AQIvaQFIQ-αMeY-LLE-Aib- | | |
| | GPSSGAPPPSGGGE-NH₂ | | |
| 1140 | Y-Aib-QGT-αMeF(2F)-TSD-4Pal-αMeS-I-αMeL-LD-Orn- | 1146 | 5055.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPSGE-NH₂ | | |
| 1141 | Y-Aib-QGT-αMeF(2F)-TSD-4Pal-αMeS-I-αMeL-LD-Orn- | 1147 | 5113.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGEPPPSGE-NH₂ | | |
| 1142 | Y-Aib-QGT-αMeF(2F)-TSD-4Pal-αMeS-I-αMeL-LD-Orn- | 1148 | 5069.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPTGE-NH₂ | | |
| 1143 | Y-Aib-QGT-αMeF(2F)-TSD-4Pal-αMeS-I-αMeL-LD-Orn- | 1149 | 5127.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGEPPPTGE-NH₂ | | |
| 1144 | Y-Aib-QGT-αMeF(2F)-TSD-4Pal-αMeS-I-αMeL-LD-Orn- | 1150 | 5025.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPGGE-NH₂ | | |
| 1145 | Y-Aib-QGT-αMeF(2F)-TSD-4Pal-αMeS-I-αMeL-LD-Orn- | 1151 | 5083.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGEPPPGGE-NH₂ | | |
| 1146 | Y-Aib-QGT-αMeF(2F)-TSD-4Pal-αMeS-I-αMeL-LD-Orn- | 1152 | 5127.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPESE-NH₂ | | |
| 1147 | Y-Aib-QGT-αMeF(2F)-TSD-4Pal-αMeS-I-αMeL-LD-Orn- | 1153 | 5185.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGEPPPESE-NH₂ | | |
| 1148 | Y-Aib-QGT-αMeF(2F)-TSD-4Pal-αMeS-I-αMeL-LD-Orn- | 1154 | 5097.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPEGE-NH₂ | | |
| 1149 | Y-Aib-QGT-αMeF(2F)-TSD-4Pal-αMeS-I-αMeL-LD-Orn- | 1155 | 5155.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGEPPPEGE-NH₂ | | |
| 1150 | Y-Aib-QGT-αMeF(2F)-TSD-4Pal-αMeS-I-αMeL-LD-Orn- | 1156 | 5039.7 |
| | K((γ-Glu)₂-CO—(CH₂)₁₆—CO₂H)AQ-Me4Pal-EFIe-αMeY- | | |
| | LIEGGPSSGAPPPSGE-NH₂ | | |
| 1151 | Y-Aib-QGT-αMeF(2F)-TSD-4Pal-αMeS-I-αMeL-LD-Orn- | 1157 | 5097.7 |
| | K((γ-Glu)₂-CO—(CH₂)₁₆—CO₂H)AQ-Me4Pal-EFIe-αMeY- | | |
| | LIEGGPSSGEPPPSGE-NH₂ | | |
| 1152 | Y-Aib-QGT-αMeF(2F)-TSD-4Pal-αMeS-I-αMeL-LD-Orn- | 1158 | 5055.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGAPPPTGE-NH₂ | | |
| 1153 | Y-Aib-QGT-αMeF(2F)-TSD-4Pal-αMeS-I-αMeL-LD-Orn- | 1159 | 5113.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGEPPPTGE-NH₂ | | |
| 1154 | Y-Aib-QGT-αMeF(2F)-TSD-4Pal-αMeS-I-αMeL-LD-Orn- | 1160 | 5011.6 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGAPPPGGE-NH₂ | | |
| 1155 | Y-Aib-QGT-αMeF(2F)-TSD-4Pal-αMeS-I-αMeL-LD-Orn- | 1161 | 5069.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGEPPPGGE-NH₂ | | |
| 1156 | Y-Aib-QGT-αMeF(2F)-TSD-4Pal-αMeS-I-αMeL-LD-Orn- | 1162 | 5113.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGAPPPESE-NH₂ | | |
| 1157 | Y-Aib-QGT-αMeF(2F)-TSD-4Pal-αMeS-I-αMeL-LD-Orn- | 1163 | 5171.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGEPPPESE-NH₂ | | |
| 1158 | Y-Aib-QGT-αMeF(2F)-TSD-4Pal-αMeS-I-αMeL-LD-Orn- | 1164 | 5083.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGAPPPEGE-NH₂ | | |
| 1159 | Y-Aib-QGT-αMeF(2F)-TSD-4Pal-αMeS-I-αMeL-LD-Orn- | 1165 | 5141.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGEPPPEGE-NH₂ | | |
| 1160 | Y-Aib-QGT-αMeF(2F)-TSD-4Pal-αMeS-I-αMeL-LD-Orn- | 1166 | 5025.6 |
| | K((γ-Glu)₂-CO—(CH₂)₁₆—CO₂H)AQ-Me4Pal-EFIe-αMeY- | | |
| | LVEGGPSSGAPPPSGE-NH₂ | | |
| 1161 | Y-Aib-QGT-αMeF(2F)-TSD-4Pal-αMeS-I-αMeL-LD-Orn- | 1167 | 5083.7 |
| | K((γ-Glu)₂-CO—(CH₂)₁₆—CO₂H)AQ-Me4Pal-EFIe-αMeY- | | |
| | LVEGGPSSGEPPPSGE-NH₂ | | |
| 1162 | Y-Aib-QGT-αMeF(2F)-TSD-4Pal-αMeS-I-αMeL-LD-Orn- | 1168 | 5112.7 |
| | K((γ-Glu)₂-CO—(CH₂)₁₆—CO₂H)AQ-Me4Pal-EFIe-αMeY- | | |
| | LVEGGPSSGAPPPSGSE-NH₂ | | |
| 1163 | Y-Aib-QGT-αMeF(2F)-TSD-4Pal-αMeS-I-αMeL-LD-Orn- | 1169 | 5170.7 |
| | K((γ-Glu)₂-CO—(CH₂)₁₆—CO₂H)AQ-Me4Pal-EFIe-αMeY- | | |
| | LVEGGPSSGEPPPSGSE-NH₂ | | |
| 1164 | Y-Aib-QGT-αMeF-TSD-4Pal-αMeS-I-αMeL-LD-Orn-K((2- | 1170 | 5037.7 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LIEGGPSSGAPPPSGE- | | |
| | NH₂ | | |
| 1165 | Y-Aib-QGT-αMeF-TSD-4Pal-αMeS-I-αMeL-LD-Orn-K((2- | 1171 | 5095.7 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LIEGGPSSGEPPPSGE- | | |
| | NH₂ | | |
| 1166 | Y-Aib-QGT-αMeF-TSD-4Pal-αMeS-I-αMeL-LD-Orn-K((2- | 1172 | 5124.8 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LIEGGPSSGAPPPSGSE- | | |
| | NH₂ | | |
| 1167 | Y-Aib-QGT-αMeF-TSD-4Pal-αMeS-I-αMeL-LD-Orn-K((2- | 1173 | 5182.8 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LIEGGPSSGEPPPSGSE- | | |
| | NH₂ | | |
| 1168 | Y-Aib-QGT-αMeF-TSD-4Pal-αMeS-I-αMeL-LD-Orn-K((2- | 1174 | 5151.8 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY- | | |
| | LIEGGPSSGAPPPSGGGE-NH₂ | | |
| 1169 | Y-Aib-QGT-αMeF-TSD-4Pal-αMeS-I-αMeL-LD-Orn-K((2- | 1175 | 5209.4 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LIEGGPSSGEPPPSGGGE- | | |
| | NH₂ | | |
| 1170 | Y-Aib-QGT-αMeF-TSD-4Pal-αMeS-I-αMeL-LD-Orn-K((γ- | 1176 | 5021.7 |
| | Glu)₂-CO—(CH₂)₁₆—CO₂H)AQ-Me4Pal-EFIe-αMeY- | | |
| | LIEGGPSSGAPPPSGE-NH₂ | | |
| 1171 | Y-Aib-QGT-αMeF-TSD-4Pal-αMeS-I-αMeL-LD-Orn-K((γ- | 1177 | 5079.7 |
| | Glu)₂-CO—(CH₂)₁₆—CO₂H)AQ-Me4Pal-EFIe-αMeY- | | |
| | LIEGGPSSGEPPPSGE-NH₂ | | |
| 1172 | Y-Aib-QGT-αMeF-TSD-4Pal-αMeS-I-αMeL-LD-Orn-K((γ- | 1178 | 5108.7 |
| | Glu)₂-CO—(CH₂)₁₆—CO₂H)AQ-Me4Pal-EFIe-αMeY- | | |
| | LIEGGPSSGAPPPSGSE-NH₂ | | |
| 1173 | Y-Aib-QGT-αMeF-TSD-4Pal-αMeS-I-αMeL-LD-Orn-K((γ- | 1179 | 5166.8 |
| | Glu)₂-CO—(CH₂)₁₆—CO₂H)AQ-Me4Pal-EFIe-αMeY- | | |
| | LIEGGPSSGEPPPSGSE-NH₂ | | |
| 1174 | Y-Aib-QGT-αMeF-TSD-4Pal-αMeS-I-αMeL-LD-Orn-K((γ- | 1180 | 5135.8 |
| | Glu)₂-CO—(CH₂)₁₆—CO₂H)AQ-Me4Pal-EFIe-αMeY- | | |
| | LIEGGPSSGAPPPSGGGE-NH₂ | | |
| 1175 | Y-Aib-QGT-αMeF-TSD-4Pal-αMeS-I-αMeL-LD-Orn-K((γ- | 1181 | 5193.8 |
| | Glu)₂-CO—(CH₂)₁₆—CO₂H)AQ-Me4Pal-EFIe-αMeY- | | |
| | LIEGGPSSGEPPPSGGGE-NH₂ | | |
| 1176 | Y-Aib-QGT-αMeF(2F)-TSD-4Pal-αMeS-I-αMeL-LD-Orn- | 1182 | 5184.8 |
| | K((2-[2-(2-Amino-ethoxy)-ethoxy]-acetyl)-(γ-Glu)₂-CO— | | |
| | (CH₂)₁₆—CO₂H)AQ-Me4Pal-EFIe-αMeY- | | |
| | LIEGGPSSGAPPPSGE-NH₂ | | |
| 1177 | Y-Aib-QGT-αMeF(2F)-TSD-4Pal-αMeS-I-αMeL-LD-Orn- | 1183 | 5242.8 |
| | K((2-[2-(2-Amino-ethoxy)-ethoxy]-acetyl)-(γ-Glu)₂-CO— | | |
| | (CH₂)₁₆—CO₂H)AQ-Me4Pal-EFIe-αMeY- | | |
| | LIEGGPSSGEPPPSGE-NH₂ | | |
| 1178 | Y-Aib-QGT-αMeF(2F)-TSD-4Pal-αMeS-I-αMeL-LD-Orn- | 1184 | 5271.9 |
| | K((2-[2-(2-Amino-ethoxy)-ethoxy]-acetyl)-(γ-Glu)₂-CO— | | |
| | (CH₂)₁₆—CO₂H)AQ-Me4Pal-EFIe-αMeY- | | |
| | LIEGGPSSGAPPPSGSE-NH₂ | | |
| 1179 | Y-Aib-QGT-αMeF(2F)-TSD-4Pal-αMeS-I-αMeL-LD-Orn- | 1185 | 5329.9 |
| | K((2-[2-(2-Amino-ethoxy)-ethoxy]-acetyl)-(γ-Glu)₂-CO— | | |
| | (CH₂)₁₆—CO₂H)AQ-Me4Pal-EFIe-αMeY- | | |
| | LIEGGPSSGEPPPSGSE-NH₂ | | |
| 1180 | Y-Aib-QGT-αMeF(2F)-TSD-4Pal-αMeS-I-αMeL-LD-Orn- | 1186 | 5298.9 |
| | K((2-[2-(2-Amino-ethoxy)-ethoxy]-acetyl)-(γ-Glu)₂-CO— | | |
| | (CH₂)₁₆—CO₂H)AQ-Me4Pal-EFIe-αMeY- | | |
| | LIEGGPSSGAPPPSGGGE-NH₂ | | |
| 1181 | Y-Aib-QGT-αMeF(2F)-TSD-4Pal-αMeS-I-αMeL-LD-Orn- | 1187 | 5356.9 |
| | K((2-[2-(2-Amino-ethoxy)-ethoxy]-acetyl)-(γ-Glu)₂-CO— | | |
| | (CH₂)₁₆—CO₂H)AQ-Me4Pal-EFIe-αMeY- | | |
| | LIEGGPSSGEPPPSGGGE-NH₂ | | |
| 1182 | Y-Aib-QGTFTSD-4Pal-αMeS-Q-αMeL-LD-Orn-K(e-(γ- | 1188 | 5163.8 |
| | Glu)-CO—(CH₂)₁₆—CO₂H)AQ-Me4Pal-QFIE-αMeY-LIA- | | |
| | Aib-GPSSGEPPPSGGGE-NH₂ | | |
| 1183 | Y-Aib-QGTFTSD-4Pal-αMeS-Q-αMeL-LD-Orn-K(e-(γ- | 1189 | 5220.8 |
| | Glu)-CO—(CH₂)₁₆—CO₂H)AQ-Me4Pal-QFIQ-αMeY-LIE- | | |
| | Aib-GPSSGEPPPSGGGE-NH₂ | | |
| 1184 | Y-Aib-QGTFTSD-4Pal-αMeS-Q-αMeL-LD-Orn-K(e-(γ- | 1190 | 5221.8 |
| | Glu)-CO—(CH₂)₁₆—CO₂H)AQ-Me4Pal-QFIE-αMeY-LIE-Aib- | | |
| | GPSSGEPPPSGGGE-NH₂ | | |
| 1185 | Y-Aib-QGTFTSD-4Pal-αMeS-Q-αMeL-LD-Orn-K(e-(γ- | 1191 | 5105.7 |
| | Glu)-CO—(CH₂)₁₆—CO₂H)AQ-Me4Pal-QFIE-αMeY-LIA- | | |
| | Aib-GPSSGAPPPSGGGE-NH₂ | | |
| 1186 | Y-Aib-QGTFTSD-4Pal-αMeS-Q-αMeL-LD-Orn-K(e-(γ- | 1192 | 5162.8 |
| | Glu)-CO—(CH₂)₁₆—CO₂H)AQ-Me4Pal-QFIQ-αMeY-LIE- | | |
| | Aib-GPSSGAPPPSGGGE-NH₂ | | |
| 1187 | Y-Aib-QGTFTSD-4Pal-αMeS-Q-αMeL-LD-Orn-K(e-(γ- | 1193 | 5163.8 |
| | Glu)-CO—(CH₂)₁₆—CO₂H)AQ-Me4Pal-QFIE-αMeY-LIE-Aib- | | |
| | GPSSGAPPPSGGGE-NH₂ | | |
| 1188 | Y-Aib-QGTFTSD-4Pal-αMeS-Q-αMeL-LD-Orn-K(e-(γ- | 1194 | 5192.8 |
| | Glu)-CO—(CH₂)₁₆—CO₂H)AQ-Me4Pal-QFIQ-αMeY- | | |
| | LIEGGPSSGEPPPSGGGE-NH₂ | | |
| 1189 | Y-Aib-QGT-αMeF(2F)-TSD-4Pal-αMeS-I-αMeL-LD-Orn- | 1195 | 5121.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPPS-Aib- | | |
| | Orn-PPP-Orn-GS-NH₂ | | |
| 1190 | Y-Aib-QGT-αMeF(2F)-TSD-4Pal-αMeS-I-αMeL-LD-Orn- | 1196 | 5193.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPPS-Aib- | | |
| | EPPPEGE-NH₂ | | |
| 1191 | Y-Aib-QGT-αMeF(2F)-TSD-4Pal-αMeS-I-αMeL-LD-Orn- | 1197 | 5165.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPPSGEPPPEGE-NH₂ | | |
| 1192 | Y-Aib-QGT-αMeF(2F)-TSD-4Pal-αMeS-I-αMeL-LD-Orn- | 1198 | 5183.8 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPSS-Aib- | | |
| | APPPEGE-NH₂ | | |
| 1193 | Y-Aib-QGT-αMeF(2F)-TSD-4Pal-αMeS-I-αMeL-LD-Orn- | 1199 | 5178.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPPS-Aib- | | |
| | EPPP-Orn-GE-NH₂ | | |
| 1194 | Y-Aib-QGT-αMeF(2F)-TSD-4Pal-αMeS-I-αMeL-LD-Orn- | 1200 | 5178.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPPS-Aib- | | |
| | Orn-PPPEGE-NH₂ | | |
| 1195 | Y-Aib-QGT-αMeF(2F)-TSD-4Pal-αMeS-I-αMeL-LD-Orn- | 1201 | 5163.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPPS-Aib- | | |
| | Orn-PPP-Orn-GE-NH₂ | | |
| 1196 | Y-Aib-QGT-αMeF(2F)-TSD-4Pal-αMeS-I-αMeL-LD-Orn- | 1202 | 5194.0 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPPS-Aib- | | |
| | Orn-PPP-Orn-SE-NH₂ | | |
| 1197 | Y-Aib-QGT-αMeF(2F)-TSD-4Pal-αMeS-I-αMeL-LD-Orn- | 1203 | 5278.0 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPPS-Aib- | | |
| | Orn-PPP-Orn-GGGE-NH₂ | | |
| 1198 | Y-Aib-QGT-αMeF(2F)-TSD-4Pal-αMeS-I-αMeL-LD-Orn- | 1204 | 5223.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPPS-Aib- | | |
| | Orn-PPPSGSE-NH₂ | | |
| 1199 | Y-Aib-QGT-αMeF(2F)-TSD-4Pal-αMeS-I-αMeL-LD-Orn- | 1205 | 5251.0 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPPS-Aib- | | |
| | Orn-PPPSGGGE-NH₂ | | |
| 1200 | Y-Aib-QGT-αMeF(2F)-TSD-4Pal-αMeS-I-αMeL-LD-Orn- | 1206 | 5238.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPPS-Aib- | | |
| | EPPPSGSE-NH₂ | | |
| 1201 | Y-Aib-QGT-αMeF(2F)-TSD-4Pal-αMeS-I-αMeL-LD-Orn- | 1207 | 5265.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY-LIEGGPPS-Aib- | | |
| | EPPPSGGGE-NH₂ | | |
| 1202 | Y-Aib-QGT-αMeF(2F)-TSD-4Pal-αMeS-I-αMeL-LD-Orn- | 1208 | 5012.7 |
| | K((2-[2-(2-Amino-ethoxy)-ethoxy]-acetyl)-(γ-Glu)-CO— | | |
| | (CH₂)₁₆—CO₂H)AQ-Me4Pal-EFIq-αMeY- | | |
| | LIEGGPSSGAPPPSGS-NH₂ | | |
| 1203 | Y-Aib-QaT-αMeF(2F)-TSD-4Pal-αMeS-I-αMeL-LD-Orn- | 1209 | 4950.6 |
| | K((2-[2-(2-Amino-ethoxy)-ethoxy]-acetyl)-(γ-Glu)-CO— | | |
| | (CH₂)₁₆—CO₂H)AQ-Aib-EFIe-αMeY- | | |
| | LIEGGPSSGAPPPSGS-NH₂ | | |
| 1204 | Y-Aib-QaT-αMeF(2F)-TSD-4Pal-αMeS-I-αMeL-LD-Orn- | 1210 | 4936.6 |
| | K((2-[2-(2-Amino-ethoxy)-ethoxy]-acetyl)-(γ-Glu)-CO— | | |
| | (CH₂)₁₆—CO₂H)AQ-Aib-EFIe-αMeY- | | |
| | LVEGGPSSGAPPPSGS-NH₂ | | |
| 1205 | Y-Aib-QaT-αMeF(2F)-TSD-4Pal-SI-αMeL-LD-Orn-K((2- | 1211 | 4999.6 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LVEGGPSSGAPPPSGS- | | |
| | NH₂ | | |
| 1206 | Y-Aib-QaT-αMeF(2F)-TSD-4Pal-SI-αMeL-LD-Orn-K((2- | 1212 | 5013.7 |
| | [2-(2-Amino-ethoxy)-ethoxy]-acetyl)-(γ-Glu)-CO—(CH₂)₁₆- | | |
| | CO₂H)AQ-Me4Pal-EFIe-αMeY-LIEGGPSSGAPPPSGS- | | |
| | NH₂ | | |
| 1207 | Y-Aib-QGT-αMeF(2F)-TSD-4Pal-αMeS-I-αMeL-LD-Orn- | 1213 | 5158.8 |
| | K((2-[2-(2-Amino-ethoxy)-ethoxy]-acetyl)₂-(γ-Glu)-CO— | | |
| | (CH₂)₁₆—CO₂H)AQ-Me4Pal-EFIe-αMeY- | | |
| | LIEGGPSSGAPPPSGS-NH₂ | | |
| 1208 | Y-Aib-QGT-αMeF(2F)-TSD-4Pal-αMeS-I-αMeL-LD-Orn- | 1214 | 5216.9 |
| | K((2-[2-(2-Amino-ethoxy)-ethoxy]-acetyl)₂-(γ-Glu)-CO— | | |
| | (CH₂)₁₆—CO₂H)AQ-Me4Pal-EFIe-αMeY- | | |
| | LIEGGPSSGEPPPSGS-NH₂ | | |
| 1209 | Y-Aib-QGT-αMeF(2F)-TSD-4Pal-SI-αMeL-LD-Orn- | 1215 | 5140.8 |
| | Orn((γ-Glu)₂-CO—(CH₂)₁₆—CO₂H))AQ-Me4Pal-Orn-FIe- | | |
| | αMeY-LIEGGPSSGEPPPETE-NH₂ | | |
| 1210 | Y-Aib-QGT-αMeF(2F)-TSD-4Pal-αMeS-I-αMeL-LD-Orn- | 1216 | 4985.6 |
| | Orn((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LVEGGPSSGAPPPSGS-NH₂ | | |
| 1211 | Y-Aib-QGT-αMeF(2F)-TSD-4Pal-αMeS-I-αMeL-LD-Orn- | 1217 | 4999.6 |
| | Orn((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPSGS-NH₂ | | |
| 1212 | Y-Aib-QGT-αMeF(2F)-TSD-4Pal-αMeS-I-αMeL-LD-Orn- | 1218 | 5128.7 |
| | Orn((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPSGSE-NH₂ | | |
| 1213 | Y-Aib-QGT-αMeF(2F)-TSD-4Pal-αMeS-I-αMeL-LD-Orn- | 1219 | 5155.8 |
| | Orn((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPSGGGE-NH₂ | | |
| 1214 | Y-Aib-QGT-αMeF(2F)-TSD-4Pal-αMeS-I-αMeL-LD-Orn- | 1220 | 5298.9 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPSGGGEE-NH₂ | | |
| 1215 | Y-Aib-QGT-αMeF(2F)-TSD-4Pal-αMeS-I-αMeL-LD-Orn- | 1221 | 5428.0 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPSGGGEEE-NH₂ | | |
| 1216 | Y-Aib-QGT-αMeF(2F)-TSD-4Pal-αMeS-I-αMeL-LD-Orn- | 1222 | 5341.0 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPEGGGEE-NH₂ | | |
| 1217 | Y-Aib-QGT-αMeF(2F)-TSD-4Pal-αMeS-I-αMeL-LD-Orn- | 1223 | 5470.1 |
| | K((2-[2-(2-Amino-ethoxy)-ethoxy]-acetyl)-(γ-Glu)-CO— | | |
| | (CH₂)₁₆—CO₂H)AQ-Me4Pal-EFIe-αMeY- | | |
| | LIEGGPSSGAPPPEGGGEEE-NH₂ | | |
| 1218 | Y-Aib-QGT-αMeF(2F)-TSD-4Pal-αMeS-I-αMeL-LD-Orn- | 1224 | 5282.9 |
| | K((γ-Glu)₂-CO—(CH₂)₁₆—CO₂H)AQ-Me4Pal-EFIe-αMeY- | | |
| | LIEGGPSSGAPPPSGGGEE-NH₂ | | |
| 1219 | Y-Aib-QGT-αMeF(2F)-TSD-4Pal-αMeS-I-αMeL-LD-Orn- | 1225 | 5412.0 |
| | K((γ-Glu)₂-CO—(CH₂)₁₆—CO₂H)AQ-Me4Pal-EFIe-αMeY- | | |
| | LIEGGPSSGAPPPSGGGEEE-NH₂ | | |
| 1220 | Y-Aib-QGT-αMeF(2F)-TSD-4Pal-αMeS-I-αMeL-LD-Orn- | 1226 | 5324.9 |
| | K((γ-Glu)₂-CO—(CH₂)₁₆—CO₂H)AQ-Me4Pal-EFIe-αMeY- | | |
| | LIEGGPSSGAPPPEGGGEE-NH₂ | | |
| 1221 | Y-Aib-QGT-αMeF(2F)-TSD-4Pal-αMeS-I-αMeL-LD-Orn- | 1227 | 5454.0 |
| | K((γ-Glu)₂-CO—(CH₂)₁₆—CO₂H)AQ-Me4Pal-EFIe-αMeY- | | |
| | LIEGGPSSGAPPPEGGGEEE-NH₂ | | |
| 1222 | Y-Aib-QaT-αMeF(2F)-TSD-4Pal-αMeS-I-αMeL-LD-Orn- | 1228 | 5079.7 |
| | K((2-[2-(2-Amino-ethoxy)-ethoxy]-acetyl)-(γ-Glu)-CO— | | |
| | (CH₂)₁₆—CO₂H)AQ-Aib-EFIe-αMeY- | | |
| | LIEGGPSSGAPPPSGSE-NH₂ | | |
| 1223 | Y-Aib-QaT-αMeF(2F)-TSD-4Pal-αMeS-I-αMeL-LD-Orn- | 1229 | 5106.7 |
| | K((2-[2-(2-Amino-ethoxy)-ethoxy]-acetyl)-(γ-Glu)-CO— | | |
| | (CH₂)₁₆—CO₂H)AQ-Aib-EFIe-αMeY- | | |
| | LIEGGPSSGAPPPSGGGE-NH₂ | | |
| 1224 | Y-Aib-QaT-αMeF(2F)-TSD-4Pal-αMeS-I-αMeL-LD-Orn- | 1230 | 5063.7 |
| | K((γ-Glu)₂-CO—(CH₂)₁₆—CO₂H)AQ-Aib-EFIe-αMeY- | | |
| | LIEGGPSSGAPPPSGSE-NH₂ | | |
| 1225 | Y-Aib-QaT-αMeF(2F)-TSD-4Pal-αMeS-I-αMeL-LD-Orn- | 1231 | 5090.7 |
| | K((γ-Glu)₂-CO—(CH₂)₁₆—CO₂H)AQ-Aib-EFIe-αMeY- | | |
| | LIEGGPSSGAPPPSGGGE-NH₂ | | |
| 1226 | Y-Aib-QGT-αMeF(2F)-TSD-4Pal-αMeS-I-αMeL-LD-Orn- | 1232 | 5105.8 |
| | K((γ-Glu)₂-CO—(CH₂)₁₆—CO₂H)AQ-Me4Pal-EFIe-αMeY- | | |
| | LIEGGPPS-Aib -- Orn-PPP-Orn-GS-NH₂ | | |
| 1227 | Y-Aib-QGT-αMeF(2F)-TSD-4Pal-αMeS-I-αMeL-LD-Orn- | 1233 | 5147.9 |
| | K((γ-Glu)₂-CO—(CH₂)₁₆—CO₂H)AQ-Me4Pal-EFIe-αMeY- | | |
| | LIEGGPPS-Aib -- Orn-PPP-Orn-GE-NH₂ | | |
| 1228 | Y-Aib-QGT-αMeF(2F)-TSD-4Pal-αMeS-I-αMeL-LD-Orn- | 1234 | 5177.9 |
| | K((γ-Glu)₂-CO—(CH₂)₁₆—CO₂H)AQ-Me4Pal-EFIe-αMeY- | | |
| | LIEGGPPS-Aib -- Orn-PPP-Orn-SE-NH₂ | | |
| 1229 | Y-Aib-QGT-αMeF(2F)-TSD-4Pal-αMeS-I-αMeL-LD-Orn- | 1235 | 5262.0 |
| | K((γ-Glu)₂-CO—(CH₂)₁₆—CO₂H)AQ-Me4Pal-EFIe-αMeY- | | |
| | LIEGGPPS-Aib -- Orn-PPP-Orn-GG-NH₂ | | |
| 1230 | Y-Aib-QGT-αMeF(2F)-TSD-4Pal-SI-αMeL-LD-Orn-QAQ- | 1236 | 4600.1 |
| | Me4Pal -- Orn-FIe-αMeY-LIEGGPSSGEPPPETE-NH₂ | | |
| 1231 | Y-Aib-QGT-αMeF(2F)-TSD-4Pal-αMeS-I-αMeL-LD-Orn- | 1237 | 4428.9 |
| | QAQ-Me4Pal-EFIe-αMeY-LVEGGPSSGAPPPSGS-NH₂ | | |
| 1232 | Y-Aib-QGT-αMeF(2F)-TSD-4Pal-αMeS-I-αMeL-LD-Orn- | 1238 | 4442.9 |
| | QAQ-Me4Pal-EFIe-αMeY-LIEGGPSSGAPPPSGS-NH₂ | | |
| 1233 | Y-Aib-QGT-αMeF(2F)-TSD-4Pal-αMeS-I-αMeL-LD-Orn- | 1239 | 4572.0 |
| | QAQ-Me4Pal-EFIe-αMeY-LIEGGPSSGAPPPSGSE-NH₂ | | |
| 1234 | Y-Aib-QGT-αMeF(2F)-TSD-4Pal-αMeS-I-αMeL-LD-Orn- | 1240 | 4599.0 |
| | QAQ-Me4Pal-EFIe-αMeY-LIEGGPSSGAPPPSGGGE-NH₂ | | |
| 1235 | Y-Aib-QGTFTSD-4Pal-SQ-αMeL-LD-Orn-K(e-(γ-Glu)- | 1241 | 5295.9 |
| | CO—(CH₂)₁₆—CO₂H)AQ-Me4Pal-EFIe-αMeY- | | |
| | LIAGGPSSGAPPPSGSEEE-NH₂ | | |
| 1236 | Y-Aib-QGT-αMeF(2F)-TSD-4Pal-SI-αMeL-LD-Orn-K((2- | 1242 | 4749.3 |
| | [2-(2-Amino-ethoxy)-ethoxy]-acetyl)-(γ-Glu)-CO—(CH₂)₁₆- | | |
| | CO₂H)AA-Aib-AFIe-αMeY-LLEGGPSSGEPPPS-NH₂ | | |

Depictions of the structures of certain examples are provided below:

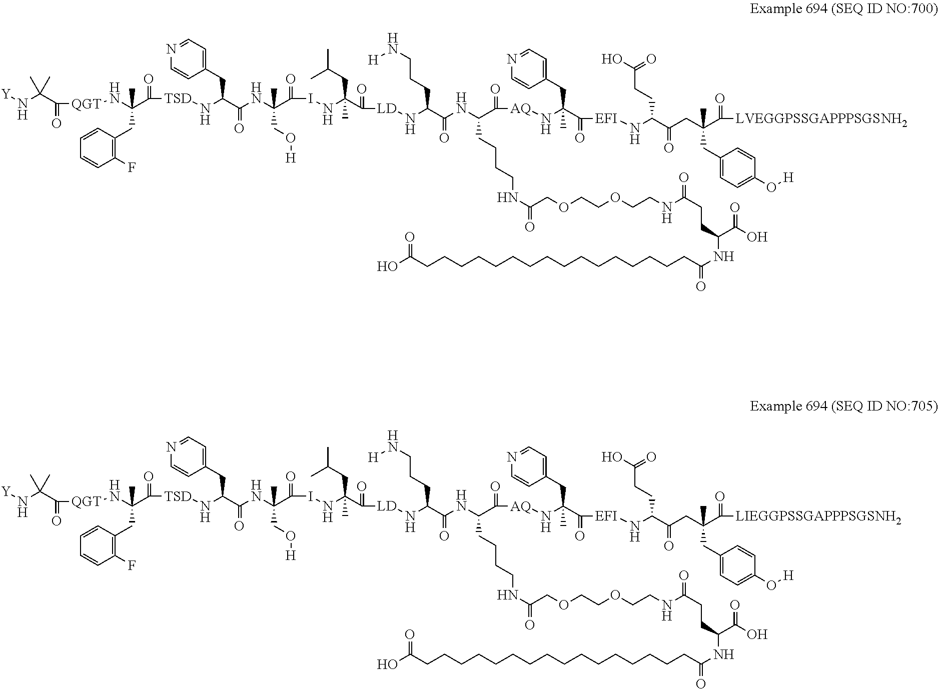

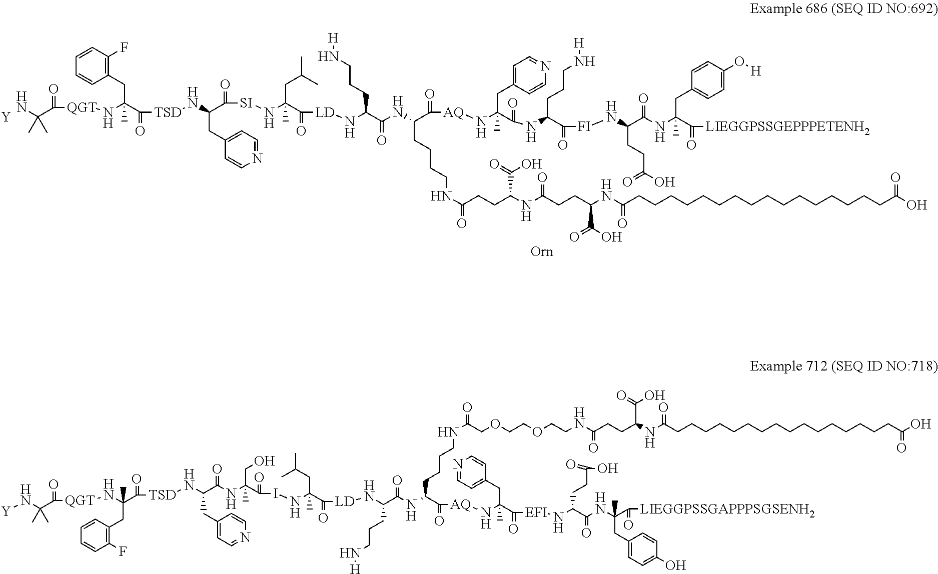

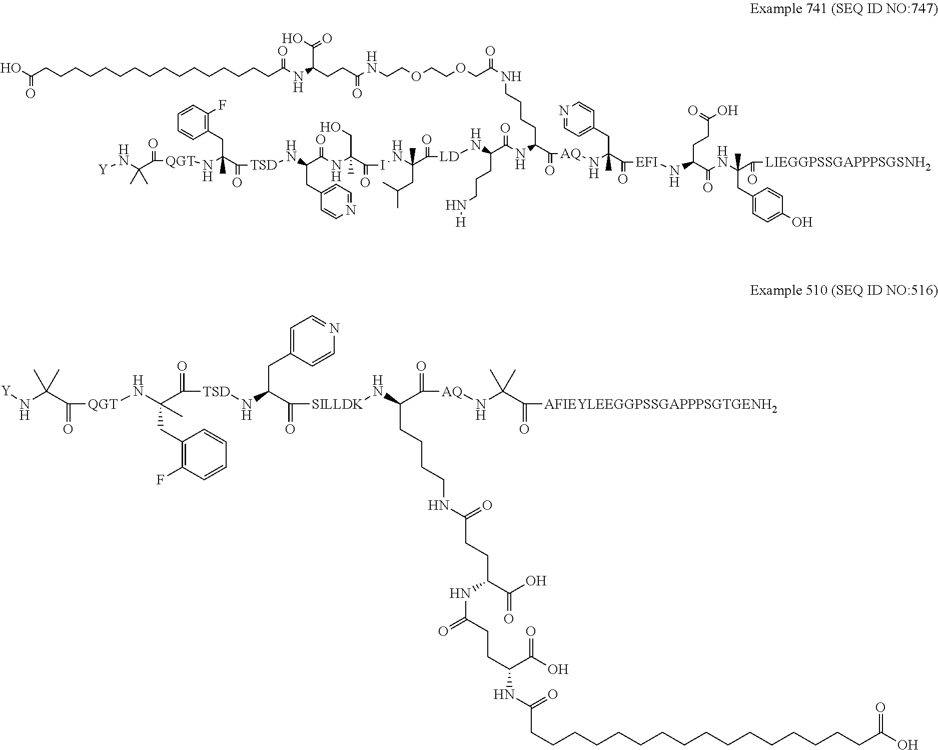

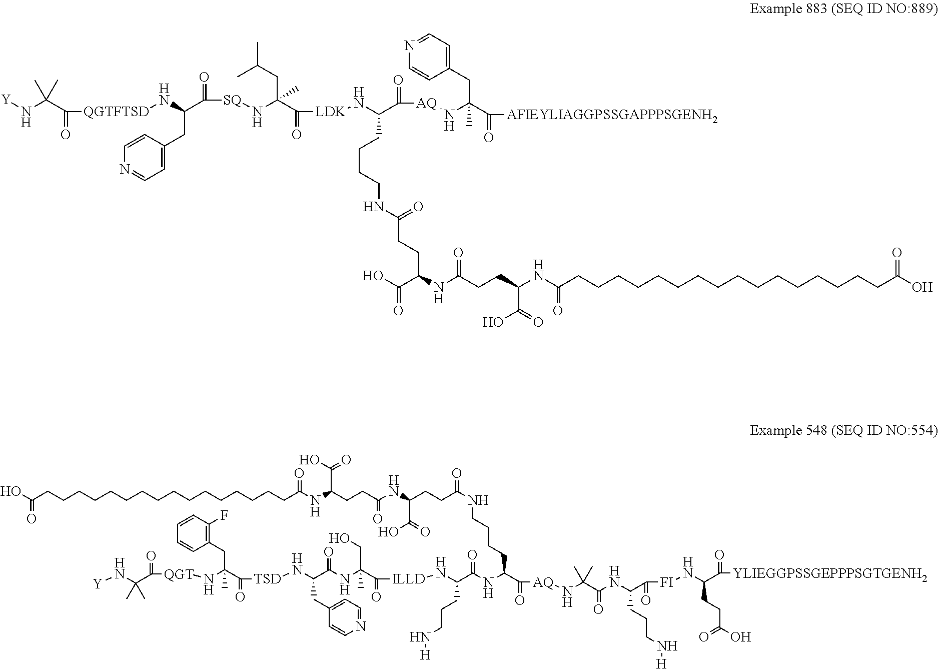

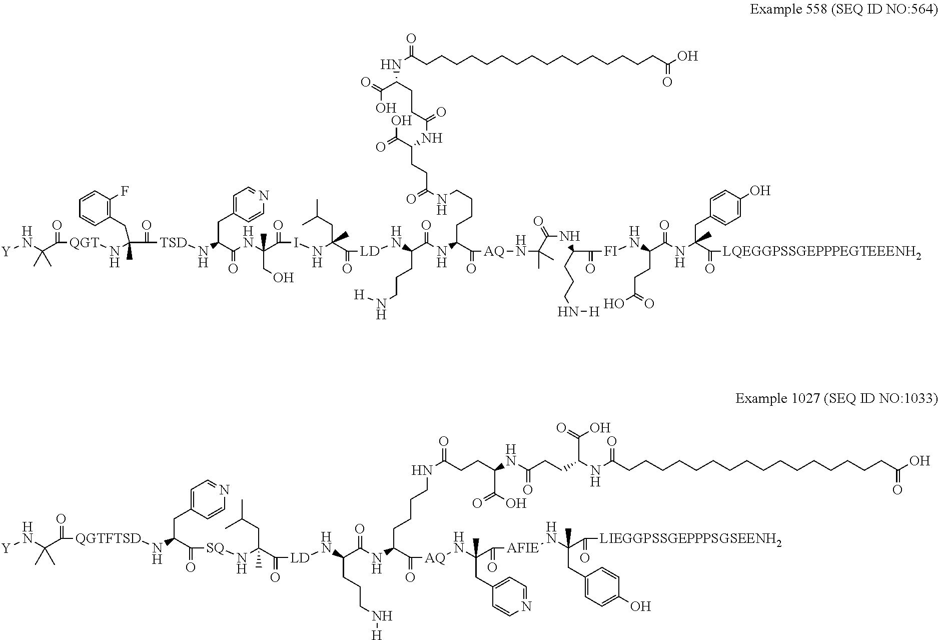

### In Vitro Function

### Functional Activity:

Functional activity is determined in GIP-R, GLP-1R, and GcgR-expressing HEK-293 clonal cell lines. Each receptor cell line is treated with peptide (20 point concentration response curves with 2.75-fold serial dilutions prepared with a Labcyte Echo Acoustic Liquid Handler) in DMEM (Gibco Cat #31053) supplemented with 1× GlutaMAX™ (L-alanyl-L-glutamine dipeptide, Gibco Cat #35050), 0.1% Casein (Sigma Cat #C4765), 1% HSA (Human Serum Albumin, Sigma Cat #A3782) 500 μM IBMX (3-isobutyl-1-methylxanthine) and 20 mM HEPES (4-(2-hydroxyethyl)-1-piperazineethanesulfonic acid) in a 20 μl assay volume.

After a 30-minute incubation at 37° C., the resulting increase in intracellular cAMP is quantitatively determined using the CisBio CAMP Dynamic 2 HTRF Assay Kit (62AM4PEJ). Briefly, cAMP levels within the cell are detected by adding the cAMP-d2 conjugate in cell lysis buffer followed by the antibody anti-cAMP-Eu³⁺-Cryptate, also in cell lysis buffer. The resulting competitive assay is incubated for at least 60 minutes at room temperature and then detected using a Pherastar Instrument (BMG Labtech) with excitation at 320 nm and emission at 665 nm and 620 nm. Raw data values (emission at 665 nm/620 nm*10,000) are inversely proportional to the amount of cAMP present and were converted to CAMP (nM) per well using a cAMP standard curve.

The amount of cAMP generated (nM) in each well is converted to a percent of the maximal response observed with either human GLP-1(7-36)NH₂, human Glucagon (Gcg), or human GIP(1-42)NH₂. A relative EC₅₀ value is derived by non-linear regression analysis using the percent maximal response vs. the concentration of peptide added, fitted to a four-parameter logistic equation.

The Geometric mean of relative EC₅₀ data for exemplary analogs and hGIP(1-42)NH₂, hGLP-1(7-36)NH₂ and hGcg are shown in Table 2 below.

TABLE 2
Functional cAMP Potency (Rel EC₅₀) for Exemplary Analogs
and Comparators in the Presence of 1% HSA.

| | cAMP Rel EC₅₀, nM | | |
|---|---|---|---|
| Example | hGcgR | hGIPR | hGLP-1R |
| hGcg | 2.2 | >300 | 160 |
| hGIP 1-42 amide | >9950 | 0.536 | >9950 |
| hGLP-1 7-36 amide | >99.5 | >99.5 | 0.346 |
| 1 | 1690 | 73.9 | 524 |
| 2 | 1630 | 75.5 | 631 |
| 3 | 1590 | 48.1 | 546 |
| 4 | 1580 | 62.3 | 869 |
| 5 | 2670 | 32.5 | 675 |
| 6 | 1380 | 46.6 | 446 |
| 7 | 1210 | 50.6 | 494 |
| 8 | 1560 | 39.4 | 603 |
| 9 | 1430 | 41.8 | 402 |
| 10 | 1640 | 43 | 375 |
| 11 | 1700 | 40.8 | 337 |
| 12 | 1490 | 30.2 | 216 |
| 13 | 1670 | 29.2 | 282 |
| 14 | 1420 | 34.7 | 232 |
| 15 | 1980 | 36.3 | 276 |
| 16 | 1660 | 35.9 | 281 |
| 17 | 2040 | 91.7 | 543 |
| 18 | 1210 | 59.6 | 561 |
| 19 | 1010 | 66.2 | 1310 |
| 20 | 587 | 42.3 | 326 |
| 21 | 1510 | 29.1 | 324 |
| 22 | 532 | 81 | 592 |
| 23 | 573 | 25.5 | 308 |
| 24 | 1390 | 102 | 447 |
| 25 | 939 | 19.8 | 520 |
| 26 | 980 | 35.4 | 623 |
| 27 | 1180 | 72.3 | 464 |
| 28 | 1550 | 63.8 | 686 |
| 29 | 1350 | 70.1 | 493 |
| 30 | 969 | 72.9 | 806 |
| 31 | 1750 | 63.6 | 737 |
| 32 | 1440 | 66.1 | 422 |
| 33 | 1310 | 70.7 | 436 |
| 34 | 855 | 44.2 | 242 |
| 35 | 1390 | 194 | 1130 |
| 36 | 1330 | 170 | 1360 |
| 37 | 1870 | 56.4 | 514 |
| 38 | 2770 | 102 | 884 |
| 39 | 1090 | 30.5 | 260 |
| 40 | 2750 | 76.7 | 138 |
| 41 | 2310 | 97.9 | 711 |
| 42 | 1520 | 32.3 | 700 |
| 43 | 850 | 29.6 | 539 |
| 44 | 1050 | 35.9 | 591 |
| 45 | 879 | 33.4 | 493 |
| 46 | 393 | 19.6 | 601 |
| 47 | 2140 | 154 | 1270 |
| 48 | 728 | 46.9 | 570 |
| 49 | 1310 | 44.9 | 151 |
| 50 | 1040 | 69.6 | 1110 |
| 51 | 856 | 98.4 | 1210 |
| 52 | 1740 | 111 | 1250 |
| 53 | 1780 | 113 | 1030 |
| 54 | 1940 | 168 | 1300 |
| 55 | 960 | 27.8 | 479 |
| 56 | 265 | 30.3 | 167 |
| 57 | 113 | 3.78 | 42.7 |
| 58 | 115 | 3.75 | 20.7 |
| 59 | 2160 | 166 | 1370 |
| 60 | 1210 | 33 | 409 |
| 61 | 1310 | 55.2 | 704 |
| 62 | 396 | 31.2 | 299 |
| 63 | 709 | 40.4 | 348 |
| 64 | 626 | 24.9 | 397 |
| 65 | 808 | 30.2 | 462 |
| 66 | 415 | 29.9 | 329 |
| 67 | 781 | 73.3 | 683 |
| 68 | 1160 | 85.1 | 612 |
| 69 | 387 | 25.6 | 279 |
| 70 | 717 | 27.4 | 620 |
| 71 | 2800 | 48.1 | 1010 |
| 72 | 1910 | 56.7 | 683 |
| 73 | 1530 | 57.9 | 810 |
| 74 | 709 | 15.9 | 627 |
| 75 | 1040 | 16.8 | 878 |
| 76 | 2240 | 45.5 | 436 |
| 77 | 2610 | 37.9 | 339 |
| 78 | 2640 | 78.4 | 560 |
| 79 | 903 | 14.3 | 437 |
| 80 | 1190 | 10.1 | 627 |
| 81 | 1580 | 50.2 | 430 |
| 82 | 1070 | 87.8 | 394 |
| 83 | 1270 | 91.8 | 653 |
| 84 | 2280 | 57.4 | 701 |
| 85 | 1270 | 22.1 | 387 |
| 86 | 1130 | 19.6 | 421 |
| 87 | 1110 | 12.8 | 290 |
| 88 | 915 | 24.7 | 363 |
| 89 | 916 | 19.3 | 458 |
| 90 | 990 | 24.9 | 371 |
| 91 | 1200 | 23.9 | 356 |
| 92 | 2540 | 62 | 735 |
| 93 | 1390 | 27.9 | 397 |
| 94 | 1220 | 21.4 | 480 |
| 95 | 1130 | 18.2 | 380 |
| 96 | 2150 | 49.3 | 836 |
| 97 | 1270 | 23.9 | 605 |
| 98 | 867 | 33.8 | 660 |
| 99 | 1130 | 29.5 | 588 |
| 100 | 2390 | 77.2 | 1190 |
| 101 | 2810 | 133 | 1090 |
| 102 | 2710 | 159 | 1060 |
| 103 | 2590 | 35.1 | 482 |
| 104 | 1260 | 97.5 | 698 |
| 105 | 2790 | 72.5 | 768 |
| 106 | 2000 | 91 | 977 |
| 107 | 1690 | 61.3 | 1110 |
| 108 | 1290 | 56.5 | 777 |
| 109 | 2760 | 22.3 | 671 |
| 110 | 627 | 19.8 | 821 |
| 111 | 1260 | 10.4 | 878 |
| 112 | 1810 | 29 | 892 |
| 113 | 2090 | 32.3 | 467 |
| 114 | 1120 | 19.6 | 1370 |
| 115 | 2210 | 73.9 | 1460 |
| 116 | 882 | 40.4 | 401 |
| 117 | 867 | 29.9 | 524 |
| 118 | 2460 | 63.4 | 1450 |
| 119 | 839 | 19.7 | 387 |
| 120 | 1540 | 33.4 | 664 |
| 121 | 442 | 24.6 | 348 |
| 122 | 548 | 32.6 | 468 |
| 123 | 1740 | 94.6 | 1500 |
| 124 | 567 | 54.7 | 801 |
| 125 | 901 | 67.8 | 1050 |
| 126 | 1120 | 40.3 | 838 |
| 127 | 1010 | 62.9 | 1000 |
| 128 | 1090 | 24.3 | 679 |
| 129 | 510 | 24.3 | 284 |
| 130 | 1080 | 60.5 | 865 |
| 131 | 976 | 91.8 | 984 |
| 132 | 632 | 25.9 | 823 |
| 133 | 642 | 23.1 | 1180 |
| 134 | 1200 | 38.3 | 549 |
| 135 | 557 | 23.4 | 472 |
| 136 | 960 | 32.9 | 625 |
| 137 | 905 | 23.7 | 447 |
| 138 | 1480 | 40.1 | 657 |
| 139 | 1250 | 64.8 | 710 |
| 140 | 2360 | 2.94 | 165 |
| 141 | 877 | 62.1 | 562 |
| 142 | 1230 | 14.1 | 391 |
| 143 | 1690 | 63.9 | 594 |
| 144 | 2600 | 46.2 | 990 |
| 145 | 693 | 108 | 1480 |
| 146 | 463 | 74.6 | 678 |
| 147 | 877 | 108 | 964 |
| 148 | 1330 | 74.2 | 870 |
| 149 | 1420 | 44.9 | 801 |
| 150 | 1920 | 50.6 | 463 |
| 151 | 1420 | 14.9 | 384 |
| 152 | 1090 | 23.2 | 491 |
| 153 | 1210 | 16.8 | 488 |
| 154 | 1040 | 29.7 | 569 |
| 155 | 1310 | 18.9 | 503 |
| 156 | 1520 | 25.8 | 695 |
| 157 | 838 | 22.2 | 496 |
| 158 | 325 | 24.7 | 184 |
| 159 | 475 | 49.7 | 588 |
| 160 | 479 | 44.4 | 482 |
| 161 | 902 | 87.3 | 963 |
| 162 | 558 | 37.5 | 786 |
| 163 | 790 | 52 | 762 |
| 164 | 411 | 59 | 962 |
| 165 | 1250 | 18.6 | 808 |
| 166 | 724 | 21 | 1500 |
| 167 | 938 | 24.7 | 1300 |
| 168 | 1270 | 31.9 | 281 |
| 169 | 439 | 21.5 | 216 |
| 170 | 1800 | 43.5 | 361 |
| 171 | 502 | 27.1 | 287 |
| 172 | 605 | 29.3 | 457 |
| 173 | 745 | 49.9 | 293 |
| 174 | 307 | 20.8 | 208 |
| 175 | 579 | 29.6 | 386 |
| 176 | 461 | 40.7 | 389 |
| 177 | 758 | 34.8 | 483 |
| 178 | 816 | 47.3 | 397 |
| 179 | 1440 | 21.3 | 904 |
| 180 | 1320 | 24 | 891 |
| 181 | 772 | 12.7 | 365 |
| 182 | 1480 | 9.95 | 398 |
| 183 | 504 | 13.7 | 295 |
| 184 | 1050 | 16.2 | 465 |
| 185 | 504 | 21.1 | 502 |
| 186 | 1280 | 18 | 690 |
| 187 | 2970 | 15.6 | 178 |
| 188 | 2920 | 43.8 | 436 |
| 189 | 853 | 22.2 | 423 |
| 190 | 331 | 59.4 | 172 |
| 191 | 344 | 47.4 | 230 |
| 192 | 560 | 9.86 | 719 |
| 193 | 1120 | 12.7 | 473 |
| 194 | 813 | 19.7 | 697 |
| 195 | 605 | 28.8 | 829 |
| 196 | 620 | 51.4 | 458 |
| 197 | 613 | 44.1 | 440 |
| 198 | 1300 | 38.8 | 761 |
| 199 | 1000 | 41.6 | 1060 |
| 200 | 360 | 13.7 | 395 |
| 201 | 969 | 14.3 | 679 |
| 202 | 697 | 11.7 | 376 |
| 203 | 1160 | 19 | 532 |
| 204 | 572 | 16.7 | 306 |
| 205 | 397 | 24.4 | 234 |
| 206 | 545 | 38.8 | 372 |
| 207 | 770 | 15.2 | 225 |
| 208 | 578 | 22 | 336 |
| 209 | 433 | 23.3 | 276 |
| 210 | 501 | 23.5 | 264 |
| 211 | 370 | 20.6 | 234 |
| 212 | 324 | 32.3 | 360 |
| 213 | 429 | 22.9 | 327 |
| 214 | 302 | 15.1 | 186 |
| 215 | 1510 | 87.3 | 162 |
| 216 | 895 | 12 | 90.6 |
| 217 | 1570 | 15 | 130 |
| 218 | 1480 | 15 | 152 |
| 219 | 1870 | 23.3 | 221 |
| 220 | 1990 | 87.1 | 189 |
| 221 | 1620 | 17.1 | 127 |
| 222 | 440 | 52.5 | 753 |
| 223 | 373 | 37.2 | 627 |
| 224 | 452 | 21.5 | 333 |
| 225 | 561 | 40.2 | 438 |
| 226 | 763 | 32.2 | 354 |
| 227 | 937 | 39.7 | 418 |
| 228 | 322 | 37.8 | 311 |
| 229 | 332 | 27.8 | 249 |
| 230 | 388 | 21.6 | 199 |
| 231 | 443 | 27.4 | 223 |
| 232 | 482 | 24.6 | 226 |
| 233 | 453 | 28.1 | 223 |
| 234 | 702 | 31.1 | 604 |
| 235 | 700 | 40.8 | 457 |
| 236 | 309 | 24.8 | 261 |
| 237 | 441 | 30.9 | 291 |
| 238 | 334 | 8.78 | 262 |
| 239 | 693 | 12 | 419 |
| 240 | 521 | 10.4 | 396 |
| 241 | 543 | 9.36 | 264 |
| 242 | 484 | 5.94 | 242 |
| 243 | 711 | 12.1 | 610 |
| 244 | 1230 | 18.8 | 645 |
| 245 | 1120 | 10.5 | 540 |
| 246 | 639 | 22 | 845 |
| 247 | 443 | 12.9 | 291 |
| 248 | 241 | 10.6 | 438 |
| 249 | 604 | 22.5 | 256 |
| 250 | 201 | 11.7 | 194 |
| 251 | 1500 | 22 | 301 |
| 252 | 528 | 24.8 | 504 |
| 253 | 963 | 26.6 | 355 |
| 254 | 767 | 21.4 | 265 |
| 255 | 883 | 21.9 | 272 |
| 256 | 625 | 17.4 | 235 |
| 257 | 1210 | 21 | 388 |
| 258 | 1280 | 23.3 | 492 |
| 259 | 317 | 18.4 | 220 |
| 260 | 162 | 12.4 | 160 |
| 261 | 520 | 38.4 | 383 |
| 262 | 720 | 40.4 | 400 |
| 263 | 893 | 25.7 | 257 |
| 264 | 1000 | 12.4 | 688 |
| 265 | 1890 | 29 | 381 |
| 266 | 923 | 17.3 | 583 |
| 267 | 1380 | 55.5 | 1020 |
| 268 | 1300 | 33.7 | 437 |
| 269 | 1100 | 21.9 | 428 |
| 270 | 1060 | 36.5 | 658 |
| 271 | 2680 | 15.7 | 887 |
| 272 | 1540 | 36.3 | 486 |
| 273 | 1770 | 21.8 | 516 |
| 274 | 1120 | 27.6 | 560 |
| 275 | 882 | 27.7 | 692 |
| 276 | 113 | 6.28 | 103 |
| 277 | 1690 | 13.3 | 639 |
| 278 | 274 | 7.69 | 241 |
| 279 | 427 | 11.6 | 367 |
| 280 | 1600 | 15.6 | 171 |
| 281 | 1370 | 25.8 | 295 |
| 282 | 1830 | 22.1 | 217 |
| 283 | 1470 | 47.2 | 233 |
| 284 | 1530 | 59.8 | 271 |
| 285 | 409 | 8.22 | 365 |
| 286 | 228 | 11.5 | 925 |
| 287 | 291 | 9.94 | 562 |
| 288 | 3580 | 59.1 | 616 |
| 289 | 1750 | 105 | 139 |
| 290 | 878 | 53.4 | 90 |
| 291 | 311 | 38 | 24.5 |
| 292 | 583 | 92.7 | 129 |
| 293 | 1740 | 9.63 | 295 |
| 294 | 518 | 54.1 | 554 |
| 295 | 605 | 61.1 | 232 |
| 296 | 517 | 21.8 | 171 |
| 297 | 2320 | 325 | 1130 |
| 298 | 1570 | 41.3 | 70.9 |
| 299 | 1700 | 40.2 | 380 |
| 300 | 976 | 12.9 | 98 |
| 301 | 1500 | 81.8 | 82.4 |
| 302 | 1370 | 91.8 | 73.6 |
| 303 | 9950 | 44.7 | 257 |
| 304 | 1720 | 48 | 172 |
| 305 | 1500 | 92.4 | 415 |
| 306 | 2820 | 95.3 | 126 |
| 307 | 1880 | 65.3 | 209 |
| 308 | 4950 | 237 | 477 |
| 309 | 3910 | 424 | 251 |
| 310 | 4000 | 15 | 177 |
| 311 | 2500 | 26.1 | 1080 |
| 312 | 4070 | 97.7 | 589 |
| 313 | 3290 | 86.4 | 135 |
| 314 | 1030 | 65.9 | 100 |
| 315 | 1560 | 85.1 | 140 |
| 316 | 2090 | 102 | 78.7 |
| 317 | 1500 | 62.8 | 234 |
| 318 | 1970 | 52.8 | 111 |
| 319 | 1650 | 60.1 | 119 |
| 320 | 1170 | 55.3 | 328 |
| 321 | 836 | 13 | 110 |
| 322 | 2620 | 89.3 | 135 |
| 323 | 1440 | 37.7 | 293 |
| 324 | 1410 | 20.7 | 1060 |
| 325 | 774 | 82.7 | 371 |
| 326 | 1500 | 83.7 | 172 |
| 327 | 1400 | 63.3 | 473 |
| 328 | 2040 | 91.1 | 462 |
| 329 | 842 | 42.6 | 204 |
| 330 | 398 | 7.33 | 91.8 |
| 331 | 339 | 47.5 | 207 |
| 332 | 3340 | 78.2 | 298 |
| 333 | 1410 | 36.3 | 182 |
| 334 | 2070 | 72.1 | 171 |
| 335 | 1410 | 46.7 | 150 |
| 336 | 1740 | 64.4 | 227 |
| 337 | 2950 | 99.1 | 272 |
| 338 | 659 | 10.9 | 172 |
| 339 | 1510 | 28.8 | 192 |
| 340 | 647 | 60.4 | 246 |
| 341 | 2930 | 49.6 | 395 |
| 342 | 2130 | 71.3 | 62 |
| 343 | 475 | 16.7 | 178 |
| 344 | 1440 | 70.7 | 523 |
| 345 | 3300 | 15.3 | 256 |
| 346 | 4310 | 28.3 | 514 |
| 347 | 3650 | 17.3 | 254 |
| 348 | 1250 | 36.4 | 136 |
| 349 | 2280 | 52.6 | 218 |
| 350 | 3760 | 33.6 | 360 |
| 351 | 2470 | 57.5 | 272 |
| 352 | 4020 | 34.1 | 407 |
| 353 | 3080 | 53.7 | 222 |
| 354 | 1440 | 23.8 | 175 |
| 355 | 3430 | 33.2 | 351 |
| 356 | 2680 | 19.2 | 206 |
| 357 | 1810 | 42.9 | 248 |
| 358 | 876 | 38.8 | 492 |
| 359 | 957 | 103 | 826 |
| 360 | 1470 | 78.3 | 989 |
| 361 | 987 | 46.3 | 376 |
| 362 | 1450 | 38.7 | 450 |
| 363 | 4080 | 91.8 | 217 |
| 364 | 3190 | 101 | 260 |
| 365 | 1490 | 14 | 75.2 |
| 366 | 1830 | 4.27 | 85.9 |
| 367 | 668 | 4.89 | 30.1 |
| 368 | 111 | 2.62 | 37.4 |
| 369 | 111 | 4.58 | 26.8 |
| 370 | 803 | 4.23 | 37.5 |
| 371 | 463 | 1.47 | 47.4 |
| 372 | 468 | 2.63 | 53.1 |
| 373 | 666 | 1.97 | 54.5 |
| 374 | 65.4 | 2.28 | 24.5 |
| 375 | 137 | 1.78 | 18 |
| 376 | 115 | 5.42 | 26 |
| 377 | 163 | 11.1 | 29.4 |
| 378 | 176 | 2.15 | 18.8 |
| 379 | 344 | 24 | 14.2 |
| 380 | 150 | 7.77 | 30.6 |
| 381 | 176 | 7.01 | 24.5 |
| 382 | 1050 | 17.7 | 61.5 |
| 383 | 530 | 32.3 | 79.1 |
| 384 | 1730 | 3.63 | 19.3 |
| 385 | 700 | 4.03 | 86.1 |
| 386 | 734 | 5.62 | 98.6 |
| 387 | 184 | 5.64 | 26.5 |
| 388 | 603 | 14.1 | 52.5 |
| 389 | 889 | 18.3 | 18.9 |
| 390 | 549 | 37.8 | 19.6 |
| 391 | 808 | 11.5 | 34.4 |
| 392 | 611 | 28.7 | 41.5 |
| 393 | 151 | 12.4 | 29.6 |
| 394 | 1090 | 7.22 | 33.2 |
| 395 | 1190 | 6.69 | 34.7 |
| 396 | 468 | 9.5 | 38.2 |
| 397 | 1110 | 10.1 | 34.9 |
| 398 | 375 | 12.4 | 42.2 |
| 399 | 560 | 19.6 | 42.8 |
| 400 | 265 | 3.86 | 29.1 |
| 401 | 111 | 1.71 | 12 |
| 402 | 1030 | 44.9 | 86.6 |
| 403 | 527 | 36.9 | 70 |
| 404 | 735 | 2.03 | 32.2 |
| 405 | 2530 | 32.1 | 285 |
| 406 | 254 | 10.5 | 27.8 |
| 407 | 485 | 11.7 | 37.2 |
| 408 | 472 | 15.3 | 37.9 |
| 409 | 1190 | 66.6 | 41.7 |
| 410 | 1950 | 49.3 | 84.4 |
| 411 | 1170 | 23.8 | 82.1 |
| 412 | 467 | 12.5 | 46 |
| 413 | 629 | 23.5 | 41.4 |
| 414 | 487 | 14.9 | 32.8 |
| 415 | 1090 | 28.9 | 52.6 |
| 416 | 348 | 27.6 | 40.8 |
| 417 | 386 | 4.46 | 67 |
| 418 | 627 | 6.02 | 36.6 |
| 419 | 1650 | 9.47 | 65.5 |
| 420 | 433 | 2.61 | 31.3 |
| 421 | 468 | 2.98 | 31.9 |
| 422 | 493 | 4.4 | 27.1 |
| 423 | 319 | 3.07 | 28.7 |
| 424 | 443 | 3.21 | 29.5 |
| 425 | 1150 | 46.9 | 20.3 |
| 426 | 740 | 29.8 | 16.6 |
| 427 | 1440 | 4.25 | 42.9 |
| 428 | 536 | 4.55 | 38.2 |
| 429 | 138 | 1.89 | 12.1 |
| 430 | 559 | 4.65 | 78.9 |
| 431 | 352 | 3.73 | 35.2 |
| 432 | 384 | 11.4 | 41.7 |
| 433 | 532 | 15.3 | 43.4 |
| 434 | 525 | 10.6 | 35 |
| 435 | 334 | 10.3 | 33.6 |
| 436 | 182 | 1.18 | 11.7 |
| 437 | 188 | 16.9 | 26.9 |
| 438 | 431 | 53.8 | 41.1 |
| 439 | 165 | 14.6 | 15.1 |
| 440 | 830 | 1.81 | 27.4 |
| 441 | 855 | 1.96 | 24.7 |
| 442 | 342 | 1.81 | 23.9 |
| 443 | 265 | 1.57 | 34.3 |
| 444 | 1280 | 1.74 | 54.6 |
| 445 | 448 | 2.41 | 23.2 |
| 446 | 516 | 3.01 | 24.5 |
| 447 | 1090 | 1.81 | 11.3 |
| 448 | 302 | 1.35 | 19.5 |
| 449 | 424 | 1.66 | 18.9 |
| 450 | 553 | 3.75 | 18.4 |
| 451 | 384 | 0.96 | 10.9 |
| 452 | 240 | 0.846 | 12.4 |
| 453 | 261 | 1.95 | 20.6 |
| 454 | 1280 | 4.31 | 135 |
| 455 | 1200 | 5.2 | 105 |
| 456 | 230 | 0.993 | 12.5 |
| 457 | 333 | 0.881 | 11.7 |
| 458 | 319 | 8.07 | 22.7 |
| 459 | 73.6 | 2.06 | 12.6 |
| 460 | 142 | 8.93 | 31.1 |
| 461 | 131 | 2.51 | 12 |
| 462 | 183 | 1.62 | 12.9 |
| 463 | 496 | 6.36 | 24.7 |
| 464 | 194 | 19.9 | 45.5 |
| 465 | 233 | 44.6 | 56.3 |
| 466 | 348 | 5.65 | 13.3 |
| 467 | 850 | 10.7 | 36.4 |
| 468 | 195 | 4.76 | 30.7 |
| 469 | 155 | 4.24 | 61.7 |
| 470 | 519 | 5.52 | 37.4 |
| 471 | 181 | 5.45 | 37.9 |
| 472 | 229 | 0.927 | 7.88 |
| 473 | 311 | 1.11 | 11.4 |
| 474 | 166 | 0.743 | 10.6 |
| 475 | 254 | 1.85 | 18.6 |
| 476 | 90.2 | 1.29 | 14.6 |
| 477 | 716 | 1.44 | 18.7 |
| 478 | 665 | 26 | 28.6 |
| 479 | 432 | 7.11 | 23.5 |
| 480 | 65.5 | 0.722 | 8.39 |
| 481 | 100 | 1.19 | 14.2 |
| 482 | 325 | 1.58 | 14.9 |
| 483 | 231 | 1.17 | 18.2 |
| 484 | 204 | 1.59 | 16 |
| 485 | 298 | 0.869 | 11.8 |
| 486 | 647 | 2.57 | 35 |
| 487 | 477 | 2.26 | 27.7 |
| 488 | 651 | 2.31 | 43.3 |
| 489 | 523 | 1.25 | 11.8 |
| 490 | 262 | 0.861 | 9.8 |
| 491 | 159 | 0.688 | 9.55 |
| 492 | 128 | 0.84 | 9.85 |
| 493 | 333 | 3.34 | 47 |
| 494 | 333 | 1.08 | 19.9 |
| 495 | 205 | 5.74 | 53.8 |
| 496 | 1500 | 60.8 | 1480 |
| 497 | 418 | 32.6 | 161 |
| 498 | 257 | 4.70 | 196 |
| 499 | 192 | 3.12 | 26.4 |
| 500 | 494 | 3.9 | 50.3 |
| 501 | 455 | 2.32 | 38 |
| 502 | 395 | 2.42 | 30.6 |
| 503 | 233 | 2.02 | 26.1 |
| 504 | 162 | 1.8 | 16.5 |
| 505 | 92 | 2.1 | 39 |
| 506 | 2330 | 28.7 | 224 |
| 507 | 209 | 1.12 | 21.3 |
| 508 | 255 | 0.856 | 17.5 |
| 509 | 617 | 5.01 | 69.2 |
| 510 | 564 | 6.4 | 74.2 |
| 511 | 594 | 6.55 | 108 |
| 512 | 891 | 9.2 | 112 |
| 513 | 1440 | 10.3 | 78.2 |
| 514 | 1150 | 10 | 82 |
| 515 | 1070 | 14.9 | 108 |
| 516 | 1410 | 13.6 | 111 |
| 517 | 337 | 9.12 | 50 |
| 518 | 265 | 7.1 | 51 |
| 519 | 356 | 10.8 | 66.5 |
| 520 | 407 | 9.09 | 60.6 |
| 521 | 275 | 0.967 | 24.8 |
| 522 | 441 | 1.95 | 35.5 |
| 523 | 234 | 0.869 | 27.1 |
| 524 | 464 | 1.46 | 32.7 |
| 525 | 460 | 2.07 | 20.2 |
| 526 | 530 | 2.12 | 22.9 |
| 527 | 580 | 2.6 | 26.8 |
| 528 | 474 | 1.55 | 24.3 |
| 529 | 398 | 1.39 | 24.5 |
| 530 | 1280 | 63.7 | 437 |
| 531 | 465 | 5.2 | 37.8 |
| 532 | 157 | 1.49 | 47.3 |
| 533 | 453 | 3.23 | 30 |
| 534 | 499 | 3.73 | 34.9 |
| 535 | 238 | 1.16 | 29.9 |
| 536 | 351 | 1.5 | 32.5 |
| 537 | 454 | 4.48 | 46.9 |
| 538 | 677 | 5.07 | 44.9 |
| 539 | 461 | 1.63 | 33.8 |
| 540 | 405 | 1.93 | 38.2 |
| 541 | 520 | 1.28 | 38.7 |
| 542 | 256 | 1.69 | 29.6 |
| 543 | 300 | 1.88 | 32 |
| 544 | 171 | 2.4 | 43 |
| 545 | 180 | 2.74 | 40.2 |
| 546 | 225 | 11.9 | 34.4 |
| 547 | 237 | 7.26 | 27.7 |
| 548 | 295 | 3.48 | 38 |
| 549 | 655 | 3.67 | 76.2 |
| 550 | 996 | 5.94 | 262 |
| 551 | 210 | 1.88 | 38.2 |
| 552 | 289 | 1.67 | 86.2 |
| 553 | 523 | 2.97 | 51 |
| 554 | 946 | 4.08 | 60.8 |
| 555 | 274 | 5.31 | 42 |
| 556 | 366 | 5.51 | 47.4 |
| 557 | 223 | 2.99 | 20.8 |
| 558 | 291 | 3.62 | 28 |
| 559 | 323 | 9.15 | 49.7 |
| 560 | 355 | 7.94 | 52 |
| 561 | 544 | 10.1 | 76.1 |
| 562 | 427 | 10.2 | 89.5 |
| 563 | 1010 | 11.8 | 98.7 |
| 564 | 667 | 10.8 | 101 |
| 565 | 1770 | 21.9 | 204 |
| 566 | 1780 | 30.1 | 252 |
| 567 | 521 | 13.4 | 102 |
| 568 | 477 | 13.2 | 116 |
| 569 | 697 | 12.4 | 84.4 |
| 570 | 648 | 13.6 | 95.1 |
| 571 | 243 | 4.23 | 42.7 |
| 572 | 330 | 3.13 | 30.4 |
| 573 | 388 | 3.15 | 31.6 |
| 574 | 435 | 20.5 | 37.2 |
| 575 | 484 | 16.2 | 37.1 |
| 576 | 646 | 8.12 | 56.8 |
| 577 | 937 | 10 | 81.8 |
| 578 | 781 | 3.73 | 54.7 |
| 579 | 679 | 3.79 | 67.3 |
| 580 | 922 | 6.29 | 55.3 |
| 581 | 1500 | 7.29 | 65.9 |
| 582 | 1060 | 10.8 | 35.5 |
| 583 | 1250 | 12.3 | 71 |
| 584 | 497 | 5.5 | 137 |
| 585 | 436 | 3.93 | 38.6 |
| 586 | 302 | 2.46 | 29.1 |
| 587 | 252 | 3.47 | 31.9 |
| 588 | 259 | 2.86 | 34.1 |
| 589 | 289 | 3.83 | 44.4 |
| 590 | 309 | 4.25 | 58.7 |
| 591 | 234 | 3 | 44.5 |
| 592 | 195 | 4.05 | 55.6 |
| 593 | 591 | 2.71 | 35.3 |
| 594 | 570 | 3.32 | 49.1 |
| 595 | 353 | 1.73 | 30.4 |
| 596 | 385 | 1.98 | 36.6 |
| 597 | 329 | 2.38 | 36.4 |
| 598 | 457 | 3.49 | 47.3 |
| 599 | 629 | 9.67 | 89.3 |
| 600 | 587 | 9.56 | 85.7 |
| 601 | 201 | 2.11 | 27.7 |
| 602 | 190 | 3.53 | 31.9 |
| 603 | 419 | 4.99 | 49.5 |
| 604 | 427 | 4.84 | 51 |
| 605 | 353 | 6.69 | 56.2 |
| 606 | 474 | 5.69 | 53.1 |
| 607 | 565 | 6.63 | 139 |
| 608 | 669 | 8.51 | 201 |
| 609 | 647 | 6.19 | 121 |
| 610 | 536 | 7.02 | 130 |
| 611 | 627 | 7.35 | 146 |
| 612 | 640 | 7.58 | 126 |
| 613 | 1410 | 0.791 | 81.6 |
| 614 | 724 | 0.597 | 33.2 |
| 615 | 697 | 0.95 | 82.9 |
| 616 | 734 | 0.467 | 28.9 |
| 617 | 547 | 0.643 | 67.4 |
| 618 | 691 | 4.63 | 88.9 |
| 619 | 819 | 6.78 | 94 |
| 620 | 665 | 5.96 | 90.7 |
| 621 | 901 | 7.31 | 111 |
| 622 | 641 | 1.19 | 52.3 |
| 623 | 832 | 1.48 | 62.2 |
| 624 | 808 | 1.17 | 63.4 |
| 625 | 657 | 1.81 | 72.1 |
| 626 | 546 | 1.75 | 60.8 |
| 627 | 813 | 1.47 | 75.1 |
| 628 | 651 | 1.87 | 79.8 |
| 629 | 898 | 2.37 | 110 |
| 630 | 872 | 2.96 | 74.5 |
| 631 | 1250 | 3.92 | 55 |
| 632 | 1600 | 5.48 | 88.2 |
| 633 | 1500 | 1.63 | 41.2 |
| 634 | 979 | 1.48 | 34.9 |
| 635 | 753 | 2.7 | 53.9 |
| 636 | 935 | 4.3 | 36.6 |
| 637 | 968 | 7.23 | 76.2 |
| 638 | 1090 | 2.39 | 36 |
| 639 | 750 | 2.04 | 27.9 |
| 640 | 730 | 0.591 | 27.8 |
| 641 | 451 | 0.832 | 68.3 |
| 642 | 519 | 0.791 | 67.1 |
| 643 | 1170 | 1.61 | 60.1 |
| 644 | 626 | 0.601 | 34.3 |
| 645 | 398 | 0.863 | 76.3 |
| 646 | 1260 | 1.09 | 90.5 |
| 647 | 1210 | 0.691 | 36.1 |
| 648 | 1170 | 1.4 | 86.5 |
| 649 | 669 | 0.76 | 62.9 |
| 650 | 574 | 3.1 | 31.4 |
| 651 | 639 | 6.3 | 53.9 |
| 652 | 764 | 1.92 | 26.8 |
| 653 | 542 | 1.38 | 25 |
| 654 | 886 | 10.9 | 116 |
| 655 | 883 | 8.86 | 76.9 |
| 656 | 662 | 7.13 | 119 |
| 657 | 226 | 6.43 | 74.5 |
| 658 | 376 | 12 | 106 |
| 659 | 735 | 5.75 | 81.5 |
| 660 | 455 | 4.64 | 76.9 |
| 661 | 160 | 4.62 | 50.9 |
| 662 | 561 | 4.69 | 84.5 |
| 663 | 226 | 10.2 | 79 |
| 664 | 179 | 1.18 | 27.7 |
| 665 | 285 | 2.1 | 32.8 |
| 666 | 224 | 1.39 | 32.3 |
| 667 | 215 | 1.46 | 33 |
| 668 | 203 | 1.69 | 27.1 |
| 669 | 173 | 1.41 | 27.3 |
| 670 | 489 | 1.9 | 104 |
| 671 | 152 | 0.657 | 17.6 |
| 672 | 162 | 1.53 | 49.4 |
| 673 | 114 | 0.419 | 10.8 |
| 674 | 168 | 2.54 | 17.3 |
| 675 | 207 | 1.16 | 25 |
| 676 | 208 | 1.07 | 24.4 |
| 677 | 190 | 1.03 | 20.8 |
| 678 | 245 | 0.929 | 22.1 |
| 679 | 188 | 1.02 | 21.6 |
| 680 | 256 | 1.04 | 28.6 |
| 681 | 104 | 0.395 | 14.2 |
| 682 | 127 | 0.415 | 14 |
| 683 | 137 | 0.492 | 14.3 |
| 684 | 143 | 0.436 | 14.2 |
| 685 | 160 | 0.497 | 18.7 |
| 686 | 169 | 0.542 | 19.1 |
| 687 | 157 | 0.595 | 21.3 |
| 688 | 182 | 0.61 | 19.5 |
| 689 | 182 | 0.951 | 25.1 |
| 690 | 142 | 0.522 | 17.4 |
| 691 | 308 | 1.17 | 28.9 |
| 692 | 380 | 1.25 | 25.2 |
| 693 | 304 | 1.1 | 18 |
| 694 | 206 | 0.787 | 25 |
| 695 | 164 | 0.772 | 19.5 |
| 696 | 173 | 0.53 | 19.1 |
| 697 | 130 | 0.607 | 18.2 |
| 698 | 124 | 0.824 | 20.6 |
| 699 | 144 | 0.566 | 25.3 |
| 700 | 132 | 0.397 | 20 |
| 701 | 226 | 0.979 | 24.9 |
| 702 | 196 | 1.25 | 28.4 |
| 703 | 201 | 0.961 | 27.1 |
| 704 | 239 | 1.2 | 33.9 |
| 705 | 234 | 1.15 | 33.7 |
| 706 | 240 | 0.913 | 23.7 |
| 707 | 199 | 1.05 | 30.6 |
| 708 | 124 | 0.562 | 22.1 |
| 709 | 107 | 0.63 | 23.4 |
| 710 | 126 | 0.589 | 28.7 |
| 711 | 197 | 0.786 | 35.2 |
| 712 | 171 | 0.584 | 30.8 |
| 713 | 180 | 0.879 | 37.7 |
| 714 | 197 | 0.754 | 33.2 |
| 715 | 126 | 0.51 | 19.7 |
| 716 | 100 | 0.557 | 24.1 |
| 717 | 130 | 0.561 | 21.4 |
| 718 | 144 | 0.531 | 23.2 |
| 719 | 181 | 0.882 | 23.2 |
| 720 | 239 | 1.02 | 28.3 |
| 721 | 183 | 1.19 | 27.1 |
| 722 | 170 | 0.953 | 27.2 |
| 723 | 179 | 1.02 | 26.5 |
| 724 | 209 | 1.06 | 26.4 |
| 725 | 244 | 1.39 | 29.1 |
| 726 | 226 | 1.22 | 33.3 |
| 727 | 287 | 1.09 | 24.1 |
| 728 | 298 | 1.45 | 26.7 |
| 729 | 276 | 1.43 | 27 |
| 730 | 216 | 1.29 | 33 |
| 731 | 291 | 1.27 | 27.4 |
| 732 | 327 | 1.26 | 27.2 |
| 733 | 207 | 1.15 | 34.3 |
| 734 | 230 | 1.3 | 38.1 |
| 735 | 236 | 1.32 | 40.7 |
| 736 | 245 | 1.23 | 46.3 |
| 737 | 122 | 0.498 | 22.3 |
| 738 | 162 | 0.64 | 28.2 |
| 739 | 155 | 0.507 | 27.1 |
| 740 | 181 | 0.629 | 29.7 |
| 741 | 200 | 0.817 | 32.4 |
| 742 | 203 | 0.857 | 33.3 |
| 743 | 133 | 0.432 | 21.2 |
| 744 | 111 | 0.488 | 22.1 |
| 745 | 266 | 1.36 | 29 |
| 746 | 255 | 1.44 | 38.6 |
| 747 | 169 | 0.651 | 20.3 |
| 748 | 156 | 0.865 | 22.9 |
| 749 | 28 | 1.47 | 33.9 |
| 750 | 205 | 1.34 | 39.1 |
| 751 | 267 | 1.27 | 32.3 |
| 752 | 228 | 1.74 | 44 |
| 753 | 351 | 1.86 | 38.2 |
| 754 | 381 | 1.94 | 33.3 |
| 755 | 274 | 1.39 | 23.9 |
| 756 | 372 | 1.61 | 28.2 |
| 757 | 310 | 1.81 | 30.8 |
| 758 | 388 | 1.59 | 31.3 |
| 759 | 223 | 1.05 | 34.8 |
| 760 | 239 | 1.29 | 43.6 |
| 761 | 162 | 0.635 | 26.1 |
| 762 | 119 | 0.692 | 27.6 |
| 763 | 222 | 1.16 | 42.5 |
| 764 | 146 | 0.649 | 38.8 |
| 765 | 111 | 44.6 | 26.1 |
| 766 | 65.7 | 14.1 | 32.5 |
| 767 | 207 | 1.51 | 56.8 |
| 768 | 2270 | 2.65 | 33.2 |
| 769 | 269 | 1.02 | 34 |
| 770 | 390 | 18.2 | 240 |
| 771 | 955 | 44.2 | 665 |
| 772 | 1880 | 21.6 | 206 |
| 773 | 2260 | 26.3 | 241 |
| 774 | 2330 | 32.8 | 404 |
| 775 | 724 | 25.8 | 202 |
| 776 | 908 | 25.7 | 191 |
| 777 | 1320 | 22.2 | 231 |
| 778 | 1370 | 30.5 | 245 |
| 779 | 1020 | 19.3 | 175 |
| 780 | 1250 | 22.1 | 211 |
| 781 | 808 | 13.1 | 244 |
| 782 | 1380 | 15.5 | 354 |
| 783 | 1300 | 16.9 | 497 |
| 784 | 711 | 11.8 | 284 |
| 785 | 1210 | 21.3 | 391 |
| 786 | 1490 | 25.9 | 536 |
| 787 | 2060 | 18.6 | 382 |
| 788 | 1420 | 20.7 | 528 |
| 789 | 1200 | 20.4 | 379 |
| 790 | 1200 | 34.1 | 858 |
| 791 | 456 | 143 | 980 |
| 792 | 376 | 108 | 619 |
| 793 | 1660 | 135 | 559 |
| 794 | 297 | 68.4 | 233 |
| 795 | 566 | 67.3 | 221 |
| 796 | 1000 | 17.5 | 125 |
| 797 | 963 | 16.7 | 102 |
| 798 | NA | NA | NA |
| 799 | 574 | 18.4 | 179 |
| 800 | 2500 | 40.2 | 164 |
| 801 | 1200 | 18.1 | 133 |
| 802 | 2520 | 16.9 | 153 |
| 803 | NA | NA | NA |
| 804 | 2310 | 17.6 | 161 |
| 805 | 1210 | 13 | 146 |
| 806 | 2760 | 15.1 | 128 |
| 807 | NA | NA | NA |
| 808 | 286 | 27.3 | 406 |
| 809 | 377 | 20.4 | 249 |
| 810 | 540 | 23.6 | 256 |
| 811 | 175 | 25.8 | 246 |
| 812 | 207 | 13.2 | 161 |
| 813 | 314 | 19.5 | 184 |
| 814 | 554 | 27.8 | 649 |
| 815 | 798 | 17.9 | 342 |
| 816 | 1100 | 15.9 | 310 |
| 817 | 690 | 12.3 | 151 |
| 818 | 516 | 13.8 | 91.7 |
| 819 | 393 | 9.79 | 134 |
| 820 | 1270 | 17.5 | 299 |
| 821 | 989 | 26.1 | 362 |
| 822 | 2100 | 18.1 | 322 |
| 823 | 1410 | 25.6 | 421 |
| 824 | 1110 | 28.9 | 343 |
| 825 | 1810 | 39.3 | 303 |
| 826 | 441 | 81.6 | 202 |
| 827 | 424 | 19.5 | 53.8 |
| 828 | 481 | 14.9 | 114 |
| 829 | 325 | 22.4 | 130 |
| 830 | 809 | 44.8 | 180 |
| 831 | 1450 | 38.2 | 237 |
| 832 | 1360 | 18.6 | 209 |
| 833 | 305 | 17.5 | 148 |
| 834 | 533 | 15.7 | 208 |
| 835 | 558 | 16.8 | 274 |
| 836 | 2100 | 26.7 | 103 |
| 837 | 2060 | 20.7 | 69.6 |
| 838 | 3070 | 23.2 | 72.2 |
| 839 | 1110 | 21.9 | 60.6 |
| 840 | 1810 | 12.5 | 61 |
| 841 | 2410 | 17.3 | 64.2 |
| 842 | 415 | 31.2 | 164 |
| 843 | 367 | 32.8 | 135 |
| 844 | 583 | 43.3 | 151 |
| 845 | 203 | 23.3 | 89.1 |
| 846 | 599 | 19.5 | 93.1 |
| 847 | 532 | 22.2 | 105 |
| 848 | 615 | 20.2 | 313 |
| 849 | 1160 | 36.6 | 111 |
| 850 | 3660 | 27 | 57.8 |
| 851 | 558 | 24.4 | 428 |
| 852 | 1230 | 45.6 | 182 |
| 853 | 4500 | 32.4 | 102 |
| 854 | 1280 | 18.7 | 570 |
| 855 | 1030 | 46.1 | 207 |
| 856 | 772 | 20.4 | 200 |
| 857 | 1050 | 36.1 | 87.3 |
| 858 | 678 | 13.1 | 258 |
| 859 | 2250 | 32.3 | 543 |
| 860 | 565 | 19.2 | 414 |
| 861 | 1350 | 18.3 | 322 |
| 862 | 1110 | 12.4 | 201 |
| 863 | 334 | 12.2 | 183 |
| 864 | 1480 | 13.2 | 110 |
| 865 | 248 | 10.8 | 351 |
| 866 | 669 | 23.8 | 483 |
| 867 | 242 | 16.2 | 474 |
| 868 | 622 | 17.7 | 822 |
| 869 | 571 | 14.1 | 114 |
| 870 | 736 | 7.29 | 71.2 |
| 871 | 2060 | 35.4 | 230 |
| 872 | 1890 | 30.1 | 287 |
| 873 | 506 | 44.2 | 138 |
| 874 | 603 | 36.2 | 201 |
| 875 | 689 | 13.8 | 78.6 |
| 876 | 940 | 7.94 | 70 |
| 877 | 1090 | 22.7 | 112 |
| 878 | 1130 | 16.7 | 137 |
| 879 | 329 | 20.1 | 591 |
| 880 | 562 | 76.2 | 943 |
| 881 | 507 | 30.1 | 321 |
| 882 | 559 | 26.6 | 361 |
| 883 | 578 | 9.41 | 120 |
| 884 | 613 | 23.5 | 298 |
| 885 | 574 | 19.7 | 326 |
| 886 | 540 | 26.5 | 271 |
| 887 | 525 | 37.1 | 403 |
| 888 | 567 | 30.8 | 238 |
| 889 | 688 | 21.6 | 371 |
| 890 | 559 | 14.2 | 290 |
| 891 | 581 | 16 | 330 |
| 892 | 529 | 21.5 | 405 |
| 893 | 508 | 21.4 | 305 |
| 894 | 2330 | 30.9 | 711 |
| 895 | 1250 | 34 | 599 |
| 896 | 1110 | 16.8 | 535 |
| 897 | 658 | 25.9 | 557 |
| 898 | 770 | 25.5 | 584 |
| 899 | 814 | 15.6 | 592 |
| 900 | 885 | 17 | 666 |
| 901 | 1150 | 34 | 193 |
| 902 | 1360 | 28.5 | 232 |
| 903 | 1630 | 31.2 | 387 |
| 904 | 1330 | 15 | 198 |
| 905 | 1390 | 10.4 | 215 |
| 906 | 1170 | 15.4 | 236 |
| 907 | 1210 | 8.86 | 123 |
| 908 | 980 | 26.7 | 174 |
| 909 | 1740 | 20.2 | 243 |
| 910 | 2640 | 25.7 | 239 |
| 911 | 2390 | 26.6 | 255 |
| 912 | 955 | 6.15 | 59.6 |
| 913 | 1140 | 5.62 | 258 |
| 914 | 983 | 5.94 | 91.7 |
| 915 | 174 | 15.3 | 226 |
| 916 | 158 | 4.78 | 166 |
| 917 | 424 | 6.2 | 195 |
| 918 | 590 | 18 | 484 |
| 919 | 467 | 25.8 | 454 |
| 920 | 1090 | 9.05 | 322 |
| 921 | 2130 | 12.4 | 261 |
| 922 | 1170 | 6.39 | 357 |
| 923 | 1840 | 18.5 | 870 |
| 924 | 910 | 15.7 | 402 |
| 925 | 2500 | 23 | 708 |
| 926 | 1380 | 13.5 | 600 |
| 927 | 1210 | 22.2 | 584 |
| 928 | 1090 | 15.1 | 604 |
| 929 | 2690 | 22.6 | 593 |
| 930 | 1700 | 19.9 | 824 |
| 931 | 2300 | 20.8 | 878 |
| 932 | 513 | 25 | 249 |
| 933 | 1530 | 35.1 | 557 |
| 934 | 1380 | 28.1 | 175 |
| 935 | 659 | 16.9 | 172 |
| 936 | 899 | 18.2 | 244 |
| 937 | 1160 | 19.7 | 261 |
| 938 | 2830 | 21.5 | 263 |
| 939 | 423 | 14.1 | 287 |
| 940 | 429 | 13.7 | 498 |
| 941 | 1430 | 22.1 | 113 |
| 942 | 1560 | 28.3 | 490 |
| 943 | 960 | 23.1 | 382 |
| 944 | 1840 | 37.2 | 985 |
| 945 | 2420 | 42.5 | 555 |
| 946 | 1950 | 57.4 | 576 |
| 947 | 727 | 16.6 | 150 |
| 948 | 537 | 22.9 | 304 |
| 949 | 317 | 26 | 721 |
| 950 | 327 | 18.1 | 328 |
| 951 | 657 | 18.2 | 382 |
| 952 | 266 | 16.6 | 439 |
| 953 | 275 | 10.9 | 347 |
| 954 | 450 | 35.3 | 357 |
| 955 | 366 | 42.3 | 1380 |
| 956 | 406 | 32.1 | 643 |
| 957 | 947 | 25 | 747 |
| 958 | 340 | 27.1 | 850 |
| 959 | 316 | 24.5 | 503 |
| 960 | 813 | 14.1 | 161 |
| 961 | 342 | 13.6 | 216 |
| 962 | 485 | 9.71 | 132 |
| 963 | 900 | 25.1 | 215 |
| 964 | 324 | 21.4 | 267 |
| 965 | 430 | 23.2 | 194 |
| 966 | 581 | 35.9 | 257 |
| 967 | 508 | 39.5 | 345 |
| 968 | 625 | 11.7 | 103 |
| 969 | 660 | 12.6 | 113 |
| 970 | 809 | 20.1 | 186 |
| 971 | 1340 | 30.2 | 294 |
| 972 | 660 | 30.4 | 327 |
| 973 | 2270 | 60.1 | 1140 |
| 974 | 870 | 80.9 | 283 |
| 975 | 374 | 30.1 | 1810 |
| 976 | 848 | 82.7 | 2860 |
| 977 | 839 | 23 | 159 |
| 978 | 824 | 33.9 | 174 |
| 979 | 359 | 19.2 | 257 |
| 980 | 239 | 15.4 | 290 |
| 981 | 477 | 21.1 | 330 |
| 982 | 307 | 19.4 | 310 |
| 983 | 576 | 19.4 | 207 |
| 984 | 409 | 23.5 | 160 |
| 985 | 374 | 24 | 217 |
| 986 | 702 | 31.3 | 380 |
| 987 | 267 | 11.7 | 515 |
| 988 | 320 | 11.8 | 743 |
| 989 | 247 | 8 | 402 |
| 990 | 276 | 9.87 | 557 |
| 991 | 405 | 14.8 | 315 |
| 992 | 863 | 12.5 | 643 |
| 993 | 1860 | 14.5 | 1210 |
| 994 | 1050 | 30.8 | 395 |
| 995 | 295 | 6.12 | 1660 |
| 996 | 1160 | 11.2 | 4810 |
| 997 | 527 | 27.8 | 1880 |
| 998 | 794 | 6.78 | 1470 |
| 999 | 2130 | 10.4 | 3020 |
| 1000 | 501 | 15.5 | 279 |
| 1001 | 1170 | 20 | 295 |
| 1002 | 197 | 11.6 | 1290 |
| 1003 | 371 | 15.9 | 1950 |
| 1004 | 237 | 35.4 | 973 |
| 1005 | 1670 | 14.5 | 171 |
| 1006 | 315 | 11.8 | 574 |
| 1007 | 636 | 12 | 95.1 |
| 1008 | 1010 | 15.2 | 126 |
| 1009 | 268 | 9.72 | 174 |
| 1010 | 214 | 15.1 | 218 |
| 1011 | 459 | 13.5 | 148 |
| 1012 | 533 | 15.9 | 223 |
| 1013 | 285 | 22.6 | 137 |
| 1014 | 277 | 5.62 | 86.4 |
| 1015 | 357 | 9.08 | 92.4 |
| 1016 | 626 | 30.6 | 156 |
| 1017 | 649 | 28.3 | 118 |
| 1018 | 568 | 14.1 | 163 |
| 1019 | 579 | 21.3 | 161 |
| 1020 | 478 | 27.3 | 146 |
| 1021 | 588 | 29 | 174 |
| 1022 | 586 | 27.9 | 203 |
| 1023 | 856 | 26 | 230 |
| 1024 | 760 | 32.5 | 330 |
| 1025 | 625 | 31.5 | 352 |
| 1026 | 2080 | 61.3 | 299 |
| 1027 | 1470 | 58.1 | 252 |
| 1028 | 3910 | 157 | 986 |
| 1029 | 511 | 22.3 | 170 |
| 1030 | 1830 | 21 | 385 |
| 1031 | 1200 | 17.3 | 152 |
| 1032 | 900 | 21.9 | 148 |
| 1033 | 2170 | 23.4 | 221 |
| 1034 | 1890 | 23 | 317 |
| 1035 | 1380 | 16 | 167 |
| 1036 | 353 | 13 | 1080 |
| 1037 | 291 | 13.7 | 625 |
| 1038 | 1300 | 29.8 | 349 |
| 1039 | 2020 | 26.8 | 413 |
| 1040 | 2020 | 20.8 | 322 |
| 1041 | 1720 | 75.7 | 316 |
| 1042 | 1890 | 84.8 | 642 |
| 1043 | 2240 | 44.9 | 268 |
| 1044 | 2410 | 88.7 | 1110 |
| 1045 | 1540 | 107 | 276 |
| 1046 | 1680 | 85 | 528 |
| 1047 | 1510 | 60.8 | 1960 |
| 1048 | 1930 | 108 | 1900 |
| 1049 | 1280 | 56.2 | 780 |
| 1050 | 2040 | 68.5 | 1240 |
| 1051 | 1800 | 32.6 | NA |
| 1052 | 1980 | 38 | NA |
| 1053 | 3470 | 19.3 | NA |
| 1054 | 1630 | 79.4 | NA |
| 1055 | 450 | 78.9 | NA |
| 1056 | 1650 | 29.6 | NA |
| 1057 | 2780 | 25.9 | NA |
| 1058 | 1450 | 45.6 | NA |
| 1059 | 748 | 48.9 | NA |
| 1060 | 4900 | 44.7 | NA |
| 1061 | 2910 | 55.6 | NA |
| 1062 | 2380 | 67.5 | NA |
| 1063 | 2290 | 26.6 | 626 |
| 1064 | 1940 | 62.5 | 912 |
| 1065 | 3080 | 39.1 | 2290 |
| 1066 | 4260 | 69.9 | 1850 |
| 1067 | 1860 | 86.4 | 2610 |
| 1068 | 295 | 20.2 | 1330 |
| 1069 | 2160 | 39.5 | 1290 |
| 1070 | 1940 | 27.3 | 321 |
| 1071 | 2990 | 40.2 | 425 |
| 1072 | 914 | 9.94 | 95.1 |
| 1073 | 1490 | 15.7 | 114 |
| 1074 | 1990 | 8.49 | 101 |
| 1075 | 3020 | 9.88 | 109 |
| 1076 | 1650 | 20.2 | 187 |
| 1077 | 3160 | 27.8 | 408 |
| 1078 | 1680 | 38.6 | 264 |
| 1079 | 4090 | 18.9 | 175 |
| 1080 | 2820 | 18.2 | 281 |
| 1081 | 2130 | 8.44 | 151 |
| 1082 | 1530 | 12.3 | 677 |
| 1083 | 1030 | 20.5 | 547 |
| 1084 | 1390 | 17.2 | 761 |
| 1085 | 1370 | 9.84 | 188 |
| 1086 | 3140 | 21.2 | 496 |
| 1087 | 2460 | 23.3 | 439 |
| 1088 | 1710 | 15.6 | 367 |
| 1089 | 2580 | 34.6 | 449 |
| 1090 | 2010 | 27.3 | 619 |
| 1091 | 550 | 5.79 | 145 |
| 1092 | 506 | 7.83 | 230 |
| 1093 | 958 | 26.8 | 141 |
| 1094 | 831 | 5.86 | 64.4 |
| 1095 | 337 | 8.73 | 159 |
| 1096 | 397 | 11.6 | 194 |
| 1097 | 752 | 27.3 | 141 |
| 1098 | 994 | 31.2 | 184 |
| 1099 | 472 | 8.64 | 75.8 |
| 1100 | 577 | 9.04 | 77.2 |
| 1101 | 827 | 6.31 | 246 |
| 1102 | 516 | 5.12 | 295 |
| 1103 | 1350 | 19.5 | 183 |
| 1104 | 1470 | 13.9 | 272 |
| 1105 | 1260 | 5.59 | 92.5 |
| 1106 | 1030 | 4.23 | 119 |
| 1107 | 864 | 5.29 | 59 |
| 1108 | 468 | 5.22 | 76.2 |
| 1109 | 510 | 20.2 | 102 |
| 1110 | 449 | 5.4 | 52 |
| 1111 | 299 | 3.66 | 51.3 |
| 1112 | 588 | 8.02 | 68.3 |
| 1113 | 343 | 3.47 | 88.2 |
| 1114 | 514 | 2.34 | 39 |
| 1115 | 420 | 1.28 | 60.4 |
| 1116 | 364 | 10.8 | 198 |
| 1117 | 355 | 8.31 | 237 |
| 1118 | 802 | 5.45 | 183 |
| 1119 | 237 | 3.95 | 152 |
| 1120 | 291 | 9.21 | 171 |
| 1121 | 566 | 4.55 | 165 |
| 1122 | 474 | 4.54 | 177 |
| 1123 | 441 | 3.89 | 191 |
| 1124 | 400 | 12 | 304 |
| 1125 | 264 | 3.95 | 243 |
| 1126 | 452 | 13.5 | 208 |
| 1127 | 425 | 7.72 | 234 |
| 1128 | 869 | 3.18 | 52.8 |
| 1129 | 796 | 4.16 | 56.6 |
| 1130 | 1240 | 3.73 | 44.5 |
| 1131 | 1140 | 5.47 | 63.7 |
| 1132 | 184 | 1.89 | 38.8 |
| 1133 | 216 | 4.24 | 47.8 |
| 1134 | 180 | 2.41 | 78.9 |
| 1135 | 142 | 0.864 | 63.2 |
| 1136 | 121 | 0.949 | 39.2 |
| 1137 | 663 | 2.05 | 36.2 |
| 1138 | 72.8 | 3.4 | 164 |
| 1139 | 269 | 2.87 | 155 |
| 1140 | 228 | 0.869 | 39.2 |
| 1141 | 169 | 0.816 | 47.5 |
| 1142 | 203 | 0.748 | 38 |
| 1143 | 182 | 0.751 | 49.6 |
| 1144 | 196 | 0.72 | 36.5 |
| 1145 | 148 | 0.848 | 39.1 |
| 1146 | 198 | 0.825 | 40.1 |
| 1147 | 167 | 0.831 | 45.4 |
| 1148 | 175 | 0.873 | 44.3 |
| 1149 | 184 | 0.96 | 52.2 |
| 1150 | 161 | 0.583 | 30 |
| 1151 | 206 | 0.287 | 40 |
| 1152 | 287 | 1.21 | 43.8 |
| 1153 | 215 | 1.39 | 52.7 |
| 1154 | 320 | 0.954 | 44.5 |
| 1155 | 288 | 0.888 | 44.4 |
| 1156 | 337 | 1.33 | 47.1 |
| 1157 | 384 | 1.28 | 57.5 |
| 1158 | 301 | 1.42 | 46.8 |
| 1159 | 238 | 1.29 | 64.1 |
| 1160 | 183 | 0.598 | 31.2 |
| 1161 | 217 | 0.733 | 35.5 |
| 1162 | 199 | 0.656 | 34.5 |
| 1163 | 163 | 0.721 | 35.4 |
| 1164 | 359 | 1.17 | 63.5 |
| 1165 | 358 | 1.36 | 87.7 |
| 1166 | 427 | 1.1 | 65.6 |
| 1167 | 274 | 1.22 | 89.2 |
| 1168 | 364 | 0.999 | 65.4 |
| 1169 | 304 | 1.18 | 80 |
| 1170 | 249 | 0.663 | 50.2 |
| 1171 | 233 | 0.718 | 52.9 |
| 1172 | 303 | 0.7 | 45.5 |
| 1173 | 254 | 0.764 | 59.1 |
| 1174 | 347 | 0.707 | 60.2 |
| 1175 | 282 | 0.768 | 60.7 |
| 1176 | 229 | 0.462 | 43.8 |
| 1177 | 156 | 0.544 | 43.6 |
| 1178 | 270 | 0.544 | 39 |
| 1179 | 197 | 0.58 | 37.9 |
| 1180 | 205 | 0.341 | 35.6 |
| 1181 | 203 | 0.666 | 48.2 |
| 1182 | 172 | 2.73 | 80.4 |
| 1183 | 127 | 3.16 | 53.6 |
| 1184 | 295 | 8.39 | 179 |
| 1185 | 206 | 2.31 | 66.1 |
| 1186 | 164 | 3.17 | 52.1 |
| 1187 | 370 | 7.11 | 154 |
| 1188 | 130 | 7.02 | 77.1 |
| 1189 | 248 | 0.584 | 45.4 |
| 1190 | 192 | 0.954 | 77.4 |
| 1191 | 243 | 0.931 | 82.3 |
| 1192 | 218 | 1.06 | 59.5 |
| 1193 | 202 | 0.803 | 66.2 |
| 1194 | 215 | 0.759 | 57.4 |
| 1195 | 242 | 0.637 | 44.2 |
| 1196 | 257 | 0.605 | 45.4 |
| 1197 | 288 | 0.639 | 46.1 |
| 1198 | 314 | 0.67 | 61 |
| 1199 | 352 | 0.733 | 63.9 |
| 1200 | 278 | 0.896 | 91.4 |
| 1201 | 234 | 1.02 | 93.9 |
| 1202 | 70.3 | 0.471 | 23.1 |
| 1203 | 436 | 8.5 | 38.9 |
| 1204 | 507 | 15.8 | 37.4 |
| 1205 | 325 | 16.3 | 50 |
| 1206 | 251 | 7.04 | 44.1 |
| 1207 | 470 | 0.633 | 39.8 |
| 1208 | 451 | 0.786 | 44.8 |
| 1209 | 124 | 0.565 | 37.7 |
| 1210 | 288 | 0.635 | 38.1 |
| 1211 | 211 | 0.489 | 33.2 |
| 1212 | 230 | 0.614 | 38.2 |
| 1213 | 268 | 0.616 | 44 |
| 1214 | 210 | 0.771 | 50 |
| 1215 | 272 | 0.937 | 58.5 |
| 1216 | 225 | 0.914 | 66.9 |
| 1217 | 253 | 1.06 | 62.7 |
| 1218 | 185 | 0.626 | 48.7 |
| 1219 | 168 | 0.629 | 46.8 |
| 1220 | 168 | 0.609 | 48.5 |
| 1221 | 185 | 0.814 | 57.8 |
| 1222 | 470 | 12.4 | 44.8 |
| 1223 | 472 | 9.25 | 40.7 |
| 1224 | 208 | 3.78 | 28.4 |
| 1225 | 275 | 3.67 | 31.4 |
| 1226 | 184 | 0.443 | 34.4 |
| 1227 | 199 | 0.527 | 39.5 |
| 1228 | 185 | 0.522 | 39.1 |
| 1229 | 175 | 0.504 | 37.7 |
| 1230 | 68.4 | 0.0152 | 1.14 |
| 1231 | 17.1 | 0.00994 | 1.22 |
| 1232 | 8.53 | 0.00687 | 0.694 |
| 1233 | 9.9 | 0.00988 | 1.06 |
| 1234 | 11.7 | 0.011 | 1.15 |
| 1235 | >9950 | 20.3 | 1800 |
| 1236 | 502 | 11.9 | 95.8 |

NOTE:
EC₅₀ determination of human GLP-1(7-36)NH₂ at human GLP-1R, human Gcg at human GcgR, and human GIP(1-42)NH₂ at human GIP-R: the peptide concentration ranges were 346 pM to 9950 nM. EC₅₀ determination of Examples at human GLP-1R, human GcgR, and human GIP-R: the peptide concentration ranges were 6.87 pM to 9.95 μM.

As seen in Table 2, in the presence of HSA, exemplary analogs have agonist activities as determined by human GIP-R, GLP-1R, and GcgR cAMP assays, which are lower than the native ligands.

To determine the intrinsic potency of exemplary analogs and comparator molecules, the CAMP assays described above were performed in the presence of 0.1% casein only (without human serum albumin). Casein is used as a nonspecific blocker in both cAMP assays and does not interact with the fatty acid moieties of the analyzed molecules.

Intracellular cAMP levels are determined by extrapolation using a standard curve. Dose response curves of compounds are plotted as the percentage of stimulation normalized to minimum (buffer only) and maximum (maximum concentration of each control ligand) values and analyzed using a four parameter non-linear regression fit with a variable slope (Genedata Screener 13). EC₅₀ is the concentration of compound causing half-maximal simulation in a dose response curve. Each relative EC₅₀ value for the Geometric mean calculation is determined from a curve fitting.

Data are provided below in Table 3.

TABLE 3
Functional Activation of hGLP-1R, hGIPR,
hGcgR in the Presence of 0.1% Casein.

| | Relative cAMP EC₅₀, nM | | |
|---|---|---|---|
| Example | hGcgR | hGIPR | hGLP-1R |
| hGcg | 2.07 | >300 | 116 |
| hGIP 1-42 amide | >99.5 | 0.498 | >9950 |
| hGLP-1 7-36 amide | >99.5 | >99.5 | 0.314 |
| 1 | 3.72 | 0.207 | 1.42 |
| 2 | 4.29 | 0.207 | 1.65 |
| 3 | 3.8 | 0.217 | 1.67 |
| 4 | 3.2 | 0.192 | 1.66 |
| 5 | 7.4 | 0.177 | 2.37 |
| 6 | 6.89 | 0.232 | 1.95 |
| 7 | 5.05 | 0.203 | 2.11 |
| 8 | 6.65 | 0.231 | 2.11 |
| 9 | 7.19 | 0.205 | 1.61 |
| 10 | 7.62 | 0.226 | 1.85 |
| 11 | 12.5 | 0.233 | 2.07 |
| 12 | 12.2 | 0.17 | 1.46 |
| 13 | 7.19 | 0.196 | 1.78 |
| 14 | 7.23 | 0.17 | 1.67 |
| 15 | 11.1 | 0.201 | 1.77 |
| 16 | 8.84 | 0.217 | 1.54 |
| 17 | 8.33 | 0.317 | 2.96 |
| 18 | 2.7 | 0.337 | 2.31 |
| 19 | 2.35 | 0.228 | 3.96 |
| 20 | 15.4 | 0.381 | 2.45 |
| 21 | 12.4 | 0.218 | 2.7 |
| 22 | 5.42 | 0.246 | 2.94 |
| 23 | 5.13 | 0.178 | 2.98 |
| 24 | 9.66 | 0.308 | 3.09 |
| 25 | 6.8 | 0.128 | 3.34 |
| 26 | 4.82 | 0.2 | 3.44 |
| 27 | 8.91 | 0.41 | 2.33 |
| 28 | 12.6 | 0.255 | 3.68 |
| 29 | 11.1 | 0.35 | 3.56 |
| 30 | 5.22 | 0.247 | 3.18 |
| 31 | 8.42 | 0.249 | 2.16 |
| 32 | 11.6 | 0.28 | 2.55 |
| 33 | 7.04 | 0.285 | 1.71 |
| 34 | 3.47 | 0.122 | 1.16 |
| 35 | 4.32 | 0.55 | 3.05 |
| 36 | 4.32 | 0.482 | 3.57 |
| 37 | 6.83 | 0.207 | 1.66 |
| 38 | 9.5 | 0.304 | 2.4 |
| 39 | 10.1 | 0.299 | 2.13 |
| 40 | 12.3 | 0.139 | 0.693 |
| 41 | 8.59 | 0.258 | 2.2 |
| 42 | 5.83 | 0.115 | 2.17 |
| 43 | 2.71 | 0.101 | 1.55 |
| 44 | 2.46 | 0.116 | 1.66 |
| 45 | 2.08 | 0.112 | 1.52 |
| 46 | 1.53 | 0.0947 | 4.04 |
| 47 | 5.61 | 0.388 | 3.4 |
| 48 | 3.8 | 0.27 | 2.99 |
| 49 | 5.54 | 0.324 | 0.706 |
| 50 | 4.25 | 0.29 | 4.65 |
| 51 | 5.15 | 0.421 | 5.8 |
| 52 | 5.84 | 0.449 | 5.18 |
| 53 | 4.19 | 0.518 | 3.31 |
| 54 | 3.77 | 0.502 | 3.95 |
| 55 | 5.2 | 0.0856 | 1.96 |
| 56 | 2.8 | 0.315 | 4.07 |
| 57 | 4.37 | 0.098 | 3.06 |
| 58 | 4.86 | 0.175 | 2.93 |
| 59 | 6.57 | 0.54 | 4.8 |
| 60 | 8.96 | 0.135 | 1.93 |
| 61 | 3.57 | 0.122 | 2.05 |
| 62 | 3.96 | 0.146 | 2.72 |
| 63 | 4.1 | 0.255 | 4.78 |
| 64 | 4.94 | 0.309 | 4.08 |
| 65 | 6.49 | 0.739 | 4.21 |
| 66 | 3.71 | 0.219 | 3.86 |
| 67 | 3.7 | 0.34 | 4.07 |
| 68 | 8.55 | 0.46 | 3.07 |
| 69 | 4.75 | 0.136 | 3.25 |
| 70 | 6.04 | 0.102 | 2.71 |
| 71 | 8.74 | 0.105 | 3.12 |
| 72 | 5.48 | 0.202 | 1.86 |
| 73 | 4.58 | 0.114 | 2.44 |
| 74 | 2.53 | 0.0708 | 3.32 |
| 75 | 4.51 | 0.0578 | 4.4 |
| 76 | 11.1 | 0.196 | 2.25 |
| 77 | 15.3 | 0.168 | 1.85 |
| 78 | 7.06 | 0.228 | 1.93 |
| 79 | 3.08 | 0.0773 | 1.8 |
| 80 | 7.44 | 0.0593 | 2.55 |
| 81 | 7.78 | 0.246 | 2.19 |
| 82 | 6.98 | 0.302 | 3.52 |
| 83 | 10.2 | 0.185 | 2.85 |
| 84 | 8.48 | 0.21 | 1.61 |
| 85 | 7.63 | 0.157 | 2.73 |
| 86 | 7.23 | 0.145 | 2.43 |
| 87 | 8.51 | 0.128 | 2.75 |
| 88 | 7.48 | 0.177 | 3.45 |
| 89 | 11.6 | 0.167 | 3.31 |
| 90 | 9.58 | 0.173 | 2.68 |
| 91 | 9.13 | 0.243 | 3.39 |
| 92 | 12 | 0.212 | 2.08 |
| 93 | 7.16 | 0.162 | 2.63 |
| 94 | 8.16 | 0.154 | 2.63 |
| 95 | 8.41 | 0.116 | 1.95 |
| 96 | 14.1 | 0.456 | 3.92 |
| 97 | 6.73 | 0.176 | 3.23 |
| 98 | 6.91 | 0.179 | 4.35 |
| 99 | 6.09 | 0.174 | 3.54 |
| 100 | 13.1 | 0.293 | 3.21 |
| 101 | 10 | 0.436 | 4.3 |
| 102 | 9.75 | 0.587 | 3.35 |
| 103 | 10.5 | 0.2 | 3.27 |
| 104 | 7.58 | 0.251 | 3.39 |
| 105 | 12.5 | 0.269 | 2.86 |
| 106 | 6.26 | 0.345 | 3.94 |
| 107 | 10.8 | 0.426 | 8.5 |
| 108 | 3.95 | 0.222 | 3.1 |
| 109 | 11.2 | 0.279 | 5.95 |
| 110 | 2.45 | 0.102 | 2.99 |
| 111 | 4.25 | 0.0888 | 5.06 |
| 112 | 11 | 0.233 | 5.77 |
| 113 | 12 | 0.24 | 3.93 |
| 114 | 5.63 | 0.14 | 6.38 |
| 115 | 10.6 | 0.457 | 7.6 |
| 116 | 3.96 | 0.382 | 7.83 |
| 117 | 4.75 | 0.267 | 6.4 |
| 118 | 7.02 | 0.43 | 6.03 |
| 119 | 5.6 | 0.179 | 3.67 |
| 120 | 5.85 | 0.44 | 4.77 |
| 121 | 4.89 | 0.172 | 2.38 |
| 122 | 3.95 | 0.189 | 3.93 |
| 123 | 4.55 | 0.531 | 4.82 |
| 124 | 2.92 | 0.344 | 6.14 |
| 125 | 3.97 | 0.503 | 4.12 |
| 126 | 6.81 | 0.264 | 3.97 |
| 127 | 4.35 | 0.312 | 4.8 |
| 128 | 6.76 | 0.307 | 3.5 |
| 129 | 2.01 | 0.134 | 1.87 |
| 130 | 3.74 | 0.411 | 4.29 |
| 131 | 2.64 | 0.416 | 5.51 |
| 132 | 2.63 | 0.21 | 5.72 |
| 133 | 1.64 | 0.15 | 3.6 |
| 134 | 7.28 | 0.152 | 3.36 |
| 135 | 2.53 | 0.246 | 3.3 |
| 136 | 8.73 | 0.221 | 3.8 |
| 137 | 4.43 | 0.269 | 3.18 |
| 138 | 4.79 | 0.262 | 3.54 |
| 139 | 2.94 | 0.244 | 3.11 |
| 140 | 4.83 | 0.205 | 7.79 |
| 141 | 1.77 | 0.21 | 1.96 |
| 142 | 3.49 | 0.216 | 4.37 |
| 143 | 4.51 | 0.211 | 2.44 |
| 144 | 14.8 | 0.23 | 4.02 |
| 145 | 3.14 | 0.539 | 4.26 |
| 146 | 2.04 | 0.362 | 2.73 |
| 147 | 4.39 | 0.343 | 4.26 |
| 148 | 3.92 | 0.352 | 2.82 |
| 149 | 12.6 | 0.46 | 3.85 |
| 150 | 5.37 | 0.346 | 2.63 |
| 151 | 3.5 | 0.0784 | 1.6 |
| 152 | 6.91 | 0.259 | 4.03 |
| 153 | 14.7 | 0.218 | 7.09 |
| 154 | 8.72 | 0.227 | 4.94 |
| 155 | 5.68 | 0.177 | 2.98 |
| 156 | 3.62 | 0.208 | 3.37 |
| 157 | 4.16 | 0.183 | 3.04 |
| 158 | 3.72 | 0.245 | 2.49 |
| 159 | 6.89 | 0.343 | 4.15 |
| 160 | 4.32 | 0.354 | 5.39 |
| 161 | 4.83 | 0.555 | 5.22 |
| 162 | 4.72 | 0.222 | 4.72 |
| 163 | 5.6 | 0.376 | 6.5 |
| 164 | 2.92 | 0.302 | 5.02 |
| 165 | 4.47 | 0.0956 | 6.52 |
| 166 | 3.82 | 0.118 | 6.96 |
| 167 | 5.05 | 0.101 | 6.19 |
| 168 | 5.54 | 0.233 | 2.44 |
| 169 | 11.7 | 0.209 | 6.17 |
| 170 | 5.29 | 0.401 | 4.11 |
| 171 | 3.86 | 0.2 | 2.89 |
| 172 | 3.38 | 0.174 | 3.5 |
| 173 | 3.6 | 0.168 | 2.32 |
| 174 | 4.24 | 0.198 | 2.33 |
| 175 | 4.92 | 0.218 | 4 |
| 176 | 4.1 | 0.212 | 2.92 |
| 177 | 4.22 | 0.213 | 2.97 |
| 178 | 7.29 | 0.34 | 4.01 |
| 179 | 4.82 | 0.104 | 5.81 |
| 180 | 2.44 | 0.14 | 5.08 |
| 181 | 6.25 | 0.133 | 4.39 |
| 182 | 5.33 | 0.0781 | 4.8 |
| 183 | 3.83 | 0.0759 | 5.03 |
| 184 | 5.23 | 0.162 | 5.02 |
| 185 | 3.5 | 0.123 | 5.46 |
| 186 | 4.67 | 0.107 | 5.26 |
| 187 | 14.8 | 0.111 | 1.11 |
| 188 | 10.8 | 0.193 | 3.43 |
| 189 | 6.28 | 0.0695 | 4.46 |
| 190 | 2.95 | 0.306 | 1.41 |
| 191 | 2.11 | 0.422 | 2.48 |
| 192 | 4.18 | 0.142 | 9.77 |
| 193 | 4.51 | 0.0609 | 5.19 |
| 194 | 4.32 | 0.212 | 4.69 |
| 195 | 3.1 | 0.145 | 6.13 |
| 196 | 8.83 | 0.42 | 5.67 |
| 197 | 3.23 | 0.31 | 4.81 |
| 198 | 6.85 | 0.182 | 4.36 |
| 199 | 5.8 | 0.232 | 4.91 |
| 200 | 2.83 | 0.0739 | 3.19 |
| 201 | 3.84 | 0.0553 | 3.16 |
| 202 | 11.4 | 0.0965 | 4.79 |
| 203 | 6.92 | 0.125 | 4.72 |
| 204 | 6.92 | 0.181 | 2.34 |
| 205 | 8.98 | 0.16 | 2.71 |
| 206 | 2.2 | 0.224 | 2.77 |
| 207 | 4.24 | 0.321 | 4.36 |
| 208 | 2.69 | 0.159 | 3.72 |
| 209 | 3.08 | 0.129 | 1.78 |
| 210 | 2.81 | 0.153 | 1.44 |
| 211 | 2.05 | 0.116 | 1.23 |
| 212 | 3.74 | 0.13 | 1.35 |
| 213 | 3.61 | 0.124 | 1.9 |
| 214 | 2.49 | 0.103 | 1.22 |
| 215 | 9.77 | 0.418 | 0.803 |
| 216 | 2.7 | 0.0887 | 0.52 |
| 217 | 5.11 | 0.0897 | 0.667 |
| 218 | 7.89 | 0.0853 | 0.76 |
| 219 | 7.77 | 0.119 | 0.835 |
| 220 | 11.2 | 0.411 | 1.01 |
| 221 | 11.3 | 0.105 | 0.629 |
| 222 | 2.33 | 0.228 | 3.64 |
| 223 | 1.57 | 0.215 | 2.83 |
| 224 | 1.91 | 0.114 | 1.89 |
| 225 | 2.36 | 0.213 | 2.44 |
| 226 | 3.08 | 0.127 | 1.97 |
| 227 | 4.22 | 0.17 | 2.34 |
| 228 | 1.38 | 0.147 | 1.52 |
| 229 | 2.3 | 0.179 | 1.58 |
| 230 | 1.61 | 0.0909 | 0.937 |
| 231 | 2.12 | 0.119 | 1.3 |
| 232 | 2.42 | 0.102 | 0.819 |
| 233 | 2.25 | 0.0884 | 0.874 |
| 234 | 7.87 | 0.322 | 4.72 |
| 235 | 4.35 | 0.388 | 3.55 |
| 236 | 2.23 | 0.309 | 3.33 |
| 237 | 3.77 | 0.228 | 3.4 |
| 238 | 2.78 | 0.0909 | 2.71 |
| 239 | 2.14 | 0.0503 | 2.9 |
| 240 | 2.02 | 0.214 | 3.62 |
| 241 | 2.71 | 0.0748 | 2.3 |
| 242 | 3.5 | 0.0916 | 3.42 |
| 243 | 6.86 | 0.178 | 6.46 |
| 244 | 4.81 | 0.0972 | 7.68 |
| 245 | 7.07 | 0.0684 | 5.99 |
| 246 | 2.38 | 0.106 | 3.91 |
| 247 | 1.76 | 0.0794 | 2.8 |
| 248 | 3.36 | 0.0861 | 3.17 |
| 249 | 5.55 | 0.173 | 2.4 |
| 250 | 3.81 | 0.154 | 2.08 |
| 251 | 6.98 | 0.153 | 2.64 |
| 252 | 2.61 | 0.147 | 3.54 |
| 253 | 6.62 | 0.115 | 2.2 |
| 254 | 4.9 | 0.121 | 3.08 |
| 255 | 5.48 | 0.107 | 1.9 |
| 256 | 5.71 | 0.176 | 2.87 |
| 257 | 7.36 | 0.135 | 2.66 |
| 258 | 7.15 | 0.154 | 2.71 |
| 259 | 2.55 | 0.114 | 1.47 |
| 260 | 5.04 | 0.251 | 3.85 |
| 261 | 5.49 | 0.29 | 3.12 |
| 262 | 9.77 | 0.232 | 4.39 |
| 263 | 7.53 | 0.169 | 1.32 |
| 264 | 6.29 | 0.0884 | 4.67 |
| 265 | 5.63 | 0.239 | 2.73 |
| 266 | 4.57 | 0.166 | 4.77 |
| 267 | 5.74 | 0.279 | 5.86 |
| 268 | 7.69 | 0.274 | 3.14 |
| 269 | 7.69 | 0.288 | 3.23 |
| 270 | 5 | 0.26 | 3.2 |
| 271 | 8.51 | 0.152 | 5 |
| 272 | 7.91 | 0.254 | 2.62 |
| 273 | 6.96 | 0.243 | 4.02 |
| 274 | 9.37 | 0.296 | 4.9 |
| 275 | 7.8 | 0.318 | 4.93 |
| 276 | 2.82 | 0.0708 | 2.61 |
| 277 | 10.6 | 0.0672 | 5.56 |
| 278 | 4.45 | 0.0963 | 4.11 |
| 279 | 5.06 | 0.105 | 5.77 |
| 280 | 8.63 | 0.105 | 0.95 |
| 281 | 13.7 | 0.154 | 1.6 |
| 282 | 6.98 | 0.163 | 1.09 |
| 283 | 9.73 | 0.225 | 1.16 |
| 284 | 9.21 | 0.347 | 1.54 |
| 285 | 2.32 | 0.0652 | 2.87 |
| 286 | 2.2 | 0.0845 | 3.45 |
| 287 | 2.53 | 0.0633 | 4.25 |
| 288 | 5.17 | 0.234 | 1.89 |
| 289 | 5.02 | 0.382 | 0.449 |
| 290 | 2.68 | 0.43 | 0.34 |
| 291 | 1.88 | 0.26 | 0.22 |
| 292 | 1.84 | 1.02 | 1.03 |
| 293 | 4.01 | 0.0587 | 0.994 |
| 294 | 1.07 | 0.318 | 2.16 |
| 295 | 2.15 | 0.126 | 0.831 |
| 296 | 1.72 | 0.0806 | 0.536 |
| 297 | 3.26 | 0.626 | 2.96 |
| 298 | 1.04 | 0.138 | 0.277 |
| 299 | 2.54 | 0.137 | 0.963 |
| 300 | 6.54 | 0.0975 | 1.05 |
| 301 | 6.73 | 0.722 | 0.641 |
| 302 | 4.47 | 0.7 | 0.621 |
| 303 | 6.1 | 0.116 | 1.13 |
| 304 | 2.31 | 0.127 | 0.551 |
| 305 | 6.73 | 1 | 1.48 |
| 306 | 5.8 | 0.142 | 0.335 |
| 307 | 1.68 | 0.446 | 0.836 |
| 308 | 14.2 | NA | 3.15 |
| 309 | 9.28 | 0.32 | 0.808 |
| 310 | 3.52 | 0.0939 | 0.586 |
| 311 | 2.08 | 0.113 | 3.36 |
| 312 | 7.5 | 0.504 | 2.66 |
| 313 | 4.11 | 0.49 | 0.304 |
| 314 | 3.7 | 0.345 | 0.274 |
| 315 | 4.05 | 0.398 | 0.508 |
| 316 | 5.22 | 0.259 | 0.362 |
| 317 | 2.42 | 0.474 | 1.32 |
| 318 | 5.03 | 0.378 | 0.698 |
| 319 | 4.81 | 0.485 | 0.947 |
| 320 | 6.87 | 0.274 | 2.58 |
| 321 | 4.49 | 0.0912 | 0.798 |
| 322 | 5.41 | 0.264 | 0.394 |
| 323 | 6.04 | 0.232 | 1.56 |
| 324 | 5.74 | 0.164 | 4.4 |
| 325 | 2.43 | 0.351 | 1.56 |
| 326 | 5.34 | 0.3 | 0.555 |
| 327 | 3.04 | 0.312 | 1.61 |
| 328 | 10.3 | 0.544 | 1.3 |
| 329 | 3.47 | 0.207 | 0.926 |
| 330 | 3.91 | 0.0783 | 0.63 |
| 331 | 1.66 | 0.148 | 0.663 |
| 332 | 11.8 | 0.413 | 1.24 |
| 333 | 7.09 | 0.265 | 0.705 |
| 334 | 6.77 | 0.223 | 0.707 |
| 335 | 6.11 | 0.292 | 0.461 |
| 336 | 7.05 | 0.306 | 0.921 |
| 337 | 5.95 | 0.332 | 0.686 |
| 338 | 4.02 | 0.0733 | 1.22 |
| 339 | 6.81 | 0.101 | 1.06 |
| 340 | 2.02 | 0.189 | 0.648 |
| 341 | 10.7 | 0.213 | 1.62 |
| 342 | 10.2 | 0.347 | 0.419 |
| 343 | 6.83 | 0.184 | 1.31 |
| 344 | 9.05 | 0.406 | 2.56 |
| 345 | 11.3 | 0.0377 | 0.658 |
| 346 | 15.6 | 0.0738 | 1.17 |
| 347 | 8.88 | 0.0634 | 0.798 |
| 348 | 3.9 | 0.127 | 0.346 |
| 349 | 5.12 | 0.152 | 0.555 |
| 350 | 10.4 | 0.106 | 0.776 |
| 351 | 12.1 | 0.243 | 0.679 |
| 352 | 16.4 | 0.245 | 0.967 |
| 353 | 11 | 0.127 | 0.634 |
| 354 | 7.91 | 0.134 | 1.21 |
| 355 | 5.87 | 0.0811 | 1.01 |
| 356 | 4.76 | 0.0613 | 0.599 |
| 357 | 6.21 | 0.077 | 0.625 |
| 358 | 2.78 | 0.182 | 2.31 |
| 359 | 2.1 | 0.281 | 4.61 |
| 360 | 5.28 | 0.45 | 5.86 |
| 361 | 2.73 | 0.214 | 2.08 |
| 362 | 6.04 | 0.262 | 1.77 |
| 363 | 13.6 | 0.375 | 1.47 |
| 364 | 10.2 | 0.457 | 1.1 |
| 365 | 4.24 | 0.0336 | 0.265 |
| 366 | 4.51 | 0.024 | 0.744 |
| 367 | 7.95 | 0.0215 | 0.421 |
| 368 | 0.481 | 0.0121 | 0.242 |
| 369 | 0.873 | 0.0263 | 0.301 |
| 370 | 7.06 | 0.027 | 0.406 |
| 371 | 4.46 | 0.0128 | 0.327 |
| 372 | 7.54 | 0.0161 | 0.333 |
| 373 | 4.48 | 0.0183 | 0.392 |
| 374 | 0.908 | 0.0186 | 0.336 |
| 375 | 1.32 | 0.0165 | 0.299 |
| 376 | 1.71 | 0.0354 | 0.445 |
| 377 | 1.36 | 0.0377 | 0.367 |
| 378 | 1.45 | 0.0213 | 0.251 |
| 379 | 2.27 | 0.0946 | 0.158 |
| 380 | 1.17 | 0.0346 | 0.354 |
| 381 | 1.39 | 0.0156 | 0.157 |
| 382 | 3.72 | 0.0368 | 0.291 |
| 383 | 1.27 | 0.0406 | 0.258 |
| 384 | 5.77 | 0.0152 | 0.133 |
| 385 | 1 | 0.0141 | 0.235 |
| 386 | 1.7 | 0.0156 | 0.278 |
| 387 | 2.14 | 0.0276 | 0.255 |
| 388 | 2.15 | 0.0462 | 0.373 |
| 389 | 4.03 | 0.0354 | 0.134 |
| 390 | 3.08 | 0.122 | 0.134 |
| 391 | 3.04 | 0.0298 | 0.199 |
| 392 | 3.02 | 0.0571 | 0.253 |
| 393 | 0.947 | 0.0458 | 0.211 |
| 394 | 5.63 | 0.018 | 0.226 |
| 395 | 5.55 | 0.0248 | 0.349 |
| 396 | 4.99 | 0.024 | 0.293 |
| 397 | 5.22 | 0.0247 | 0.2 |
| 398 | 2.16 | 0.0318 | 0.186 |
| 399 | 1.51 | 0.0413 | 0.302 |
| 400 | 2.53 | 0.0158 | 0.253 |
| 401 | 1.99 | 0.0183 | 0.213 |
| 402 | 5.66 | 0.123 | 0.725 |
| 403 | 1.59 | 0.0827 | 0.396 |
| 404 | 4.1 | 0.0102 | 0.217 |
| 405 | 15.5 | 0.28 | 1.48 |
| 406 | 1.23 | 0.0233 | 0.208 |
| 407 | 1.49 | 0.0362 | 0.259 |
| 408 | 1.93 | 0.0329 | 0.253 |
| 409 | 7.92 | 0.363 | 0.484 |
| 410 | 9.94 | 0.0708 | 0.354 |
| 411 | 3.94 | 0.058 | 0.709 |
| 412 | 2.36 | 0.0351 | 0.282 |
| 413 | 6.3 | 0.0881 | 0.564 |
| 414 | 4.56 | 0.0303 | 0.176 |
| 415 | 8.64 | 0.0496 | 0.38 |
| 416 | 2.08 | 0.0393 | 0.331 |
| 417 | 2.83 | 0.0144 | 0.328 |
| 418 | 4.7 | 0.0153 | 0.264 |
| 419 | 8.52 | 0.0235 | 0.615 |
| 420 | 2.49 | 0.0131 | 0.205 |
| 421 | 3.25 | 0.0132 | 0.21 |
| 422 | 14.5 | 0.0216 | 0.252 |
| 423 | 1.92 | 0.0114 | 0.194 |
| 424 | 3.12 | 0.0154 | 0.318 |
| 425 | 5.49 | 0.0997 | 0.15 |
| 426 | 4.33 | 0.079 | 0.139 |
| 427 | 3.81 | 0.0121 | 0.282 |
| 428 | 2.35 | 0.01 | 0.207 |
| 429 | 1.19 | 0.0142 | 0.178 |
| 430 | 2.11 | 0.0144 | 0.438 |
| 431 | 3.32 | 0.0157 | 0.392 |
| 432 | 1.6 | 0.0297 | 0.431 |
| 433 | 5.78 | 0.0206 | 0.273 |
| 434 | 2.84 | 0.0226 | 0.236 |
| 435 | 3.01 | 0.0239 | 0.311 |
| 436 | 2.27 | 0.0294 | 0.173 |
| 437 | 1.76 | 0.0872 | 0.421 |
| 438 | 3.62 | 0.225 | 0.815 |
| 439 | 4.38 | 0.0634 | 0.293 |
| 440 | 7.14 | 0.0226 | 0.467 |
| 441 | 8 | 0.0239 | 0.365 |
| 442 | 5.66 | 0.0237 | 0.267 |
| 443 | 6.09 | 0.0291 | 0.572 |
| 444 | 6.49 | 0.0106 | 0.352 |
| 445 | 10.1 | 0.0206 | 0.731 |
| 446 | 8.34 | 0.0351 | 0.696 |
| 447 | 9.71 | 0.0177 | 0.214 |
| 448 | 4.54 | 0.0171 | 0.336 |
| 449 | 11.5 | 0.0205 | 0.568 |
| 450 | 4.84 | 0.0448 | 0.43 |
| 451 | 4.12 | 0.0101 | 0.256 |
| 452 | 2.44 | 0.0118 | 0.235 |
| 453 | 7.37 | 0.0184 | 0.374 |
| 454 | 9.11 | 0.0154 | 0.821 |
| 455 | 4.36 | 0.0182 | 0.49 |
| 456 | 3.48 | 0.00913 | 0.19 |
| 457 | 2.86 | 0.00918 | 0.226 |
| 458 | 5.29 | 0.0448 | 0.347 |
| 459 | 1.21 | 0.0122 | 0.179 |
| 460 | 1.89 | 0.0624 | 0.636 |
| 461 | 1.53 | 0.0171 | 0.194 |
| 462 | 1.69 | 0.0148 | 0.204 |
| 463 | 4.27 | 0.0558 | 0.362 |
| 464 | 1.25 | 0.072 | 0.466 |
| 465 | 1.5 | 0.148 | 0.575 |
| 466 | 5.84 | 0.0466 | 0.207 |
| 467 | 9.34 | 0.0855 | 0.358 |
| 468 | 1.95 | 0.0222 | 0.466 |
| 469 | 1.34 | 0.031 | 0.562 |
| 470 | 2.35 | 0.0263 | 0.323 |
| 471 | 2.19 | 0.0296 | 0.317 |
| 472 | 2.73 | 0.015 | 0.171 |
| 473 | 2.72 | 0.0101 | 0.173 |
| 474 | 3.88 | 0.0125 | 0.236 |
| 475 | 2.24 | 0.0173 | 0.254 |
| 476 | 1.34 | 0.0131 | 0.164 |
| 477 | 5.26 | 0.0127 | 0.173 |
| 478 | 8.07 | 0.106 | 0.243 |
| 479 | 5.98 | 0.0905 | 0.314 |
| 480 | 1.47 | 0.0107 | 0.215 |
| 481 | 1.22 | 0.0164 | 0.204 |
| 482 | 1.41 | 0.0139 | 0.177 |
| 483 | 1.77 | 0.0133 | 0.188 |
| 484 | 3.99 | 0.0152 | 0.201 |
| 485 | 4.29 | 0.0194 | 0.277 |
| 486 | 4.43 | 0.0262 | 0.375 |
| 487 | 5.36 | 0.0286 | 0.639 |
| 488 | 5.13 | 0.0263 | 0.682 |
| 489 | 2.31 | 0.0121 | 0.172 |
| 490 | 3.05 | 0.0159 | 0.183 |
| 491 | 1.58 | 0.0126 | 0.17 |
| 492 | 1.35 | 0.0119 | 0.146 |
| 493 | 6.25 | 0.0406 | 0.644 |
| 494 | 1.26 | 0.014 | 0.211 |
| 495 | 1.75 | 0.0361 | 0.274 |
| 496 | 8.25 | 0.387 | 9.69 |
| 497 | 2.70 | 0.297 | 1.08 |
| 498 | 4.11 | 0.0711 | 1.53 |
| 499 | 1.39 | 0.0274 | 0.219 |
| 500 | 4.04 | 0.0375 | 0.623 |
| 501 | 4.54 | 0.0373 | 0.401 |
| 502 | 3.8 | 0.0337 | 0.326 |
| 503 | 3.98 | 0.0518 | 0.525 |
| 504 | 2.83 | 0.0364 | 0.253 |
| 505 | 0.445 | 0.00707 | 0.205 |
| 506 | 17.1 | 0.308 | 2.14 |
| 507 | 1.86 | 0.0127 | 0.187 |
| 508 | 1.82 | 0.00883 | 0.173 |
| 509 | 4.66 | 0.0557 | 0.65 |
| 510 | 5.44 | 0.0627 | 0.71 |
| 511 | 8.75 | 0.0914 | 1.05 |
| 512 | 7.34 | 0.0697 | 1.32 |
| 513 | 9.58 | 0.0956 | 0.634 |
| 514 | 6.68 | 0.0744 | 0.623 |
| 515 | 6.47 | 0.0893 | 0.825 |
| 516 | 8.45 | 0.112 | 0.907 |
| 517 | 4.69 | 0.158 | 0.646 |
| 518 | 3.93 | 0.129 | 0.867 |
| 519 | 4.71 | 0.212 | 1.04 |
| 520 | 6.09 | 0.224 | 1.04 |
| 521 | 4.04 | 0.0164 | 0.341 |
| 522 | 6.32 | 0.033 | 0.51 |
| 523 | 3.79 | 0.021 | 0.482 |
| 524 | 5.11 | 0.0266 | 0.487 |
| 525 | 10 | 0.0351 | 0.426 |
| 526 | 8.62 | 0.0364 | 0.421 |
| 527 | 6.99 | 0.0386 | 0.446 |
| 528 | 7 | 0.0333 | 0.434 |
| 529 | 6.59 | 0.0273 | 0.467 |
| 530 | 17.1 | 0.874 | 4.91 |
| 531 | 3.79 | 0.0694 | 0.356 |
| 532 | 1.37 | 0.0159 | 0.577 |
| 533 | 5.02 | 0.0388 | 0.334 |
| 534 | 4.58 | 0.0523 | 0.395 |
| 535 | 3.16 | 0.0183 | 0.418 |
| 536 | 5.83 | 0.0211 | 0.521 |
| 537 | 5.9 | 0.0641 | 0.538 |
| 538 | 6.93 | 0.0672 | 0.533 |
| 539 | 4.97 | 0.0247 | 0.521 |
| 540 | 5.62 | 0.0296 | 0.686 |
| 541 | 4.26 | 0.0213 | 0.74 |
| 542 | 5.03 | 0.0336 | 0.388 |
| 543 | 4.56 | 0.0319 | 0.41 |
| 544 | 2.71 | 0.041 | 0.623 |
| 545 | 2.71 | 0.0432 | 0.602 |
| 546 | 3.67 | 0.25 | 0.517 |
| 547 | 3.8 | 0.152 | 0.337 |
| 548 | 3.94 | 0.0547 | 0.478 |
| 549 | 3.41 | 0.0255 | 0.395 |
| 550 | 11.7 | 0.0876 | 2.12 |
| 551 | 1.74 | 0.0173 | 0.303 |
| 552 | 2.53 | 0.0177 | 0.804 |
| 553 | 4.53 | 0.0371 | 0.398 |
| 554 | 6.83 | 0.0323 | 0.423 |
| 555 | 3.56 | 0.0601 | 0.445 |
| 556 | 4.14 | 0.0866 | 0.561 |
| 557 | 3.42 | 0.0486 | 0.45 |
| 558 | 4.42 | 0.0564 | 0.526 |
| 559 | 4.07 | 0.119 | 0.542 |
| 560 | 3.8 | 0.113 | 0.883 |
| 561 | 5.43 | 0.212 | 1.13 |
| 562 | 6 | 0.284 | 1.67 |
| 563 | 8.3 | 0.132 | 1.01 |
| 564 | 10.2 | 0.176 | 1.33 |
| 565 | 10.3 | 0.107 | 1.24 |
| 566 | 7.18 | 0.137 | 1.3 |
| 567 | 5.89 | 0.165 | 1.07 |
| 568 | 6.13 | 0.125 | 0.986 |
| 569 | 4.34 | 0.199 | 1.12 |
| 570 | 5.88 | 0.186 | 1.03 |
| 571 | 3.27 | 0.0746 | 0.603 |
| 572 | 3.48 | 0.0465 | 0.366 |
| 573 | 4.02 | 0.0342 | 0.416 |
| 574 | 5.04 | 0.235 | 0.499 |
| 575 | 3.69 | 0.321 | 0.438 |
| 576 | 6.37 | 0.0723 | 0.528 |
| 577 | 6.47 | 0.103 | 0.931 |
| 578 | 6.26 | 0.0208 | 0.233 |
| 579 | 2.36 | 0.0171 | 0.252 |
| 580 | 7.27 | 0.0426 | 0.615 |
| 581 | 8.18 | 0.0812 | 0.406 |
| 582 | 10.5 | 0.103 | 0.298 |
| 583 | 6.32 | 0.0692 | 0.542 |
| 584 | 5.29 | 0.0461 | 1 |
| 585 | 6.25 | 0.0801 | 0.601 |
| 586 | 4.58 | 0.0461 | 0.593 |
| 587 | 5.38 | 0.0635 | 0.611 |
| 588 | 4.72 | 0.0777 | 0.613 |
| 589 | 4.11 | 0.0672 | 0.713 |
| 590 | 4.34 | 0.105 | 0.89 |
| 591 | 4.67 | 0.0579 | 0.773 |
| 592 | 4.25 | 0.0971 | 1.12 |
| 593 | 6.57 | 0.0484 | 0.495 |
| 594 | 8.35 | 0.0541 | 0.561 |
| 595 | 5.06 | 0.0338 | 0.53 |
| 596 | 5.71 | 0.0455 | 0.592 |
| 597 | 5.2 | 0.0643 | 0.6 |
| 598 | 8.79 | 0.0927 | 0.886 |
| 599 | 2.9 | 0.0558 | 0.409 |
| 600 | 2.97 | 0.0478 | 0.377 |
| 601 | 3.88 | 0.0688 | 0.443 |
| 602 | 4.21 | 0.0726 | 0.775 |
| 603 | 5.43 | 0.102 | 0.688 |
| 604 | 5.5 | 0.161 | 0.705 |
| 605 | 5.49 | 0.132 | 0.795 |
| 606 | 5.01 | 0.17 | 0.786 |
| 607 | 8.91 | 0.0879 | 1.15 |
| 608 | 7.82 | 0.124 | 1.87 |
| 609 | 6.46 | 0.0784 | 1.07 |
| 610 | 5.75 | 0.133 | 1.66 |
| 611 | 6.8 | 0.126 | 1.63 |
| 612 | 7.13 | 0.087 | 1.21 |
| 613 | 20.4 | 0.00925 | 0.698 |
| 614 | 6.36 | 0.0065 | 0.251 |
| 615 | 3.93 | 0.00825 | 0.575 |
| 616 | 6.58 | 0.00727 | 0.29 |
| 617 | 4.56 | 0.00849 | 0.534 |
| 618 | 5.57 | 0.0418 | 1.53 |
| 619 | 6.34 | 0.0685 | 0.992 |
| 620 | 8.26 | 0.0706 | 0.981 |
| 621 | 6.81 | 0.0661 | 1.28 |
| 622 | 7.7 | 0.0148 | 0.776 |
| 623 | 7.94 | 0.0224 | 0.907 |
| 624 | 8.42 | 0.0222 | 0.95 |
| 625 | 8.53 | 0.0291 | 1.16 |
| 626 | 8.72 | 0.0192 | 0.889 |
| 627 | 8.82 | 0.0261 | 1.22 |
| 628 | 7.47 | 0.0278 | 1.11 |
| 629 | 9.75 | 0.029 | 1.58 |
| 630 | 8.07 | 0.0233 | 0.762 |
| 631 | 16.8 | 0.0375 | 0.658 |
| 632 | 13.5 | 0.068 | 0.951 |
| 633 | 10.2 | 0.0186 | 0.421 |
| 634 | 6.49 | 0.0195 | 0.403 |
| 635 | 7.09 | 0.0405 | 0.625 |
| 636 | 9.28 | 0.054 | 0.504 |
| 637 | 11.7 | 0.108 | 1.08 |
| 638 | 14 | 0.0354 | 0.4 |
| 639 | 8 | 0.0231 | 0.374 |
| 640 | 9.5 | 0.00696 | 0.288 |
| 641 | 3.72 | 0.00819 | 0.489 |
| 642 | 3.49 | 0.00796 | 0.474 |
| 643 | 16 | 0.0223 | 0.741 |
| 644 | 5.91 | 0.00649 | 0.309 |
| 645 | 3.25 | 0.00724 | 0.596 |
| 646 | 23.1 | 0.0336 | 2.58 |
| 647 | 18.8 | 0.0177 | 0.872 |
| 648 | 20.3 | 0.0392 | 1.76 |
| 649 | 21.2 | 0.0221 | 1.35 |
| 650 | 16.2 | 0.0814 | 0.649 |
| 651 | 10.7 | 0.112 | 1.02 |
| 652 | 15.3 | 0.0342 | 0.426 |
| 653 | 7.29 | 0.0303 | 0.426 |
| 654 | 2.66 | 0.0471 | 0.349 |
| 655 | 3.79 | 0.0533 | 0.351 |
| 656 | 2.04 | 0.0269 | 0.318 |
| 657 | 0.891 | 0.0393 | 0.429 |
| 658 | 1.05 | 0.0379 | 0.34 |
| 659 | 5.89 | 0.0673 | 1.31 |
| 660 | 7.38 | 0.0646 | 1.27 |
| 661 | 1.78 | 0.0665 | 1.13 |
| 662 | 4.81 | 0.0704 | 0.946 |
| 663 | 1.95 | 0.0893 | 1.29 |
| 664 | 2.12 | 0.0159 | 0.356 |
| 665 | 2.94 | 0.0223 | 0.284 |
| 666 | 2 | 0.0203 | 0.337 |
| 667 | 2.34 | 0.0217 | 0.42 |
| 668 | 2.03 | 0.0198 | 0.325 |
| 669 | 1.76 | 0.0145 | 0.27 |
| 670 | 3.28 | 0.0135 | 0.683 |
| 671 | 1.81 | 0.00807 | 0.166 |
| 672 | 1.19 | 0.014 | 0.389 |
| 673 | 2.31 | 0.00656 | 0.174 |
| 674 | 2.09 | 0.0264 | 0.21 |
| 675 | 2.15 | 0.0111 | 0.254 |
| 676 | 2.34 | 0.0106 | 0.267 |
| 677 | 2.21 | 0.0111 | 0.252 |
| 678 | 2.28 | 0.0132 | 0.251 |
| 679 | 3.22 | 0.0123 | 0.255 |
| 680 | 2 | 0.01 | 0.246 |
| 681 | 1.35 | 0.00663 | 0.257 |
| 682 | 1.91 | 0.00661 | 0.212 |
| 683 | 1.57 | 0.00798 | 0.214 |
| 684 | 2.16 | 0.00843 | 0.28 |
| 685 | 2.18 | 0.00809 | 0.308 |
| 686 | 2.43 | 0.00776 | 0.298 |
| 687 | 2.41 | 0.00903 | 0.277 |
| 688 | 2.06 | 0.00881 | 0.304 |
| 689 | 1.81 | 0.0104 | 0.214 |
| 690 | 1.65 | 0.00892 | 0.231 |
| 691 | 2.46 | 0.0117 | 0.26 |
| 692 | 2.14 | 0.0106 | 0.252 |
| 693 | 2.39 | 0.0104 | 0.161 |
| 694 | 2.04 | 0.00781 | 0.253 |
| 695 | 1.93 | 0.0136 | 0.254 |
| 696 | 1.75 | 0.00688 | 0.233 |
| 697 | 1.71 | 0.0109 | 0.235 |
| 698 | 1.53 | 0.0112 | 0.252 |
| 699 | 1.35 | 0.00663 | 0.196 |
| 700 | 1.31 | 0.00758 | 0.245 |
| 701 | 1.48 | 0.01 | 0.22 |
| 702 | 1.5 | 0.0116 | 0.243 |
| 703 | 1.56 | 0.0101 | 0.233 |
| 704 | 1.85 | 0.013 | 0.287 |
| 705 | 1.7 | 0.0128 | 0.305 |
| 706 | 2.18 | 0.00911 | 0.19 |
| 707 | 1.06 | 0.0107 | 0.248 |
| 708 | 1.44 | 0.00907 | 0.262 |
| 709 | 1.23 | 0.0129 | 0.323 |
| 710 | 1.52 | 0.00578 | 0.274 |
| 711 | 1.29 | 0.00861 | 0.355 |
| 712 | 1.36 | 0.00689 | 0.258 |
| 713 | 1.71 | 0.00928 | 0.331 |
| 714 | 1.76 | 0.00764 | 0.289 |
| 715 | 1 | 0.00644 | 0.267 |
| 716 | 1.12 | 0.00853 | 0.321 |
| 717 | 1.14 | 0.00721 | 0.283 |
| 718 | 1.2 | 0.00706 | 0.214 |
| 719 | 1.44 | 0.00998 | 0.22 |
| 720 | 1.78 | 0.0103 | 0.245 |
| 721 | 1.78 | 0.00774 | 0.233 |
| 722 | 1.71 | 0.01 | 0.252 |
| 723 | 1.82 | 0.0118 | 0.255 |
| 724 | 1.74 | 0.0102 | 0.256 |
| 725 | 1.49 | 0.0126 | 0.258 |
| 726 | 1.71 | 0.013 | 0.247 |
| 727 | 2.38 | 0.0109 | 0.271 |
| 728 | 2.45 | 0.0116 | 0.277 |
| 729 | 2.11 | 0.0113 | 0.288 |
| 730 | 2.46 | 0.0122 | 0.248 |
| 731 | 2.35 | 0.0117 | 0.267 |
| 732 | 2.18 | 0.0118 | 0.268 |
| 733 | 1.6 | 0.00894 | 0.293 |
| 734 | 2.08 | 0.0132 | 0.417 |
| 735 | 1.97 | 0.012 | 0.386 |
| 736 | 2.85 | 0.014 | 0.489 |
| 737 | 1.34 | 0.00804 | 0.285 |
| 738 | 1.87 | 0.00991 | 0.368 |
| 739 | 1.59 | 0.015 | 0.348 |
| 740 | 5.34 | 0.012 | 0.453 |
| 741 | 2.01 | 0.00612 | 0.239 |
| 742 | 1.25 | 0.00688 | 0.266 |
| 743 | 1.49 | 0.00694 | 0.259 |
| 744 | 1.04 | 0.00844 | 0.274 |
| 745 | 1.86 | 0.012 | 0.212 |
| 746 | 1.62 | 0.0119 | 0.261 |
| 747 | 1.71 | 0.00955 | 0.229 |
| 748 | 1.42 | 0.0105 | 0.258 |
| 749 | 2.21 | 0.00718 | 0.293 |
| 750 | 2 | 0.00784 | 0.302 |
| 751 | 1.9 | 0.00759 | 0.302 |
| 752 | 1.9 | 0.00689 | 0.274 |
| 753 | 2.7 | 0.0127 | 0.271 |
| 754 | 2.3 | 0.0102 | 0.222 |
| 755 | 2.31 | 0.00805 | 0.175 |
| 756 | 2.26 | 0.0096 | 0.212 |
| 757 | 2.32 | 0.00959 | 0.236 |
| 758 | 3.99 | 0.0212 | 0.195 |
| 759 | 1.29 | 0.00676 | 0.262 |
| 760 | 1.59 | 0.00722 | 0.395 |
| 761 | 1.5 | 0.00686 | 0.226 |
| 762 | 3.09 | 0.00882 | 0.273 |
| 763 | 1.57 | 0.00719 | 0.302 |
| 764 | 1.63 | 0.00626 | 0.289 |
| 765 | 0.952 | 0.0642 | 0.25 |
| 766 | 0.689 | 0.0323 | 0.273 |
| 767 | 1.13 | 0.00792 | 0.299 |
| 768 | 10.3 | 0.0294 | 0.259 |
| 769 | 1.33 | 0.00852 | 0.247 |
| 770 | 5.37 | 0.244 | 3.91 |
| 771 | 5.05 | 0.232 | 4.38 |
| 772 | 15.9 | 0.115 | 1.38 |
| 773 | 15.3 | 0.0974 | 1.97 |
| 774 | 13.7 | 0.128 | 1.89 |
| 775 | 4.36 | 0.105 | 0.993 |
| 776 | 3.73 | 0.112 | 0.889 |
| 777 | 3.89 | 0.113 | 0.913 |
| 778 | 4.39 | 0.136 | 1.16 |
| 779 | 4.58 | 0.112 | 0.949 |
| 780 | 4.84 | 0.111 | 1.26 |
| 781 | 4.46 | 0.065 | 1.41 |
| 782 | 4.96 | 0.0711 | 1.94 |
| 783 | 3.81 | 0.0806 | 2.13 |
| 784 | 5.21 | 0.0587 | 2.09 |
| 785 | 4.51 | 0.0859 | 2.3 |
| 786 | 4.46 | 0.0986 | 2.8 |
| 787 | 7.81 | 0.0675 | 2.55 |
| 788 | 3.36 | 0.0923 | 2.5 |
| 789 | 3.3 | 0.101 | 2.74 |
| 790 | 3.87 | 0.131 | 3.19 |
| 791 | 2.36 | 0.979 | 4.68 |
| 792 | 1.91 | 0.55 | 2.99 |
| 793 | 3.79 | 0.834 | 3.19 |
| 794 | 1.21 | 0.468 | 1.26 |
| 795 | 1.77 | 0.345 | 0.972 |
| 796 | 5.3 | 0.098 | 0.602 |
| 797 | 3.78 | 0.0781 | 0.449 |
| 798 | 12.1 | 0.0653 | 0.628 |
| 799 | 1.64 | 0.0668 | 0.462 |
| 800 | 21 | 0.283 | 0.486 |
| 801 | 5.51 | 0.147 | 0.938 |
| 802 | 16.6 | 0.124 | 1.41 |
| 803 | 9.75 | 0.232 | 2.02 |
| 804 | 11.5 | 0.0751 | 0.915 |
| 805 | 3.97 | 0.0875 | 0.715 |
| 806 | 8.25 | 0.124 | 0.706 |
| 807 | 12.4 | 0.108 | 1.72 |
| 808 | 1.65 | 0.187 | 3.58 |
| 809 | 1.74 | 0.194 | 1.87 |
| 810 | 3.3 | 0.156 | 2.09 |
| 811 | 0.724 | 0.14 | 1.48 |
| 812 | 1.05 | 0.0934 | 1.19 |
| 813 | 2.5 | 0.124 | 0.93 |
| 814 | 3.92 | 0.337 | 3.76 |
| 815 | 4.13 | 0.181 | 2.79 |
| 816 | 6.57 | 0.297 | 2.69 |
| 817 | 4.32 | 0.173 | 1.18 |
| 818 | 2.31 | 0.121 | 0.51 |
| 819 | 1.53 | 0.0428 | 0.847 |
| 820 | 6.83 | 0.121 | 2.78 |
| 821 | 6.74 | 0.152 | 2.79 |
| 822 | 11.6 | 0.186 | 2.51 |
| 823 | 7.75 | 0.171 | 2.96 |
| 824 | 7.92 | 0.123 | 2.43 |
| 825 | 5.53 | 0.12 | 3.4 |
| 826 | 5.59 | 0.581 | 2.75 |
| 827 | 4.23 | 0.12 | 0.593 |
| 828 | 3.58 | 0.149 | 1.17 |
| 829 | 2.41 | 0.124 | 1.15 |
| 830 | 3.21 | 0.132 | 0.791 |
| 831 | 4.63 | 0.604 | 0.672 |
| 832 | 9.1 | 0.187 | 1.07 |
| 833 | 3.2 | 0.114 | 1.28 |
| 834 | 3.36 | 0.094 | 2.64 |
| 835 | 3.83 | 0.382 | 2.84 |
| 836 | 17.1 | 0.294 | 0.794 |
| 837 | 7.24 | 0.209 | 0.551 |
| 838 | 20.7 | 0.225 | 0.379 |
| 839 | 5.05 | 0.221 | 0.469 |
| 840 | 8.38 | 0.147 | 0.481 |
| 841 | 9.71 | 0.194 | 0.336 |
| 842 | 2.35 | 0.284 | 1.08 |
| 843 | 3.41 | 0.334 | 0.874 |
| 844 | 2.83 | 0.27 | 0.61 |
| 845 | 0.626 | 0.178 | 0.161 |
| 846 | 1.19 | 0.204 | 0.82 |
| 847 | 1.44 | 0.182 | 0.598 |
| 848 | 3.22 | 0.148 | 2.29 |
| 849 | 6.66 | 0.306 | 0.661 |
| 850 | 28.4 | 0.346 | 0.451 |
| 851 | 3.38 | 0.215 | 3.06 |
| 852 | 2.26 | 0.34 | 0.871 |
| 853 | 26.5 | 0.224 | 0.507 |
| 854 | 4.31 | 0.158 | 3.32 |
| 855 | 2.67 | 0.271 | 0.908 |
| 856 | 4.41 | 0.237 | 3.37 |
| 857 | 6.21 | 0.441 | 0.433 |
| 858 | 3.44 | 0.0995 | 1.83 |
| 859 | 8.45 | 0.12 | 2.38 |
| 860 | 11 | 0.202 | 3.95 |
| 861 | 8.59 | 0.195 | 2.62 |
| 862 | 5.87 | 0.0896 | 1.7 |
| 863 | 2.22 | 0.136 | 1.98 |
| 864 | 3.56 | 0.048 | 1.05 |
| 865 | 1.48 | 0.0797 | 3.02 |
| 866 | 4.48 | 0.148 | 4.01 |
| 867 | 2.47 | 0.216 | 7.38 |
| 868 | 7.4 | 0.208 | 5.82 |
| 869 | 9.94 | 0.142 | 2 |
| 870 | 4.4 | 0.0698 | 0.962 |
| 871 | 12.6 | 0.2 | 2.92 |
| 872 | 4.18 | 0.205 | 1.01 |
| 873 | 2.07 | 0.306 | 0.907 |
| 874 | 2.19 | 0.142 | 0.991 |
| 875 | 3.65 | 0.101 | 0.771 |
| 876 | 3.28 | 0.053 | 0.456 |
| 877 | 3.6 | 0.0816 | 0.601 |
| 878 | 2.85 | 0.0743 | 0.748 |
| 879 | 2.16 | 0.169 | 4 |
| 880 | 3.53 | 0.116 | 7.69 |
| 881 | 4.09 | 0.211 | 2.87 |
| 882 | 2.73 | 0.129 | 2.33 |
| 883 | 4.02 | 0.0779 | 1.05 |
| 884 | 3.82 | 0.155 | 4.17 |
| 885 | 2.42 | 0.149 | 3.29 |
| 886 | 4.13 | 0.149 | 2.7 |
| 887 | 5.28 | 0.145 | 3 |
| 888 | 30.1 | 0.168 | 5.02 |
| 889 | 4.09 | 0.106 | 5.06 |
| 890 | 4.73 | 0.113 | 3.19 |
| 891 | 3.14 | 0.155 | 3.25 |
| 892 | 3.85 | 0.149 | 4.41 |
| 893 | 5.8 | 0.166 | 2.9 |
| 894 | 6.09 | 0.0752 | 2.01 |
| 895 | 4.68 | 0.111 | 3.72 |
| 896 | 10.6 | 0.12 | 4.64 |
| 897 | 3.62 | 0.157 | 3.29 |
| 898 | 3.39 | 0.287 | 4.62 |
| 899 | 4.4 | 0.133 | 4.11 |
| 900 | 4.74 | 0.173 | 3.99 |
| 901 | 9.09 | 0.17 | 1.26 |
| 902 | 15.9 | 0.19 | 2.58 |
| 903 | 12.9 | 0.277 | 2.7 |
| 904 | 9.88 | 0.194 | 1.94 |
| 905 | 12.3 | 0.217 | 2.52 |
| 906 | 14.7 | 0.212 | 2.48 |
| 907 | 5.79 | 0.103 | 1.15 |
| 908 | 8.11 | 0.246 | 1.2 |
| 909 | 6.82 | 0.121 | 1.72 |
| 910 | 9.76 | 0.111 | 1.49 |
| 911 | 9.53 | 0.197 | 1.09 |
| 912 | 3.76 | 0.0387 | 0.414 |
| 913 | 3.8 | 0.0349 | 1.05 |
| 914 | 10.6 | 0.0582 | 0.888 |
| 915 | 0.882 | 0.0988 | 1.5 |
| 916 | 1.09 | 0.0715 | 1.38 |
| 917 | 3.61 | 0.0724 | 1.52 |
| 918 | 3.35 | 0.173 | 2.85 |
| 919 | 3.51 | 0.182 | 3.91 |
| 920 | 4.61 | 0.0905 | 2.28 |
| 921 | 10.4 | 0.228 | 3.73 |
| 922 | 8.49 | 0.112 | 6.77 |
| 923 | 9.1 | 0.123 | 6.23 |
| 924 | 6.05 | 0.0798 | 3.12 |
| 925 | 8.19 | 0.147 | 4.19 |
| 926 | 13.2 | 0.166 | 7.71 |
| 927 | 15.8 | 0.213 | 8.34 |
| 928 | 6.85 | 0.0883 | 3.58 |
| 929 | 12.6 | 0.0953 | 7.51 |
| 930 | 13 | 0.244 | 11 |
| 931 | 12.2 | 0.256 | 11.3 |
| 932 | 2.67 | 0.188 | 1.61 |
| 933 | 5.51 | 0.259 | 2.32 |
| 934 | 2.73 | 0.0738 | 0.649 |
| 935 | 4.04 | 0.123 | 1.71 |
| 936 | 5.08 | 0.192 | 2.16 |
| 937 | 5.52 | 0.222 | 2.52 |
| 938 | 13.3 | 0.13 | 1.84 |
| 939 | 2.41 | 0.143 | 2.75 |
| 940 | 2.84 | 0.137 | 4.08 |
| 941 | 11.7 | 0.183 | 0.764 |
| 942 | 6.32 | 0.188 | 3.52 |
| 943 | 6.58 | 0.233 | 3.58 |
| 944 | 6.16 | 0.216 | 4.86 |
| 945 | 18 | 0.246 | 3.9 |
| 946 | 16.5 | 0.353 | 2.05 |
| 947 | 6.99 | 0.289 | 1.7 |
| 948 | 2.89 | 0.17 | 2.67 |
| 949 | 1.62 | 0.203 | 3.37 |
| 950 | 1.92 | 0.18 | 2.5 |
| 951 | 5.73 | 0.156 | 2.61 |
| 952 | 2.4 | 0.166 | 4.85 |
| 953 | 2.75 | 0.111 | 2.63 |
| 954 | 3.66 | 0.268 | 4.79 |
| 955 | 2.65 | 0.386 | 7.74 |
| 956 | 2.17 | 0.26 | 4.83 |
| 957 | 4.18 | 0.213 | 5.01 |
| 958 | 3.17 | 0.343 | 9.51 |
| 959 | 2.61 | 0.217 | 5.06 |
| 960 | 5.5 | 0.124 | 1.51 |
| 961 | 2.96 | 0.167 | 2.52 |
| 962 | 3.13 | 0.0972 | 1.36 |
| 963 | 5.81 | 0.244 | 1.77 |
| 964 | 2.75 | 0.288 | 2.67 |
| 965 | 3.35 | 0.13 | 1.39 |
| 966 | 1.87 | 0.15 | 1.23 |
| 967 | 1.94 | 0.205 | 1.75 |
| 968 | 4.72 | 0.0928 | 1.04 |
| 969 | 5.48 | 0.142 | 1.21 |
| 970 | 5.24 | 0.178 | 2 |
| 971 | 8.26 | 0.298 | 3.46 |
| 972 | 4.19 | 0.34 | 2.05 |
| 973 | 17.6 | 0.503 | 6.1 |
| 974 | 3.7 | 0.607 | 1.57 |
| 975 | 2.61 | 0.344 | 10.2 |
| 976 | 8.75 | 0.441 | 21.8 |
| 977 | 7.18 | 0.189 | 1.1 |
| 978 | 8.91 | 0.284 | 1.6 |
| 979 | 3.34 | 0.181 | 2.72 |
| 980 | 2.41 | 0.18 | 2.26 |
| 981 | 3.7 | 0.243 | 4.45 |
| 982 | 3.91 | 0.328 | 4.94 |
| 983 | 4.75 | 0.289 | 2.35 |
| 984 | 4.5 | 0.232 | 2.07 |
| 985 | 5.41 | 0.34 | 2.93 |
| 986 | 4.94 | 0.299 | 3.06 |
| 987 | 3.62 | 0.194 | 6.33 |
| 988 | 2.52 | 0.198 | 9.5 |
| 989 | 2.27 | 0.103 | 3.92 |
| 990 | 2.47 | 0.115 | 5.78 |
| 991 | 3.03 | 0.119 | 3.91 |
| 992 | 10.8 | 0.165 | 7.86 |
| 993 | 20.8 | 0.138 | 17.6 |
| 994 | 9.51 | 0.359 | 4.42 |
| 995 | 3.18 | 0.118 | 27.7 |
| 996 | 14.9 | 0.2 | 98.1 |
| 997 | 5.01 | 0.373 | 20.7 |
| 998 | 8.96 | 0.0906 | 14.3 |
| 999 | 19.4 | 0.128 | 34.8 |
| 1000 | 4.62 | 0.176 | 3.55 |
| 1001 | 22.5 | 0.4 | 4.59 |
| 1002 | 1.89 | 0.119 | 9.09 |
| 1003 | 5.07 | 0.272 | 42.4 |
| 1004 | 2.67 | 0.519 | 9.34 |
| 1005 | 9.46 | 0.152 | 1.48 |
| 1006 | 4.28 | 0.109 | 5.12 |
| 1007 | 5.34 | 0.116 | 0.668 |
| 1008 | 7.47 | 0.14 | 1.09 |
| 1009 | 4.21 | 0.13 | 2.1 |
| 1010 | 4.5 | 0.159 | 2.89 |
| 1011 | 5.27 | 0.21 | 2.13 |
| 1012 | 4.36 | 0.206 | 1.81 |
| 1013 | 2 | 0.208 | 0.449 |
| 1014 | 3.62 | 0.0412 | 0.718 |
| 1015 | 3.53 | 0.0822 | 1.01 |
| 1016 | 5.57 | 0.218 | 1.55 |
| 1017 | 6.47 | 0.228 | 1.16 |
| 1018 | 5.29 | 0.51 | 1.69 |
| 1019 | 4.36 | 0.169 | 1.33 |
| 1020 | 5.87 | 0.309 | 2.29 |
| 1021 | 4.39 | 0.22 | 1.3 |
| 1022 | 6.93 | 0.35 | 3.28 |
| 1023 | 3.82 | 0.235 | 1.47 |
| 1024 | 10.6 | 0.441 | 4.79 |
| 1025 | 3.22 | 0.18 | 2.45 |
| 1026 | 6.32 | 0.308 | 1.77 |
| 1027 | 4.99 | 0.208 | 1.23 |
| 1028 | 5.77 | 0.189 | 1.34 |
| 1029 | 5.35 | 0.163 | 1.79 |
| 1030 | 9.42 | 0.122 | 1.72 |
| 1031 | 6.98 | 0.146 | 1.15 |
| 1032 | 5.94 | 0.204 | 1.19 |
| 1033 | 15.4 | 0.144 | 1.21 |
| 1034 | 12.3 | 0.214 | 2.49 |
| 1035 | 14.5 | 0.131 | 1.29 |
| 1036 | 2.05 | 0.114 | 6.87 |
| 1037 | 3.31 | 0.221 | 5.95 |
| 1038 | 5.93 | 0.134 | 1.45 |
| 1039 | 7.46 | 0.118 | 1.65 |
| 1040 | 11.1 | 0.0717 | 1.69 |
| 1041 | 7.71 | 0.365 | 1.24 |
| 1042 | 27.5 | 1.3 | 10.3 |
| 1043 | 17.1 | 0.282 | 1.52 |
| 1044 | 18.8 | 0.896 | 10.7 |
| 1045 | 7.35 | 0.781 | 1.51 |
| 1046 | 10 | 0.352 | 1.94 |
| 1047 | 8.18 | 0.28 | 16.2 |
| 1048 | 6.9 | 0.356 | 6.23 |
| 1049 | 6.33 | 0.24 | 2.23 |
| 1050 | 6.8 | 0.152 | 7.65 |
| 1051 | 10.3 | 0.172 | 8.3 |
| 1052 | 7.41 | 0.167 | 3.16 |
| 1053 | 10.4 | 0.123 | 6.2 |
| 1054 | 8.06 | 0.797 | 9.53 |
| 1055 | 1.88 | 0.366 | 14.6 |
| 1056 | 3.98 | 0.0929 | 4.25 |
| 1057 | 5.29 | 0.128 | 5.16 |
| 1058 | 5.8 | 0.139 | 4.48 |
| 1059 | 7.52 | 0.468 | 4.61 |
| 1060 | 9.08 | 0.223 | 2.85 |
| 1061 | 17.3 | 0.656 | 4.56 |
| 1062 | 8.77 | 0.726 | 7.75 |
| 1063 | 10.6 | 0.318 | 6.96 |
| 1064 | 13.7 | 0.556 | 7.89 |
| 1065 | 19.7 | 0.293 | 11.6 |
| 1066 | 25.4 | 0.446 | 9.8 |
| 1067 | 12.6 | 0.471 | 17.6 |
| 1068 | 4.8 | 0.211 | 11.6 |
| 1069 | 21.2 | 0.417 | 8.87 |
| 1070 | 20.2 | 0.167 | 1.54 |
| 1071 | 23.5 | 0.261 | 2.06 |
| 1072 | 10.5 | 0.157 | 0.889 |
| 1073 | 12.3 | 0.201 | 1.09 |
| 1074 | 22.8 | 0.0745 | 0.794 |
| 1075 | 25 | 0.0778 | 0.878 |
| 1076 | 59 | 0.174 | 1.47 |
| 1077 | 18.8 | 0.23 | 1.48 |
| 1078 | 7.58 | 0.356 | 1.62 |
| 1079 | 30 | 0.0923 | 1.22 |
| 1080 | 20.9 | 0.176 | 1.59 |
| 1081 | 19.7 | 0.105 | 1.29 |
| 1082 | 12.3 | 0.099 | 4.13 |
| 1083 | 15 | 0.328 | 7.76 |
| 1084 | 11.5 | 0.0975 | 10.3 |
| 1085 | 11.6 | 0.0827 | 1.28 |
| 1086 | 12 | 0.117 | 1.23 |
| 1087 | 15.4 | 0.207 | 2.63 |
| 1088 | 14.8 | 0.182 | 2.99 |
| 1089 | 14 | 0.213 | 2.96 |
| 1090 | 15.6 | 0.272 | 5.44 |
| 1091 | 4.67 | 0.0592 | 1.23 |
| 1092 | 3.45 | 0.0648 | 1.33 |
| 1093 | 7.72 | 0.152 | 0.954 |
| 1094 | 5.84 | 0.0515 | 0.623 |
| 1095 | 2.75 | 0.065 | 1.17 |
| 1096 | 3.94 | 0.0799 | 1.47 |
| 1097 | 6.16 | 0.136 | 1.59 |
| 1098 | 6.2 | 0.201 | 1.22 |
| 1099 | 3.47 | 0.076 | 0.73 |
| 1100 | 5.28 | 0.0694 | 0.514 |
| 1101 | 4.72 | 0.0592 | 1.75 |
| 1102 | 4.37 | 0.0418 | 2.18 |
| 1103 | 11.1 | 0.0723 | 1.06 |
| 1104 | 11.1 | 0.09 | 1.7 |
| 1105 | 10.7 | 0.0427 | 0.875 |
| 1106 | 7.71 | 0.0312 | 0.87 |
| 1107 | 5.66 | 0.0447 | 0.51 |
| 1108 | 4.96 | 0.0507 | 0.752 |
| 1109 | 10.8 | 0.0626 | 2.3 |
| 1110 | 5.91 | 0.059 | 0.508 |
| 1111 | 5.28 | 0.0356 | 1.14 |
| 1112 | 10.7 | 0.254 | 0.957 |
| 1113 | 14.4 | 0.0414 | 1.12 |
| 1114 | 11.5 | 0.0478 | 0.84 |
| 1115 | 12.7 | 0.0246 | 2 |
| 1116 | 8.2 | 0.134 | 4.63 |
| 1117 | 4.93 | 0.124 | 3.51 |
| 1118 | 6.52 | 0.128 | 5.42 |
| 1119 | 7.61 | 0.0939 | 3.57 |
| 1120 | 5.17 | 0.096 | 4.5 |
| 1121 | 8.83 | 0.115 | 5.23 |
| 1122 | 10.3 | 0.109 | 5.66 |
| 1123 | 5.53 | 0.101 | 4.47 |
| 1124 | 6.97 | 0.187 | 10.1 |
| 1125 | 5.83 | 0.173 | 8.26 |
| 1126 | 3.92 | 0.174 | 4.56 |
| 1127 | 5.95 | 0.167 | 6.16 |
| 1128 | 9.88 | 0.0378 | 0.486 |
| 1129 | 6.11 | 0.048 | 0.501 |
| 1130 | 13.3 | 0.0441 | 0.641 |
| 1131 | 8.42 | 0.0472 | 0.483 |
| 1132 | 2.11 | 0.0335 | 0.681 |
| 1133 | 2.95 | 0.0719 | 0.768 |
| 1134 | 2.57 | 0.0391 | 0.957 |
| 1135 | 1.76 | 0.0126 | 0.746 |
| 1136 | 1.26 | 0.0131 | 0.453 |
| 1137 | 14.2 | 0.0334 | 0.582 |
| 1138 | 1.01 | 0.0491 | 1.93 |
| 1139 | 5.29 | 0.0431 | 1.85 |
| 1140 | 1.56 | 0.00821 | 0.323 |
| 1141 | 1.15 | 0.00921 | 0.412 |
| 1142 | 1.34 | 0.00706 | 0.329 |
| 1143 | 1.42 | 0.0087 | 0.381 |
| 1144 | 1.32 | 0.00649 | 0.282 |
| 1145 | 1.07 | 0.00689 | 0.313 |
| 1146 | 1.22 | 0.00628 | 0.281 |
| 1147 | 1.17 | 0.00753 | 0.347 |
| 1148 | 1.6 | 0.00801 | 0.299 |
| 1149 | 1.37 | 0.00804 | 0.352 |
| 1150 | 1.17 | 0.0069 | 0.278 |
| 1151 | 1.31 | 0.00339 | 0.262 |
| 1152 | 2.32 | 0.0101 | 0.371 |
| 1153 | 2.69 | 0.0109 | 0.33 |
| 1154 | 6.42 | 0.00879 | 0.344 |
| 1155 | 1.83 | 0.00917 | 0.434 |
| 1156 | 2.94 | 0.0127 | 0.444 |
| 1157 | 2.04 | 0.0124 | 0.408 |
| 1158 | 2.19 | 0.0126 | 0.419 |
| 1159 | 1.86 | 0.0118 | 0.45 |
| 1160 | 2.41 | 0.00773 | 0.365 |
| 1161 | 2.19 | 0.00973 | 0.39 |
| 1162 | 2.55 | 0.00818 | 0.372 |
| 1163 | 1.75 | 0.00951 | 0.449 |
| 1164 | 2.83 | 0.0104 | 0.495 |
| 1165 | 2.68 | 0.0121 | 0.632 |
| 1166 | 2.94 | 0.00999 | 0.478 |
| 1167 | 2.16 | 0.011 | 0.636 |
| 1168 | 2.63 | 0.00839 | 0.422 |
| 1169 | 2.43 | 0.011 | 0.613 |
| 1170 | 2.43 | 0.00783 | 0.395 |
| 1171 | 2.19 | 0.00953 | 0.529 |
| 1172 | 2.52 | 0.00886 | 0.44 |
| 1173 | 2.31 | 0.00943 | 0.604 |
| 1174 | 2.58 | 0.00896 | 0.454 |
| 1175 | 2.08 | 0.00962 | 0.572 |
| 1176 | 1.8 | 0.00717 | 0.344 |
| 1177 | 4.23 | 0.0114 | 0.681 |
| 1178 | 3.08 | 0.00913 | 0.437 |
| 1179 | 2.87 | 0.0102 | 0.652 |
| 1180 | 3.16 | 0.00602 | 0.445 |
| 1181 | 3.34 | 0.0115 | 0.664 |
| 1182 | 1.24 | 0.0316 | 0.797 |
| 1183 | 1.38 | 0.0484 | 0.769 |
| 1184 | 1.5 | 0.071 | 1.18 |
| 1185 | 1.64 | 0.0266 | 0.762 |
| 1186 | 1.46 | 0.0404 | 0.594 |
| 1187 | 1.81 | 0.0648 | 1.06 |
| 1188 | 1.16 | 0.0914 | 1.01 |
| 1189 | 1.59 | 0.00597 | 0.292 |
| 1190 | 1.68 | 0.0109 | 0.765 |
| 1191 | 3.06 | 0.00992 | 0.945 |
| 1192 | 1.54 | 0.00924 | 0.499 |
| 1193 | 1.56 | 0.00897 | 0.544 |
| 1194 | 2.02 | 0.00725 | 0.51 |
| 1195 | 1.74 | 0.00708 | 0.367 |
| 1196 | 1.9 | 0.00756 | 0.418 |
| 1197 | 1.88 | 0.00666 | 0.407 |
| 1198 | 1.8 | 0.00663 | 0.475 |
| 1199 | 2.05 | 0.0076 | 0.5 |
| 1200 | 1.5 | 0.00883 | 0.727 |
| 1201 | 1.76 | 0.00959 | 0.888 |
| 1202 | 0.5 | 0.00635 | 0.237 |
| 1203 | 2.58 | 0.0774 | 0.292 |
| 1204 | 3.5 | 0.21 | 0.338 |
| 1205 | 3.33 | 0.152 | 0.523 |
| 1206 | 1.78 | 0.0671 | 0.352 |
| 1207 | 7.71 | 0.00647 | 0.299 |
| 1208 | 3.52 | 0.00907 | 0.526 |
| 1209 | 0.97 | 0.00819 | 0.41 |
| 1210 | 2.25 | 0.00704 | 0.375 |
| 1211 | 1.45 | 0.00592 | 0.276 |
| 1212 | 1.71 | 0.00702 | 0.357 |
| 1213 | 1.69 | 0.00721 | 0.423 |
| 1214 | 1.85 | 0.00851 | 0.55 |
| 1215 | 2.09 | 0.0106 | 0.668 |
| 1216 | 1.83 | 0.00921 | 0.65 |
| 1217 | 1.98 | 0.0101 | 0.751 |
| 1218 | 1.88 | 0.00839 | 0.58 |
| 1219 | 1.64 | 0.0101 | 0.624 |
| 1220 | 1.57 | 0.0099 | 0.674 |
| 1221 | 2.27 | 0.0111 | 0.839 |
| 1222 | 2.55 | 0.0858 | 0.346 |
| 1223 | 2.61 | 0.0916 | 0.337 |
| 1224 | 2.32 | 0.048 | 0.379 |
| 1225 | 2.45 | 0.0531 | 0.399 |
| 1226 | 1.65 | 0.00539 | 0.264 |
| 1227 | 1.99 | 0.00621 | 0.387 |
| 1228 | 1.98 | 0.0067 | 0.379 |
| 1229 | 2.47 | 0.0065 | 0.408 |
| 1230 | 64.7 | 0.022 | 1.19 |
| 1231 | 15.7 | 0.0146 | 1.23 |
| 1232 | 6.69 | 0.00893 | 0.723 |
| 1233 | 9.48 | 0.012 | 1.09 |
| 1234 | 11.6 | 0.0135 | 1.17 |
| 1235 | 455 | 0.241 | 25.3 |
| 1236 | 0.947 | 0.052 | 0.304 |

As seen in Table 3, exemplary analogs stimulate CAMP from human GIP. GLP-1 and glucagon receptors in the presence of 0.1% casein.

### In Vivo Studies

### Pharmacokinetics in Male Sprague Dawley Rats:

The pharmacokinetics of the exemplary analogs are evaluated following a single subcutaneous (SC) administration of 10 nmol/kg (dissolved in 40 mM Tris pH8) or single 4 mg/kg (mixed with 250 mM sodium decanoate/C₁₀ in 40 mM Tris pH8) intrajejunal (IJ) administration to male Sprague Dawley rats. Blood samples are collected over 96 hours following SC administration and 72 hours following IJ dosing, and resulting individual plasma concentrations are used to calculate pharmacokinetic parameters. Peptide plasma (K₃ EDTA) concentrations are determined using a qualified LC/MS method that measured the intact mass of the analog. Each peptide and an analog as an internal standard are extracted from rat plasma using methanol. A High Resolution Instrument was used for LC/MS detection. Mean pharmacokinetic parameters are shown in Tables 4 and 5.

TABLE 4
Mean Pharmacokinetic Parameters of Peptides
Following a Single Subcutaneous Administration
of 10 nmol/kg to Male Sprague Dawley Rats.

| | T_{1/2} (hr) | Cl/F (mL/hr/Kg) |
|---|---|---|
| Example 1 | 16.1 | 12.6 |
| Example 2 | 12.1 | 8.6 |
| Example 289 | 22.4 | 3.87 |
| Example 321 | 26.2 | 8.2 |
| Example 329 | 12.6 | 19.3 |

Abbreviations: T_{1/2} = half-life, CL/F = apparent clearance
NOTE:
Data are the mean, where n = 3/group.

TABLE 5
Mean Pharmacokinetic Parameters of Peptides Following a single
IJ Administration of 4 mg/kg to Male Sprague Dawley Rats.

| | Cmax/D | | |
|---|---|---|---|
| | (nM/mg/kg) | T_{1/2} (hr) | Cl/F (L/hr/Kg) |
| Example 368 | 80 | 9.6 | 0.44 |
| Example 369 | 70 | 8.2 | 0.68 |
| Example 378 | 151 | 8.2 | 0.23 |
| Example 381 | 60 | 8.3 | 0.59 |
| Example 387 | 43 | 9.2 | 0.84 |
| Example 401 | 36 | 6.6 | 0.98 |
| Example 429 | 52 | 7.8 | 0.64 |
| Example 462 | 78 | 11 | 0.6 |
| Example 463 | 35 | 8.3 | 0.97 |
| Example 472 | 90 | 9.4 | 0.44 |
| Example 475 | 105 | 13.7 | 0.21 |

Abbreviations: Cmax/D = dose normalized maximum plasma concentrations; T_{1/2} = half-life, CL/F = apparent clearance.
NOTE:
Data are the mean, where n = 4/group.

Results from this study for Examples tested are consistent with an extended pharmacokinetic profile.

### Pharmacokinetics in Cynomolgus Monkeys

A study is designed to evaluate the oral bioavailability of Example polypeptides in cynomolgus monkeys. High resolution liquid chromatography/mass spectrometry (HR-LC/MS) is used to measure the concentrations of Examples 510, 548, 558, 694, 699, 686 712, 741, 883, and 1027 in cynomolgus monkey plasma. Standards and controls are prepared in cynomolgus monkey plasma, and any dilutions required to bring samples into the quantitative range are also performed in control cynomolgus monkey plasma. To control assay variability, an internal standard (IS) is added to all the standards and samples. The Examples and IS are extracted from 100% monkey plasma (50 μL) by protein precipitation using isopropyl alcohol and methanol (50:50 v/v). The samples are then centrifuged (3000 rpm for 10 minutes) and the supernatant is transferred to a Siricco Protein Precipitation Plate. The samples are loaded on a Sep-Pak tC18 SPE microelution plate that is conditioned with 2% formic acid in acetonitrile and water. The compounds are then washed with 2% formic acid in water and eluted using 2% formic acid in acetonitrile into a plate containing 5× Invitrosol and 1% formic acid in water prior to injecting an aliquot (10 μL) on to Xselect CSH C18, 3.5 μm, 2.1×20 mm for LC/MS analysis.

The plasma pharmacokinetics (PK) of Examples 510, 548, 558, 694, 699, 686 712, 741, 883, and 1027 are evaluated in male and female cynomolgus monkeys following a single intravenous (IV) dose (10 nmol/kg). Blood samples are collected over 504 hours. Plasma is harvested from blood samples by centrifugation and stored frozen (−70° C.) until analysis. Plasma concentrations of the molecules are detected using the bioanalytical method described above.

TABLE 6
Pharmacokinetic Parameters (Mean ± SD) Following a Single IV Dose of
Example polypeptides (10 nmol/kg) to male and female Cynomolgus Monkeys (n = 3).

| | Dose | T_{1/2} | AUC_{0-inf} | CL |
|---|---|---|---|---|
| Compound | (nmol/kg) | (hr) | (hr*nmol/L) | (mL/kg/hr) |
| Example 883 | 10 | 62.8 ± 53.9 | 8690 ± 623 | 1.15 ± 0.083 |
| Example 510 | 10 | 55.9 ± 13.2 | 14500 ± 3420 | 0.716 ± 0.181 |
| Example 548 | 10 | 53.2 ± 0.603 | 10500 ± 1580 | 0.971 ± 0.159 |
| Example 558 | 10 | 107 ± 22.0 | 6230 ± 273 | 1.60 ± 0.071 |
| Example 1027 | 10 | 76.4 ± 31.4 | 8570 ± 1550 | 1.20 ± 0.239 |
| Example 741 | 10 | 32.1 ± 4.22 | 9550 ± 2280 | 1.09 ± 0.24 |
| Example 712 | 10 | 54.0 ± 6.09 | 12400 ± 862 | 0.81 ± 0.06 |
| Example 694 | 10 | 50.7 ± 16.8 | 10700 ± 2750 | 0.98 ± 0.26 |
| Example 699 | 10 | 50.8 ± 13.7 | 9230 ± 1640 | 1.11 ± 0.19 |
| Example 686 | 10 | 41.5 ± 2.35 | 7040 ± 1290 | 1.46 ± 0.29 |

Abbreviations: AUC_{0-inf} = area under the curve from time 0 hours to infinity; CL = clearance; T_{1/2} = half-life.

PK parameters of TG-2474, TG-2728, TG-2565, TG-2698, TG-2708, TG-3329, TG-3270, TG-3169, TG-3211, and TG-3120 are determined after a single 10 mg oral dose to male and female cynomolgus monkeys. Blood samples are collected up to 504 hours post-dose. Plasma is harvested from blood samples by centrifugation and stored frozen (−70° C.) until analysis. Plasma concentrations of the molecules are detected using the bioanalytical method described above.

TABLE 7
Pharmacokinetic Parameters (Mean ± SD) Following a Single Oral Dose of Example
polypeptides (10 mg per animal) with Sodium Salcaprozate ((N-[8-(2-hydroxybenzoyl)
amino] caprylate) (SNAC), 300 mg) to male and female Cynomolgus Monkeys (n = 4).

| | Dose | T_{1/2} | Tₘₐₓ | Cₘₐₓ | AUC_{0-inf} | CL/F | F |
|---|---|---|---|---|---|---|---|
| Compound | (nmol/kg) | (hr) | (hr) | (nmol/L) | (hr*nmol/L) | (mL/kg/hr) | (%) |
| Example | 519 ± | 29.0 ± | 2.0 ± | 43.6 ± | 1280 ± | 666 ± | 0.3 ± |
| 883 | 57 | 13.4 | 1.15 | 22.3 | 705 | 666 | 0.2 |
| Example | 535 ± | 47.0 ± | 2.0 ± | 126 ± | 5090 ± | 310 ± | 0.7 ± |
| 510 | 69 | 25.0 | 1.15 | 87.1 | 4290 | 419 | 0.6 |
| Example | 558 ± | 45.0 ± | 1.5 ± | 75.7 ± | 3580 ± | 162 ± | 0.6 ± |
| 548 | 77 | 11.9 | 1.0 | 6.19 | 817 | 37 | 0.1 |
| Example | 448 ± | 46.2 ± | 2.75 ± | 147 ± | 4990 ± | 124 ± | 1.8 ± |
| 558 | 20 | 8.01 | 2.36 | 86.9 | 2890 | 85 | 1.0 |
| Example | 504 ± | 55.7 ± | 2.5 ± | 135 ± | 5070 ± | 166 ± | 1.2 ± |
| 1027 | 53 | 25.4 | 1.0 | 93.1 | 4080 | 132 | 0.9 |
| Example | 564 ± | 58.1 ± | 3.75 ± | 95.8 ± | 4200 ± | 137 ± | 0.8 ± |
| 741 | 93 | 22.8 | 1.5 | 8.61 | 679 | 21.2 | 0.1 |
| Example | 563 ± | 51.8 | 3.75 ± | 124 ± | 6180 ± | 111 ± | 0.9 ± |
| 712 | 54 | 16.4 | 1.5 | 49.3 | 2670 | 62.9 | 0.4 |
| Example | 572 ± | 50.7 ± | 3.50 ± | 353 ± | 14800 ± | 52.4 ± | 2.4 ± |
| 694 | 59 | 13.3 | 2.89 | 192 | 10600 | 28.1 | 1.7 |
| Example | 575 ± | 34.3 ± | 2.75 ± | 187 ± | 7150 ± | 90.7 ± | 1.3 ± |
| 699 | 82 | 7.8 | 2.36 | 102 | 2890 | 35.6 | 0.5 |
| Example | 554 ± | 44.6 ± | 1.0 ± | 249 ± | 14200 ± | 176 ± | 3.6 ± |
| 686 | 54 | 6.49 | 0.0 | 319 | 22300 | 158 | 5.7 |

Abbreviations: AUC_{0-inf} = area under the curve from time 0 hours to infinity; CL/F = apparent clearance; Cₘₐₓ = maximum concentration; Tₘₐₓ = time to maximal concentration; T_{1/2} = half-life; F = bioavailability.

Percent F results in Table 7 demonstrate relative bioavailability of example polypeptides when administered orally as compared to IV administration.

### Studies in Diet-Induced Obese C₅₇BL/6 Mice:

To investigate the effect of the polypeptides as described herein on weight loss, exemplary polypeptides are dosed to C₅₇BL/6 diet-induced obese (DIO) mice.

Specifically, DIO male C₅₇BL/6 mice (Taconic, Germantown, NY) maintained on a calorie-rich diet are used in the following studies. Mice are individually housed in a temperature-controlled (24° C.) facility with 12-hour light/dark cycle (lights on 22:00) and free access to food (TD95217) and water. After a minimum of 2 weeks acclimation to the facility, the mice are randomized according to their body weight, so each experimental group of animals would have similar starting body weight. The body weights range from 41 to 50 g.

All groups contain 5 mice. Mice are treated with vehicle (40 mM Tris-HCl at pH 8.0), or example polypeptides at 10 nmol/kg. Treatments are administered by subcutaneous (SC) injection (10 mL/kg) to ad libitum fed DIO mice 30 to 90 minutes prior to the onset of the dark cycle either once daily (QD) or once every 3 days (Q3D) for 9-16 days. Body weight and food intake are measured daily throughout the study. Body weight is presented as percent of the starting weight. The vehicle-treated mice (control group) maintain their body weight ranging from 98.00±0.84% to 101.45±2.39% throughout the study.

Data are presented as mean±SEM of 5 animals per group in Table 7 below. Statistical analysis is performed using repeated measures ANOVA, followed by Dunnett's method comparison test.

TABLE 8
% Body Weight After Treatment with Exemplary polypeptides

| | Dosing | Duration | |
|---|---|---|---|
| Example | frequency | (days) | Body weight (%) |
| 2 | Q3D | 14 | 77.90 +/− 1.90**** |
| 4 | Q3D | 14 | 83.00 +/− 0.88**** |
| 21 | Q3D | 14 | 83.70 +/− 0.86**** |
| 23 | Q3D | 14 | 80.86 +/− 1.25**** |
| 24 | Q3D | 14 | 86.10 +/− 1.98**** |
| 29 | Q3D | 14 | 75.96 +/− 3.77**** |
| 30 | Q3D | 14 | 90.12 +/− 0.30** |
| 62 | Q3D | 14 | 88.05 +/− 1.08*** |
| 75 | Q3D | 14 | 96.28 +/− 1.51 |
| 93 | Q3D | 14 | 86.64 +/− 1.94**** |
| 94 | Q3D | 14 | 84.92 +/− 1.79**** |
| 98 | Q3D | 14 | 90.78 +/− 1.26* |
| 121 | Q3D | 14 | 85.58 +/− 3.20**** |
| 125 | Q3D | 14 | 97.90 +/− 0.32 |
| 126 | Q3D | 14 | 95.68 +/− 0.90 |
| 132 | Q3D | 14 | 98.66 +/− 0.47 |
| 134 | Q3D | 14 | 93.28 +/− 1.38 |
| 288 | Q3D | 14 | 86.18 ± 0.67**** |
| 289 | QD | 16 | 60.94 ± 1.87**** |
| 289 | Q3D | 14 | 82.32 ± 1.25**** |
| 290 | QD | 16 | 65.40 ± 3.45**** |
| 295 | QD | 16 | 51.58 ± 3.24**** |
| 296 | QD | 16 | 49.64 ± 1.97**** |
| 297 | QD | 16 | 48.42 ± 2.86**** |
| 315 | Q3D | 14 | 79.76 +/− 1.72**** |
| 321 | Q3D | 14 | 87.14 +/− 0.59*** |
| 334 | Q3D | 14 | 84.78 +/− 2.74**** |
| 339 | Q3D | 14 | 82.36 +/− 3.01**** |
| 368 | QD | 14 | 52.52 +/− 2.49**** |
| 369 | QD | 14 | 52.91 +/− 2.17**** |
| 378 | QD | 15 | 68.84 +/− 1.37**** |
| 401 | QD | 15 | 71.54 ± 3.32**** |
| 429 | QD | 15 | 70.20 ± 1.63**** |
| 429 | QD | 9 | 74.66 ± 3.87**** |
| 452 | QD | 15 | 74.13 ± 4.11**** |
| 462 | QD | 9 | 73.68 ± 2.82**** |
| 463 | QD | 9 | 83.31 ± 2.10**** |
| 472 | QD | 9 | 76.13 ± 2.28**** |
| 475 | QD | 9 | 69.34 ± 2.99**** |
| 490 | QD | 9 | 80.75 ± 3.23**** |
| 686 | QD | 14 | 72.73 ± 3.65**** |
| 694 | QD | 7 | 71.92 ± 1.28**** |
| 694 | QD | 14 | 69.77 ± 2.96**** |
| 699 | QD | 7 | 68.34 ± 0.85**** |
| 699 | QD | 14 | 56.85 ± 2.42**** |
| 700 | QD | 7 | 75.9 ± 3.77**** |
| 712 | QD | 7 | 69.4 ± 0.60**** |
| 712 | QD | 14 | 63.61 ± 3.73**** |
| 741 | QD | 14 | 59.42 ± 2.94**** |

*p < 0.05;
**p < 0.01;
***p < 0.001;
****p < 0.0001 compared to Control group,
One way ANOVA,
Dunnett's multiple comparison test

### GLP1-R Internalization Assay

The potency of peptides to stimulate ligand-induced internalization of the GLP-1R is determined using HEK293 cells expressing the human GLP1-R.

HEK293 cells were seeded in white, 384-well plates the day before transfection at a density of 20,000 cells/well. The cells were transfected with Lipofectamine 2000 (Invitrogen) for SNAP-GLP-1R. The following day, the media was removed and the tagged receptors were labeled with 100 nM Tag-Lite SNAP-Lumi4-Tb (donor, Cisbio), in OptiMEM for 75 minutes at 37° C. Afterward, the cells were washed with internalization buffer (HBBS supplemented with 1 mM CaCl2, 2.5 mM MgCl₂, 20 mM HEPES, and 0.1% Pluronic F-68, pH 7.4) followed by addition of 100 μM preheated fluorescein-O′-acetic acid (acceptor, Sigma-Aldrich). The plate was placed in a 37° C. incubator for 5 minutes prior to ligand addition to adjust the temperature. Then, the cells were stimulated with 37° C. preheated ligand, and internalization of GLP1-R was measured every 3 minutes for 60 minutes at 37° C. by an EnVision plate reader. Data were normalized to maximum concentration of GLP-1 (100%) and no ligand (0%) and plotted using GraphPad Prism 7 software.

The potency of an exemplary polypeptide to stimulate ligand-induced internalization of the GLP-1R is reported in Table 9. Assay results identify whether a polypeptide is a partial agonist on the GLP-1R with respect to GLP-1R intemalization.

TABLE 9

| | GLP1R | |
|---|---|---|
| Compound or | Internalization | Internalization |
| Example Tested | EC₅₀, nM | % TOP |
| GLP1 7-36 amide | 7.17 | 99.4 |
| Semaglutide | 12.4 | 103 |
| Tirzepatide | 60.8 | 60.3 |
| 1 | 44.7 | 69.1 |
| 2 | 49.5 | 66.8 |
| 3 | 49.2 | 69.7 |
| 4 | 49.1 | 67.8 |
| 5 | 53.4 | 75 |
| 7 | 52.6 | 76.3 |
| 8 | 63.2 | 74 |
| 10 | 79.2 | 71.9 |
| 11 | 75.1 | 73.8 |
| 12 | 48.5 | 75.3 |
| 13 | 53.3 | 74.7 |
| 15 | 79 | 74.9 |
| 16 | 37.8 | 74.6 |
| 17 | 66.2 | 72.2 |
| 18 | 91.8 | 74.1 |
| 19 | 52.4 | 72.6 |
| 21 | 87.6 | 72.5 |
| 22 | 41.9 | 64.7 |
| 23 | 51.5 | 67.2 |
| 24 | 79.9 | 71.9 |
| 26 | 96.1 | 75.6 |
| 28 | 81.8 | 76.2 |
| 29 | 129 | 57 |
| 30 | 70.4 | 72.8 |
| 31 | 91.9 | 67.4 |
| 32 | 86.1 | 64.4 |
| 33 | 106 | 64.6 |
| 37 | 51.6 | 68.3 |
| 41 | 83.7 | 62.3 |
| 42 | 61.4 | 58.3 |
| 43 | 58.2 | 68.3 |
| 44 | 51.5 | 65 |
| 45 | 52.6 | 62.1 |
| 46 | 41 | 58.1 |
| 48 | 44 | 55.2 |
| 49 | 37.2 | 53.2 |
| 50 | 30.1 | 31.1 |
| 51 | 89.2 | 55.7 |
| 55 | 79.5 | 70.7 |
| 57 | 76.6 | 73.1 |
| 58 | 56.2 | 78.1 |
| 62 | 40.3 | 44.9 |
| 66 | 42.1 | 46.3 |
| 67 | 50.9 | 31.1 |
| 68 | 95.2 | 33.8 |
| 69 | 71.9 | 58.2 |
| 70 | 60 | 61.3 |
| 71 | 58.8 | 68.6 |
| 74 | 72.7 | 53.3 |
| 75 | 61.8 | 51.9 |
| 78 | 78.2 | 55 |
| 79 | 56.6 | 45.3 |
| 80 | 35.8 | 48.2 |
| 85 | 326 | 48.3 |
| 86 | 57.8 | 52.5 |
| 87 | 61.8 | 45.4 |
| 90 | 70.8 | 49.8 |
| 93 | 30.4 | 50.8 |
| 94 | 37.1 | 47.2 |
| 95 | 37.8 | 49.3 |
| 97 | 51.9 | 50.7 |
| 98 | 56 | 49.3 |
| 99 | 56.9 | 46.1 |
| 107 | 150 | 45.1 |
| 108 | 67.7 | 41.1 |
| 109 | 28.8 | 39 |
| 110 | 28.3 | 42.5 |
| 111 | 49.1 | 46.4 |
| 112 | 30.1 | 59.1 |
| 113 | 39.5 | 64.6 |
| 114 | 47.2 | 62.9 |
| 115 | 255 | 56.5 |
| 116 | 80.7 | 46.2 |
| 117 | 45.5 | 51 |
| 118 | 82.3 | 46.4 |
| 119 | 73.5 | 43.4 |
| 121 | 38.1 | 46.3 |
| 122 | 57.4 | 43.7 |
| 125 | 86.4 | 42.6 |
| 126 | 63.2 | 38.8 |
| 129 | 22.5 | 40.6 |
| 132 | 72.7 | 67.2 |
| 134 | 67.9 | 22.3 |
| 136 | 36.1 | 37.8 |
| 137 | 60.2 | 47.7 |
| 139 | 62.8 | 39.9 |
| 141 | 95.3 | 64.1 |
| 143 | 337 | 43 |
| 148 | 95.5 | 37.4 |
| 151 | 77.8 | 55 |
| 154 | 47.7 | 65.9 |
| 155 | 85.1 | 54.5 |
| 157 | 60.3 | 53.3 |
| 158 | 104 | 52.4 |
| 162 | 48.8 | 44.8 |
| 163 | 143 | 37.8 |
| 164 | 45.9 | 46.5 |
| 165 | 57.5 | 44.5 |
| 166 | 74.6 | 44.2 |
| 167 | 75.9 | 44.7 |
| 168 | 74.8 | 38.7 |
| 169 | 178 | 49.4 |
| 171 | 61.4 | 36.1 |
| 172 | 121 | 41.4 |
| 173 | 97.1 | 38.6 |
| 174 | 123 | 52.8 |
| 175 | 116 | 49.2 |
| 176 | 103 | 48 |
| 177 | 79.4 | 44 |
| 179 | 63.4 | 48.4 |
| 181 | 116 | 47.5 |
| 183 | 100 | 45.4 |
| 185 | 144 | 43.7 |
| 186 | 124 | 41.1 |
| 187 | 17.2 | 57 |
| 188 | 44.7 | 40.2 |
| 189 | 103 | 45.8 |
| 192 | 115 | 48.7 |
| 193 | 132 | 50.7 |
| 200 | 86.1 | 39.2 |
| 204 | 66.4 | 45.5 |
| 205 | 49.4 | 67.7 |
| 208 | 55.7 | 49.6 |
| 209 | 83.1 | 41.2 |
| 210 | 80.2 | 49.2 |
| 211 | 57.4 | 26.9 |
| 212 | 79.7 | 43 |
| 213 | 85.9 | 45.2 |
| 214 | 50.1 | 41.7 |
| 217 | 27.6 | 58.2 |
| 218 | 80.5 | 35 |
| 219 | 48 | 37.2 |
| 222 | 34.6 | 52.4 |
| 223 | 45.8 | 59.3 |
| 224 | 32.2 | 61.9 |
| 225 | 26 | 56.7 |
| 226 | 29.1 | 59.3 |
| 227 | 114 | 59.4 |
| 228 | 39 | 35 |
| 229 | 42.4 | 31.4 |
| 230 | 34.4 | 31.2 |
| 231 | 36.1 | 27.7 |
| 232 | 44.7 | 45.5 |
| 233 | 24.8 | 41.8 |
| 238 | 32.1 | 38.2 |
| 239 | 72.6 | 42.9 |
| 241 | 64.4 | 37.6 |
| 242 | 56 | 38.2 |
| 248 | 67.4 | 39.5 |
| 249 | 86.2 | 34.8 |
| 250 | 97 | 45 |
| 253 | 81.6 | 55.2 |
| 254 | 31.9 | 47.8 |
| 255 | 47.1 | 51.8 |
| 256 | 87.8 | 43.5 |
| 276 | 237 | 68.4 |
| 278 | 251 | 68.6 |
| 279 | 115 | 37.5 |
| 280 | 39.4 | 74.9 |
| 282 | 28.7 | 71.9 |
| 285 | 59.9 | 73.6 |
| 288 | 30.3 | 72.4 |
| 289 | 10.9 | 86.5 |
| 295 | 31.4 | 88.4 |
| 296 | 19.2 | 87.2 |
| 321 | 27.1 | 92.8 |
| 323 | 28.2 | 84.8 |
| 329 | 34 | 91.1 |
| 334 | 28.3 | 96.8 |
| 358 | 21.5 | 83.1 |
| 359 | 49.6 | 74.4 |
| 360 | 214 | 66.1 |
| 361 | 50.4 | 73 |
| 362 | 22.8 | 77.5 |
| 510 | 11.8 | 113 |
| 548 | 17.8 | 109 |
| 558 | 8.14 | 116 |
| 686 | 33.2 | 101 |
| 694 | 21.8 | 97.3 |
| 699 | 14.9 | 102 |
| 712 | 35.3 | 109 |
| 741 | 50.1 | 103 |
| 883 | 23.9 | 77.1 |
| 1026 | 47.4 | 80 |

### GLP-1R CHO Cell β-Arrestin Recruitment Assay

Activated G-protein coupled receptors can interact with the β-arrestin family of signaling proteins. The potency of peptides for GLP-1R induced arrestin recruitment is determined using the PathHunter Enzyme Fragment Complementation approach substantially as described (von Degenfeld et al., FASEB J., 2007 (14):3819-26 and Hamdouchi et al., J. Med Chem., 2016 59(24): 10891-10916).

CHO-KI cells expressing Pro-Link-tagged Human GLP-1R and enzyme-acceptor-tagged β-arrestin-2 may be obtained from DiscoveRx and prepared as assay-ready frozen cells. Test peptides are solubilized in DMSO and serial dilutions are performed using the Echo acoustic dispenser (LabCyte). Assay media is the PathHunter Cell Assay Buffer (DiscoveRx) containing 0.1% w/v hydrolyzed Casein (Sigma). 100 nl of peptide is dispensed into 10 μl of assay media in a 384 well plate and then 10 μl of cells in assay media are added to give 5000 cells per well. Plates are incubated for 90 minutes in a 37′C/5% C0₂ incubator and 10 μl of PathHunter detection reagent is added (DiscoveRx) and plates are incubated at room temperature for 60 minutes. Luminescence signal is measured. Peptide concentration-response curves fit to a four-parameter logistic model to calculate potency as an EC₅₀. Data normalization to % stimulation is performed using DMSO and GLP-1(7-36) as minimum and maximum controls (Campbell et al., Assay Guidance Manual 2017).

The potency of a sample peptide to stimulate GLP-1R induced ß-arrestin recruitment is reported in Table 10. The assay results identify whether a peptide is a partial and biased agonist on the GLP-1R with respect to ß-arrestin-2 recruitment.

TABLE 10

| Compound or | hGLP1R | |
|---|---|---|
| Example | β-arrestin Rel | % Stimulation |
| Tested | EC₅₀, μM | Max |
| hGLP-1 7- | 0.00223 | 102 |
| 36 amide | | |
| Semaglutide | 0.00468 | 99.6 |
| Tirzepatide | >10 | 10.5 |
| 1 | >10 | 17.3 |
| 2 | >10 | 12.3 |
| 3 | >10 | 16.5 |
| 4 | 0.0232 | 15.8 |
| 5 | >10 | 18.4 |
| 288 | 0.0163 | 18 |
| 289 | 0.00596 | 47.5 |
| 294 | 0.0277 | 35.3 |
| 293 | 0.0119 | 54.6 |
| 290 | 0.00394 | 83.5 |
| 295 | 0.0164 | 44.1 |
| 296 | 0.014 | 55.2 |
| 297 | 0.0602 | 33.5 |
| 298 | 0.00329 | 86.4 |
| 300 | 0.0157 | 40.3 |
| 302 | 0.0112 | 56.6 |
| 304 | 0.00921 | 47.8 |
| 301 | 0.0132 | 59.1 |
| 307 | 0.00986 | 70.8 |
| 305 | 0.0233 | 58.3 |
| 311 | 0.0192 | 39.6 |
| 308 | 0.0169 | 41.4 |
| 309 | 0.00672 | 47.2 |
| 315 | 0.00376 | 29.7 |
| 321 | 0.00878 | 90.1 |
| 323 | 0.0108 | 44 |
| 329 | 0.00611 | 74.5 |

### Cell Based In Vitro Permeability (Papp) Assay

Intestinal organoid from Göttingen minipig jejunum tissue and its 2D monolayer are generated using the protocol previously described (van der Hee, B.; Loonen, L. M. P.;

Taverne, N.; Taverne-Thiele, J. J.; Smidt, H.; Wells, J. M., Optimized procedures for generating an enhanced, near physiological 2D culture system from porcine intestinal organoids. Stem Cell Res 2018, 28, 165-171). After 7 days of culture, organoids are dissociated into single cells by TrypLE (GIbco), and the single cell suspension is added in 24-well transparent transwell inserts (0.3 cm², Falcon, BD). When the transepithelial electrical resistance reached >600 52·cm², the peptide permeability is examined in the presence of 10 or 20 mM C10. Samples from the basal compartment are collected at 20 min in the presence of C10. Peptide concentrations in the apical and basal compartments are measured by LC/MS, and the apparent permeability coefficient (Papp) is calculated as previously described (Twarog, C.; Liu, K.; O'Brien, P. J.; Dawson, K. A.; Fattal, E.; Illel, B.; Brayden, D. J., A head-to-head Caco-2 assay comparison of the mechanisms of action of the intestinal permeation enhancers: SNAC and sodium caprate (C10). Eur J Pharm Biopharm 2020, 152, 95-107).

The Papp values for exemplary polypeptides are reported in Table 11.

TABLE 11

| Compound or | Relative Papp (Compared to internal standard) | |
|---|---|---|
| Example Tested | with 10 mM C10 | with 20 mM C10 |
| Semaglutide | 0.35 | |
| Amycretin | 0.18 | 0.22 |
| Example 406 | 0.90 | 0.59 |
| Example 475 | 0.79 | 0.73 |
| Example 62 | | 0.03 |
| Example 94 | | 0.02 |
| Example 119 | 0.15 | |
| Example 121 | 0.08 | |
| Example 130 | | 0.06 |
| Example 134 | 0.07 | |
| Example 135 | 0.04 | |
| Example 137 | 0.04 | |
| Example 361 | 0.10 | |
| Example 154 | 0.12 | |
| Example 155 | 0.07 | |
| Example 165 | 0.09 | |
| Example 169 | 0.12 | |
| Example 174 | 0.12 | |
| Example 182 | 0.12 | |
| Example 184 | 0.09 | |
| Example 186 | 0.10 | |
| Example 187 | 0.48 | |
| Example 188 | 0.46 | |
| Example 189 | 0.17 | |
| Example 193 | 0.17 | |
| Example 198 | 0.12 | |
| Example 204 | 0.16 | |
| Example 208 | 0.29 | |
| Example 209 | 0.08 | |
| Example 210 | 0.06 | |
| Example 211 | 0.06 | |
| Example 212 | 0.22 | |
| Example 213 | 0.26 | |
| Example 214 | 0.07 | |
| Example 223 | 0.29 | |
| Example 224 | 0.35 | |
| Example 225 | 0.48 | |
| Example 226 | 0.36 | |
| Example 227 | 0.42 | |
| Example 231 | 0.06 | |
| Example 232 | 0.13 | |
| Example 233 | 0.21 | |
| Example 238 | 0.06 | |
| Example 241 | 0.10 | |
| Example 242 | 0.07 | |
| Example 249 | 0.04 | |
| Example 250 | 0.11 | |
| Example 255 | 0.23 | |
| Example 784 | | 0.16 |
| Example 808 | | 0.29 |
| Example 552 | | 0.53 |
| Example 1033 | | 0.14 |
| Example 595 | | 0.55 |
| Example 666 | | 0.85 |
| Example 670 | | 0.46 |
| Example 673 | | 0.59 |
| Example 675 | | 0.36 |
| Example 686 | | 0.60 |
| Example 694 | | 0.60 |
| Example 696 | | 0.77 |
| Example 699 | | 0.51 |
| Example 700 | | 0.29 |
| Example 710 | | 0.35 |
| Example 712 | | 0.60 |
| Example 737 | | 0.69 |
| Example 741 | | 0.73 |
| Example 743 | | 0.56 |
| Example 761 | | 0.53 |

### In Vivo Ileum (Distal Loop) Absorption Assay

Ileum absorption of the exemplary polypeptides in the presence of C₁₀ in rats is evaluated using a previously reported intestinal closed loop model (Lawrence, S. A.; Blankenship, R.; Brown, R.; Estwick, S.; Ellis, B.; Thangaraju, A.; Datta-Mannan, A., Influence of FcRn binding properties on the gastrointestinal absorption and exposure profile of Fc molecules. Bioorg Med Chem 2021, 32, 115942).

Overnight fasted Sprague Dawley rats with body weights between 250-280 grams are anesthetized by inhaling isoflurane and placed on a warm surgical table maintained by circulating water at 37ºC. The surgical area is shaved, and the skin is disinfected with betadine scrub followed by 70% isopropyl alcohol. An approximately 2 cm ventral midline incision is made to expose intestine, and 10 cm of an ileum segment is tied off. The peptides are formulated with 100 mM C₁₀ in Tris buffer (pH 8.0, 50 mM) at a final concentration of 300 μM. The formulations are administered directly into the ileum loop. Blood samples (0.2 mL) are collected from tail vein before peptide dosing and 10, 20, 40, 60 minutes post dosing. Blood samples are collected into tubes containing KsEDTA (5%) and processed to plasma for subsequent analyses. Peptide concentrations in the plasma are determined by LC/MS.

TABLE 12

| | | 10 min IL | 20 min IL | 40 min IL | 60 min IL |
|---|---|---|---|---|---|
| | 0-1 h IL | plasma | plasma | plasma | plasma |
| Example | AUC, | concentration, | concentration, | concentration, | concentration, |
| No. | nM*hr | nM | nM | nM | nM |
| 62 | 9078 | 116 | 169 | 200 | 197 |
| 93 | 11118 | 143 | 188 | 242 | 274 |
| 94 | 9441 | 106 | 164 | 212 | 220 |
| 121 | 23626 | 247 | 359 | 498 | 704 |
| 125 | 6772 | 102 | 125 | 148 | 143 |
| 126 | 14074 | 162 | 256 | 299 | 344 |
| 129 | 11525 | 151 | 191 | 262 | 267 |
| 130 | 5339 | 67 | 93 | 116 | 128 |
| 132 | 17358 | 214 | 270 | 410 | 403 |
| 134 | 15907 | 207 | 287 | 355 | 346 |
| 151 | 5314 | 69 | 95 | 115 | 124 |
| 183 | 21763 | 241 | 372 | 471 | 556 |
| 204 | 24673 | 235 | 399 | 551 | 650 |
| 213 | 16102 | 175 | 258 | 362 | 412 |
| 224 | 15934 | 237 | 311 | 331 | 346 |
| 226 | 26272 | 333 | 454 | 618 | 545 |
| 227 | 35445 | 513 | 656 | 779 | 746 |
| 232 | 12128 | 186 | 235 | 255 | 256 |

### Proteolytic Stability

A pepsin stability assay is utilized to determine the relative stability of Example polypeptides in a simulated gastric proteolytic environment. Pepsin A is dissolved in simulated gastric fluid (SGF) with Example polypeptides or comparators and the solution is sampled and quenched at TO, 15, 30, and 60 minutes. Comparators are semaglutide and example compound number 4 from US Patent Application 2020/0024322 (“Cmpd 4”). The relative amount of intact peptide is quantified by mass spectrometry (MS).

Proteolytic stability of example polypeptides relative to comparators was performed in simulated gastric fluid (SGF 2 g/L NaCl, pH 1.2). The examples were dissolved in 50 mM Tris buffer pH 8.0 at a concentration of 10 mg/mL as a stock solution. Pepsin A was reconstituted in SGF at 10 mg/mL. A reaction was prepared by diluting peptide in SGF and spiking in the Pepsin A to have final concentration of example polypeptide at 0.4 mg/mL and 1 mg/mL of pepsin. The example peptide and pepsin solutions were then incubated at 37° C. in a shaking incubator set at 100 rpm. Samples (25 μL) were withdrawn at different time intervals and quenched with 100 mM ammonium bicarbonate pH 9 (50 μL) to stop proteolytic activity. All samples were centrifuged, and the supernatants were analyzed by LC/MS to determine the remaining intact peptide.

Results are provided in Tables 12-14 below:

TABLE 12
Stability in 0.1 mg/mL pepsin A solution.

| | % Intact (SD) | | | |
|---|---|---|---|---|
| Test Article | 0 min | 15 min | 30 min | 60 min |
| Cmpd 4 | 100 (0) | 101.9 (8.7) | 96.0 (12.1) | 107.9 (25.5) |
| Example 677 | 100 (0) | 90.8 (9.6) | 91.3 (3.0) | 102.7 (11.1) |
| Example 685 | 100 (0) | 85.1 (7.6) | 95.2 (11.6) | 94.6 (6.7) |
| Example 686 | 100 (0) | 99.2 (18.5) | 103.8 (26.2) | 103.8 (24.5) |
| Example 688 | 100 (0) | 97.6 (10.0) | 95.6 (6.1) | 89.9 (8.8) |
| Example 694 | 100 (0) | 100.9 (8.6) | 101.1 (8.0) | 113.4 (6.4) |
| Example 696 | 100 (0) | 101.9 (9.3) | 106.6 (5.8) | 116.9 (2.2) |
| Example 699 | 100 (0) | 121.4 (0.8) | 115.6 (7.5) | 111.9 (5.3) |
| Example 711 | 100 (0) | 95.0 (12.3) | 91.6 (18.0) | 91.9 (13.8) |
| Example 712 | 100 (0) | 92.4 (7.0) | 91.4 (1.3) | 93.5 (6.2) |
| Example 714 | 100 (0) | 99.2 (10.8) | 102.5 (10.2) | 96.9 (5.5) |
| Example 718 | 100 (0) | 102.7 (3.9) | 101.2(11.1) | 112.5(16.9) |
| Example 733 | 100 (0) | 108.8(10.6) | 106.6 (6.9) | 129.5(12.8) |
| Example 741 | 100 (0) | 132.4(49.9) | 103.9 (6.6) | 105.2 (9.3) |
| Example 742 | 100 (0) | 100.5(10.1) | 112.6 (17.9) | 103.3(13.8) |
| Example 700 | 100 (0) | 89.9 (17.8) | 105.5 (7.6) | 98.9 (20.3) |

TABLE 13
Stability in 1 mg/mL pepsin A solution.

| | % Intact (SD) | | | |
|---|---|---|---|---|
| Test Article | 0 min | 15 min | 30 min | 60 min |
| Cmpd 4 | 100 (0) | 78.2 (8.7) | 50.9 (1.6) | 26.2 (0.8) |
| Example 677 | 100 (0) | 58.3 (6.9) | 39.2 (4.7) | 15.5 (2.0) |
| Example 685 | 100 (0) | 59.9 (7.2) | 39.7 (1.6) | 15.0 (0.8) |
| Example 686 | 100 (0) | 66.27 (3.7) | 37.3 (4.4) | 18.1 (6.7) |
| Example 688 | 100 (0) | 67.9 (3.5) | 42.4 (1.6) | 17.9 (1.3) |
| Example 694 | 100 (0) | 87.9 (4.1) | 77.8 (1.5) | 76.9 (3.1) |
| Example 696 | 100 (0) | 91.0 (4.5) | 91.1 (2.5) | 77.6 (5.5) |
| Example 699 | 100 (0) | 97.9 (4.2) | 91.8 (6.2) | 83.9 (6.1) |
| Example 711 | 100 (0) | 98.8 (6.7) | 97.2 (6.2) | 89.7 (8.6) |
| Example 712 | 100 (0) | 104.2 (6.2) | 103.7 (7.1) | 91.6 (5.2) |
| Example 714 | 100 (0) | 106.5 (18.8) | 111.9(8.1) | 79.7 (3) |
| Example 718 | 100 (0) | 108.6 (5.0) | 95.9 (2.2) | 80.6 (3.9) |
| Example 733 | 100 (0) | 97.3 (20.1) | 118.9 (17.8) | 109.5 (23.3) |
| Example 741 | 100 (0) | 111.3 (9.4) | 100.0(18.1) | 113.7 (14.7) |
| Example 742 | 100 (0) | 87.5 (5.9) | 98.0 (10.1) | 101.7 (10.7) |
| Example 700 | 100 (0) | 95.8 (13.0) | 102.5 (30.0) | 115.2 (24.7) |

TABLE 14
Stability in 1 mg/mL pepsin A solution.

| | % Intact (SD) | | | |
|---|---|---|---|---|
| Test Article | 0 min | 15 min | 30 min | 60 min |
| Cmpd 4 | 100 (0) | 61.7 (5.2) | 70.2 (1.8) | 55.9 (7.4) |
| Semaglutide | 100 (0) | 0 (0) | 0 (0) | 0 (0) |
| Example 403 | 100 (0) | 155.4 (70.4) | 143.3 (48.0) | 365.6 (353.2) |
| Example 457 | 100 (0) | 78.8 (6.0) | 68.9 (48.0) | 60.1 (3.5) |
| Example 499 | 100 (0) | 117.4 (8.8) | 118.1 (3.8) | 115.0(8.5) |
| Example 500 | 100 (0) | 10.6 (1.2) | 1.0 (0.1) | 0 (0) |
| Example 505 | 100 (0) | 91.6 (1.2) | 78.9 (1.4) | 53.5 (2.2) |
| Example 1235 | 100 (0) | 1.4 (0.1) | 0 (0) | 0 (0) |

The data in Tables 12-14 support improvements in stability for certain Example polypeptides.

### Sequences

SEQ ID NO: 1 - Human GIP amide
YAEGTFISDYSIAMDKIHQQDFVNWLLAQKGKKNDWKHNITQ-NH₂
SEQ ID NO: 2 - Human GLP-1 (7-36) amide
HAEGTFTSDVSSYLEGQAAKEFIAWLVKGR-NH₂
SEQ ID NO: 3 - Human glucagon
HSQGTFTSDYSKYLDSRRAQDFVQWLMNT

SEO ID NO:4—Polypeptide of Formula I
SEQ ID NO:5—Polypeptide of Formula II
SEQ ID NO:6—Polypeptide of Formula III
SEQ ID NO:7-1242—Polypeptides of Examples 1-1236
SEQ ID NO: 1243—Polypeptide of Formula I′
SEQ ID NO: 1244—Polypeptide of Formula II′
SEQ ID NO: 1245—Polypeptide of Formula III′

The invention claimed is:

1. A polypeptide comprising:
X₁X₂QX₄LTX₆TSDX₁₀X₁₁X₁₂X₁₃LX₁₅X₁₆X₁₇AX₁₉X₂₀X₂₁FX₂₃
X₂₄X₂₅LX₂₇X₂₈X₂₉GX₃₁X₃₂SX₃₄X₃₅PX₃₇PX₃₉X₄₀X₄₁X₄₂X₄₃
X₄₄X₄₅X₄₆,
wherein
X₁ is Y, NMeY or H,
X₂ is Aib,
X₄ is G or D-Ala,
X₆ is F, αMeF or αMeF(2F),
X₁₀ is F, 3-Pal, 4-Pal, F(4CN), F(4NO₂) or Y,
X₁₁ is S or αMeS,
X₁₂ is Orn, K, R, Q, Dap, S, E or I,
X₁₃ is αMeL, I or L,
X₁₅ is D or E,
X₁₆ is K, Orn, A or E,
X₁₇ is any amino acid with a functional group available for conjugation to a fatty acid, A, I or Q or Orn,
X₁₉ is A or Q,
X₂₀ is any amino acid with a functional group available for conjugation to a fatty acid, Aib, Q, R or αMe-4-Pal,
X₂₁ is A, Aad, Aib, S, N, Q, E, T or Orn,
X₂₃ is I or V,
X₂₄ is any amino acid with a functional group available for conjugation to a fatty acid E, D-Glu, Q, N or D-Gln,
X₂₅ is W, Y, F, 4-Pal, αMeY or αMe-4-Pal,
X₂₇ is L, I, E, V, A, Aad, T, Q or S,
X₂₈ is any amino acid with a functional group available for conjugation to a fatty acid, E or A,
X₂₉ is G, Aib, T, D-Ala or A,
X₃₁ is P or E,
X₃₂ is S or P
X₃₄ is G or Aib,
X₃₅ is A, D or E,
X₃₇ is P or E,
X₃₉ is E, S, G, A, T or Orn,
X₄₀ is absent or G, E, S, A or T,
wherein if X₄₀ is G, E, S, A, T or D-Glu, then X₄₁ is absent or is E, S, D, G, Q, T, A, γE or D-Glu,
wherein if X₄₁ is E, S, D, G, Q, T, A, γE or D-Glu, then X₄₂ is absent or G, E, D-Glu, or γE,
wherein if X₄₂ is G, E, D-Glu or γE, then X₄₃ is absent or E, γE, or D-Glu,
wherein if X₄₃ is E, γE, or D-Glu, then X₄₄ is absent or E,
wherein if X₄₄ is E then X₄₅ is absent or E,
wherein if X₄₅ is E then X₄₆ is absent or E,
wherein if X₄₀ is absent, then X₄₁ through X₄₆ are also absent,
wherein if X₄₁ is absent, then X₄₂, through X₄₆ are also absent,
wherein if X₄₂ is absent, then X₄₃ through X₄₆ are also absent,
wherein if X₄₃ is absent, then X₄₄ through X₄₆ are also absent,
wherein if X₄₄ is absent, then X₄₅ and X₄₆ are also absent,
wherein if X₄₅ is absent then X₄₆ is also absent,
wherein the polypeptide comprises at least one of the following: X₆ is αMeF or αMeF(2F); X₁₀ is F, 3-Pal, 4-Pal, F(4CN) or F(4NO₂); X₁₁ is αMeS; X₁₃ is αMeL; X₂₄ is D-Glu; and/or X₂₅ is αMeY;
wherein if X₁₀ is F, 3-Pal, 4-Pal, F(4CN), or F(4NO₂), then X₁₂ is I,
and wherein at least one of X₁₇, X₂₀, X₂₄ or X₂₈ is an amino acid with a functional group available for conjugation to a fatty acid,
wherein the C-terminal amino acid is optionally amidated; or a pharmaceutically acceptable salt thereof.

2. The polypeptide of claim 1, or a pharmaceutically acceptable salt thereof, wherein the polypeptide comprises at least two of the following: X₁₀ is F, 3-Pal, 4-Pal, F(4CN) or F(4NO₂); X₁₁ is αMeS; X₁₃ is αMeL; X₁₆ is Orn; X₂₄ is D-Glu; and/or X₂₅ is αMeY.

3. The polypeptide of claim 1, or a pharmaceutically acceptable salt thereof, wherein the polypeptide comprises at least three of the following: X₁₀ is F, 3-Pal, 4-Pal, F(4CN) or F(4NO₂); X₁₁ is αMeS; X₁₃ is αMeL; X₁₆ is Orn; X₂₄ is D-Glu; and/or X₂₅ is αMeY.

4. The polypeptide of claim 1, or a pharmaceutically acceptable salt thereof, wherein: X₁₀ is F, 3-Pal, 4-Pal, F(4CN) or F(4NO₂).

5. The polypeptide of claim 1, or a pharmaceutically acceptable salt thereof, wherein: X₁ is Y; X₄ is G; X₆ is αMeF(2F); X₁₀ is 4-Pal; X₁₂ is I; X₁₃ is αMeL; X₁₅ is D; X₁₆ is Om; X₁₉ is Q; X₂₀ is αMe-4-Pal; X₂₁ is E or Orn; X₂₃ is I; X₂₄ is D-Glu; X₂₅ is αMeY; X₂₇ is I or V; X₂₈ is E; X₂₉ is G; X₃₁ is P; X₃₄ is G; X₃₅ is A or E; X₃₇ is P; X₃₉ is E or S; X₄₀ is G or T; X₄₁ is E, S, or G; and X₄₂ is absent.

6. The polypeptide of claim 1, or a pharmaceutically acceptable salt thereof, wherein: X₁₁ is S, X₂₁ is Orn, X₂₇ is I, X₃₅ is E, X₃₉ is E, X₄₀ is T and X₄₁ is E.

7. The polypeptide of claim 1, or a pharmaceutically acceptable salt thereof, wherein: X₁₁ is αMeS, X₂₁ is E, X₂₇ is V, X₃₅ is A, X₃₉ is S, X₄₀ is G and X₄₁ is S.

8. The polypeptide of claim 1, or a pharmaceutically acceptable salt thereof, wherein: X₁₁ is αMeS, X₂₁ is E, X₂₇ is I, X₃₅ is A, X₃₉ is S, X₄₀ is G and X₄₁ is S.

9. The polypeptide of claim 1, or a pharmaceutically acceptable salt thereof, wherein: X₁₁ is αMeS, X₂₁ is E, X₂₇ is I, X₃₅ is A, X₃₉ is S, X₄₀ is G and X₄₁ is G.

10. The polypeptide of claim 1, or a pharmaceutically acceptable salt thereof, wherein: X₁₇ is K and is conjugated to a C₁₆-C₂₂ fatty acid via a linker between the amino acid and the C₁₆-C₂₂ fatty acid.

11. The polypeptide of claim 10, or a pharmaceutically acceptable salt thereof, wherein the linker comprises one to four amino acids.

12. The polypeptide of claim 11, or a pharmaceutically acceptable salt thereof, wherein the amino acids comprised in the linker are Glu, γGlu or a combination thereof.

13. The polypeptide of claim 12, or a pharmaceutically acceptable salt thereof, wherein the linker comprises one to four (2-[2-(2-amino-ethoxy)-ethoxy]-acetyl) moieties.

14. The polypeptide of claim 13, or a pharmaceutically acceptable salt thereof, wherein the linker comprises a structure of (γGlu)ₐ-(2-[2-(2-amino-ethoxy)-ethoxy]-acetyl)_{b}-(γGlu)_{c}-CO—(CH₂)ₚ—CO₂H, wherein a is 0 or 1, bis 0, 1 or 2, c is 1, 2 or 3, and p is an integer between 14 to 20.

15. The polypeptide of claim 14, or a pharmaceutically acceptable salt thereof, wherein a is 0, b is 1 and c is 1.

16. A polypeptide selected from the group consisting of SEQ ID NO's: 294-775, 1146-1240, or a pharmaceutically acceptable salt thereof.

17. A polypeptide comprising a sequence identity of more than 90% to the polypeptide of any of SEQ ID NO's: 692, 700, 702, 705, 706, 716, 718, 743, 747, 749, 767.

18. The polypeptide of claim 17, or a pharmaceutically acceptable salt thereof, selected from the group consisting of: SEQ ID NO's: 692, 700, 705, 718 and 747.

19. The polypeptide of claim 18 consisting of SEQ ID NO. 692, or a pharmaceutically acceptable salt thereof.

20. The polypeptide of claim 18 consisting of SEQ ID NO. 700, or a pharmaceutically acceptable salt thereof.

21. The polypeptide of claim 18 consisting of SEQ ID NO. 705, or a pharmaceutically acceptable salt thereof.

22. The polypeptide of claim 18 consisting of SEQ ID NO. 718, or a pharmaceutically acceptable salt thereof.

23. The polypeptide of claim 18 consisting of SEQ ID NO. 747, or a pharmaceutically acceptable salt thereof.

24. The polypeptide of claim 1, or a pharmaceutically acceptable salt thereof, wherein the C-terminal is amidated.

25. The polypeptide of claim 1, or a pharmaceutically acceptable salt thereof, wherein the pharmaceutically acceptable salt is selected from sodium, potassium, trifluoroacetate, hydrochloride or acetate.

26. The polypeptide of claim 1, or a pharmaceutically acceptable salt thereof, wherein one or more hydrogen atoms are replaced by deuterium.

27. The polypeptide of claim 1, or a pharmaceutically acceptable salt thereof, wherein the polypeptide has greater potency at each of the glucagon, GIP and GLP-1 receptors as compared to native glucagon (SEQ ID NO:3), GIP (SEQ ID NO:1) and GLP-17-36 (SEQ ID NO:2).

28. A pharmaceutical composition comprising the polypeptide or a pharmaceutically acceptable salt thereof, of claim 1 and at least one pharmaceutically acceptable carrier, diluent, or excipient.

29. The pharmaceutical composition of claim 28, wherein the composition is formulated for oral administration.

30. The pharmaceutical composition of claim 28, wherein the composition is formulated for subcutaneous administration.

31. A method of treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA) polycystic ovary syndrome (PCOS), Parkinson's disease and Alzheimer's disease, the method comprising a step of: administering to an individual in need thereof an effective amount of a polypeptide, or a pharmaceutically acceptable salt thereof, of any one of claims 1 to 23.
